(19) United States
(12) Patent Application Publication
(10) Pub. No.: US 2011/0065694 A1
(43) Pub. Date: Mar. 17, 2011

(54) Histamine H3 Inverse Agonists and Antagonists and Methods of Use Thereof

(76) Inventors: Milan Chytil, Clinton, MA (US); Sharon R. Engel, Hudson, MA (US); Qun Kevin Fang, Wellesley, MA (US)

(21) Appl. No.: 12/878,887

(22) Filed: Sep. 9, 2010

Related U.S. Application Data
(60) Provisional application No. 61/241,840, filed on Sep. 11, 2009.

(51) Int. Cl. A61K 31/551; C07D 487/18; A61K 31/4188; A61K 31/437; A61K 31/5377; A61K 31/497; C07D 487/14; A61P 25/00; A61P 25/28; A61P 3/00; A61P 3/04; A61P 3/10

(52) U.S. Cl. 514/219; 540/477; 514/393; 514/300; 514/233.2; 514/255.05; 540/555; 540/471

(57) ABSTRACT

Provided herein are fused imidazolyl compounds, methods of synthesis, and methods of use thereof. The compounds provided herein are useful for the treatment, prevention, and/or management of various disorders, including, e.g., neurological disorders and metabolic disorders. Compounds provided herein inhibit the activity of histamine H3 receptors and modulate the release of various neurotransmitters, such as, e.g., histamine, acetylcholine, norepinephrine, and dopamine (e.g. at the synapse). Pharmaceutical compositions containing the compounds and their methods of use are also provided herein.

This application claims priority to U.S. Provisional Patent Application No. 61/241,840, filed Sep. 11, 2009, the content of which is hereby incorporated by reference in its entirety.

### I. FIELD

Provided herein are compounds useful as histamine H3 receptor inverse agonists or antagonists, compositions comprising the compounds, and methods of their use.

### II. BACKGROUND

Histamine producing cells locate in the tuberomammillary nucleus (TMN) and project throughout the brain and the spinal cord to form a histamine neurotransmitter system. Four histamine receptors, histamine H1, H2, H3, and H4 receptors, have been identified to date. The human H3 receptor was cloned in 1999. See, e.g., Lovenberg et al., Mol. Pharmacol. 55(6): 1101-07 (1999).

Histamine H3 receptors (also referred to as H3 receptors or H3 herein) are expressed on neurons throughout the CNS, particularly the forebrain. H3 receptors are primarily localized at the pre-synaptic site of the neurons and act as auto-receptors to regulate neurotransmitter release. H3 receptor is a G-protein coupled receptor (GPCR) that signals primarily through the Gi/o pathway. Activation of the pre-synaptic H3 receptors located on histaminergic neurons leads to a decrease in histamine release; whereas inhibition of H3 receptors with an antagonist or inverse agonist leads to an increase in histamine at the synapse. Thus H3 receptor ligands are capable of modifying histaminergic neurotransmission in the brain: agonists decrease it, and antagonists or inverse agonists increase it. H3 receptors from the brain have significant constitutive activity in the absence of agonists. Consequently, inverse agonists will reduce receptor activity, increase histamine release, and activate histaminergic neurons. See, e.g., Goodman & Gilman's Pharmacological Basis of Therapeutics, 629 (11^{th} ed. 2006).

H3 receptors are also found on the terminals of other neurotransmitter producing neurons, where they serve as pre-synaptic hetero-receptors to regulate the release of other neurotransmitters. H3 receptor antagonists have been shown to increase acetylcholine, norepinephrine, and dopamine in the extra-cellular fluid. The ability for H3 receptors to modulate the release of a variety of neurotransmitters suggests a wide range of therapeutic indications for H3 antagonists and inverse agonists.

H3 receptor antagonists or inverse agonists that cross the blood-brain barrier have a range of central effects through the activation of histaminergic neurons. For example, in animal experiments, H3 antagonists or inverse agonists induced marked arousal and wakefulness, improved attention and learning, and demonstrated beneficial effects in animal models of convulsions. Thus these compounds may be used to treat conditions such as cognitive impairment, pathological diurnal somnolence, and epilepsy without sedative side effects. The ability of these compounds to improve wakefulness could also lead to an improved sleep pattern, and therefore H3 antagonists or inverse agonists may also be useful in treating sleeping disorders, such as insomnia.

Preclinical research with H3 antagonists and inverse agonists suggests that this class of ligands may offer novel treatments for a variety of disorders, including but not limited to, cognitive impairments (such as those associated with Alzheimer's and Parkinson's diseases), schizophrenia, attention deficit hyperactivity disorder (ADHD), pain, and obesity. Additionally, these ligands have been shown to possess wake-promoting properties in both pre-clinical and clinical studies and may be useful in disorders associated with excessive daytime sleepiness. Additional uses of H3 ligands include, but are not limited to, disorders of the mood such as anxiety and depression, seizures, vertigo, movement disorders, and gastrointestinal (GI) motility disorders.

In addition, it is reported that H3 receptors may be associated with other various neurological disorders. Therefore, there is a great need for effective H3 inverse agonists and antagonists as therapeutics for treatment of various disorders, such as neurological disorders.

### III. SUMMARY

Provided herein are compounds of formula (I), or pharmaceutically acceptable salt or stereoisomer thereof:

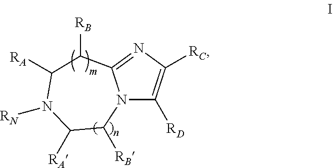

wherein R_{N}, R_{A}, R_{A}′, R_{B}, R_{B}′, R_{C}, R_{D}, m, and n are defined herein elsewhere. The compounds are useful as histamine H3 receptor inverse agonists or antagonists.

Also provided herein are compositions and dosage forms comprising compounds provided herein. Compositions and dosage forms provided herein may comprise one or more additional active ingredients.

Also provided herein are methods for the treatment, prevention, and/or management of one or more disorder(s) using the compounds provided herein. Also provided herein are methods for the treatment, prevention, and/or management of one or more disorder(s) using the compositions provided herein. Also provided herein are methods for the treatment, prevention, and/or management of one or more symptoms of a disorder provided herein comprising administering a compound provided herein. Also provided herein are methods for the treatment, prevention, and/or management of one or more symptoms of a disorder provided herein comprising administering a composition provided herein. Also provided herein are uses of the compounds provided herein in the manufacture of a medicament for the treatment, prevention, and/or management of one or more disorder(s) provided herein. Also provided herein are uses of the compositions provided herein in the manufacture of a medicament for the treatment, prevention, and/or management of one or more disorder(s) provided herein. Also provided herein are compounds for use in the treatment, prevention, and/or management of one or more disorder(s) provided herein. Also provided herein are compositions for use in the treatment, prevention, and/or management of one or more disorder(s) provided herein. Disorders that may be treated, prevented, and/or managed include, but are not limited to, neurological disorders; neurodegenerative diseases; schizophrenia; Alzheimer's disease; Parkinson's disease; affective disorders; attention deficit hyperactivity disorder (ADHD); psychosis; convulsion; seizures; vertigo; epilepsy; narcolepsy; pain (e.g. neuropathic pain); sensitization that accompanies many neuropathic pain disorders; mood disorders such as depression and anxiety; excessive daytime sleepiness such as that seen in narcolepsy, Parkinson's disease, multiple sclerosis, shift workers, and jet lag, or as a relief of side effects of other medications; insomnia; substance abuse; cognitive impairments, impairments of learning, impairments of memory, impairments of attention, vigilance or speed of response, such as those associated with Alzheimer's disease, Parkinson's disease, schizophrenia, mild cognitive impairment (MCI), and ADHD; metabolic disorders such as diabetes and obesity; disorders related to satiety and gastric activity, or as a side effects of other medications; diseases affecting the enteric system, such as acid secretion, digestion, and gut motility; and movement disorders such as Parkinson's disease, restless leg syndrome (RLS), Huntington's disease; and any other neurological disorders described herein elsewhere.

In another embodiment, provided herein is a method of inhibiting or reducing the activity of histamine H3 receptors. The method comprises contacting the H3 receptor with a compound provided herein.

Also provided herein is a method of regulating the release of neurotransmitters, including but not limited to, histamine, acetylcholine, norepinephrine, and dopamine, at the synapse. The method comprises contacting the cell with a compound provided herein. In an exemplary embodiment, the cell is a brain cell, such as, for example, a neuronal cell or a glial cell.

### IV. DETAILED DESCRIPTION

Unless defined otherwise, all technical and scientific terms used herein have the same meaning as those commonly understood by one of ordinary skill in the art. All publications and patents referred to herein are incorporated by reference herein in their entireties.

### A. DEFINITIONS

As used herein, and unless otherwise indicated, the term “alkyl” refers to a linear or branched saturated monovalent hydrocarbon radical, wherein the alkyl may optionally be substituted with one or more substituents. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 12 (C₁₋₁₂), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms.

As used herein, and unless otherwise specified, the term “alkenyl” refers to a linear or branched monovalent hydrocarbon radical, which contains one or more, in one embodiment, one to five, carbon-carbon double bonds. The alkenyl may be optionally substituted one or more substituents. The term “alkenyl” also encompasses radicals having “cis” and “trans” configurations, or alternatively, “E” and “Z” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 12 (C₂₋₁₂), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl.

As used herein, and unless otherwise specified, the term “alkynyl” refers to a linear or branched monovalent hydrocarbon radical, which contains one or more, in one embodiment, one to five, carbon-carbon triple bonds. The alkynyl may be optionally substituted one or more substituents. The term “alkynyl” also encompasses both linear and branched alkynyl, unless otherwise specified. In certain embodiments, the alkynyl is a linear monovalent hydrocarbon radical of 2 to 20 (C₂₋₂₀), 2 to 15 (C₂₋₁₅), 2 to 12 (C₂₋₁₂), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. Examples of alkynyl groups include, but are not limited to, ethynyl (—C≡CH) and propargyl (—CH₂C≡CH). For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms.

As used herein, and unless otherwise specified, the term “cycloalkyl” refers to a cyclic saturated bridged and/or non-bridged monovalent hydrocarbon radical, which may be optionally substituted one or more substituents as described herein. In certain embodiments, the cycloalkyl has from 3 to 20 (C₃₋₂₀), from 3 to 15 (C₃₋₁₅), from 3 to 12 (C₃₋₁₂), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, decalinyl, and adamantyl.

As used herein, and unless otherwise specified, the term “heteroalkyl” refers to a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms are optionally oxidized and the nitrogen heteroatom can optionally be quaternized. The heteroatom(s) O, N and S may be placed at any interior position of the heteroalkyl group. The heteroatom Si can be placed at any position of the heteroalkyl group, including the position at which the heteroalkyl group is attached to the remainder of the molecule. In one embodiment, the heteroatom(s) O, N, and S can be placed at the external position distal to where the heteroalkyl group is attached to the remainder of the molecule. In one embodiment, the heteroatom(s) O, N, and S cannot be placed at the position at which the heteroalkyl group is attached to the remainder of the molecule. In one embodiment, the heteroatom(s) O, N, and S can be placed at the position at which the heteroalkyl group is attached to the remainder of the molecule. Examples include —O—CH₃, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms can be consecutive, such as, for example, —CH2-NH—OCH₃ and —CH₂—O—Si(CH₃)₃. Also included in the term “heteroalkyl” are those radicals described as “heteroalkylene” and “heterocycloalkyl.” The term “heteroalkylene” by itself or as part of another substituent means a divalent radical derived from heteroalkyl, as exemplified by —CH₂—CH₂—S—CH₂—CH₂— and —CH₂—S—CH₂—CH₂—NH—CH₂—. In one embodiment, for heteroalkylene groups, heteroatoms can also occupy either or both of the chain termini. In one embodiment, for heteroalkylene linking groups, as well as all other linking group provided herein, no orientation of the linking group is implied.

As used herein, and unless otherwise specified, the term “aryl” refers to a monocyclic aromatic group and/or multicyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In certain embodiments, aryl may also be optionally substituted with one or more substituents.

As used herein, and unless otherwise specified, the term “arylalkyl” or “aralkyl” refers to a monovalent alkyl group substituted with aryl. In certain embodiments, both alkyl and aryl may be optionally substituted with one or more substituents.

As used herein, and unless otherwise specified, the term “heteroaryl” refers to a monocyclic aromatic group and/or multicyclic aromatic group that contain at least one aromatic ring, wherein at least one aromatic ring contains one or more heteroatoms independently selected from O, S, and N. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzothiophenyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may be optionally substituted with one or more substituents.

As used herein, and unless otherwise specified, the term “heterocycloalkyl,” “heterocyclyl,” or “heterocyclic” refers to a monocyclic non-aromatic ring system and/or multicyclic ring system that contains at least one non-aromatic ring, wherein at least one non-aromatic ring contains one or more heteroatoms independently selected from O, S, and N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may include a fused or bridged ring system, and in which the nitrogen or sulfur atoms may be optionally oxidized, the nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic radicals include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, heterocyclic may be optionally substituted with one or more substituents.

As used herein, and unless otherwise specified, the term “halogen”, “halide” or “halo” refers to fluorine, chlorine, bromine, and/or iodine.

As used herein, and unless otherwise specified, “isotopic composition” refers to the amount of each isotope present for a given atom; “natural isotopic composition” refers to the naturally occurring isotopic composition or abundance for a given atom. In one embodiment, as used herein, and unless otherwise specified, the term “hydrogen” encompasses proton (¹H), deuterium (²H), tritium (³H), and/or mixtures thereof. In one embodiment, the hydrogen in a given position of the compounds provided herein may have a natural isotopic composition or an isotopic composition enriched with one or more isotope(s) (e.g., proton, deuterium, and/or tritium). Unless otherwise designated, the atoms of the compounds recited herein are meant to represent any known isotope of that atom or an isotopic composition thereof, including, without limitation, ¹²C, ¹³C and/or ¹⁴C; ³²S, ³³S, ³⁴S, and/or ³⁶S; ¹⁴N and/or ¹⁵N; and ¹⁶O, ¹⁷O and/or ¹⁸O).

As used herein, and unless otherwise specified, the term “optionally substituted” is intended to mean that a group, such as an alkyl, alkenyl, alkynyl, cycloalkyl, heteroalkyl, aryl, aralkyl, heteroaryl, or heterocyclyl, may be substituted with one or more substituents independently selected from, e.g., (a) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl, each optionally substituted with one or more, in one embodiment, one, two, three, or four, substituents Q¹; and (b) halo, cyano (—CN), nitro (—NO₂), —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q¹; or (iii) R^{b} and R^{c} together with the N atom to which they are attached form heteroaryl or heterocyclyl, optionally substituted with one or more, in one embodiment, one, two, three, or four, substituents Q¹. As used herein, all groups that can be substituted are “optionally substituted,” unless otherwise specified.

In one embodiment, each Q¹ is independently selected from the group consisting of (a) cyano, halo, and nitro; and (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —C(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{f} and R^{g} together with the N atom to which they are attached form heteroaryl or heterocyclyl.

As used herein, and unless otherwise specified, the term “pharmaceutically acceptable salts” refers to salts prepared from pharmaceutically acceptable non-toxic acids, including inorganic acids and organic acids. Suitable non-toxic acids include inorganic and organic acids such as, but not limited to, acetic, alginic, anthranilic, benzenesulfonic, benzoic, camphorsulfonic, citric, ethenesulfonic, formic, fumaric, furoic, gluconic, glutamic, glucorenic, galacturonic, glycidic, hydrobromic, hydrochloric, isethionic, lactic, maleic, malic, mandelic, methanesulfonic, mucic, nitric, pamoic, pantothenic, phenylacetic, propionic, phosphoric, salicylic, stearic, succinic, sulfanilic, sulfuric, tartaric acid, p-toluenesulfonic and the like. In some embodiments, the salt is formed from hydrochloric, hydrobromic, phosphoric, or sulfuric acid. In one embodiment, the salt is formed from hydrochloride salt.

As used herein, and unless otherwise specified, the term “solvate” refers to a compound provided herein or a salt thereof, which further includes a stoichiometric or non-stoichiometric amount of solvent bound by non-covalent intermolecular forces. Where the solvent is water, the solvate is a hydrate.

As used herein, and unless otherwise specified, the term “stereoisomer” encompasses all enantiomerically/stereomerically pure and enantiomerically/stereomerically enriched compounds provided herein. In certain embodiments, the term “stereoisomer” encompasses a single enantiomer or a single diastereomer. In certain embodiments, the term “stereoisomer” encompasses a mixture of two or more enantiomers and/or diastereomers.

As used herein and unless otherwise specified, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound.

As used herein and unless otherwise indicated, the term “stereomerically enriched” means a composition that comprises greater than about 55% by weight of one stereoisomer of a compound, greater than about 60% by weight of one stereoisomer of a compound, greater than about 70% by weight, or greater than about 80% by weight of one stereoisomer of a compound.

As used herein, and unless otherwise indicated, the term “enantiomerically pure” means a stereomerically pure composition of a compound having one chiral center. Similarly, the term “enantiomerically enriched” means a stereomerically enriched composition of a compound having one chiral center.

In certain embodiments, as used herein, and unless otherwise specified, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of the desired enantiomer and about 5% or less of the less preferred enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) or (−), is not related to the absolute configuration of the molecule, R or S.

As used herein, and unless otherwise specified, the term “compound” referred to herein, such as, e.g., a compound of formula (I), (Ia), (Iaa), (Ib), (Ic), (Id), (II), (IIa), (IIb), (III), or (IV), is intended to encompass one or more of the following: a free base of the compound or a salt thereof, or a stereoisomer, a mixture of two or more stereoisomers, a solid form (e.g., a crystal form or an amorphous form), a mixture of two or more solid forms, a solvate (e.g., a hydrate), a cocrystal, a complex, or a prodrug thereof. In certain embodiments, the term “compound” referred to herein is intended to encompass a pharmaceutical acceptable form of the compound, such as, e.g., a free base of the compound or a pharmaceutically acceptable salt thereof, or a stereoisomer, a mixture of two or more stereoisomers, a solid form (e.g., a crystal form or an amorphous form), a mixture of two or more solid forms, a solvate (e.g., a hydrate), a cocrystal, a complex, or a prodrug thereof. In certain embodiments, the term “compound” referred to herein is intended to encompass a free base of the compound or a salt thereof, or a stereoisomer, a mixture of two or more stereoisomers, a solid form (e.g., a crystal form or an amorphous form), a mixture of two or more solid forms, or a solvate (e.g., a hydrate) thereof. In certain embodiments, the term “compound” referred to herein is intended to encompass a solid form (e.g., a crystal form or an amorphous form) or a mixture of two or more solid forms of a free base of the compound or a salt thereof. In certain embodiments, the term “compound” referred to herein is intended to encompass a solvate (e.g., a hydrate) of a free base of the compound or a salt thereof. In one embodiment, a salt of the compound provided herein contains a suitable acid as provided herein as the counterion of the compound to form the salt. In one embodiment, the salt is a pharmaceutically acceptable salt as described herein elsewhere.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, and unless otherwise specified, the term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, Pa., 2005; Handbook of Pharmaceutical Excipients, 5th Edition, Rowe et al., eds., The Pharmaceutical Press and the American Pharmaceutical Association: 2005; and Handbook of Pharmaceutical Additives, 3rd Edition, Ash and Ash eds., Gower Publishing Company: 2007; Pharmaceutical Preformulation and Formulation, 2nd Edition, Gibson ed., CRC Press LLC: Boca Raton, Fla., 2009.

As used herein, and unless otherwise specified, the terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer of a compound described herein.

As used herein, and unless otherwise specified, the terms “drug” and “therapeutic agent” refer to a compound, or a pharmaceutical composition thereof, which is administered to a subject for treating, preventing, managing, or ameliorating one or more symptoms of a condition, disorder, or disease.

As used herein, and unless otherwise indicated, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound provided herein, with or without other additional active agent, after the onset of symptoms of the particular disease.

As used herein, and unless otherwise indicated, the terms “prevent,” “preventing” and “prevention” refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of disease or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

As used herein, and unless otherwise specified, the terms “manage,” “managing,” and “management” refer to preventing or slowing the progression, spread or worsening of a disease or disorder, or of one or more symptoms thereof. Often, the beneficial effects that a subject derives from a prophylactic and/or therapeutic agent do not result in a cure of the disease or disorder. In this regard, the term “managing” encompasses treating a patient who had suffered from the particular disease in an attempt to prevent or minimize the recurrence of the disease.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the term “subject” is defined herein to include animals such as mammals, including, but not limited to, primates (e.g., humans), cows, sheep, goats, horses, dogs, cats, rabbits, rats, mice and the like. In specific embodiments, the subject is a human.

As used herein, and unless otherwise specified, the term “histamine receptor ligand” refers to any compound, which binds to a histamine receptor. Unless otherwise specified, the histamine receptor includes, but is not limited to, histamine H3 receptor. Ligands include endogenous ligands for a given histamine receptor as well as drug molecules and other compounds, such as synthetic molecules known to bind to a particular histamine receptor. In one example, the ligands include those labeled with one or more radioisotopes, such as tritium, or otherwise (e.g., fluorescently) labeled. It is within the abilities of the skilled person to select an appropriate ligand for a given histamine receptor. For example, known ligands for the histamine receptor include histamine, R-γ-Me-histamine, imetit, thioperamide, clobenpropit, and the like.

As used herein, and unless otherwise specified, the term “neurological disorder” refers to any condition of the central or peripheral nervous system of a mammal. The term “neurological disorder” includes, but is not limited to, neurodegenerative diseases (e.g., Alzheimer's disease, Parkinson's disease and amyotrophic lateral sclerosis), neuropsychiatric diseases (e.g., schizophrenia and anxieties, such as general anxiety disorder), and affective disorders (e.g., depression and attention deficit disorder). Exemplary neurological disorders include, but are not limited to, MLS (cerebellar ataxia), Huntington's disease, Down syndrome, multi-infarct dementia, status epilecticus, contusive injuries (e.g., spinal cord injury and head injury), viral infection induced neurodegeneration, (e.g., AIDS, encephalopathies), epilepsy, benign forgetfulness, closed head injury, sleep disorders, depression (e.g., bipolar disorder), dementias, movement disorders, psychoses, alcoholism, post-traumatic stress disorder and the like. “Neurological disorder” also includes any condition associated with the disorder. In one embodiment, a method of treating a neurodegenerative disorder includes methods of treating loss of memory and/or loss of cognition associated with a neurodegenerative disorder. In one embodiment, a method of treating a neurodegenerative disorder includes methods of treating cognitive function, memory performance, learning performance, speed of reaction, and/or time to respond associated with a neurodegenerative disorder. An exemplary method would also include treating or preventing loss of neuronal function characteristic of neurodegenerative disorder. “Neurological disorder” also includes any disease or condition that is implicated, at least in part, in monoamine (e.g., norepinephrine) signaling pathways (e.g., cardiovascular disease).

As used herein, and unless otherwise specified, the term “affective disorder” includes depression, attention deficit disorder, attention deficit disorder with hyperactivity, bipolar and manic conditions, and the like. The terms “attention deficit disorder” (ADD) and “attention deficit disorder with hyperactivity” (ADDH), or attention deficit/hyperactivity disorder (AD/HD), are used herein in accordance with the accepted meanings as found in the Diagnostic and Statistical Manual of Mental Disorders, 4^{th} ed., American Psychiatric Association (1997) (DSM-IV™).

As used herein, and unless otherwise specified, the term “depression” includes all forms of depression including, but not limited to, major depressive disorder (MDD), bipolar disorder, seasonal affective disorder (SAD) and dysthymia. “Major depressive disorder” is used herein interchangeably with “unipolar depression” and “major depression.” “Depression” may also includes any condition commonly associated with depression, such as all forms of fatigue (e.g., chronic fatigue syndrome) and cognitive deficits.

As used herein, and unless otherwise specified, the terms “obsessive-compulsive disorder,” “substance abuse,” “pre-menstrual syndrome,” “anxiety,” “eating disorders” and “migraine” are used herein in a manner consistent with their accepted meanings in the art. See, e.g., DSM-IV™. For example, the term “eating disorder,” as used herein, refers to abnormal compulsions to avoid eating or uncontrollable impulses to consume abnormally large amounts of food. These disorders may affect not only the social well-being, but also the physical well-being of sufferers. Examples of eating disorders include, but are not limited to, anorexia nervosa, bulimia, and binge eating.

As used herein, and unless otherwise specified, the term “pain” refers to an unpleasant sensory and emotional experience. The term “pain,” as used herein, refers to all categories of pain, including pain that is described in terms of stimulus or nerve response, e.g., somatic pain (normal nerve response to a noxious stimulus) and neuropathic pain (abnormal response of a injured or altered sensory pathway, often without clear noxious input); pain that is categorized temporally, e.g., chronic pain and acute pain; pain that is categorized in terms of its severity, e.g., mild, moderate, or severe; and pain that is a symptom or a result of a disease state or syndrome, e.g., inflammatory pain, cancer pain, AIDS pain, arthropathy, migraine, trigeminal neuralgia, cardiac ischaemia, and diabetic peripheral neuropathic pain (See, e.g., Harrison's Principles of Internal Medicine, pp. 93-98 (Wilson et al., eds., 12th ed. 1991); Williams et al., J. of Med. Chem. 42: 1481-1485 (1999), herein each incorporated by reference in their entirety). “Pain” is also meant to include mixed etiology pain, dual mechanism pain, allodynia, causalgia, central pain, hyperesthesia, hyperpathia, dysesthesia, and hyperalgesia. In addition, The term “pain” includes pain resulting from dysfunction of the nervous system: organic pain states that share clinical features of neuropathic pain and possible common pathophysiology mechanisms, but are not initiated by an identifiable lesion in any part of the nervous system.

The term “somatic pain,” as used herein, refers to a normal nerve response to a noxious stimulus such as injury or illness, e.g., trauma, burn, infection, inflammation, or disease process such as cancer, and includes both cutaneous pain (e.g., skin, muscle or joint derived) and visceral pain (e.g., organ derived).

The term “neuropathic pain,” as used herein, refers to a heterogeneous group of neurological conditions that result from damage to the nervous system. The term also refers to pain resulting from injury to or dysfunctions of peripheral and/or central sensory pathways, and from dysfunctions of the nervous system, where the pain often occurs or persists without an obvious noxious input. This includes pain related to peripheral neuropathies as well as central neuropathic pain. Common types of peripheral neuropathic pain include diabetic neuropathy (also called diabetic peripheral neuropathic pain, or DN, DPN, or DPNP), post-herpetic neuralgia (PHN), and trigeminal neuralgia (TGN). Central neuropathic pain, involving damage to the brain or spinal cord, can occur following stroke, spinal cord injury, and as a result of multiple sclerosis, and is also encompassed by the term. Other types of pain that are meant to be included in the definition of neuropathic pain include, but are not limited to, pain from neuropathic cancer pain, HIV/AIDS induced pain, phantom limb pain, and complex regional pain syndrome.

The term also encompasses the common clinical features of neuropathic pain including, but not limited to, sensory loss, allodynia (non-noxious stimuli produce pain), hyperalgesia and hyperpathia (delayed perception, summation, and painful after sensation). Pain is often a combination of nociceptive and neuropathic types, for example, mechanical spinal pain and radiculopathy or myelopathy.

As used herein, and unless otherwise specified, the term “acute pain” refers to the normal, predicted physiological response to a noxious chemical, thermal or mechanical stimulus typically associated with invasive procedures, trauma and disease. It is generally time-limited, and may be viewed as an appropriate response to a stimulus that threatens and/or produces tissue injury. The term also refers to pain which is marked by short duration or sudden onset.

As used herein, and unless otherwise specified, the term “chronic pain” encompasses the pain occurring in a wide range of disorders, for example, trauma, malignancies and chronic inflammatory diseases such as rheumatoid arthritis. Chronic pain may last more than about six months. In addition, the intensity of chronic pain may be disproportionate to the intensity of the noxious stimulus or underlying process. The term also refers to pain associated with a chronic disorder, or pain that persists beyond resolution of an underlying disorder or healing of an injury, and that is often more intense than the underlying process would predict. It may be subject to frequent recurrence.

As used herein, and unless otherwise specified, the term “inflammatory pain” is pain in response to tissue injury and the resulting inflammatory process. Inflammatory pain is adaptive in that it elicits physiologic responses that promote healing. However, inflammation may also affect neuronal function. Inflammatory mediators, including PGE₂ induced by the COX2 enzyme, bradykinins, and other substances, bind to receptors on pain-transmitting neurons and alter their function, increasing their excitability and thus increasing pain sensation. Much chronic pain has an inflammatory component. The term also refers to pain which is produced as a symptom or a result of inflammation or an immune system disorder.

As used herein, and unless otherwise specified, the term “visceral pain” refers to pain which is located in an internal organ.

As used herein, and unless otherwise specified, the term “mixed etiology pain” refers to pain that contains both inflammatory and neuropathic components.

As used herein, and unless otherwise specified, the term “dual mechanism pain” refers to pain that is amplified and maintained by both peripheral and central sensitization.

As used herein, and unless otherwise specified, the term “causalgia” refers to a syndrome of sustained burning, allodynia, and hyperpathia after a traumatic nerve lesion, often combined with vasomotor and sudomotor dysfunction and later trophic changes. As used herein, and unless otherwise specified, the term “central pain” refers to pain initiated by a primary lesion or dysfunction in the central nervous system.

As used herein, and unless otherwise specified, the term “hyperesthesia” refers to increased sensitivity to stimulation, excluding the special senses.

As used herein, and unless otherwise specified, the term “hyperpathia” refers to a painful syndrome characterized by an abnormally painful reaction to a stimulus, especially a repetitive stimulus, as well as an increased threshold. It may occur with allodynia, hyperesthesia, hyperalgesia, or dysesthesia.

As used herein, and unless otherwise specified, the term “dysesthesia” refers to an unpleasant abnormal sensation, whether spontaneous or evoked. In certain embodiments, dysesthesia include hyperalgesia and allodynia.

As used herein, and unless otherwise specified, the term “hyperalgesia” refers to an increased response to a stimulus that is normally painful. It reflects increased pain on suprathreshold stimulation.

As used herein, and unless otherwise specified, the term “allodynia” refers to pain due to a stimulus that does not normally provoke pain.

As used herein, and unless otherwise specified, the term “Diabetic Peripheral Neuropathic Pain” (DPNP), also called diabetic neuropathy, DN or diabetic peripheral neuropathy), refers to chronic pain caused by neuropathy associated with diabetes mellitus. The classic presentation of DPNP is pain or tingling in the feet that can be described not only as “burning” or “shooting” but also as severe aching pain. Less commonly, patients may describe the pain as itching, tearing, or like a toothache. The pain may be accompanied by allodynia and hyperalgesia and an absence of symptoms, such as numbness.

As used herein, and unless otherwise specified, the term “Post-Herpetic Neuralgia”, also called “Postherpetic Neuralgia (PHN)”, refers to a painful condition affecting nerve fibers and skin. Without being limited by a particular theory, it is a complication of shingles, a second outbreak of the varicella zoster virus (VZV), which initially causes chickenpox.

As used herein, and unless otherwise specified, the term “neuropathic cancer pain” refers to peripheral neuropathic pain as a result of cancer, and can be caused directly by infiltration or compression of a nerve by a tumor, or indirectly by cancer treatments such as radiation therapy and chemotherapy (chemotherapy-induced neuropathy).

As used herein, and unless otherwise specified, the term “HIV/AIDS peripheral neuropathy” or “HIV/AIDS related neuropathy” refers to peripheral neuropathy caused by HIV/AIDS, such as acute or chronic inflammatory demyelinating neuropathy (AIDP and CIDP, respectively), as well as peripheral neuropathy resulting as a side effect of drugs used to treat HIV/AIDS.

As used herein, and unless otherwise specified, the term “Phantom Limb Pain” refers to pain appearing to come from where an amputated limb used to be. Phantom limb pain can also occur in limbs following paralysis (e.g., following spinal cord injury). “Phantom Limb Pain” is usually chronic in nature.

As used herein, and unless otherwise specified, the term “Trigeminal Neuralgia (TN)” refers to a disorder of the fifth cranial (trigeminal) nerve that causes episodes of intense, stabbing, electric-shock-like pain in the areas of the face where the branches of the nerve are distributed (lips, eyes, nose, scalp, forehead, upper jaw, and lower jaw). It is also known as the “suicide disease”.

As used herein, and unless otherwise specified, the term “Complex Regional Pain Syndrome (CRPS),” formerly known as Reflex Sympathetic Dystrophy (RSD), refers to a chronic pain condition whose key symptom is continuous, intense pain out of proportion to the severity of the injury, which gets worse rather than better over time. The term encompasses type 1 CRPS, which includes conditions caused by tissue injury other than peripheral nerve, and type 2 CRPS, in which the syndrome is provoked by major nerve injury, and is sometimes called causalgia.

As used herein, and unless otherwise specified, the term “fibromyalgia” refers to a chronic condition characterized by diffuse or specific muscle, joint, or bone pain, along with fatigue and a range of other symptoms. Previously, fibromyalgia was known by other names such as fibrositis, chronic muscle pain syndrome, psychogenic rheumatism and tension myalgias.

As used herein, and unless otherwise specified, the term “convulsion” refers to a neurological disorder and is used interchangeably with “seizure,” although there are many types of seizure, some of which have subtle or mild symptoms instead of convulsions. Seizures of all types may be caused by disorganized and sudden electrical activity in the brain. In some embodiments, convulsions are a rapid and uncontrollable shaking during which the muscles contract and relax repeatedly.

### B. COMPOUNDS

In one embodiment, provided herein is a compound of formula (I):

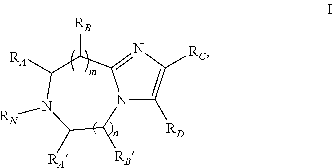

or a pharmaceutically acceptable salt or stereoisomer thereof, wherein

R_{N}, R_{A}, R_{A}′, R_{B}, and R_{B}′ are each independently a bond, hydrogen, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, or (5 to 10 membered)heteroaryl, each of which may be optionally substituted with one or more R′;

optionally, R_{N} and R_{A}, or R_{N} and R_{A}′, or R_{A} and R_{B}, or R_{A}′ and R_{B}′ together with the atoms to which they are attached form an optionally substituted 3-, 4-, 5-, 6-, or 7-membered ring;

optionally, R_{N} and R_{B}, or R_{N} and R_{B}′, or R_{A} and R_{A}′, or R_{A} and R_{B}′, or R_{B} and R_{A}′, or R_{B} and R_{B}′ are taken together to form a 1-, 2-, or 3-atom bridge;

R_{C} and R_{D} are each independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)-heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R″; or R_{C} and R_{D} together may form a ring;

each occurrence of R′ is independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₂; or two R′ substituents together may form a 3 to 10 membered ring;

each occurrence of R″ is independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₁; or two R″ substituents together may form a 3 to 10 membered ring;

each occurrence of R₁ is independently hydrogen, halogen, cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, —S(O)₂NR₃R₄, (C₁-C₁₀)alkyl optionally substituted with one or more R₂, (C₃-C₁₀)cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂;

each occurrence of R₂ is independently hydrogen, (C₁-C₆)alkyl optionally substituted with one or more R₃, (C₃-C₆)cycloalkyl optionally substituted with one or more R₃, halogen, cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, or —S(O)₂NR₃R₄;

R₃ and R₄ are each independently hydrogen, (C₁-C₆)alkyl, (C₃-C₆)cycloalkyl, (C₇-C₁₀)aralkyl; (C₁-C₆)heteroalkyl, (C₃-C₆)heterocycloalkyl, (6 to 10 membered)aryl, or (5 to 10 membered)heteroaryl; or R₃ and R₄ together may form a 3 to 10 membered ring;

q is 0, 1, or 2;

m is 0, 1, or 2; and

n is 1, 2, or 3.

In one embodiment, provided herein is a compound of formula (I):

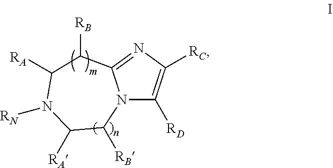

or a pharmaceutically acceptable salt or stereoisomer thereof, wherein

R_{N}, R_{A}, R_{A}′, R_{B}, and R_{B}′ are each independently a bond, hydrogen, (C₁-C₁₀)alkyl, (C₁-C₁₀)alkenyl, (C₃-C₁₀)cycloalkyl, (6 to 10 membered)aryl, (C₁-C₁₀)hetero alkyl, (C₃-C₁₀)heterocycloalkyl, or (5 to 10 membered)heteroaryl, each of which may be optionally substituted with one or more R′; and (i) one pair of R_{N} and R_{A}, or R_{N} and R_{A}′, or R_{A} and R_{B}, or R_{A}′ and R_{B}′ together with the atoms to which they are attached form an optionally substituted 3-, 4-, 5-, 6-, or 7-membered non-aromatic ring (e.g., a fully or partially saturated ring); or (ii) one pair of R_{N} and R_{B}, or R_{N} and R_{B}′, or R_{A} and R_{A}′, or R_{A} and R_{B}′, or R_{B} and R_{A}′, or R_{B} and R_{B}′ are taken together to form a 1-, 2-, or 3-atom bridge;

R_{C} and R_{D} are each independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)-heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R″; or R_{C} and R_{D} together may form a ring;

each occurrence of R′ is independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₂; or two R′ substituents together may form a 3 to 10 membered ring;

each occurrence of R″ is independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₁; or two R″ substituents together may form a 3 to 10 membered ring;

each occurrence of R₁ is independently hydrogen, halogen, cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, —S(O)₂NR₃R₄, (C₁-C₁₀)alkyl optionally substituted with one or more R₂, (C₃-C₁₀)cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂;

each occurrence of R₂ is independently hydrogen, (C₁-C₆)alkyl optionally substituted with one or more R₃, (C₃-C₆)cycloalkyl optionally substituted with one or more R₃, halogen, cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, or —S(O)₂NR₃R₄;

R₃ and R₄ are each independently hydrogen, (C₁-C₆)alkyl, (C₃-C₆)cycloalkyl, (C₇-C₁₀)aralkyl; (C₁-C₆)heteroalkyl, (C₃-C₆)heterocycloalkyl, (6 to 10 membered)aryl, or (5 to 10 membered)heteroaryl; or R₃ and R₄ together may form a 3 to 10 membered ring;

q is 0, 1, or 2;

m is 1 or 2; and

n is 1, 2, or 3.

In one embodiment, R_{N} is a bond. In another embodiment, R_{N} is hydrogen. In another embodiment, R_{N} is (C₁-C₁₀)alkyl. In another embodiment, R_{N} is (C₁-C₁₀)alkenyl. In another embodiment, R_{N} is (C₃-C₁₀)cycloalkyl. In another embodiment, R_{N} is (6 to 10 membered)aryl. In another embodiment, R_{N} is (C₁-C₁₀)heteroalkyl. In another embodiment, R_{N} is (C₃-C₁₀)heterocycloalkyl. In another embodiment, R_{N} is (5 to 10 membered)heteroaryl. Each occurrence of R_{N} may be optionally substituted with one or more R′. In one embodiment, when R_{N} is a bond, R_{N} is substituted with R′ (i.e., R′ is directly attached to the nitrogen atom to which R_{N} is attached).

In one embodiment, R_{N} is cyclopropyl optionally substituted with one or more R′. In another embodiment, R_{N} is cyclobutyl optionally substituted with one or more R′. In another embodiment, R_{N} is cyclopentyl optionally substituted with one or more R′. In another embodiment, R_{N} is cyclohexyl optionally substituted with one or more R′.

In one embodiment, R_{A} is a bond. In another embodiment, R_{A} is hydrogen. In another embodiment, R_{A} is (C₁-C₁₀)alkyl. In certain embodiments, R_{A} is methyl. In another embodiment, R_{A} is (C₁-C₁₀)alkenyl. In another embodiment, R_{A} is (C₃-C₁₀)cycloalkyl. In another embodiment, R_{A} is (6 to 10 membered)aryl. In another embodiment, R_{A} is (C₁-C₁₀)heteroalkyl. In another embodiment, R_{A} is (C₃-C₁₀)heterocycloalkyl. In another embodiment, R_{A} is (5 to 10 membered)heteroaryl. Each occurrence of R_{A} may be optionally substituted with one or more R′. In one embodiment, when R_{A} is a bond, R_{A} is substituted with R′ (i.e., R′ is directly attached to the carbon atom to which R_{A} is attached).

In one embodiment, R_{A}′ is a bond. In another embodiment, R_{A}′ is hydrogen. In another embodiment, R_{A}′ is (C₁-C₁₀)alkyl. In certain embodiments, R_{A}′ is methyl. In another embodiment, R_{A}′ is (C₁-C₁₀)alkenyl. In another embodiment, R_{A}′ is (C₃-C₁₀)cycloalkyl. In another embodiment, R_{A}′ is (6 to 10 membered)aryl. In another embodiment, R_{A}′ is (C₁-C₁₀)heteroalkyl. In another embodiment, R_{A}′ is (C₃-C₁₀)heterocycloalkyl. In another embodiment, R_{A}′ is (5 to 10 membered)heteroaryl. Each occurrence of R_{A}′ may be optionally substituted with one or more R′. In one embodiment, when R_{A}′ is a bond, R_{A}′ is substituted with R′ (i.e., R′ is directly attached to the carbon atom to which R_{A}′ is attached).

In one embodiment, R_{B} is a bond. In another embodiment, R_{B} is hydrogen. In another embodiment, R_{B} is (C₁-C₁₀)alkyl. In another embodiment, R_{B} is (C₁-C₁₀)alkenyl. In another embodiment, R_{B} is (C₃-C₁₀)cycloalkyl. In another embodiment, R_{B} is (6 to 10 membered)aryl. In another embodiment, R_{B} is (C₁-C₁₀)heteroalkyl. In another embodiment, R_{B} is (C₃-C₁₀)heterocycloalkyl. In another embodiment, R_{B} is (5 to 10 membered)heteroaryl. Each occurrence of R_{B} may be optionally substituted with one or more R′. In one embodiment, when R_{B} is a bond, R_{B} is substituted with R′ (i.e., R′ is directly attached to the carbon atom to which R_{B} is attached).

In one embodiment, R_{B}′ is a bond. In another embodiment, R_{B}′ is hydrogen. In another embodiment, R_{B}′ is (C₁-C₁₀)alkyl. In another embodiment, R_{B}′ is (C₁-C₁₀)alkenyl. In another embodiment, R_{B}′ is (C₃-C₁₀)cycloalkyl. In another embodiment, R_{B}′ is (6 to 10 membered)aryl. In another embodiment, R_{B}′ is (C₁-C₁₀)heteroalkyl. In another embodiment, R_{B}′ is (C₃-C₁₀)heterocycloalkyl. In another embodiment, R_{B}′ is (5 to 10 membered)heteroaryl. Each occurrence of R_{B}′ may be optionally substituted with one or more R′. In one embodiment, when R_{B}′ is a bond, R_{B}′ is substituted with R′ (i.e., R′ is directly attached to the carbon atom to which R_{B}′ is attached).

In one embodiment, R_{N} and R_{A} together with the atoms to which they are attached form an optionally substituted 3-membered ring. In another embodiment, R_{N} and R_{A} together with the atoms to which they are attached form an optionally substituted 4-membered ring. In another embodiment, R_{N} and R_{A} together with the atoms to which they are attached form an optionally substituted 5-membered ring. In certain embodiments, R_{N} and R_{A} together with the atoms to which they are attached form an optionally substituted pyrrolidine ring. In another embodiment, R_{N} and R_{A} together with the atoms to which they are attached form an optionally substituted 6-membered ring. In certain embodiments, R_{N} and R_{A} together with the atoms to which they are attached form an optionally substituted piperidine ring. In another embodiment, R_{N} and R_{A} together with the atoms to which they are attached form an optionally substituted 7-membered ring. In another embodiment, R_{N} and R_{A} together with the atoms to which they are attached form an optionally substituted non-aromatic ring (e.g., a fully or partially saturated ring). In another embodiment, R_{N} and R_{A} together with the atoms to which they are attached form a 3-, 4-, 5-, 6-, or 7-membered ring, which is optionally substituted with one or more R′. In another embodiment, R_{N} and R_{A} together with the atoms to which they are attached form a 3-, 4-, 5-, 6-, or 7-membered non-aromatic ring, which is optionally substituted with one or more R′.

In one embodiment, R_{N} and R_{A}′ together with the atoms to which they are attached form an optionally substituted 3-membered ring. In another embodiment, R_{N} and R_{A}′ together with the atoms to which they are attached form an optionally substituted 4-membered ring. In another embodiment, R_{N} and R_{A}′ together with the atoms to which they are attached form an optionally substituted 5-membered ring. In certain embodiments, R_{N} and R_{A}′ together with the atoms to which they are attached form an optionally substituted pyrrolidine ring. In another embodiment, R_{N} and R_{A}′ together with the atoms to which they are attached form an optionally substituted 6-membered ring. In certain embodiments, R_{N} and R_{A}′ together with the atoms to which they are attached form an optionally substituted piperidine ring. In another embodiment, R_{N} and R_{A}′ together with the atoms to which they are attached form an optionally substituted 7-membered ring. In another embodiment, R_{N} and R_{A}′ together with the atoms to which they are attached form an optionally substituted non-aromatic ring (e.g., a fully or partially saturated ring). In another embodiment, R_{N} and R_{A}′ together with the atoms to which they are attached form a 3-, 4-, 5-, 6-, or 7-membered ring, which is optionally substituted with one or more R′. In another embodiment, R_{N} and R_{A}′ together with the atoms to which they are attached form a 3-, 4-, 5-, 6-, or 7-membered non-aromatic ring, which is optionally substituted with one or more R′.

In one embodiment, R_{A} and R_{B} together with the atoms to which they are attached form an optionally substituted 3-membered ring. In another embodiment, R_{A} and R_{B} together with the atoms to which they are attached form an optionally substituted 4-membered ring. In another embodiment, R_{A} and R_{B} together with the atoms to which they are attached form an optionally substituted 5-membered ring. In another embodiment, R_{A} and R_{B} together with the atoms to which they are attached form an optionally substituted 6-membered ring. In another embodiment, R_{A} and R_{B} together with the atoms to which they are attached form an optionally substituted 7-membered ring. In another embodiment, R_{A} and R_{B} together with the atoms to which they are attached form an optionally substituted non-aromatic ring (e.g., a fully or partially saturated ring). In another embodiment, R_{A} and R_{B} together with the atoms to which they are attached form a 3-, 4-, 5-, 6-, or 7-membered ring, which is optionally substituted with one or more R′. In another embodiment, R_{A} and R_{B} together with the atoms to which they are attached form a 3-, 4-, 5-, 6-, or 7-membered non-aromatic ring, which is optionally substituted with one or more R′. In one embodiment, when m is 1, R_{A} and R_{B} together with the atoms to which they are attached form an optionally substituted 3-, 4-, 5-, 6-, or 7-membered ring as described herein elsewhere. In one embodiment, when m is 2 and there are two occurrences of R_{B}, R_{A} and one occurrence of R_{B} together with the atoms to which they are attached form an optionally substituted 3-, 4-, 5-, 6-, or 7-membered ring as described herein elsewhere, and the other occurrence of R_{B} is as defined herein elsewhere. In certain embodiments, R_{A} and R_{B} together with the atoms to which they are attached form an optionally substituted 3-, 4-, 5-, 6-, or 7-membered cycloalkyl ring. In certain embodiments, R_{A} and R_{B} together with the atoms to which they are attached form an optionally substituted 3-, 4-, 5-, 6-, or 7-membered heterocycloalkyl ring.

In one embodiment, R_{A}′ and R_{B}′ together with the atoms to which they are attached form an optionally substituted 3-membered ring. In another embodiment, R_{A} ′ and R_{B}′ together with the atoms to which they are attached form an optionally substituted 4-membered ring. In another embodiment, R_{A}′ and R_{B}′ together with the atoms to which they are attached form an optionally substituted 5-membered ring. In another embodiment, R_{A}′ and R_{B}′ together with the atoms to which they are attached form an optionally substituted 6-membered ring. In another embodiment, R_{A}′ and R_{B}′ together with the atoms to which they are attached form an optionally substituted 7-membered ring. In another embodiment, R_{A}′ and R_{B}′ together with the atoms to which they are attached form an optionally substituted non-aromatic ring (e.g., a fully or partially saturated ring). In another embodiment, R_{A}′ and R_{B}′ together with the atoms to which they are attached form a 3-, 4-, 5-, 6-, or 7-membered ring, which is optionally substituted with one or more R′. In another embodiment, R_{A}′ and R_{B}′ together with the atoms to which they are attached form a 3-, 4-, 5-, 6-, or 7-membered non-aromatic ring, which is optionally substituted with one or more R′. In one embodiment, when n is 1, R_{A}′ and R_{B}′ together with the atoms to which they are attached form an optionally substituted 3-, 4-, 5-, 6-, or 7-membered ring as described herein elsewhere. In one embodiment, when n is 2 and there are two occurrences of R_{B}′, R_{A}′ and one occurrence of R_{B}′ together with the atoms to which they are attached form an optionally substituted 3-, 4-, 5-, 6-, or 7-membered ring as described herein elsewhere, and the other occurrence of R_{B}′ is as defined herein elsewhere. In one embodiment, when n is 3 and there are three occurrences of R_{B}′, R_{A}′ and one occurrence of R_{B}′ together with the atoms to which they are attached form an optionally substituted 3-, 4-, 5-, 6-, or 7-membered ring as described herein elsewhere, and the other two occurrences of R_{B}′ are independently as defined herein elsewhere. In certain embodiments, R_{A}′ and R_{B}′ together with the atoms to which they are attached form an optionally substituted 3-, 4-, 5-, 6-, or 7-membered cycloalkyl ring. In certain embodiments, R_{A}′ and R_{B}′ together with the atoms to which they are attached form an optionally substituted 3-, 4-, 5-, 6-, or 7-membered heterocycloalkyl ring.

In one embodiment, R_{N} and R_{B} are taken together to form a 1-atom bridge. In another embodiment, R_{N} and R_{B} are taken together to form a 2-atom bridge. In another embodiment, R_{N} and R_{B} are taken together to form a 3-atom bridge. In certain embodiments, R_{N} and R_{B} are taken together to form a methylene, ethylene, or propylene bridge. In one embodiment, the 1-, 2-, or 3-atom bridge is optionally substituted with one or more R′. In one embodiment, when m is 1, R_{N} and R_{B} are taken together to form a 1-, 2-, or 3-atom bridge as described herein elsewhere. In one embodiment, when m is 2 and there are two occurrences of R_{B}, R_{N} and one occurrence of R_{B} are taken together to form a 1-, 2-, or 3-atom bridge as described herein elsewhere, and the other occurrence of R_{B} is as defined herein elsewhere.

In one embodiment, R_{N} and R_{B}′ are taken together to form a 1-atom bridge. In another embodiment, R_{N} and R_{B}′ are taken together to form a 2-atom bridge. In another embodiment, R_{N} and R_{B}′ are taken together to form a 3-atom bridge. In certain embodiments, R_{N} and R_{B}′ are taken together to form a methylene, ethylene, or propylene bridge. In one embodiment, the 1-, 2-, or 3-atom bridge is optionally substituted with one or more R′. In one embodiment, when n is 1, R_{N} and R_{B}′ are taken together to form a 1-, 2-, or 3-atom bridge as described herein elsewhere. In one embodiment, when n is 2 and there are two occurrences of R_{B}′, R_{N} and one occurrence of R_{B}′ are taken together to form a 1-, 2-, or 3-atom bridge as described herein elsewhere, and the other occurrence of R_{B}′ is as defined herein elsewhere. In one embodiment, when n is 3 and there are three occurrences of R_{B}′, R_{N} and one occurrence of R_{B}′ are taken together to form a 1-, 2-, or 3-atom bridge as described herein elsewhere, and the other two occurrences of R_{B}′ are independently as defined herein elsewhere.

In one embodiment, R_{A} and R_{A}′ are taken together to form a 1-atom bridge. In another embodiment, R_{A} and R_{A}′ are taken together to form a 2-atom bridge. In another embodiment, R_{A} and R_{A}′ are taken together to form a 3-atom bridge. In certain embodiments, R_{A} and R_{A}′ are taken together to form a methylene, ethylene, or propylene bridge. In one embodiment, the 1-, 2-, or 3-atom bridge is optionally substituted with one or more R′.

In one embodiment, R_{A} and R_{B}′ are taken together to form a 1-atom bridge. In another embodiment, R_{A} and R_{B}′ are taken together to form a 2-atom bridge. In another embodiment, R_{A} and R_{B}′ are taken together to form a 3-atom bridge. In certain embodiments, R_{A} and R_{B}′ are taken together to form a methylene, ethylene, or propylene bridge. In one embodiment, the 1-, 2-, or 3-atom bridge is optionally substituted with one or more R′. In one embodiment, when n is 1, R_{A} and R_{B}′ are taken together to form a 1-, 2-, or 3-atom bridge as described herein elsewhere. In one embodiment, when n is 2 and there are two occurrences of R_{B}′, R_{A} and one occurrence of R_{B}′ are taken together to form a 1-, 2-, or 3-atom bridge as described herein elsewhere, and the other occurrence of R_{B}′ is as defined herein elsewhere. In one embodiment, when n is 3 and there are three occurrences of R_{B}′, R_{A} and one occurrence of R_{B}′ are taken together to form a 1-, 2-, or 3-atom bridge as described herein elsewhere, and the other two occurrences of R_{B}′ are independently as defined herein elsewhere.

In one embodiment, R_{B} and R_{A}′ are taken together to form a 1-atom bridge. In another embodiment, R_{B} and R_{A}′ are taken together to form a 2-atom bridge. In another embodiment, R_{B} and R_{A}′ are taken together to form a 3-atom bridge. In certain embodiments, R_{B} and R_{A}′ are taken together to form a methylene, ethylene, or propylene bridge. In one embodiment, the 1-, 2-, or 3-atom bridge is optionally substituted with one or more R′. In one embodiment, when m is 1, R_{A}′ and R_{B} are taken together to form a 1-, 2-, or 3-atom bridge as described herein elsewhere. In one embodiment, when m is 2 and there are two occurrences of R_{B}, R_{A}′ and one occurrence of R_{B} are taken together to form a 1-, 2-, or 3-atom bridge as described herein elsewhere, and the other occurrence of R_{B} is as defined herein elsewhere.

In one embodiment, R_{B} and R_{B}′ are taken together to form a 1-atom bridge. In another embodiment, R_{B} and R_{B}′ are taken together to form a 2-atom bridge. In another embodiment, R_{B} and R_{B}′ are taken together to form a 3-atom bridge. In certain embodiments, R_{B} and R_{B}′ are taken together to form a methylene, ethylene, or propylene bridge. In one embodiment, the 1-, 2-, or 3-atom bridge is optionally substituted with one or more R′. In one embodiment, when m is 2 and there are two occurrences of R_{B}, one occurrence of R_{B} is taken together with one occurrence of R_{B}′ to form a 1-, 2-, or 3-atom bridge, and the other occurrence of R_{B} is as defined herein elsewhere. In one embodiment, when n is 2 and there are two occurrences of R_{B}′, one occurrence of R_{B}′ is taken together with one occurrence of R_{B} to form a 1-, 2-, or 3-atom bridge, and the other occurrence of R_{B}′ is as defined herein elsewhere. In one embodiment, when n is 3 and there are three occurrences of R_{B}′, one occurrence of R_{B}′ is taken together with one occurrence of R_{B} to form a 1-, 2-, or 3-atom bridge, and the other two occurrences of R_{B}′ are independently as defined herein elsewhere.

In one embodiment, R_{C} is hydrogen. In another embodiment, R_{C} is halogen. In another embodiment, R_{C} is cyano. In another embodiment, R_{C} is optionally substituted (C₁-C₁₀)alkyl. In another embodiment, R_{C} is optionally substituted (C₁-C₁₀)-alkenyl. In another embodiment, R_{C} is optionally substituted (C₃-C₁₀)cycloalkyl. In another embodiment, R_{C} is optionally substituted (6 to 10 membered)aryl. In another embodiment, R_{C} is optionally substituted (C₁-C₁₀)heteroalkyl. In another embodiment, R_{C} is optionally substituted (C₃-C₁₀)heterocycloalkyl. In another embodiment, R_{C} is optionally substituted (5 to 10 membered)heteroaryl. In another embodiment, R_{C} is optionally substituted hydroxyl. In another embodiment, R_{C} is optionally substituted alkoxyl. In another embodiment, R_{C} is optionally substituted aminoalkyl. In another embodiment, R_{C} is optionally substituted amino. In another embodiment, R_{C} is optionally substituted imino. In another embodiment, R_{C} is optionally substituted amido. In another embodiment, R_{C} is optionally substituted carbonyl. In another embodiment, R_{C} is optionally substituted thiol. In another embodiment, R_{C} is optionally substituted sulfinyl. In another embodiment, R_{C} is optionally substituted sulfonyl. Each occurrence of R_{C} may be substituted with one or more R″.

In one embodiment, R_{D} is hydrogen. In another embodiment, R_{D} is halogen. In another embodiment, R_{D} is cyano. In another embodiment, R_{D} is optionally substituted (C₁-C₁₀)alkyl. In another embodiment, R_{D} is optionally substituted (C₁-C₁₀)-alkenyl. In another embodiment, R_{D} is optionally substituted (C₃-C₁₀)cycloalkyl. In another embodiment, R_{D} is optionally substituted (6 to 10 membered)aryl. In another embodiment, R_{D} is optionally substituted (C₁-C₁₀)heteroalkyl. In another embodiment, R_{D} is optionally substituted (C₃-C₁₀)heterocycloalkyl. In another embodiment, R_{D} is optionally substituted (5 to 10 membered)heteroaryl. In another embodiment, R_{D} is optionally substituted hydroxyl. In another embodiment, R_{D} is optionally substituted alkoxyl. In another embodiment, R_{D} is optionally substituted aminoalkyl. In another embodiment, R_{D} is optionally substituted amino. In another embodiment, R_{D} is optionally substituted imino. In another embodiment, R_{D} is optionally substituted amido. In another embodiment, R_{D} is optionally substituted carbonyl. In another embodiment, R_{D} is optionally substituted thiol. In another embodiment, R_{D} is optionally substituted sulfinyl. In another embodiment, R_{D} is optionally substituted sulfonyl. Each occurrence of R_{D} may be substituted with one or more R″.

In one embodiment, R_{C} and R_{D} together form a ring, which is optionally substituted with one or more R″. In another embodiment, R_{C} and R_{D} together form a (C₃-C₁₀)cycloalkyl ring, which is optionally substituted with one or more R″. In another embodiment, R_{C} and R_{D} together form a 6- to 10-membered aryl ring, which is optionally substituted with one or more R″. In another embodiment, R_{C} and R_{D} together form a (C₃-C₁₀)heterocycloalkyl ring, which is optionally substituted with one or more R″. In another embodiment, R_{C} and R_{D} together form a 5- to 10-membered heteroaryl ring, which is optionally substituted with one or more R″. In another embodiment, R_{C} and R_{D} together form a phenyl ring, which is optionally substituted with one or more R″. In another embodiment, R_{C} and R_{D} together form a thiophene ring, which is optionally substituted with one or more R″. In another embodiment, R_{C} and R_{D} together form a furan ring, which is optionally substituted with one or more R″. In another embodiment, R_{C} and R_{D} together form a pyrrole ring, which is optionally substituted with one or more R″. In another embodiment, R_{C} and R_{D} together form a pyridine ring, which is optionally substituted with one or more R″. In another embodiment, R_{C} and R_{D} together form a pyrimidine ring, which is optionally substituted with one or more R″. In another embodiment, R_{C} and R_{D} together form a pyrazine ring, which is optionally substituted with one or more R″. In another embodiment, R_{C} and R_{D} together form a tetrahydro-pyridine ring, which is optionally substituted with one or more R″. In another embodiment, R_{C} and R_{D} together form a pyridone ring, which is optionally substituted with one or more R″. In another embodiment, R_{C} and R_{D} together form a pyrimidone ring, which is optionally substituted with one or more R″. In another embodiment, R_{C} and R_{D} together form a pyridazinone ring, which is optionally substituted with one or more R″. In another embodiment, R_{C} and R_{D} together form a pyrazinone ring, which is optionally substituted with one or more R″.

In one embodiment, R′ is hydrogen. In another embodiment, R′ is halogen. In another embodiment, R′ is cyano. In another embodiment, R′ is optionally substituted (C₁-C₁₀)alkyl. In another embodiment, R′ is optionally substituted (C₁-C₁₀)alkenyl. In another embodiment, R′ is optionally substituted (C₃-C₁₀)cycloalkyl. In another embodiment, R′ is optionally substituted (6 to 10 membered)aryl. In another embodiment, R′ is optionally substituted (C₁-C₁₀)heteroalkyl. In another embodiment, R′ is optionally substituted (C₃-C₁₀)heterocycloalkyl. In another embodiment, R′ is optionally substituted (5 to 10 membered)heteroaryl. In another embodiment, R′ is optionally substituted hydroxyl. In another embodiment, R′ is optionally substituted alkoxyl. In another embodiment, R′ is optionally substituted aminoalkyl. In another embodiment, R′ is optionally substituted amino. In another embodiment, R′ is optionally substituted imino. In another embodiment, R′ is optionally substituted amido. In another embodiment, R′ is optionally substituted carbonyl. In another embodiment, R′ is optionally substituted thiol. In another embodiment, R′ is optionally substituted sulfinyl. In another embodiment, R′ is optionally substituted sulfonyl. In another embodiment, two R′ substituents together form a 3 to 10 membered ring. In another embodiment, two geminal R′ substituents together form a 3 to 10 membered ring. In another embodiment, two vicinal R′ substituents together form a 3 to 10 membered ring. In one embodiment, the 3 to 10 membered ring is optionally substituted with one or more R₂. R′ may be substituted with one or more R₂.

In one embodiment, R″ is hydrogen. In another embodiment, R″ is halogen. In another embodiment, R″ is cyano. In another embodiment, R″ is optionally substituted (C₁-C₁₀)alkyl. In another embodiment, R″ is optionally substituted (C₁-C₁₀)-alkenyl. In another embodiment, R″ is optionally substituted (C₃-C₁₀)cycloalkyl. In another embodiment, R″ is optionally substituted (6 to 10 membered)aryl. In another embodiment, R″ is optionally substituted (C₁-C₁₀)heteroalkyl. In another embodiment, R″ is optionally substituted (C₃-C₁₀)heterocycloalkyl. In another embodiment, R″ is optionally substituted (5 to 10 membered)heteroaryl. In another embodiment, R″ is optionally substituted hydroxyl. In another embodiment, R″ is optionally substituted alkoxyl. In another embodiment, R″ is optionally substituted aminoalkyl. In another embodiment, R″ is optionally substituted amino. In another embodiment, R″ is optionally substituted imino. In another embodiment, R″ is optionally substituted amido. In another embodiment, R″ is optionally substituted carbonyl. In another embodiment, R″ is optionally substituted thiol. In another embodiment, R″ is optionally substituted sulfinyl. In another embodiment, R″ is optionally substituted sulfonyl. In another embodiment, two R″ substituents together form a 3 to 10 membered ring. In another embodiment, two geminal R″ substituents together form a 3 to 10 membered ring. In another embodiment, two vicinal R″ substituents together form a 3 to 10 membered ring. In one embodiment, the 3 to 10 membered ring is optionally substituted with one or more R₁. R″ may be substituted with one or more R₁.

In one embodiment, R₁ is hydrogen. In another embodiment, R₁ is halogen. In another embodiment, R₁ is cyano. In another embodiment, R₁ is •O. In another embodiment, R₁ is —OR₃. In another embodiment, R₁ is —NR₃R₄. In another embodiment, R₁ is —N(R₃)C(O)R₄. In another embodiment, R₁ is —C(O)NR₃R₄. In another embodiment, R₁ is —C(O)R₃. In another embodiment, R₁ is —C(O)OR₃. In another embodiment, R₁ is —OC(O)R₃. In another embodiment, R₁ is —S(O)_{q}R₃. In another embodiment, R₁ is —S(O)₂NR₃R₄. In another embodiment, R₁ is (C₁-C₁₀)alkyl optionally substituted with one or more R₂. In another embodiment, R₁ is (C₃-C₁₀)-cycloalkyl optionally substituted with one or more R₂. In another embodiment, R₁ is (C₆-C₁₂)aralkyl optionally substituted with one or more R₂. In another embodiment, R₁ is (6 to 10 membered)aryl optionally substituted with one or more R₂. In another embodiment, R₁ is (C₁-C₁₀)heteroalkyl optionally substituted with one or more R₂. In another embodiment, R₁ is (C₃-C₁₀)heterocycloalkyl optionally substituted with one or more R₂. In another embodiment, R₁ is (5 to 10 membered)heteroaryl optionally substituted with one or more R₂.

In one embodiment, R₂ is hydrogen. In another embodiment, R₂ is (C₁-C₆)-alkyl optionally substituted with one or more R₃. In another embodiment, R₂ is (C₃-C₆)-cycloalkyl optionally substituted with one or more R₃. In another embodiment, R₂ is halogen. In another embodiment, R₂ is cyano. In another embodiment, R₂ is ═O. In another embodiment, R₂ is —OR₃. In another embodiment, R₂ is —NR₃R₄. In another embodiment, R₂ is —N(R₃)C(O)R₄. In another embodiment, R₂ is —C(O)NR₃R₄. In another embodiment, R₂ is —C(O)R₃. In another embodiment, R₂ is —C(O)OR₃. In another embodiment, R₂ is —OC(O)R₃. In another embodiment, R₂ is —S(O)_{q}R₃. In another embodiment, R₂ is —S(O)₂NR₃R₄.

In one embodiment, R₃ is hydrogen. In another embodiment, R₃ is (C₁-C₆)-alkyl. In another embodiment, R₃ is (C₃-C₆)cycloalkyl. In another embodiment, R₃ is (C₇-C₁₀)aralkyl. In another embodiment, R₃ is (C₁-C₆)heteroalkyl. In another embodiment, R₃ is (C₃-C₆)heterocycloalkyl. In another embodiment, R₃ is (6 to 10 membered)aryl. In another embodiment, R₃ is (5 to 10 membered)heteroaryl.

In one embodiment, R₄ is hydrogen. In another embodiment, R₄ is (C₁-C₆)-alkyl. In another embodiment, R₄ is (C₃-C₆)cycloalkyl. In another embodiment, R₄ is (C₇-C₁₀)aralkyl. In another embodiment, R₄ is (C₁-C₆)heteroalkyl. In another embodiment, R₄ is (C₃-C₆)heterocycloalkyl. In another embodiment, R₄ is (6 to 10 membered)aryl. In another embodiment, R₄ is (5 to 10 membered)heteroaryl.

In one embodiment, R₃ and R₄ together form a 3 to 10 membered ring. In another embodiment, two geminal instances of R₃ and R₄ together form a 3 to 10 membered ring. In another embodiment, two vicinal instances of R₃ and R₄ together form a 3 to 10 membered ring. In one embodiment, the 3 to 10 membered ring is optionally substituted.

In one embodiment, q is 0. In another embodiment, q is 1. In another embodiment, q is 2.

In one embodiment, m is 0. In another embodiment, m is 1. In another embodiment, m is 2. In one embodiment, m is 1 or 2.

In one embodiment, n is 1. In another embodiment, n is 2. In another embodiment, n is 3. In one embodiment, n is 1 or 2.

In one embodiment, provided herein is a compound of formula (I) as provided herein, or a pharmaceutically acceptable salt or stereoisomer thereof, wherein one pair of R_{N} and R_{A}, or R_{N} and R_{A}′, or R_{A} and R_{B}, or R_{A}′ and R_{B}′ together with the atoms to which they are attached form a 3-, 4-, 5-, 6-, or 7-membered non-aromatic ring (e.g., a fully or partially saturated ring), each of which is optionally substituted with one or more R′.

In one embodiment, provided herein is a compound of formula (I) as provided herein, or a pharmaceutically acceptable salt or stereoisomer thereof, wherein one pair of R_{N} and R_{A}, or R_{N} and R_{A}′ together with the atoms to which they are attached form a 3-, 4-, 5-, 6-, or 7-membered non-aromatic ring (e.g., a fully or partially saturated ring), each of which is optionally substituted with one or more R′.

In one embodiment, provided herein is a compound of formula (I) as provided herein, or a pharmaceutically acceptable salt or stereoisomer thereof, wherein one pair of R_{N} and R_{A} together with the atoms to which they are attached form a 3-, 4-, 5-, 6-, or 7-membered non-aromatic ring (e.g., a fully or partially saturated ring), each of which is optionally substituted with one or more R′.

In one embodiment, provided herein is a compound of formula (I) as provided herein, or a pharmaceutically acceptable salt or stereoisomer thereof, wherein one pair of R_{N} and R_{A}′ together with the atoms to which they are attached form a 3-, 4-, 5-, 6-, or 7-membered non-aromatic ring (e.g., a fully or partially saturated ring), each of which is optionally substituted with one or more R′.

In one embodiment, provided herein is a compound of formula (I) as provided herein, or a pharmaceutically acceptable salt or stereoisomer thereof, wherein one pair of R_{N} and R_{B}, or R_{N} and R_{B}′, or R_{A} and R_{A}′, or R_{A} and R_{B}′, or R_{B} and R_{A}′, or R_{B} and R_{B}′ are taken together to form a 1-, 2-, or 3-atom bridge, each of which is optionally substituted with one or more R′.

In one embodiment, provided herein is a compound of formula (I) as provided herein, or a pharmaceutically acceptable salt or stereoisomer thereof, wherein one pair of R_{A} and R_{A}′, or R_{A} and R_{B}′, or R_{B} and R_{A}′, or R_{B} and R_{B}′ are taken together to form a 1-, 2-, or 3-atom bridge, each of which is optionally substituted with one or more R′.

In one embodiment, provided herein is a compound of formula (I) as provided herein, or a pharmaceutically acceptable salt or stereoisomer thereof, wherein R_{A} and R_{A}′ are taken together to form a 1-, 2-, or 3-atom bridge, each of which is optionally substituted with one or more R′.

In one embodiment, provided herein is a compound of formula (I) as provided herein, or a pharmaceutically acceptable salt or stereoisomer thereof, wherein R_{A} and R_{B}′ are taken together to form a 1-, 2-, or 3-atom bridge, each of which is optionally substituted with one or more R′.

In one embodiment, provided herein is a compound of formula (I) as provided herein, or a pharmaceutically acceptable salt or stereoisomer thereof, wherein R_{B} and R_{A}′ are taken together to form a 1-, 2-, or 3-atom bridge, each of which is optionally substituted with one or more R′.

In one embodiment, provided herein is a compound of formula (I) as provided herein, or a pharmaceutically acceptable salt or stereoisomer thereof, wherein R_{B} and R_{B}′ are taken together to form a 1-, 2-, or 3-atom bridge, each of which is optionally substituted with one or more R′.

In one embodiment, provided herein is a compound of formula (I) as provided herein, or a pharmaceutically acceptable salt or stereoisomer thereof; wherein m is 1; n is 1; and R_{A} and R_{A}′, or R_{A} and R_{B}′, or R_{B} and R_{A}′, or R_{B} and R_{B}′ are taken together to form a 1-, 2-, or 3-atom bridge, each of which is optionally substituted with one or more R′.

In one embodiment, provided herein is a compound of formula (I) as provided herein, or a pharmaceutically acceptable salt or stereoisomer thereof; wherein m is 1; n is 1; and R_{A} and R_{A}′ are taken together to form a 1-, 2-, or 3-atom bridge, each of which is optionally substituted with one or more R′.

In one embodiment, provided herein is a compound of formula (I) as provided herein, or a pharmaceutically acceptable salt or stereoisomer thereof; wherein m is 1; n is 1; and R_{A} and R_{B}′ are taken together to form a 1-, 2-, or 3-atom bridge, each of which is optionally substituted with one or more R′.

In one embodiment, provided herein is a compound of formula (I) as provided herein, or a pharmaceutically acceptable salt or stereoisomer thereof; wherein m is 1; n is 1; and R_{B} and R_{A}′ are taken together to form a 1-, 2-, or 3-atom bridge, each of which is optionally substituted with one or more R′.

In one embodiment, provided herein is a compound of formula (I) as provided herein, or a pharmaceutically acceptable salt or stereoisomer thereof; wherein m is 1; n is 1; and R_{B} and R_{B}′ are taken together to form a 1-, 2-, or 3-atom bridge, each of which is optionally substituted with one or more R′.

In one embodiment, R_{N} is (C₃-C₁₀)cycloalkyl optionally substituted with one or more R′.

Any of the combinations of R_{N}, R_{A}, R_{A}′, R_{B}, R_{B}′, R_{C}, R_{D}, R′, R″, R₁, R₂, R₃, R₄, q, m, and n are encompassed by this disclosure and specifically provided herein.

In one embodiment, R_{C} and R_{D} together form a phenyl ring, which is optionally substituted with one or more R″.

In certain embodiments, provided herein is a compound of formula (Ia):

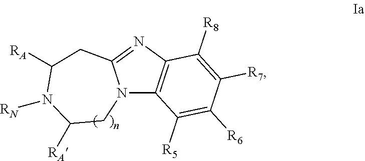

or a pharmaceutically acceptable salt or stereoisomer thereof, wherein

R₅, R₆, R₇ and R₈ are each independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)-heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₁; or two adjacent R₅, R₆, R₇, and R₈ may together form a 3 to 10 membered ring; and R_{N}, R_{A}, R_{A}′, R₁, and n are defined herein elsewhere.

In one embodiment, R₅ is hydrogen. In another embodiment, R₅ is halogen. In another embodiment, R₅ is cyano. In another embodiment, R₅ is (C₁-C₁₀)alkyl optionally substituted with one or more R₁. In another embodiment, R₅ is (C₁-C₁₀)-alkenyl optionally substituted with one or more R₁. In another embodiment, R₅ is (C₃-C₁₀)cycloalkyl optionally substituted with one or more R₁. In another embodiment, R₅ is (6 to 10 membered)aryl optionally substituted with one or more R₁. In another embodiment, R₅ is (C₁-C₁₀)heteroalkyl optionally substituted with one or more R₁. In another embodiment, R₅ is (C₃-C₁₀)heterocycloalkyl optionally substituted with one or more R₁. In another embodiment, R₅ is (5 to 10 membered)heteroaryl optionally substituted with one or more R₁. In another embodiment, R₅ is hydroxyl optionally substituted with R₁. In another embodiment, R₅ is alkoxyl optionally substituted with one or more R₁. In another embodiment, R₅ is aminoalkyl optionally substituted with one or more R₁. In another embodiment, R₅ is amino optionally substituted with one or more R₁. In another embodiment, R₅ is imino optionally substituted with one or more R₁. In another embodiment, R₅ is amido optionally substituted with one or more R₁. In another embodiment, R₅ is carbonyl optionally substituted with one or more R₁. In another embodiment, R₅ is thiol optionally substituted with one or more R₁. In another embodiment, R₅ is sulfinyl optionally substituted with one or more R₁. In another embodiment, R₅ is sulfonyl optionally substituted with one or more R₁. R₁ is defined herein elsewhere.

In one embodiment, R₆ is hydrogen. In another embodiment, R₆ is halogen. In another embodiment, R₆ is cyano. In another embodiment, R₆ is (C₁-C₁₀)alkyl optionally substituted with one or more R₁. In another embodiment, R₆ is (C₁-C₁₀)-alkenyl optionally substituted with one or more R₁. In another embodiment, R₆ is (C₃-C₁₀)cycloalkyl optionally substituted with one or more R₁. In another embodiment, R₆ is (6 to 10 membered)aryl optionally substituted with one or more R₁. In another embodiment, R₆ is (C₁-C₁₀)heteroalkyl optionally substituted with one or more R₁. In another embodiment, R₆ is (C₃-C₁₀)heterocycloalkyl optionally substituted with one or more R₁. In another embodiment, R₆ is (5 to 10 membered)heteroaryl optionally substituted with one or more R₁. In another embodiment, R₆ is hydroxyl optionally substituted with R₁. In another embodiment, R₆ is alkoxyl optionally substituted with one or more R₁. In another embodiment, R₆ is aminoalkyl optionally substituted with one or more R₁. In another embodiment, R₆ is amino optionally substituted with one or more R₁. In another embodiment, R₆ is imino optionally substituted with one or more R₁. In another embodiment, R₆ is amido optionally substituted with one or more R₁. In another embodiment, R₆ is carbonyl optionally substituted with one or more R₁. In another embodiment, R₆ is thiol optionally substituted with one or more R₁. In another embodiment, R₆ is sulfinyl optionally substituted with one or more R₁. In another embodiment, R₆ is sulfonyl optionally substituted with one or more R₁. R₁ is defined herein elsewhere.

In one embodiment, R₇ is hydrogen. In another embodiment, R₇ is halogen. In another embodiment, R₇ is cyano. In another embodiment, R₇ is (C₁-C₁₀)alkyl optionally substituted with one or more R₁. In another embodiment, R₇ is (C₁-C₁₀)-alkenyl optionally substituted with one or more R₁. In another embodiment, R₇ is (C₃-C₁₀)cycloalkyl optionally substituted with one or more R₁. In another embodiment, R₇ is (6 to 10 membered)aryl optionally substituted with one or more R₁. In another embodiment, R₇ is (C₁-C₁₀)heteroalkyl optionally substituted with one or more R₁. In another embodiment, R₇ is (C₃-C₁₀)heterocycloalkyl optionally substituted with one or more R₁. In another embodiment, R₇ is (5 to 10 membered)heteroaryl optionally substituted with one or more R₁. In another embodiment, R₇ is hydroxyl optionally substituted with R₁. In another embodiment, R₇ is alkoxyl optionally substituted with one or more R₁. In another embodiment, R₇ is aminoalkyl optionally substituted with one or more R₁. In another embodiment, R₇ is amino optionally substituted with one or more R₁. In another embodiment, R₇ is imino optionally substituted with one or more R₁. In another embodiment, R₇ is amido optionally substituted with one or more R₁. In another embodiment, R₇ is carbonyl optionally substituted with one or more R₁. In another embodiment, R₇ is thiol optionally substituted with one or more R₁. In another embodiment, R₇ is sulfinyl optionally substituted with one or more R₁. In another embodiment, R₇ is sulfonyl optionally substituted with one or more R₁. R₁ is defined herein elsewhere.

In one embodiment, R₈ is hydrogen. In another embodiment, R₈ is halogen. In another embodiment, R₈ is cyano. In another embodiment, R₈ is (C₁-C₁₀)alkyl optionally substituted with one or more R₁. In another embodiment, R₈ is (C₁-C₁₀)-alkenyl optionally substituted with one or more R₁. In another embodiment, R₈ is (C₃-C₁₀)cycloalkyl optionally substituted with one or more R₁. In another embodiment, R₈ is (6 to 10 membered)aryl optionally substituted with one or more R₁. In another embodiment, R₈ is (C₁-C₁₀)heteroalkyl optionally substituted with one or more R₁. In another embodiment, R₈ is (C₃-C₁₀)heterocycloalkyl optionally substituted with one or more R₁. In another embodiment, R₈ is (5 to 10 membered)heteroaryl optionally substituted with one or more R₁. In another embodiment, R₈ is hydroxyl optionally substituted with R₁. In another embodiment, R₈ is alkoxyl optionally substituted with one or more R₁. In another embodiment, R₈ is aminoalkyl optionally substituted with one or more R₁. In another embodiment, R₈ is amino optionally substituted with one or more R₁. In another embodiment, R₈ is imino optionally substituted with one or more R₁. In another embodiment, R₈ is amido optionally substituted with one or more R₁. In another embodiment, R₈ is carbonyl optionally substituted with one or more R₁. In another embodiment, R₈ is thiol optionally substituted with one or more R₁. In another embodiment, R₈ is sulfinyl optionally substituted with one or more R₁. In another embodiment, R₈ is sulfonyl optionally substituted with one or more R₁. R₁ is defined herein elsewhere.

In one embodiment, R₅ and R₆ together form a 3 to 10 membered ring, which is optionally substituted with one or more R₁. In another embodiment, R₆ and R₇ together form a 3 to 10 membered ring, which is optionally substituted with one or more R₁. In another embodiment, R₇ and R₈ together form a 3 to 10 membered ring, which is optionally substituted with one or more R₁. R₁ is defined herein elsewhere.

In one embodiment, R_{N} and R_{A}′ together with the atoms to which they are attached form a 3-, 4-, 5-, 6-, or 7-membered non-aromatic ring (e.g., a fully or partially saturated ring), which is optionally substituted with one or more R′. In one embodiment, R_{N} and R_{A}′ together with the atoms to which they are attached form an optionally substituted pyrrolidine ring. In one embodiment, R_{N} and R_{A}′ together with the atoms to which they are attached form an optionally substituted piperidine ring. Specific examples include, but are not limited to, the following compounds:

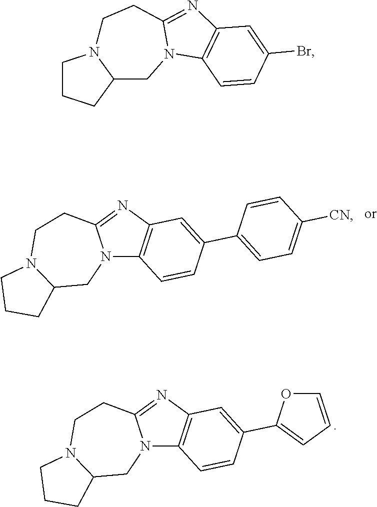

In one embodiment, R_{N} is (C₃-C₁₀)cycloalkyl optionally substituted with one or more R′, and at least one of R_{A} and R_{A}′ is not hydrogen. In one embodiment, R_{N} is (C₃-C₁₀)cycloalkyl optionally substituted with one or more R′; at least one of R_{A} and R_{A}′ is not hydrogen; and at least one of R₅, R₆, R₇, and R₈ is not hydrogen. In one embodiment, at least one of R_{A} and R_{A}′ is (C₁-C₁₀)alkyl optionally substituted with one or more R′. In one embodiment, at least one of R_{A} and R_{A}′ is (C₁-C₄)alkyl optionally substituted with one or more R′. In one embodiment, at least one of R_{A} and R_{A}′ is methyl. Specific examples include, but are not limited to, the following compounds:

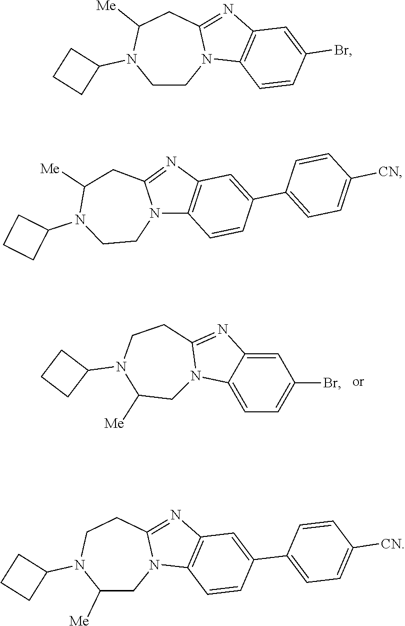

In one embodiment, m is 0 and n is 1. In one embodiment, R_{N} is (C₃-C₁₀)cycloalkyl optionally substituted with one or more R′, m is 0, and n is 1. Specific examples include, but are not limited to, the following compounds:

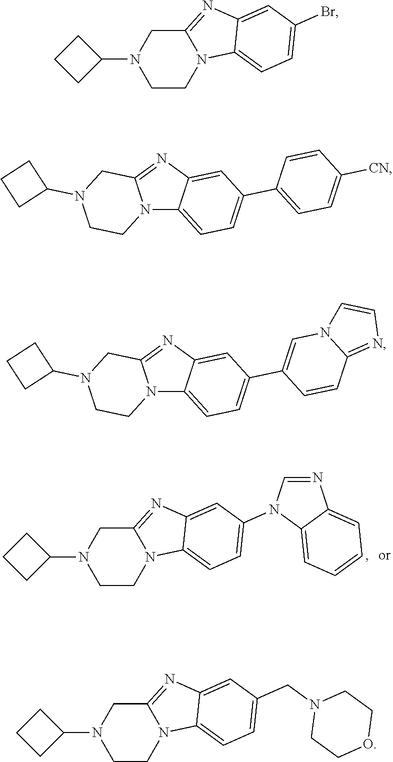

In one embodiment, R_{N} is (C₃-C₁₀)cycloalkyl optionally substituted with one or more R′; and R_{A} and R_{A}′ are taken together to form a 1-, 2-, or 3-atom bridge, which is optionally substituted with one or more R′. In certain embodiments, provided herein is a compound of formula (Iaa),

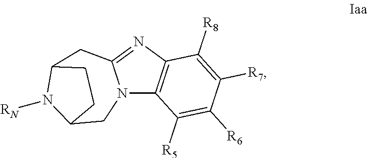

or a pharmaceutically acceptable salt or stereoisomer thereof, wherein

R₅, R₆, R₇ and R₈ are each independently hydrogen, halogen, cyano, (C₁-C₁₀)alkyl, (C₁-C₁₀)alkenyl, (C₃-C₁₀)cyclo alkyl, (6 to 10 membered)aryl, (C₁-C₁₀)heteroalkyl, (C₃-C₁₀)-heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₁; or two adjacent R₅, R₆, R₇, and R₈ may together form a 3 to 10 membered ring; and R_{N} and R₁ are defined herein elsewhere. Specific examples include, but are not limited to, the following compounds:

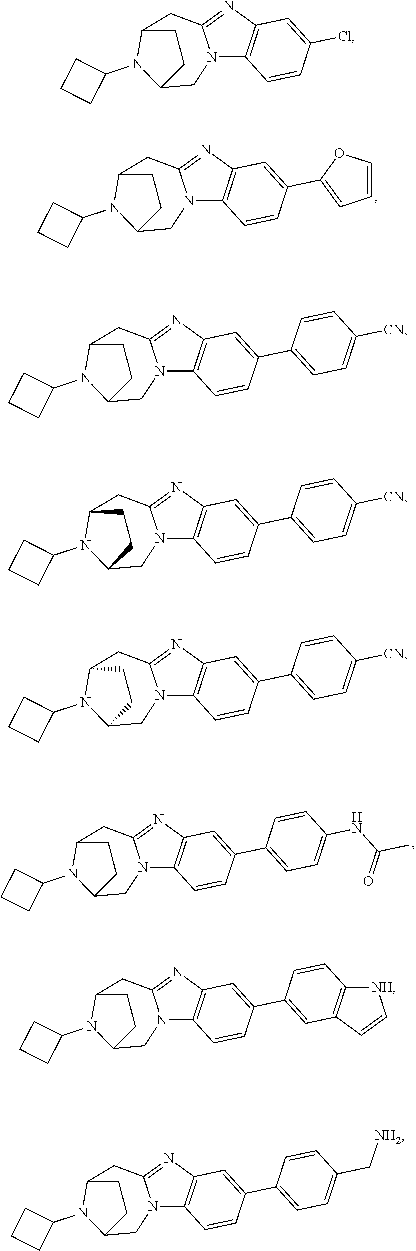

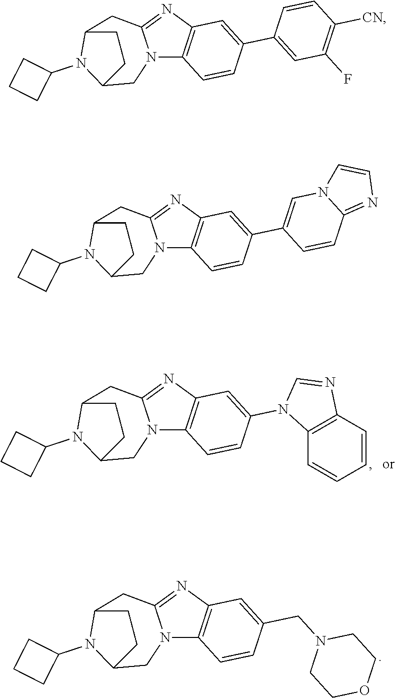

In certain embodiments, provided herein is a compound of formula (Ib),

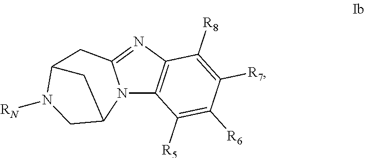

or a pharmaceutically acceptable salt or stereoisomer thereof, wherein

R₅, R₆, R₇ and R₈ are each independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)-heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₁; or two adjacent R₅, R₆, R₇, and R₈ may together form a 3 to 10 membered ring; and R_{N} and R₁ are defined herein elsewhere. Specific examples include, but are not limited to, the following compounds:

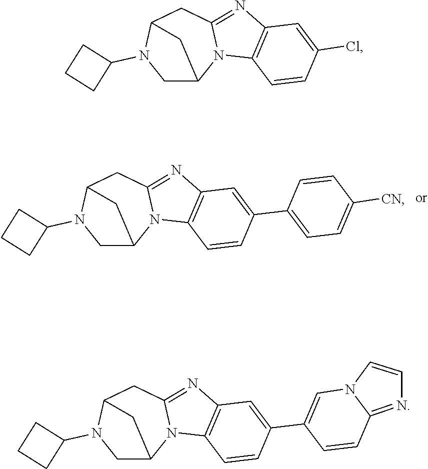

In certain embodiments, provided herein is a compound of formula (Ic),

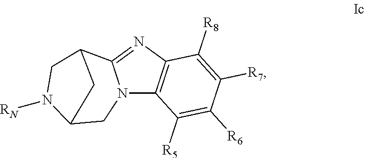

or a pharmaceutically acceptable salt or stereoisomer thereof, wherein

R₅, R₆, R₇ and R₈ are each independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)-heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₁; or two adjacent R₅, R₆, R₇, and R₈ may together form a 3 to 10 membered ring; and R_{N} and R₁ are defined herein elsewhere. Specific examples include, but are not limited to, the following compounds:

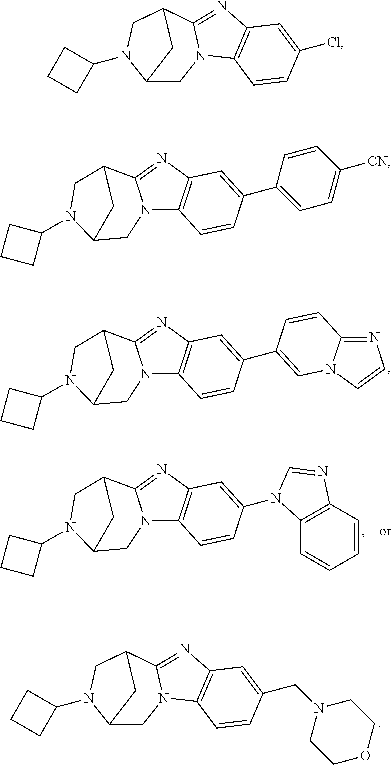

In certain embodiments, provided herein is a compound of formula (Id),

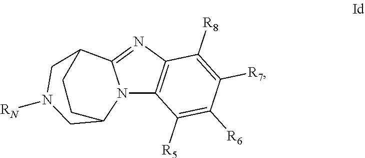

or a pharmaceutically acceptable salt or stereoisomer thereof, wherein

R₅, R₆, R₇ and R₈ are each independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)-heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₁; or two adjacent R₅, R₆, R₇, and R₈ may together form a 3 to 10 membered ring; and R_{N} and R₁ are defined herein elsewhere. Specific examples include, but are not limited to, the following compounds:

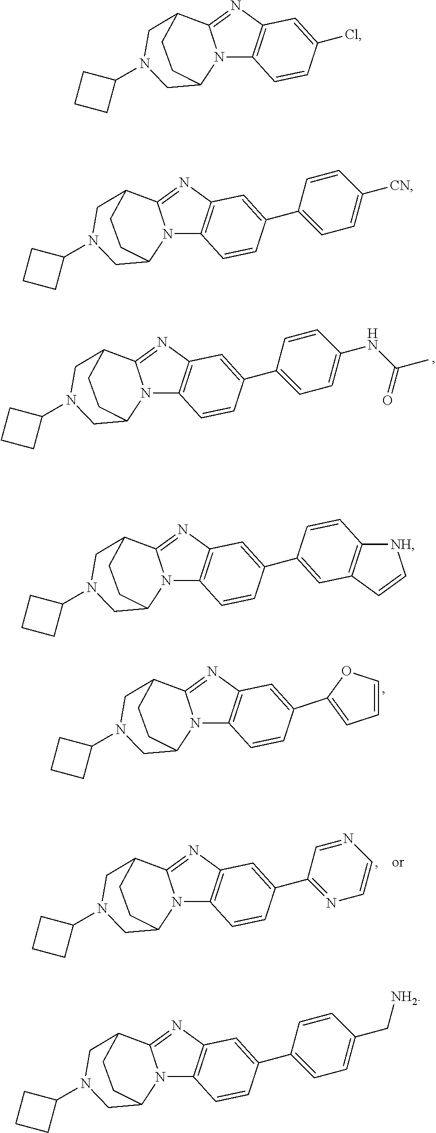

In one embodiment, R_{N} and R_{A} together with the atoms to which they are attached form a 3-, 4-, 5-, 6-, or 7-membered non-aromatic ring (e.g., a fully or partially saturated ring), which is optionally substituted with one or more R′. In one embodiment, R_{N} and R_{A} together with the atoms to which they are attached form an optionally substituted pyrrolidine ring. In one embodiment, R_{N} and R_{A} together with the atoms to which they are attached form an optionally substituted piperidine ring.

Any of the combinations of R₅, R₆, R₇, R₈, R_{N}, R_{A}, R_{A}′, R₁, and n are encompassed by this disclosure and specifically provided herein.

In one embodiment, provided herein is a compound of formula (II):

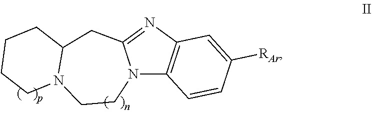

or a pharmaceutically acceptable salt, solvate, or stereoisomer thereof, wherein

R_{Ar} is hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered) heteroaryl, hydroxyl, alkoxyl, aminoalkyl, amino, imino, amido, carbonyl, thiol, sulfinyl, or sulfonyl, each of which may be optionally substituted with one or more R₁; p is 0, 1, or 2; n is 1 or 2; and R₁ is defined herein elsewhere.

In one embodiment, R_{Ar} is hydrogen. In another embodiment, R_{Ar} is halogen. In another embodiment, R_{Ar} is cyano. In another embodiment, R_{Ar} is (C₁-C₁₀)alkyl optionally substituted with one or more R₁. In another embodiment, R_{Ar} is (C₁-C₁₀)-alkenyl optionally substituted with one or more R₁. In another embodiment, R_{Ar} is (C₃-C₁₀)cycloalkyl optionally substituted with one or more R₁. In another embodiment, R_{Ar} is (6 to 10 membered)aryl optionally substituted with one or more R₁. In another embodiment, R_{Ar} is (C₁-C₁₀)heteroalkyl optionally substituted with one or more R₁. In another embodiment, R_{Ar} is (C₃-C₁₀)heterocycloalkyl optionally substituted with one or more R₁. In another embodiment, R_{Ar} is (5 to 10 membered)heteroaryl optionally substituted with one or more R₁. In another embodiment, R_{Ar} is hydroxyl optionally substituted with one or more R₁. In another embodiment, R_{Ar} is alkoxyl optionally substituted with one or more R₁. In another embodiment, R_{Ar} is aminoalkyl optionally substituted with one or more R₁. In another embodiment, R_{Ar} is amino optionally substituted with one or more R₁. In another embodiment, R_{Ar} is imino optionally substituted with one or more R₁. In another embodiment, R_{Ar} is amido optionally substituted with one or more R₁. In another embodiment, R_{Ar} is carbonyl optionally substituted with one or more R₁. In another embodiment, R_{Ar} is thiol optionally substituted with one or more R₁. In another embodiment, R_{Ar} is sulfinyl optionally substituted with one or more R₁. In another embodiment, R_{Ar} is sulfonyl optionally substituted with one or more R₁. In one embodiment, R_{Ar} is fluorine. In another embodiment, R_{Ar} is chlorine. In another embodiment, R_{Ar} is bromine. In another embodiment, R_{Ar} is iodine. In another embodiment, R_{Ar} is cyano. In another embodiment, R_{Ar} is —OR₁. In another embodiment, R_{Ar} is —OCH₂R₁. In another embodiment, R_{Ar} is —NHR₁. In another embodiment, R_{Ar} is —NHCH₂R₁. In another embodiment, R_{Ar} is —N(R₁)₂. In another embodiment, R_{Ar} is —C(O)R₁. In another embodiment, R_{Ar} is —C(O)N(R₁)₂. In another embodiment, R_{Ar} is —CH₂R₁. In another embodiment, R_{Ar} is —CH₂N(R₁)₂. In another embodiment, R_{Ar} is —CH₂OR₁. R₁ is defined herein elsewhere.

In one embodiment, p is 0. In another embodiment, p is 1. In another embodiment, p is 2. In one embodiment, p is 0 or 1.

In one embodiment, n is 1. In another embodiment, n is 2. In another embodiment, n is 3. In one embodiment, n is 1 or 2.

In one embodiment, p is 1 and n is 1. In one embodiment, p is 1 and n is 2. In one embodiment, p is 0 and n is 1. In one embodiment, p is 0 and n is 2.

In one embodiment, n is 1, and thus, provided herein is a compound of formula (IIa):

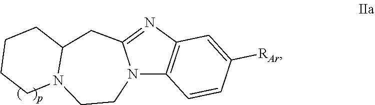

or a pharmaceutically acceptable salt or stereoisomer thereof, wherein

R_{Ar} is hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₁; p is 0, 1, or 2; and R₁ is defined herein elsewhere.

In one embodiment, when n is 1, p is 0. Specific examples include, but are not limited to, the following compounds:

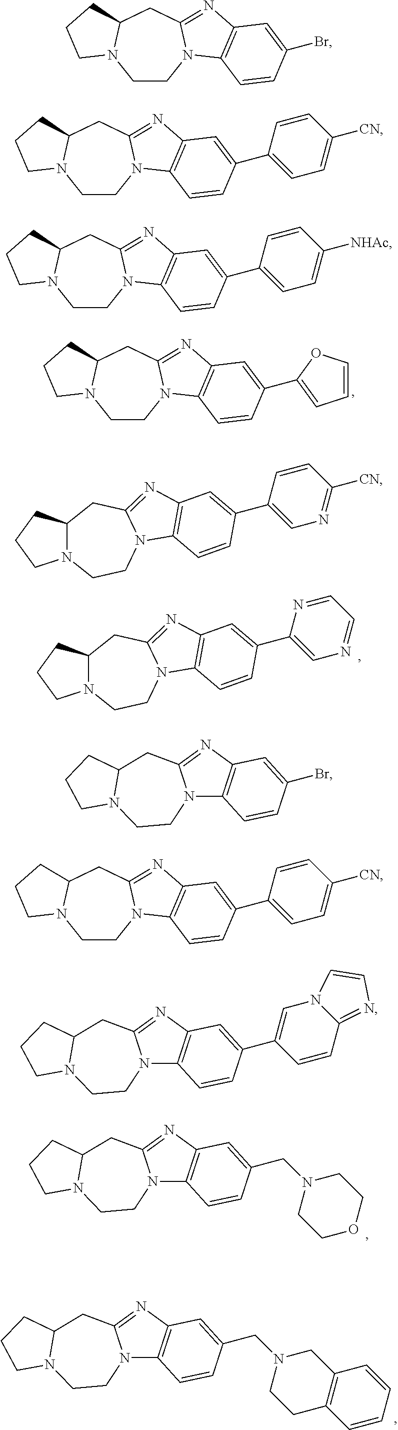

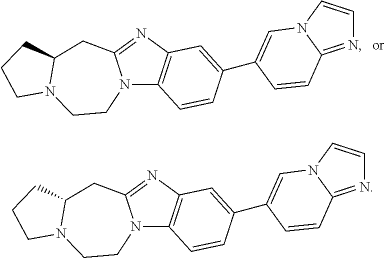

In one embodiment, when n is 1, p is 1. Specific examples include, but are not limited to, the following compounds:

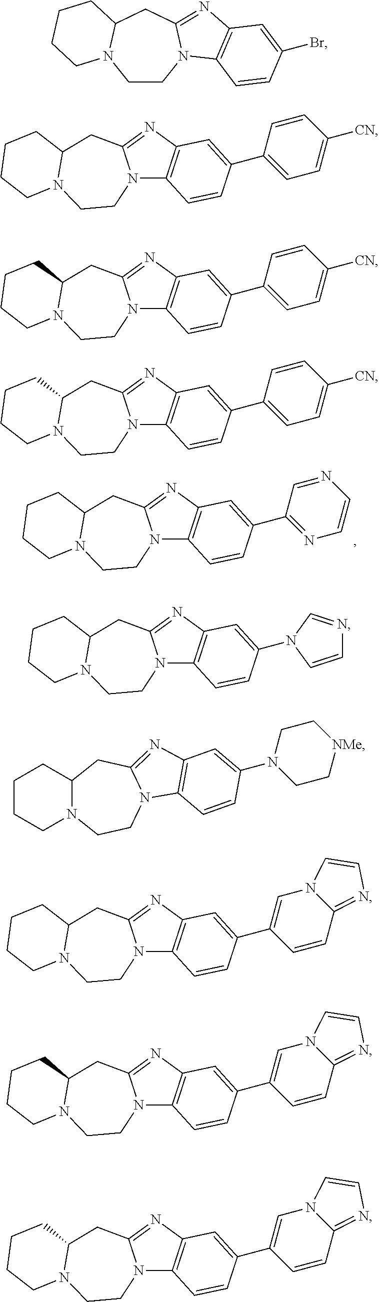

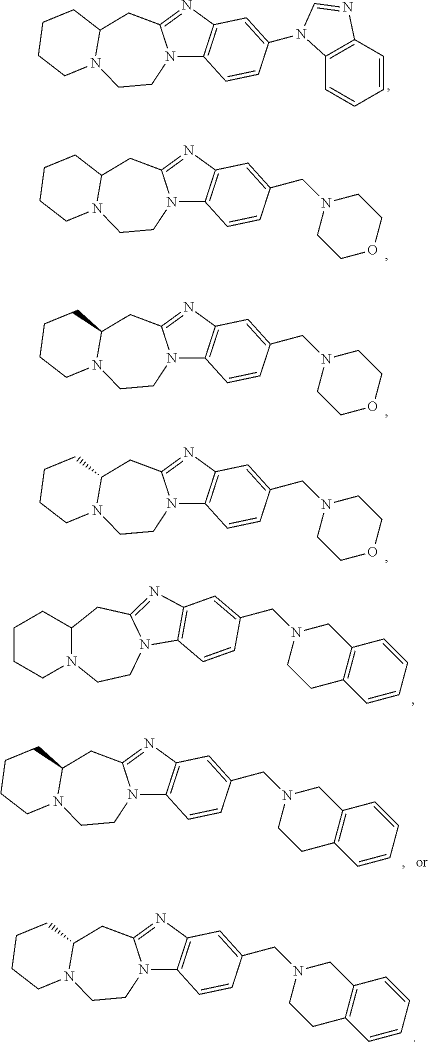

In one embodiment, n is 2, and thus, provided herein is a compound of formula (IIb):

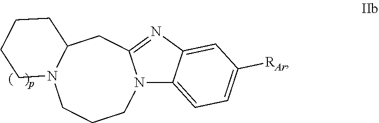

or a pharmaceutically acceptable salt or stereoisomer thereof, wherein

R_{Ar} is hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₁; p is 0, 1, or 2; and R₁ is defined herein elsewhere.

In one embodiment, when n is 2, p is 1. Specific examples include, but are not limited to, the following compounds:

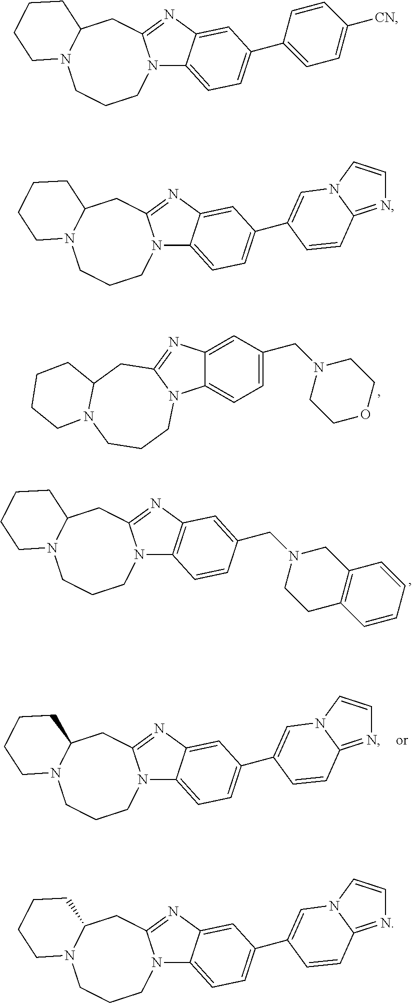

In one embodiment, when n is 2, p is 0. Specific examples include, but are not limited to, the following compounds:

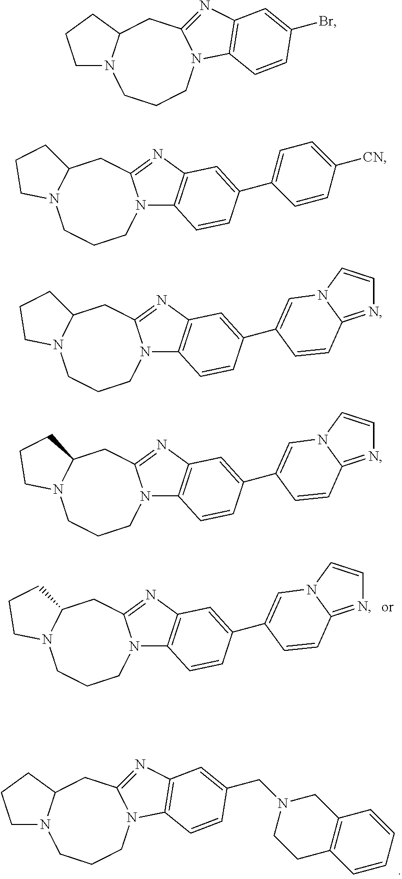

Any of the combinations of R_{Ar}, p, n, and R₁ are encompassed by this disclosure and specifically provided herein.

In one embodiment, provided herein is a compound of formula (Ia) as provided herein, or a pharmaceutically acceptable salt or stereoisomer thereof, wherein R_{A} and R_{A}′ are hydrogen, and n is 2. Accordingly, in one embodiment, provided herein is a compound of formula (III):

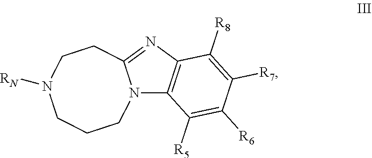

or a pharmaceutically acceptable salt or stereoisomer thereof, wherein

R₅, R₆, R₇ and R₈ are each independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)-heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₁; or two adjacent R₅, R₆, R₇, and R₈ may together form a 3 to 10 membered ring; and R_{N} and R₁ are defined herein elsewhere.

In one embodiment, R_{N} is a bond, hydrogen, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, or (5 to 10 membered)heteroaryl, each of which may be optionally substituted with one or more R′. In one embodiment, R_{N} is cyclobutyl optionally substituted with one or more R′. R′ is defined herein elsewhere.

In one embodiment, R₅ is hydrogen. In another embodiment, R₅ is halogen. In another embodiment, R₅ is cyano. In another embodiment, R₅ is (C₁-C₁₀)alkyl optionally substituted with one or more R₁. In another embodiment, R₅ is (C₁-C₁₀)-alkenyl optionally substituted with one or more R₁. In another embodiment, R₅ is (C₃-C₁₀)cycloalkyl optionally substituted with one or more R₁. In another embodiment, R₅ is (6 to 10 membered)aryl optionally substituted with one or more R₁. In another embodiment, R₅ is (C₁-C₁₀)heteroalkyl optionally substituted with one or more R₁. In another embodiment, R₅ is (C₃-C₁₀)heterocycloalkyl optionally substituted with one or more R₁. In another embodiment, R₅ is (5 to 10 membered)heteroaryl optionally substituted with one or more R₁. In another embodiment, R₅ is hydroxyl optionally substituted with R₁. In another embodiment, R₅ is alkoxyl optionally substituted with one or more R₁. In another embodiment, R₅ is aminoalkyl optionally substituted with one or more R₁. In another embodiment, R₅ is amino optionally substituted with one or more R₁. In another embodiment, R₅ is imino optionally substituted with one or more R₁. In another embodiment, R₅ is amido optionally substituted with one or more R₁. In another embodiment, R₅ is carbonyl optionally substituted with one or more R₁. In another embodiment, R₅ is thiol optionally substituted with one or more R₁. In another embodiment, R₅ is sulfinyl optionally substituted with one or more R₁. In another embodiment, R₅ is sulfonyl optionally substituted with one or more R₁. R₁ is defined herein elsewhere.

In one embodiment, R₆ is hydrogen. In another embodiment, R₆ is halogen. In another embodiment, R₆ is cyano. In another embodiment, R₆ is (C₁-C₁₀)alkyl optionally substituted with one or more R₁. In another embodiment, R₆ is (C₁-C₁₀)-alkenyl optionally substituted with one or more R₁. In another embodiment, R₆ is (C₃-C₁₀)cycloalkyl optionally substituted with one or more R₁. In another embodiment, R₆ is (6 to 10 membered)aryl optionally substituted with one or more R₁. In another embodiment, R₆ is (C₁-C₁₀)heteroalkyl optionally substituted with one or more R₁. In another embodiment, R₆ is (C₃-C₁₀)heterocycloalkyl optionally substituted with one or more R₁. In another embodiment, R₆ is (5 to 10 membered)heteroaryl optionally substituted with one or more R₁. In another embodiment, R₆ is hydroxyl optionally substituted with R₁. In another embodiment, R₆ is alkoxyl optionally substituted with one or more R₁. In another embodiment, R₆ is aminoalkyl optionally substituted with one or more R₁. In another embodiment, R₆ is amino optionally substituted with one or more R₁. In another embodiment, R₆ is imino optionally substituted with one or more R₁. In another embodiment, R₆ is amido optionally substituted with one or more R₁. In another embodiment, R₆ is carbonyl optionally substituted with one or more R₁. In another embodiment, R₆ is thiol optionally substituted with one or more R₁. In another embodiment, R₆ is sulfinyl optionally substituted with one or more R₁. In another embodiment, R₆ is sulfonyl optionally substituted with one or more R₁. R₁ is defined herein elsewhere.

In one embodiment, R₇ is hydrogen. In another embodiment, R₇ is halogen. In another embodiment, R₇ is cyano. In another embodiment, R₇ is (C₁-C₁₀)alkyl optionally substituted with one or more R₁. In another embodiment, R₇ is (C₁-C₁₀)-alkenyl optionally substituted with one or more R₁. In another embodiment, R₇ is (C₃-C₁₀)cycloalkyl optionally substituted with one or more R₁. In another embodiment, R₇ is (6 to 10 membered)aryl optionally substituted with one or more R₁. In another embodiment, R₇ is (C₁-C₁₀)heteroalkyl optionally substituted with one or more R₁. In another embodiment, R₇ is (C₃-C₁₀)heterocycloalkyl optionally substituted with one or more R₁. In another embodiment, R₇ is (5 to 10 membered)heteroaryl optionally substituted with one or more R₁. In another embodiment, R₇ is hydroxyl optionally substituted with R₁. In another embodiment, R₇ is alkoxyl optionally substituted with one or more R₁. In another embodiment, R₇ is aminoalkyl optionally substituted with one or more R₁. In another embodiment, R₇ is amino optionally substituted with one or more R₁. In another embodiment, R₇ is imino optionally substituted with one or more R₁. In another embodiment, R₇ is amido optionally substituted with one or more R₁. In another embodiment, R₇ is carbonyl optionally substituted with one or more R₁. In another embodiment, R₇ is thiol optionally substituted with one or more R₁. In another embodiment, R₇ is sulfinyl optionally substituted with one or more R₁. In another embodiment, R₇ is sulfonyl optionally substituted with one or more R₁. R₁ is defined herein elsewhere.

In one embodiment, R₈ is hydrogen. In another embodiment, R₈ is halogen. In another embodiment, R₈ is cyano. In another embodiment, R₈ is (C₁-C₁₀)alkyl optionally substituted with one or more R₁. In another embodiment, R₈ is (C₁-C₁₀)-alkenyl optionally substituted with one or more R₁. In another embodiment, R₈ is (C₃-C₁₀)cycloalkyl optionally substituted with one or more R₁. In another embodiment, R₈ is (6 to 10 membered)aryl optionally substituted with one or more R₁. In another embodiment, R₈ is (C₁-C₁₀)heteroalkyl optionally substituted with one or more R₁. In another embodiment, R₈ is (C₃-C₁₀)heterocycloalkyl optionally substituted with one or more R₁. In another embodiment, R₈ is (5 to 10 membered)heteroaryl optionally substituted with one or more R₁. In another embodiment, R₈ is hydroxyl optionally substituted with R₁. In another embodiment, R₈ is alkoxyl optionally substituted with one or more R₁. In another embodiment, R₈ is aminoalkyl optionally substituted with one or more R₁. In another embodiment, R₈ is amino optionally substituted with one or more R₁. In another embodiment, R₈ is imino optionally substituted with one or more R₁. In another embodiment, R₈ is amido optionally substituted with one or more R₁. In another embodiment, R₈ is carbonyl optionally substituted with one or more R₁. In another embodiment, R₈ is thiol optionally substituted with one or more R₁. In another embodiment, R₈ is sulfinyl optionally substituted with one or more R₁. In another embodiment, R₈ is sulfonyl optionally substituted with one or more R₁. R₁ is defined herein elsewhere.

In one embodiment, R₅ and R₆ together form a 3 to 10 membered ring, which is optionally substituted with one or more R₁. In another embodiment, R₆ and R₇ together form a 3 to 10 membered ring, which is optionally substituted with one or more R₁. In another embodiment, R₇ and R₈ together form a 3 to 10 membered ring, which is optionally substituted with one or more R₁. R₁ is defined herein elsewhere.

In another embodiment, R₅, R₆, R₇, and R₈ are each independently (i) hydrogen, halogen, or cyano; (ii) (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered)heteroaryl, alkoxyl, aminoalkyl, amino, imino, amido, carbonyl, thiol, sulfinyl, or sulfonyl, each of which is optionally substituted with one or more R₁; (iii) hydroxyl substituted with one or more R₁′; or (iv) two adjacent R₅, R₆, R₇, and R₈ together form a 3 to 10 membered ring optionally substituted with one or more R₁; wherein each occurrence of R₁′ is independently —C(O)NR₃R₄, —C(O)R₃, (C₃-C₁₀)cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂; and R₂, R₃, and R₄ are defined herein elsewhere.

In one embodiment, R₁′ is independently —C(O)NR₃R₄, —C(O)R₃, (C₃-C₁₀)cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. R₂, R₃, and R₄ are defined herein elsewhere.

In one embodiment, R₁′ is —C(O)NR₃R₄. In another embodiment, R₁′ is —C(O)R₃. In another embodiment, R₁′ is (C₃-C₁₀)cycloalkyl optionally substituted with one or more R₂. In another embodiment, R₁′ is (C₆-C₁₂)aralkyl optionally substituted with one or more R₂. In another embodiment, R₁′ is (6 to 10 membered)aryl optionally substituted with one or more R₂. In another embodiment, R₁′ is (C₁-C₁₀)heteroalkyl optionally substituted with one or more R₂. In another embodiment, R₁′ is (C₃-C₁₀)heterocycloalkyl optionally substituted with one or more R₂. In another embodiment, R₁′ is (5 to 10 membered) heteroaryl optionally substituted with one or more R₂. R₂, R₃, and R₄ are defined herein elsewhere.

In one embodiment, R₅ is hydrogen. In another embodiment, R₅ is halogen. In another embodiment, R₅ is cyano. In another embodiment, R₅ is (C₁-C₁₀)alkyl optionally substituted with one or more R₁. In another embodiment, R₅ is (C₁-C₁₀)alkyl substituted with one or more R₁′. In another embodiment, R₅ is (C₁-C₁₀) alkenyl optionally substituted with one or more R₁. In another embodiment, R₅ is (C₃-C₁₀)cycloalkyl optionally substituted with one or more R₁. In another embodiment, R₅ is (6 to 10 membered)aryl optionally substituted with one or more R₁. In another embodiment, R₅ is (C₁-C₁₀)heteroalkyl optionally substituted with one or more R₁. In another embodiment, R₅ is (C₃-C₁₀)heterocycloalkyl optionally substituted with one or more R₁. In another embodiment, R₅ is (5 to 10 membered)heteroaryl optionally substituted with one or more R₁. In another embodiment, R₅ is hydroxyl optionally substituted with R₁. In another embodiment, R₅ is hydroxyl substituted with R₁′. In another embodiment, R₅ is alkoxyl optionally substituted with one or more R₁. In another embodiment, R₅ is alkoxyl substituted with one or more R₁′. In another embodiment, R₅ is aminoalkyl optionally substituted with one or more R₁. In another embodiment, R₅ is amino optionally substituted with one or more R₁. In another embodiment, R₅ is imino optionally substituted with one or more R₁. In another embodiment, R₅ is amido optionally substituted with one or more R₁. In another embodiment, R₅ is carbonyl optionally substituted with one or more R₁. In another embodiment, R₅ is thiol optionally substituted with one or more R₁. In another embodiment, R₅ is sulfinyl optionally substituted with one or more R₁. In another embodiment, R₅ is sulfonyl optionally substituted with one or more R₁. R₁ and R₁′ are defined herein elsewhere.

In one embodiment, R₆ is hydrogen. In another embodiment, R₆ is halogen. In another embodiment, R₆ is cyano. In another embodiment, R₆ is (C₁-C₁₀)alkyl optionally substituted with one or more R₁. In another embodiment, R₆ is (C₁-C₁₀)alkyl substituted with one or more R₁′. In another embodiment, R₆ is (C₁-C₁₀)-alkenyl optionally substituted with one or more R₁. In another embodiment, R₆ is (C₃-C₁₀)cycloalkyl optionally substituted with one or more R₁. In another embodiment, R₆ is (6 to 10 membered)aryl optionally substituted with one or more R₁. In another embodiment, R₆ is (C₁-C₁₀)heteroalkyl optionally substituted with one or more R₁. In another embodiment, R₆ is (C₃-C₁₀)heterocycloalkyl optionally substituted with one or more R₁. In another embodiment, R₆ is (5 to 10 membered)heteroaryl optionally substituted with one or more R₁. In another embodiment, R₆ is hydroxyl optionally substituted with R₁. In another embodiment, R₆ is hydroxyl substituted with R₁′. In another embodiment, R₆ is alkoxyl optionally substituted with one or more R₁. In another embodiment, R₆ is alkoxyl substituted with one or more R₁′. In another embodiment, R₆ is aminoalkyl optionally substituted with one or more R₁. In another embodiment, R₆ is amino optionally substituted with one or more R₁. In another embodiment, R₆ is imino optionally substituted with one or more R₁. In another embodiment, R₆ is amido optionally substituted with one or more R₁. In another embodiment, R₆ is carbonyl optionally substituted with one or more R₁. In another embodiment, R₆ is thiol optionally substituted with one or more R₁. In another embodiment, R₆ is sulfinyl optionally substituted with one or more R₁. In another embodiment, R₆ is sulfonyl optionally substituted with one or more R₁. R₁ and R₁′ are defined herein elsewhere.

In one embodiment, R₇ is hydrogen. In another embodiment, R₇ is halogen. In another embodiment, R₇ is cyano. In another embodiment, R₇ is (C₁-C₁₀)alkyl optionally substituted with one or more R₁. In another embodiment, R₇ is (C₁-C₁₀)alkyl substituted with one or more R₁′. In another embodiment, R₇ is (C₁-C₁₀)-alkenyl optionally substituted with one or more R₁. In another embodiment, R₇ is (C₃-C₁₀)cycloalkyl optionally substituted with one or more R₁. In another embodiment, R₇ is (6 to 10 membered)aryl optionally substituted with one or more R₁. In another embodiment, R₇ is (C₁-C₁₀)heteroalkyl optionally substituted with one or more R₁. In another embodiment, R₇ is (C₃-C₁₀)heterocycloalkyl optionally substituted with one or more R₁. In another embodiment, R₇ is (5 to 10 membered)heteroaryl optionally substituted with one or more R₁. In another embodiment, R₇ is hydroxyl optionally substituted with R₁. In another embodiment, R₇ is hydroxyl substituted with R₁′. In another embodiment, R₇ is alkoxyl optionally substituted with one or more R₁. In another embodiment, R₇ is alkoxyl substituted with one or more R₁′. In another embodiment, R₇ is aminoalkyl optionally substituted with one or more R₁. In another embodiment, R₇ is amino optionally substituted with one or more R₁. In another embodiment, R₇ is imino optionally substituted with one or more R₁. In another embodiment, R₇ is amido optionally substituted with one or more R₁. In another embodiment, R₇ is carbonyl optionally substituted with one or more R₁. In another embodiment, R₇ is thiol optionally substituted with one or more R₁. In another embodiment, R₇ is sulfinyl optionally substituted with one or more R₁. In another embodiment, R₇ is sulfonyl optionally substituted with one or more R₁. R₁ and R₁′ are defined herein elsewhere.

In one embodiment, R₈ is hydrogen. In another embodiment, R₈ is halogen. In another embodiment, R₈ is cyano. In another embodiment, R₈ is (C₁-C₁₀)alkyl optionally substituted with one or more R₁. In another embodiment, R₈ is (C₁-C₁₀)alkyl substituted with one or more R₁′. In another embodiment, R₈ is (C₁-C₁₀)alkyl optionally substituted with one or more R₁. In another embodiment, R₈ is (C₁-C₁₀)-alkenyl optionally substituted with one or more R₁. In another embodiment, R₈ is (C₃-C₁₀)cycloalkyl optionally substituted with one or more R₁. In another embodiment, R₈ is (6 to 10 membered)aryl optionally substituted with one or more R₁. In another embodiment, R₈ is (C₁-C₁₀)heteroalkyl optionally substituted with one or more R₁. In another embodiment, R₈ is (C₃-C₁₀)heterocycloalkyl optionally substituted with one or more R₁. In another embodiment, R₈ is (5 to 10 membered)heteroaryl optionally substituted with one or more R₁. In another embodiment, R₈ is hydroxyl optionally substituted with R₁. In another embodiment, R₈ is hydroxyl substituted with R₁′. In another embodiment, R₈ is alkoxyl optionally substituted with one or more R₁. In another embodiment, R₈ is alkoxyl substituted with one or more R₁′. In another embodiment, R₈ is aminoalkyl optionally substituted with one or more R₁. In another embodiment, R₈ is amino optionally substituted with one or more R₁. In another embodiment, R₈ is imino optionally substituted with one or more R₁. In another embodiment, R₈ is amido optionally substituted with one or more R₁. In another embodiment, R₈ is carbonyl optionally substituted with one or more R₁. In another embodiment, R₈ is thiol optionally substituted with one or more R₁. In another embodiment, R₈ is sulfinyl optionally substituted with one or more R₁. In another embodiment, R₈ is sulfonyl optionally substituted with one or more R₁. R₁ and R₁′ are defined herein elsewhere.

In one embodiment, R₅ is (C₁-C₁₀)alkyl or alkoxyl, each of which is substituted with one or more cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, —S(O)₂NR₃R₄, (C₃-C₁₀)cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. In one embodiment, R₆ is (C₁-C₁₀)alkyl or alkoxyl, each of which is substituted with one or more cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, —S(O)₂NR₃R₄, (C₃-C₁₀)cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. In one embodiment, R₇ is (C₁-C₁₀)alkyl or alkoxyl, each of which is substituted with one or more cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, —S(O)₂NR₃R₄, (C₃-C₁₀)cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. In one embodiment, R₈ is (C₁-C₁₀)alkyl or alkoxyl, each of which is substituted with one or more cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, —S(O)₂NR₃R₄, (C₃-C₁₀)cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂.

In one embodiment, R₅ is hydroxyl substituted with one or more —C(O)NR₃R₄, —C(O)R₃, (C₃-C₁₀)cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. In one embodiment, R₆ is hydroxyl substituted with one or more —C(O)NR₃R₄, —C(O)R₃, (C₃-C₁₀)cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. In one embodiment, R₇ is hydroxyl substituted with one or more —C(O)NR₃R₄, —C(O)R₃, (C₃-C₁₀)cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. In one embodiment, R₈ is hydroxyl substituted with one or more —C(O)NR₃R₄, —C(O)R₃, (C₃-C₁₀)cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂.

In one embodiment, R₅ is (C₁-C₁₀)alkyl or alkoxyl, each of which is substituted with one or more cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, —S(O)₂NR₃R₄, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. In one embodiment, R₆ is (C₁-C₁₀)alkyl or alkoxyl, each of which is substituted with one or more cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, —S(O)₂NR₃R₄, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. In one embodiment, R₇ is (C₁-C₁₀)alkyl or alkoxyl, each of which is substituted with one or more cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, —S(O)₂NR₃R₄, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. In one embodiment, R₈ is (C₁-C₁₀)alkyl or alkoxyl, each of which is substituted with one or more cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, —S(O)₂NR₃R₄, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂.

In one embodiment, R₅ is hydroxyl substituted with one or more —C(O)NR₃R₄, —C(O)R₃, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. In one embodiment, R₆ is hydroxyl substituted with one or more —C(O)NR₃R₄, —C(O)R₃, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. In one embodiment, R₇ is hydroxyl substituted with one or more —C(O)NR₃R₄, —C(O)R₃, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. In one embodiment, R₈ is hydroxyl substituted with one or more —C(O)NR₃R₄, —C(O)R₃, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂.

Any of the combinations of R₅, R₆, R₇, R₈, R_{N}, R′, R₁, R₁′, R₂, R₃, and R₄ are encompassed by this disclosure and specifically provided herein.

In one embodiment, provided herein is a compound of formula (IV):

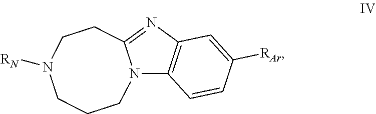

or a pharmaceutically acceptable salt or stereoisomer thereof, wherein R_{N} and R_{Ar} are defined herein elsewhere.

In one embodiment, R_{Ar} is hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₁; and R₁ is defined herein elsewhere. In one embodiment, R_{N} is a bond, hydrogen, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocyclo-alkyl, or (5 to 10 membered)heteroaryl, each of which may be optionally substituted with one or more R′. In one embodiment, R_{N} is cyclobutyl optionally substituted with one or more R′. R′ is defined herein elsewhere.

In one embodiment, R_{Ar} is halogen, (6 to 10 membered)aryl optionally substituted with one or more R₁, or (5 to 10 membered)heteroaryl optionally substituted with one or more R₁. In one embodiment, R_{Ar} is halogen. In one embodiment, R_{Ar} is (6 to 10 membered)aryl or (5 to 10 membered)heteroaryl, each of which is optionally substituted with one or more R₁. In one embodiment, R_{Ar} is halogen, (6-membered)aryl optionally substituted with one or more R₁, or (5 to 10 membered)-heteroaryl optionally substituted with one or more R₁. In one embodiment, R_{Ar} is halogen, phenyl optionally substituted with one or more R₁, or (5 to 10 membered)-heteroaryl optionally substituted with one or more R₁. In one embodiment, R_{Ar} is halogen, phenyl optionally substituted with one or more R₁, or (9 to 10 membered)-heteroaryl optionally substituted with one or more R₁. Specific examples include, but are not limited to, the following compounds:

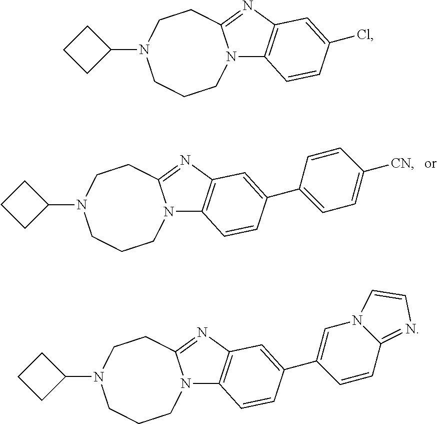

In one embodiment, R_{Ar} is (C₁-C₁₀)alkyl or alkoxyl, each of which is substituted with one or more halogen, cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, —S(O)₂NR₃R₄, (C₃-C₁₀) cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂. In one embodiment, R_{Ar} is (C₁-C₁₀)alkyl substituted with one or more (C₃-C₁₀)cycloalkyl optionally substituted with one or more R₂, (6 to 10 membered)aryl optionally substituted with one or more R₂, (C₃-C₁₀)heterocycloalkyl optionally substituted with one or more R₂, or (5 to 10 membered)heteroaryl optionally substituted with one or more R₂. In one embodiment, R_{Ar} is (C₁-C₁₀)alkyl substituted with (C₃-C₁₀)heterocycloalkyl optionally substituted with one or more R₂ or (5 to 10 membered)heteroaryl optionally substituted with one or more R₂. In one embodiment, R_{Ar} is methyl substituted with (C₃-C₁₀)heterocycloalkyl optionally substituted with one or more R₂ or (5 to 10 membered)heteroaryl optionally substituted with one or more R₂. Specific examples include, but are not limited to, the following compounds:

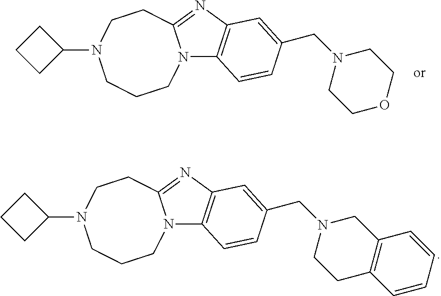

Any of the combinations of R_{N} and R_{Ar} are encompassed by this disclosure and specifically provided herein.

In one embodiment, R_{Ar} is (i) hydrogen, halogen, or cyano; (ii) (C₁-C₁₀)alkyl, (C₁-C₁₀)alkenyl, (C₃-C₁₀)cycloalkyl, (6 to 10 membered)aryl, (C₁-C₁₀)hetero alkyl, (C₃-C₁₀)heterocycloalkyl, (5 to 10 membered)heteroaryl, alkoxyl, aminoalkyl, amino, imino, amido, carbonyl, thiol, sulfinyl, or sulfonyl, each of which is optionally substituted with one or more R₁; or (iii) hydroxyl substituted with one or more R₁′; and R₁ and R₁′ are defined herein elsewhere.

In one embodiment, R_{Ar} is (i) hydrogen, halogen, or cyano; (ii) (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered)heteroaryl, aminoalkyl, amino, imino, amido, carbonyl, thiol, sulfinyl, or sulfonyl, each of which is optionally substituted with one or more R₁; or (iii) (C₁-C₁₀)alkyl, hydroxyl, or alkoxyl, each of which is substituted with one or more R₁′; and R₁ and R₁′ are defined herein elsewhere.

In one embodiment, R_{Ar} is (i) cyano; (ii) (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered)heteroaryl, alkoxyl, aminoalkyl, amino, imino, amido, carbonyl, thiol, sulfinyl, or sulfonyl, each of which is optionally substituted with one or more R₁; or (iii) hydroxyl substituted with one or more R₁′. In one embodiment, R_{Ar} is not (C₁-C₄)alkyl or (C₁-C₄)alkoxyl optionally substituted with one or more halogen. R₁ and R₁′ are defined herein elsewhere.

In one embodiment, R_{Ar} is not (C₁-C₄)alkyl. In one embodiment, R_{Ar} is not (C₁-C₄)alkyl optionally substituted with one or more halogen. In one embodiment, R_{Ar} is not (C₁-C₄)alkyl optionally substituted with cycloalkyl. In one embodiment, R_{Ar} is not (C₁-C₄)alkoxyl optionally substituted with one or more halogen. In one embodiment, R_{Ar} is not (C₁-C₄)alkoxyl optionally substituted with cycloalkyl.

In one embodiment, R_{Ar} is (i) cyano; (ii) (6 to 10 membered)aryl, (C₁-C₁₀)heteroalkyl, (C₃-C₁₀)heterocycloalkyl, (5 to 10 membered)heteroaryl, aminoalkyl, amino, amido, or carbonyl, each of which is optionally substituted with one or more R₁; or (iii) (C₁-C₁₀)alkyl, alkoxyl, or hydroxyl, each of which is substituted with one or more R₁′.

In one embodiment, R_{Ar} is (C₁-C₁₀)alkyl or alkoxyl, each of which is substituted with one or more halogen, cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, —S(O)₂NR₃R₄, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂.

In one embodiment, R_{Ar} is hydrogen. In another embodiment, R_{Ar} is halogen. In another embodiment, R_{Ar} is cyano. In another embodiment, R_{Ar} is (C₁-C₁₀)alkyl optionally substituted with one or more R₁. In another embodiment, R_{Ar} is (C₁-C₁₀)alkyl substituted with one or more R₁′. In another embodiment, R_{Ar} is (C₁-C₁₀)alkenyl optionally substituted with one or more R₁. In another embodiment, R_{Ar} is (C₃-C₁₀)cycloalkyl optionally substituted with one or more R₁. In another embodiment, R_{Ar} is (6 to 10 membered)aryl optionally substituted with one or more R₁. In another embodiment, R_{Ar} is (C₁-C₁₀)heteroalkyl optionally substituted with one or more R₁. In another embodiment, R_{Ar} is (C₃-C₁₀)heterocycloalkyl optionally substituted with one or more R₁. In another embodiment, R_{Ar} is (5 to 10 membered)heteroaryl optionally substituted with one or more R₁. In another embodiment, R_{Ar} is hydroxyl optionally substituted with one or more R₁. In another embodiment, R_{Ar} is hydroxyl substituted with one or more R₁′. In another embodiment, R_{Ar} is alkoxyl optionally substituted with one or more R₁. In another embodiment, R_{Ar} is alkoxyl substituted with one or more R₁′. In another embodiment, R_{Ar} is aminoalkyl optionally substituted with one or more R₁. In another embodiment, R_{Ar} is amino optionally substituted with one or more R₁. In another embodiment, R_{Ar} is imino optionally substituted with one or more R₁. In another embodiment, R_{Ar} is amido optionally substituted with one or more R₁. In another embodiment, R_{Ar} is carbonyl optionally substituted with one or more R₁. In another embodiment, R_{Ar} is thiol optionally substituted with one or more R₁. In another embodiment, R_{Ar} is sulfinyl optionally substituted with one or more R₁. In another embodiment, R_{Ar} is sulfonyl optionally substituted with one or more R₁. In one embodiment, R_{Ar} is fluorine. In another embodiment, R_{Ar} is chlorine. In another embodiment, R_{Ar} is bromine. In another embodiment, R_{Ar} is iodine. In another embodiment, R_{Ar} is cyano. In another embodiment, R_{Ar} is —OR₁. In another embodiment, R_{Ar} is —OR₁′. In another embodiment, R_{Ar} is —OCH₂R₁. In another embodiment, R_{Ar} is —OCH₂R₁′. In another embodiment, R_{Ar} is —NHR₁. In another embodiment, R_{Ar} is —NHCH₂R₁. In another embodiment, R_{Ar} is —N(R₁)₂. In another embodiment, R_{Ar} is —C(O)R₁. In another embodiment, R_{Ar} is —C(O)N(R₁)₂. In another embodiment, R_{Ar} is —CH₂R₁. In another embodiment, R_{Ar} is —CH₂R₁′. In another embodiment, R_{Ar} is —CH₂N(R₁)₂. In another embodiment, R_{Ar} is —CH₂OR₁. In another embodiment, R_{Ar} is —CH₂OR₁′. R₁ and R₁′ are defined herein elsewhere.

In one embodiment, R_{Ar} is fluorine. In another embodiment, R_{Ar} is chlorine. In another embodiment, R_{Ar} is bromine. In another embodiment, R_{Ar} is iodine. In another embodiment, R_{Ar} is cyano. In another embodiment, R_{Ar} is optionally substituted phenyl. In another embodiment, R_{Ar} is optionally substituted six-membered heteroaryl. In another embodiment, R_{Ar} is optionally substituted five-membered heteroaryl. In another embodiment, R_{Ar} is optionally substituted 8 to 10-membered heteroaryl. In another embodiment, R_{Ar} is optionally substituted six-membered heterocycloalkyl. In another embodiment, R_{Ar} is optionally substituted five-membered heterocycloalkyl. In another embodiment, R_{Ar} is —OR₁. In another embodiment, R_{Ar} is —OR₁′. In another embodiment, R_{Ar} is —OCH₂R₁. In another embodiment, R_{Ar} is —OCH₂R₁′. In another embodiment, R_{Ar} is —NHR₁. In another embodiment, R_{Ar} is —NHCH₂R₁. In another embodiment, R_{Ar} is —N(R₁)₂. In another embodiment, R_{Ar} is —C(O)R₁. In another embodiment, R_{Ar} is —C(O)N(R₁)₂. In another embodiment, R_{Ar} is —CH₂R₁. In another embodiment, R_{Ar} is —CH₂R₁′. In another embodiment, R_{Ar} is —CH₂N(R₁)₂. In another embodiment, R_{Ar} is —CH₂OR₁. In another embodiment, R_{Ar} is —CH₂OR₁′.

In one embodiment, R_{Ar} is cyano, optionally substituted phenyl, optionally substituted six-membered heteroaryl, optionally substituted five-membered heteroaryl, optionally substituted (8 to 10)membered heteroaryl, optionally substituted six-membered heterocycloalkyl, optionally substituted five-membered heterocycloalkyl, —OR₁′, —OCH₂R₁′, —NHR₁, —NHCH₂R₁, —N(R₁)₂, —C(O)R₁, —C(O)N(R₁)₂, —CH₂R₁′, —CH₂N(R₁)₂, —CH₂OH, or —CH₂OR₁′.

In one embodiment, R_{Ar} is a five-membered heteroaryl optionally substituted with one or more R₁.

In one embodiment, R_{Ar} is 8 to 10 membered heteroaryl optionally substituted with one or more R₁. In one embodiment, R_{Ar} is an 9 to 10 membered heteroaryl optionally substituted with one or more R₁. In one embodiment, R_{Ar} is an 9 membered heteroaryl optionally substituted with one or more R₁.

In one embodiment, R_{Ar} is (C₃-C₁₀) heterocycloalkyl optionally substituted with one or more R₁. In one embodiment, R_{Ar} is 5 to 6 membered heterocycloalkyl optionally substituted with one or more R₁. In one embodiment, R_{Ar} is 9 to 10 membered heterocycloalkyl optionally substituted with one or more R₁.

In one embodiment, R_{Ar} is halogen, cyano, (C₁-C₁₀)alkyl, (C₁-C₁₀)heteroalkyl, hydroxyl, alkoxyl, aminoalkyl, amino, amido, or carbonyl, each of which is optionally substituted with one or more R₁.

In one embodiment, R_{Ar} is 10-membered aryl optionally substituted with one or more R₁. In one embodiment, R_{Ar} is naphthyl.

In one embodiment, R_{Ar} is phenyl or naphthyl, each of which is optionally substituted with one or more R₁. In one embodiment, R_{Ar} is six-membered heteroaryl, optionally substituted with one or more R₁.

In one embodiment, R_{Ar} is (i) cyano; (ii) (C₁-C₁₀)alkyl, (C₁-C₁₀)heteroalkyl, alkoxyl, aminoalkyl, amino, amido, or carbonyl, each of which is optionally substituted with one or more R₁; or (iii) hydroxyl substituted with one or more R₁′. In one embodiment, R_{Ar} is (i) cyano; (ii) (C₁-C₁₀)heteroalkyl, aminoalkyl, amino, amido, or carbonyl, each of which is optionally substituted with one or more R₁; or (iii) (C₁-C₁₀)alkyl, hydroxyl, or alkoxyl, each of which is substituted with one or more R₁′. In one embodiment, R_{Ar} is (i) cyano; (ii) (C₁-C₁₀)alkyl, (C₁-C₁₀)heteroalkyl, alkoxyl, aminoalkyl, amino, amido, or carbonyl, each of which is optionally substituted with one or more R₁, or (iii) hydroxyl substituted with one or more R₁′. In one embodiment, R_{Ar} is (i) cyano; (ii) (C₁-C₁₀)heteroalkyl, aminoalkyl, amino, amido, or carbonyl, each of which is optionally substituted with one or more R₁, or (iii) (C₁-C₁₀)alkyl, hydroxyl, or alkoxyl, each of which is substituted with one or more R₁′. R′, R₁, and R₁′ are defined herein elsewhere.

In one embodiment, R_{Ar} is fluorine. In another embodiment, R_{Ar} is chlorine. In another embodiment, R_{Ar} is bromine. In another embodiment, R_{Ar} is iodine.

In one embodiment, R_{Ar} is cyano. In another embodiment, R_{Ar} is (C₁-C₁₀) alkyl substituted with one or more R₁. In another embodiment, R_{Ar} is (C₁-C₁₀) alkyl substituted with one or more R₁′. In another embodiment, R_{Ar} is —CH₂R₁. In another embodiment, R_{Ar} is —CH₂R₁′. In another embodiment, R_{Ar} is —CH(R₁)₂. In another embodiment, R_{Ar} is —CH(R₁′)₂. In another embodiment, R_{Ar} is —CH(OH)R₁. In another embodiment, R_{Ar} is 'CH(OH)R₁′. In another embodiment, R_{Ar} is —CH₂OR₁. In another embodiment, R_{Ar} is —CH₂OR₁′. In another embodiment, R_{Ar} is —CH₂OH. In another embodiment, R_{Ar} is hydroxyl or alkoxyl substituted with one or more R₁. In another embodiment, R_{Ar} is hydroxyl or alkoxyl substituted with one or more R₁′. In another embodiment, R_{Ar} is —OR₁. In another embodiment, R_{Ar} is —OR₁′. In another embodiment, R_{Ar} is —OCH₂R₁. In another embodiment, R_{Ar} is —OCH₂R₁′. In another embodiment, R_{Ar} is amino, amido, or carbonyl, each of which is optionally substituted with one or more R₁. In another embodiment, R_{Ar} is —NHR₁. In another embodiment, R_{Ar} is —NHCH₂R₁. In another embodiment, R_{Ar} is —N(R₁)₂. In another embodiment, R_{Ar} is —C(O)R₁. In another embodiment, R_{Ar} is —C(O)N(R₁)₂. In another embodiment, R_{Ar} is —CH₂N(R₁)₂.

Any of the combinations of R_{N}, R_{A}, R_{A}′, R_{B}, R_{B}′, R_{C}, R_{D}, R′, R″, R₁, R₁′, R₂, R₃, R₄, R₅, R₆, R₇, R₈, R_{Ar}, p, q, m, and n are encompassed by this disclosure and specifically provided herein.

It should be noted that if there is a discrepancy between a depicted structure and a chemical name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it and mixtures of two or more stereoisomers of it. Where the compound provided herein contains an alkenyl or alkenylene group, the compound may exist as one or mixture of geometric cis/trans (or Z/E) isomers. Where structural isomers are inter-convertible, the compound may exist as a single tautomer or a mixture of tautomers. This can take the form of proton tautomerism in the compound that contains, for example, an imino, keto, or oxime group; or so-called valence tautomerism in the compound that contain an aromatic moiety. It follows that a single compound may exhibit more than one type of isomerism.

The compounds provided herein may be enantiomerically pure, such as a single enantiomer or a single diastereomer, or be stereoisomeric mixtures, such as a mixture of enantiomers, e.g., a racemic mixture of two enantiomers; an enantio-enriched mixture of two enantiomers; or a mixture of two or more diastereomers. In one embodiment, for compounds that undergo epimerization in vivo, one of skill in the art will recognize that administration of a compound in its (R) form is equivalent to administration of the compound in its (S) form, and vice versa. Conventional techniques for the preparation/isolation of individual enantiomers include synthesis from a suitable optically pure precursor, asymmetric synthesis from achiral starting materials, or resolution of an enantiomeric mixture, for example, by chiral chromatography, recrystallization, resolution, diastereomeric salt formation, or derivatization into diastereomeric adducts followed by separation.

When the compound provided herein contains an acidic or basic moiety, it may also be provided as a pharmaceutically acceptable salt (See, e.g., Berge et al., J. Pharm. Sci. 1977, 66, 1-19; and Handbook of Pharmaceutical Salts, Properties, and Use, Stahl and Wermuth, ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

In certain embodiments, the compounds provided herein are pharmacologically acceptable salts of the compounds with one or more of hydrochloric, sulfuric, phosphoric, acetic, citric, oxalic, malonic, salicylic, malic, fumaric, succinic, ascorbic, maleic, methanesulfonic, and isoethonic acids; or with one or more of potassium carbonate, sodium or potassium hydroxide, ammonia, triethylamine, and triethanolamine.

The compound provided herein may also be provided as a prodrug, which is a functional derivative of the compound, for example, of Formula I and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See, e.g., Harper, Progress in Drug Research 1962, 4, 221-294; Morozowich et al. in Design of Biopharmaceutical Properties through Prodrugs and Analogs, Roche ed., APHA Acad. Pharm. Sci. 1977; Bioreversible Carriers in Drug in Drug Design, Theory and Application, Roche ed., APHA Acad. Pharm. Sci. 1987; Design of Prodrugs, Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in Transport Processes in Pharmaceutical Systems, Amidon et al., ed., Marcell Dekker, 185-218, 2000; Balant et al., Eur. J. Drug Metab. Pharmacokinet. 1990, 15, 143-53; Balimane & Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; and Waller et al., Br. J. Clin. Pharmac. 1989, 28, 497-507.

### C. SYNTHETIC SCHEMES

The schemes below provide exemplary synthetic methods for the preparation of the compounds provided herein. One of ordinary skill in the art will understand that similar methods may be employed to prepare the compounds provided herein. In other words, one of ordinary skill in the art will recognize that suitable adjustments to reagents, protecting groups, reaction conditions, and reaction sequences may be employed to prepare a desired embodiment. The reactions may be scaled upwards or downwards to suit the amount of material to be prepared.

In one embodiment, a compound of formula (I) (e.g., a compound of formula (A) in Scheme 1) may be prepared following, e.g., Scheme 1. For example, 4-bromo-1-fluoro-2-nitrobenzene (I-1) is treated with 2-aminoethanol in n-butanol to yield 2-((4-bromo-2-nitrophenyl)amino)ethanol (I-2). I-2 is reduced, such as with Raney Nickel in methanol, to provide the corresponding aniline I-3. I-3 is coupled with 2-(1-(tert-butoxycarbonyl)-pyrrolidin-2-yl)acetic acid to yield I-4. I-4 is cyclized under acid conditions, such as in AcOH, to yield benzimidazole I-5. The alcohol I-5 is converted to the corresponding tosylate, such as by treatment with TosCl in Et₃N and DCM, to yield I-6. The Boc protecting group in I-6 is removed, such as by TFA, to yield I-7. I-7 is treated with base, such as K₂CO₃ in 20% aqueous 2-propanol, to render Compound 1. Compound 1 may be converted via one or more reactions to other compounds according to formula (A) with suitable R_{Ar} substituent. The bromide in Compound 1 may also be converted via known reactions to other suitable R_{Ar}, and further converted to suitable compounds according to formula (A), such as via alkylation. Specific examples of reactions and conditions converting Compound 1 to compounds according to formula (A) are provided herein below. In one embodiment, (S)-2-(1-(tert-butoxycarbonyl)-pyrrolidin-2-yl)acetic acid, (R)-2-(1-(tert-butoxycarbonyl)-pyrrolidin-2-yl)acetic acid, or racemic 2-(1-(tert-butoxycarbonyl)-pyrrolidin-2-yl)acetic acid is used as the starting material to produce the corresponding stereoisomer or racemate of a compound of formula (I), under similar reactions and conditions as those of Scheme 1.

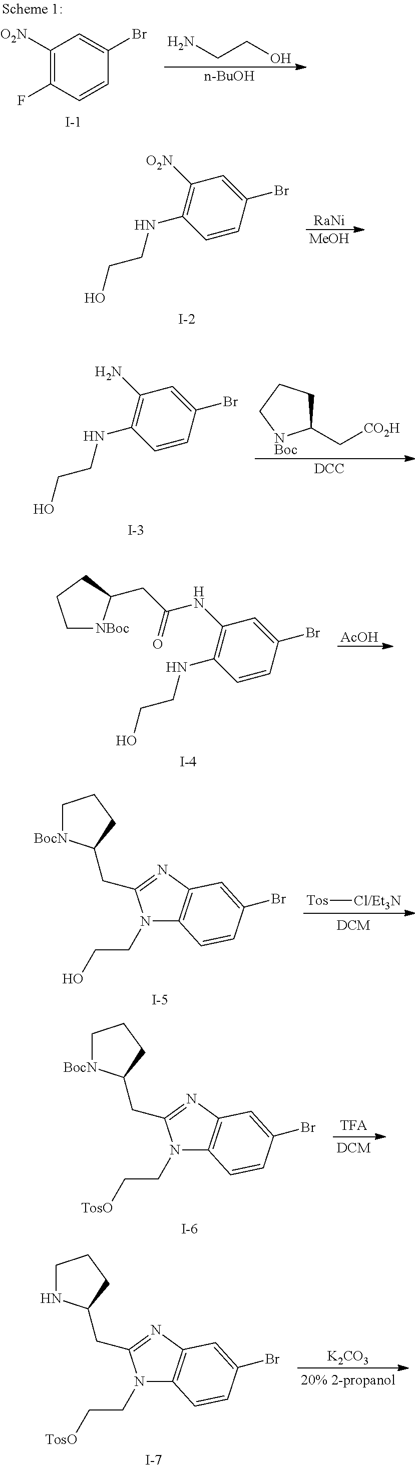

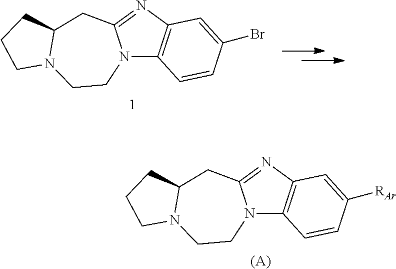

A compound of formula (I) (e.g., a compound of formula (B) in Scheme 2) may also be prepared following, e.g., Scheme 2. For example, 4-bromo-1-fluoro-2-nitrobenzene (I-1) is treated with tert-butyl 2-(aminomethyl)-pyrrolidine-1-carboxylate in n-butanol to yield I-15. I-15 is reduced, such as with hydrazine/Raney Nickel, to provide the corresponding aniline I-16. I-16 is coupled with potassium 3-methoxy-3-oxopropanoate to yield amide I-17. I-17 is treated with acid, such as HOAc, to yield benzimidazole I-18. The methyl ester of benzimidazole I-18 is reduced to the corresponding alcohol I-19 with a reducing agent, such LiAlH₄. The alcohol in I-19 is converted to the corresponding tosylate, such as by treatment with TosCl in Et₃N and DCM, to yield I-20. The Boc protecting group in I-20 is removed, such as by TFA, to yield I-21. I-21 is treated with base, such as K₂CO₃ in 20% aqueous 2-propanol, to render Compound 12. The bromide in Compound 12 may also be converted via known reactions to other suitable R_{Ar}, and further converted to suitable compounds according to Formula (B), such as via alkylation. Specific examples of reactions and conditions converting Compound 12 to compounds according to Formula (B) are provided herein below.

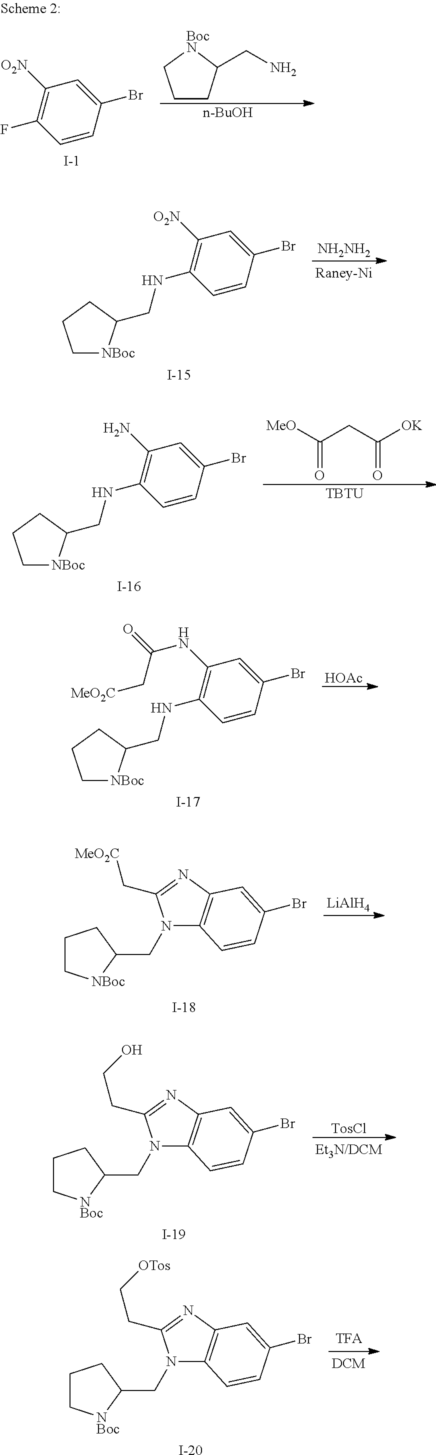

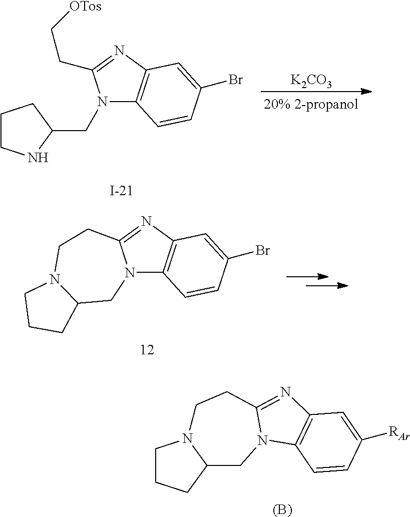

In one embodiment, a compound of Formula (I) (e.g., a compound of formula (C) of Scheme 3) may be prepared following, e.g., Scheme 3. For example, 2,5-dimethoxytetrahydrofuran (I-24) is treated with 3-oxopentanedioic acid and benzylamine to yield I-25. The ketone in I-25 is converted to an amide with NaN₃ under acidic conditions, such as in the presence of sulfuric acid, to provide the corresponding lactam I-26. I-26 is coupled with 1-bromo-4-chloro-2-nitrobenzene to yield the N-substituted lactam I-27. Lactam I-27 is treated with elemental iron under acidic conditions, such as in the presence of HOAc, and is cyclized to yield benzimidazole I-28. The benzyl protecting group in I-28 is removed, such as by hydrogenation, to provide I-29. I-29 is converted to C-1 in one or more steps, such as, via reductive alkylation by ketones or aldehydes, or alkylation by alkyl halides. C-1 may be converted via one or more reactions to other compounds according to formula (C) with suitable R_{Ar}. The chloride in C-1 may also be converted via known reactions to other suitable R_{Ar}, and further converted to suitable compounds according to formula (C), such as via alkylation. Specific examples of reactions and conditions converting C-1 to compounds according to formula (C) are provided herein below.

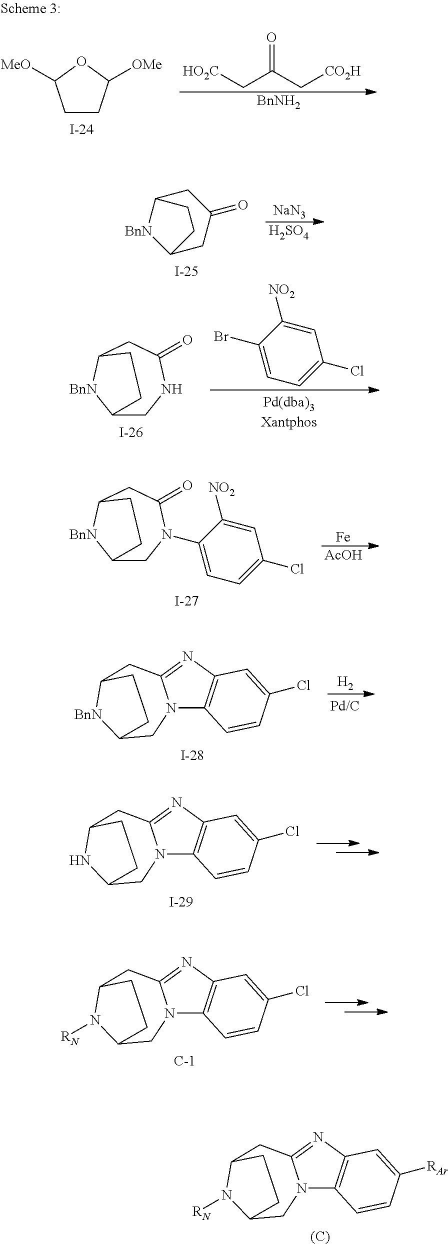

In one embodiment, a compound of formula (III) or (IV) may be prepared following, e.g., Scheme 4. For example, boc-mono-protected hydrazine (I-77) is treated with CbzCl to yield I-78. I-78 is treated with 1,3-dibromopropane to yield I-79, the Boc protecting group of which is removed by treatment with TFA to yield I-80. I-80 is treated with 3-chloropropanoyl chloride to provide I-81, the Cbz protecting group of which is removed by catalytic hydrogenation giving rise to bicyclic I-82. I-82 is reduced, such as with Raney Nickel, to provide the corresponding lactam I-83. I-83 is converted to compound IV-A in one or more steps, such as via reductive alkylation by ketones or aldehydes or alkylation by alkyl halides. IV-A is coupled with 1-bromo-4-chloro-2-nitrobenzene using catalytic palladium to give compound IV-B which is reduced to the corresponding aniline by treatment with excess elemental iron in acetic acid and the aniline intermediate is cyclized in situ to provide compound IV-C. IV-C may be converted via one or more reactions to other compounds according to Formula (IV) with suitable R_{Ar}. The chloride in IV-C may also be converted via known reactions to other suitable R_{Ar}, and further converted to suitable compounds according to Formula (IV), such as via alkylation. Specific examples of reactions and conditions converting IV-C to compounds according to Formula (IV) are provided herein below.

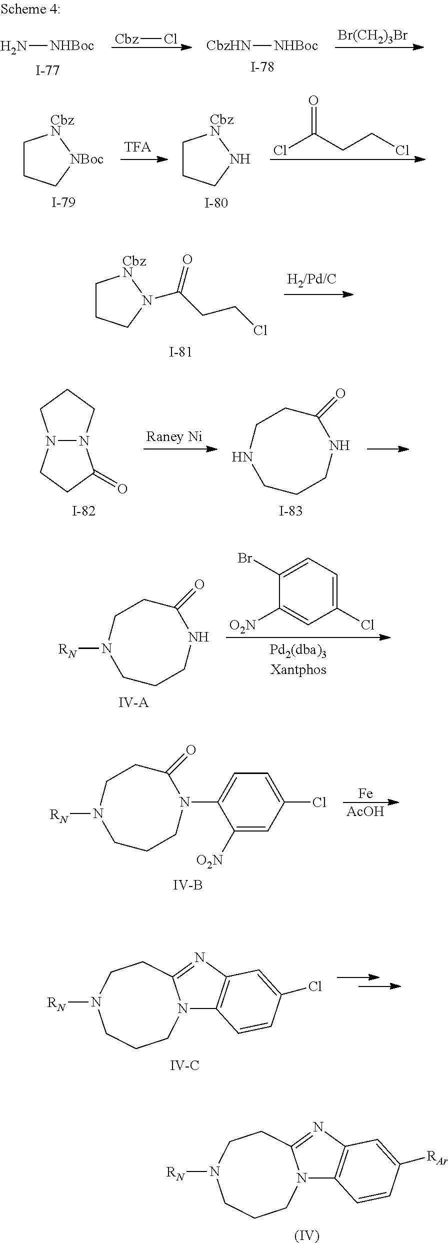

### D. METHODS OF TREATMENT, PREVENTION, AND/OR MANAGEMENT

### 1. Binding to Histamine Receptor

In various embodiments, provided herein is a method of binding a compound provided herein to a histamine receptor, such as, a histamine H3 receptor. The method comprises contacting the histamine receptor with a compound provided herein.

In other embodiments, provided herein is a method of inhibiting the binding of a histamine receptor ligand to a histamine receptor, such as, a histamine H3 receptor. The method comprises contacting the histamine receptor with a compound provided herein. In one embodiment, the histamine receptor ligand is an endogenous ligand. In another embodiment, the ligand is a drug molecule or another small molecule known to have binding affinity to the histamine receptor. In another embodiment, the histamine receptor ligand is a radioactively labeled compound, known to bind to the histamine receptor. In another embodiment, the ligand is an agonist, partial agonist, antagonist, or inverse agonist of the histamine receptor.

In one embodiment, inhibition of ligand binding is assessed using an in vitro binding assay, such as those described herein. In another embodiment, the compound provided herein inhibits mean binding by about 1%, about 5%, about 10%, about 20%, about 30%, about 40%, about 50%, about 60%, about 70%, about 80%, about 90%, about 95%, about 99%, or more, as compared to vehicle. In one embodiment, the inhibition of mean binding is dose dependent.

### 2. Inhibition of Histamine Receptor Activity

In various embodiments, provided herein is a method of modulating (e.g., inhibiting or augmenting) the activity of a histamine receptor, such as a histamine H3 receptor. The method comprises contacting the histamine receptor, such as histamine H3 receptor, with a compound provided herein, in vitro or in vivo. In one embodiment, the histamine receptor, such as histamine H3 receptor, is contacted with a compound provided herein by administering to a subject a therapeutically effective amount of the compound provided herein, or a pharmaceutically acceptable salt or stereoisomer thereof. The subject may be a human. In another embodiment, the histamine receptor is histamine H3 receptor.

In other embodiments, the compound provided herein inhibits or reduces the activity of a histamine receptor, such as histamine H3 receptor. Inhibition of histamine receptor activity may be measured using assays known in the art. In some embodiments, the activity of a histamine receptor is inhibited or reduced by about 1%, about 5%, about 10%, about 20%, about 30%, about 40%, about 50%, about 60%, about 70%, about 80%, about 90%, about 95%, about 99% or more, as compared with the activity obtained without contacting with the compounds provided herein. In one embodiment, the inhibition or reduction of receptor activity is dose dependent. Exemplary assay methods include, but are not limited to, in vitro functional assays. In one embodiment, the functional assay utilizes an appropriate cell-line expression a desired histamine receptor. In other embodiments, the functional assay utilizes synaptosomes isolated from brain tissue of an appropriate organism. In other embodiments, inhibition of histamine receptor activity may be assessed using receptor binding experiments known in the art, e.g. utilizing appropriate membrane preparations. In one embodiment, the assay involves treatment of a test subject (e.g., a rat) with a compound provided herein as well as a reference compound, followed by isolation of brain tissue and ex vivo analysis of receptor occupancy.

In certain embodiments, provided herein are methods of inhibiting or reducing the activity of a histamine receptor, e.g., H3 receptor, in a subject (e.g., human) comprising administering to the subject an effective amount of a compound provided herein. In some embodiments, the activity of histamine receptor is inhibited or reduced by about 1%, about 5%, about 10%, about 20%, about 30%, about 40%, about 50%, about 60%, about 70%, about 80%, about 90%, about 95%, about 99% or more, when measured using an assay described herein elsewhere.

In one embodiment, provided herein is a method of inhibiting or reducing the activity of a histamine receptor, such as a histamine H3 receptor, by a histamine receptor ligand. In one embodiment, the method comprises contacting the histamine receptor with an antagonist or an inverse agonist of the histamine receptor. In another embodiment, an antagonist or an inverse agonist of the histamine receptor is a compound provided herein.

### 3. Modulation of Histamine Release

In some embodiments, provided herein is a method of inhibiting a histamine receptor to increase the histamine release by a cell. The method includes contacting the cell with a compound provided herein. In one embodiment, the cell is a brain cell, such as a neuron or a glial cell. In one embodiment, the histamine release occurs in vivo. Thus, in certain embodiments, provided herein are methods of increasing the level of histamine release comprising administering to a subject (e.g., human) an effective amount of a compound provided herein. In an organism, the histamine release may occur, for example, at the synapse. Thus, in one embodiment, the neuronal cell is in contact with the synapse of a mammal. In another embodiment, the histamine release occurs in vitro. In some embodiments, the cell may be a brain cell, such as a neuronal cell or a cell type which expresses a histamine receptor, such as a histamine H3 receptor.

Stimulation of histamine release can be shown, for example, by performing various in vitro functional assays utilizing a cell type which expresses a certain type of histamine receptor, such as a histamine H3 receptor, together with an appropriate labeled histamine receptor ligand. In some embodiments, inhibition of the histamine receptor is demonstrated when an antagonist or inverse agonist (e.g., a compound provided herein) has an IC₅₀ of, for example, between about 0.1 nM and about 10 μM, between about 1 nM and about 1 μM, between about 1 nM and about 500 nM, and between about 1 nM and about 100 nM, in a functional histamine receptor assay, such as those described herein.

### 4. Treatment, Prevention, and/or Management of H3 Receptor Related Disorders

In one embodiment, provided herein are methods for the treatment, prevention, and/or management of a disorder provided herein, such as, e.g., a disorder related to histamine H3 receptor, such as, e.g., a neurological disorder provided herein. In one embodiment, provided herein are methods for the treatment, prevention, and/or management of one or more symptoms of a disorder provided herein, such as, e.g., a disorder related to histamine H3 receptor, such as, e.g., a neurological disorder provided herein. In one embodiment, the method provided herein comprises administering a compound provided herein. In one embodiment, the method provided herein comprises administering a compound provided herein, or a pharmaceutically acceptable salt or stereoisomer thereof. In one embodiment, the method provided herein comprises administering a composition provided herein. In one embodiment, the method provided herein comprises administering a pharmaceutical composition provided herein. In one embodiment, the method provided herein comprises administering a therapeutically effective amount of a compound provided herein. In one embodiment, the method provided herein comprises administering a prophylactically effective amount of a compound provided herein. In one embodiment, the method provided herein comprises administering a therapeutically effective or prophylactically effective amount of a compound provided herein, or a pharmaceutically acceptable salt or stereoisomer thereof.

In one embodiment, provided herein are uses of a compound provided herein in the manufacture of a medicament for the treatment, prevention, and/or management of a disorder provided herein, such as, e.g., a disorder related to histamine H3 receptor, such as, e.g., a neurological disorder. In one embodiment, provided herein are uses of a compound provided herein, or a pharmaceutically acceptable salt or stereoisomer thereof in the manufacture of a medicament for the treatment, prevention, and/or management of a disorder provided herein, such as, e.g., a disorder related to histamine H3 receptor, such as, e.g., a neurological disorder. In one embodiment, provided herein are uses of a composition provided herein in the manufacture of a medicament for the treatment, prevention, and/or management of a disorder provided herein, such as, e.g., a disorder related to histamine H3 receptor, such as, e.g., a neurological disorder. In one embodiment, provided herein are uses of a pharmaceutical composition provided herein in the manufacture of a medicament for the treatment, prevention, and/or management of a disorder provided herein, such as, e.g., a disorder related to histamine H3 receptor, such as, e.g., a neurological disorder.

In one embodiment, provided herein is a compound for use in the treatment, prevention, and/or management of a disorder provided herein, such as, e.g., a disorder related to histamine H3 receptor, such as, e.g., a neurological disorder provided herein. In one embodiment, provided herein is a compound, or a pharmaceutically acceptable salt or stereoisomer thereof, for use in the treatment, prevention, and/or management of a disorder provided herein, such as, e.g., a disorder related to histamine H3 receptor, such as, e.g., a neurological disorder provided herein. In one embodiment, provided herein is a composition for use in the treatment, prevention, and/or management of a disorder provided herein, such as, e.g., a disorder related to histamine H3 receptor, such as, e.g., a neurological disorder provided herein. In one embodiment, provided herein is a pharmaceutical composition for use in the treatment, prevention, and/or management of a disorder provided herein, such as, e.g., a disorder related to histamine H3 receptor, such as, e.g., a neurological disorder provided herein.

In one embodiment, provided herein are compounds, or pharmaceutically acceptable salts or stereoisomers thereof, for use in the treatment, prevention, and/or management of a disorder provided herein. In one embodiment, provided herein are compositions for use in the treatment, prevention, and/or management of a disorder provided herein. In one embodiment, provided herein are pharmaceutical compositions for use in the treatment, prevention, and/or management of a disorder provided herein. In one embodiment, provided herein are kits for use in the treatment, prevention, and/or management of a disorder provided herein.

In some embodiments, provided herein is a method of treating, preventing, and/or managing a disorder related to histamine H3 receptor, such as a neurological disorder. Without being limited by a particular theory, the treatment, prevention, and/or management is done by inhibiting or reducing the activity of histamine H3 receptor. Histamine H3 receptors modulate the release of neurotransmitters, including but not limited to, histamine, acetylcholine, norepinephrine, and dopamine, implicating a wide range of therapeutic indications. See, e.g., Haas et al., Physio. Rev. 88: 1183-241 (2008); Brown et al., Prog. Neurobio. 63: 637-72 (2001); Esbenshade et al., Mol. Interven. 6(2): 77-88 (2006); Esbenshade et al., British J. Pharmacol. 154(6): 1166-81 (2008); Sander et al., Bio. Pharm. Bull. 21: 2163-81 (2008).

In one embodiment, the method comprises administering to a subject (e.g., human) a therapeutically or prophylactically effective amount of a composition or compound provided herein. In one embodiment, the subject is a human. In another embodiment, the compound provided herein inhibits the activity of a histamine receptor. In another embodiment, the compound provided herein inhibits the activity of histamine H3 receptor. In certain embodiments, the compounds provided herein are inverse agonists of histamine H3 receptor. In other embodiments, the compounds provided herein are antagonists of histamine H3 receptors. In certain embodiments, the compounds provided herein are selective for histamine H3 receptor over other CNS-related targets. In one embodiment, the compounds provided herein are highly brain penetrable in animals, such as rodents, and human. In some embodiments, inhibition of the histamine receptor activity may be assessed by functional assays as described herein elsewhere. In certain embodiments, the efficacious concentration of the compounds provided herein is less than 10 nM, less than 100 nM, less than 1 μM, less than 10 μM, less than 100 μM, or less than 1 mM. In other embodiments, compound's activity may be assessed in various art-recognized animal models as described herein elsewhere.

In some embodiments, provided herein is a method of treating, preventing, and/or managing a disorder associated with excessive daytime sleepiness, such as narcolepsy, Parkinson's disease, Multiple Sclerosis, shift workers, jet lag, relief of side effects of other medications, and the like, comprising administering to a subject an effective amount of a compound provided herein. For example, without being limited by a particular theory, H3 antagonists or inverse agonists may have wake promoting effects. See, e.g., Lin et al., Br. Res. 523: 325-30 (1990); Barbier et al., Br. J. Pharm. 143: 649-61 (2004); Lin et al., Neurobiol. Dis. 30(1): 74-83 (2008).

In another embodiment, provided herein is a method of treating, preventing, and/or managing a sleeping disorder, such as insomnia, comprising administering to a subject an effective amount of a compound provided herein. For example, without being limited by a particular theory, H3 antagonists or inverse agonists may improve wakefulness and lead to an improved sleep pattern, and therefore H3 antagonists or inverse agonists may be useful in treating insomnia.

In another embodiment, provided herein is a method of treating, preventing, and/or managing substance abuse, comprising administering to a subject an effective amount of a compound provided herein. For example, without being limited by a particular theory, H3 antagonists can alter methamphetamine self-administration in rats, and therefore H3 antagonists may ameliorate the craving for addictive drugs. See, e.g., Munzar et al, Neuropsychopharmacology 29:705-17 (2004).

In another embodiment, provided herein is a method of treating, preventing, and/or managing a disorder related to cognitive impairments, impairments of learning, impairments of memory, and/or impairments of attention, vigilance and/or speed of response, such as those associated with Alzheimer's disease, Parkinson's disease, schizophrenia, mild cognitive impairment (MCI), and attention deficit hyperactivity disorder (ADHD), and the like, comprising administering to a subject an effective amount of a compound provided herein. For example, without being limited by a particular theory, H3 antagonists or inverse agonists may have pro-cognitive effects, such as, e.g., those measured by passive avoidance, novel object recognition, social recognition, and attention-set shifting. See, e.g., Medhurst et al., JPET 321: 1032-45 (2007); Medhurst et al., Biochem. Pharmcol. 73: 1182-94 (2007); Fox et al., JPET 313:176-190 (2005); Fox et al., JPET 305: 897-908 (2003). Further, without being limited by a particular theory, H3 receptor antagonists or inverse agonists may improve social memory, increase the acquisition of a test paradigm, and reverse scopolamine-induced deficits. H3 antagonists or inverse agonists may also reverse scopolamine-induced deficits in a passive avoidance memory test.

In another embodiment, provided herein is a method of treating, preventing, and/or managing a disorder related to psychosis, schizophrenia, ADHD, and/or mood disorders, such as depression and/or anxiety, comprising administering to a subject an effective amount of a compound provided herein. For example, without being limited by a particular theory, H3 antagonists or inverse agonists may improve the gating deficits of DBA/2 mice seen in the pre-pulse inhibition (PPI) test and reverse the methamphe-tamine-induced hyperlocomotor activity. See, e.g., Fox et al., JPET 313:176-190 (2005). Without being limited to a particular theory, H3 antagonists or inverse agonists may: 1) reverse the amphetamine-induced hyper-locomotor activity (See, e.g., Clapham et al., Eur. J. Pharmacol. 259: 107-14 (1994)); 2) be useful as antipsychotic agents and dosed sparing (See, e.g., Zhang et al., Br. Res. 1045: 142-49 (2005)); 3) improve attention and modulate impulsivity (See, e.g., Day et al., Biochem. Pharmacol. 73:1123-34 (2007)); 4) improve learning parameters in ADHD (See, e.g., Fox et al., JPET 313:176-90 (2005); Fox et al., JPET 305: 897-908 (2003); Fox et al., Behay. Br. Res. 131: 151-61 (2002); Komater et al., Psychopharm. 167: 363-72 (2003); Esbenshade et al., Biochem. Pharmacol. 68: 933-45 (2004)); 5) enhance learning ability and reduce anxiety in behavioral tests (See, e.g., Rizk et al., Eur. J. Neurosci. 19: 1992-96 (2004)); and 6) have an anti-depressant effect (See, e.g., Pérez-García et al., Psychopharm. 142(2): 215-20 (1999)).

In another embodiment, provided herein is a method of using the compounds provided herein as psycho-stimulants, which may lack the abuse liabilities generally associated with other classes of psycho-stimulants. Without being limited by a particular theory, H3 antagonists or inverse agonists increase the levels of histamine, dopamine, norepinephrine, and acetylcholine in the prefrontal cortical area, which is consistent with their pro-cognitive effects and their wake promoting effects seen in animal models. For example, H3 antagonists or inverse agonists may increase dopamine in the frontal cortex but not the striatum. H3 antagonists or inverse agonists may not induce increased locomotor activity or sensitization that is associated with other psycho-stimulus. See, e.g., Komater et al., Psychopharm. 167: 363-72 (2003).

In another embodiment, provided herein is a method of treating, preventing, and/or managing a disorder such as convulsion (e.g. epilepsy), seizures, vertigo, and pain, comprising administering to a subject an effective amount of a compound provided herein. For example, without being limited by a particular theory, H3 antagonists or inverse agonists may be protective against pentylenetetrazole (PTZ) and electrical-induced seizures. See, e.g., Vohora et al., Life Sci. 22: 297-301 (2000); Vohora et al., Pharmacol. Biochem. Behay. 68(4): 735-41 (2001); Zhang et al., Eur. J. Pharmacol. 15(581): 169-75 (2003). H3 antagonists or inverse agonists may increase the seizure threshold in humans. See, e.g., WO 2006/084833. H3 antagonists or inverse agonists may decrease electrical discharge from afferent neurons in an inner ear preparation. See, e.g., Chavez et al., Brain Res. 1064(1-2): 1-9 (2005). Further, H3 receptors are localized on neurons in the dorsal horn of the spinal cord, an area important for the transmission of nociceptive information in humans, and have shown efficacy in preclinical pain models. Thus, without being limited by a particular theory, H3 receptor antagonists or inverse agonists may increase the threshold for neuropathic pain, which was shown in models such as the chronic constriction injure (CCl) model, herpes virus-induced model, and capsaicin-induced allodynia model. See, e.g., Medhurst et al., Pain 138: 61-69 (2008); Medhurst et al., Biochem. Pharmacol. 73: 1182-94 (2007). Therefore, in some embodiments, the compounds provided herein are employed for their analgesic effects to treat, prevent, and/or manage disorders involving pain and the sensitization that accompanies many neuropathic pain disorders.

In yet another embodiment, provided herein is a method of treating, preventing, and/or managing a disorder related to satiety, gastric activity, irritable bowel syndrome (IBS), chronic constipation (CC), and/or metabolic disorders such as diabetes and obesity, comprising administering to a subject an effective amount of a compound provided herein. In other embodiments, provided herein is a method of mitigating the weight gain associated with other therapeutic agents, comprising administering to a subject an effective amount of a compound provided herein. For example, without being limited to a particular theory, H3 receptor plays a role in satiety. See, e.g., Masaki et al., Curr. Diabetes Rev. 3: 212-16 (2007); Ishizuka et al., Behay. Br. Res. 188: 250-54 (2008). H3 antagonists or inverse agonists may decrease food intake, reduce weight gain, reduce plasma triglyceride levels, modulate energy expenditure, reduce body weight and body fat, and normalize insulin tolerance. See, e.g., Malmlof et al., Obesity 14: 2154-62 (2006); Hancock et al., Eur J. Pharm. 487: 183-97 (2004). H3 antagonists or inverse agonists may also block olanzepine-induced decrease in satiety. See, e.g., WO 2006/084833.

In another embodiment, provided herein is a method of treating, preventing, and/or managing a disorder of enteric system and/or exocrine pancreatic system, such as acid secretion, digestion, and gut motility, comprising administering to a subject an effective amount of a compound provided herein. See, e.g., Breunig et al., J. Physiol. 583(2): 731-42 (2007); Singh et al., Inflamm. Res. 46: 159-65 (1997); Bertaccini et al., Dig. Dis. Sci. 40: 2052-63 (1995).

In another embodiment, provided herein is a method of treating, preventing, and/or managing movement disorders, such as Parkinson's disease, restless leg syndrome (RLS), and Huntington's disease, comprising administering to a subject an effective amount of a compound provided herein. For example, without being limited by a particular theory, an increased expression of H3 receptors have been found in the postmortem brain of subjects with Parkinson's disease. See, e.g., Anichtchik et al., Neurobiol. Dis. 8: 707-16 (2001); Anichtchik et al., Eur. J. Pharm. 12: 3823-32 (2000). Further, it was reported that a polymorphism in the primary enzyme that metabolizes histamine in the brain, the Thr105Ile polymorphism, results in a functional alteration in activity of the enzyme. This polymorphism has been associated with movement disorders such as Parkinson's disease and essential tremor. See, e.g., Preuss et al., JPET 53: 708-17 (1998); Agundez et al., Neuromol. Med. 10(1): 10-16 (2008); Ledesma et al., Neuromol. Med. 10(4): 356-61 (2008). Thus, H3 antagonists or inverse agonists may be useful in the treatment of Parkinson's disease. See, e.g., Gomez-Ramirez et al., Mov. Disord. 21: 839-46 (2006).

In some embodiments, the compounds provided herein are active in at least one model, which can be used to measure the activity of the compounds and estimate their efficacy in treating a neurological disorder. For example, when the model is for depression (e.g., mean immobility), the compounds are active when they inhibit mean immobility of a test subject by about 5%, about 10%, about 20%, about 30%, about 40%, about 50%, about 60%, about 70%, about 80%, about 90%, about 95%, about 99%, or more, when compared to vehicle. In some embodiments, the compounds provided herein produce a similar disparity in measured endpoint between treated animals and animals administered vehicle.

In other embodiments, provided herein is a method of effecting a therapeutic effect as described herein elsewhere. The method comprises administering to a subject (e.g., a mammal) a therapeutically effective amount of a compound or composition provided herein. The particular therapeutic effects may be measured using any model system known in the art and described herein, such as those involving an animal model of a disease.

In some embodiments, the neurological disorder is: depression (e.g., major depressive disorder, bipolar disorder, unipolar disorder, dysthymia and seasonal affective disorder); cognitive deficits; fibromyalgia; pain (e.g., neuropathic pain); sleep related disorders (e.g., sleep apnea, insomnia, narcolepsy, cataplexy) including those sleep disorders which are produced by psychiatric conditions; chronic fatigue syndrome; attention deficit disorder (ADD); attention deficit hyperactivity disorder (ADHD); restless leg syndrome; schizophrenia; anxieties (e.g., general anxiety disorder, social anxiety disorder, panic disorder); obsessive compulsive disorder; posttraumatic stress disorder; seasonal affective disorder (SAD); premenstrual dysphoria; post-menopausal vasomotor symptoms (e.g., hot flashes, night sweats); neurodegenerative disease (e.g., Parkinson's disease, Alzheimer's disease and amyotrophic lateral sclerosis); manic conditions; dysthymic disorder; cyclothymic disorder; obesity; or substance abuse or dependency (e.g., cocaine addiction, nicotine addiction). In another embodiment, the compounds provided herein are useful to treat, prevent, and/or manage two or more conditions/disorders, which are co-morbid, such as cognitive deficit and depression.

Neurological disorders include cerebral function disorders, including without limitation, senile dementia, Alzheimer's type dementia, cognition, memory loss, amnesia/amnestic syndrome, epilepsy, disturbances of consciousness, coma, lowering of attention, speech disorders, Lennox syndrome, autism, and hyperkinetic syndrome.

Neuropathic pain includes without limitation post herpetic (or post-shingles) neuralgia, reflex sympathetic dystrophy/causalgia or nerve trauma, phantom limb pain, carpal tunnel syndrome, and peripheral neuropathy (such as diabetic neuropathy or neuropathy arising from chronic alcohol use).

Other exemplary diseases and conditions that may be treated, prevented, and/or managed using the methods, compounds, and/or compositions provided herein include, but are not limited to: obesity; migraine or migraine headache; urinary incontinence, including without limitation involuntary voiding of urine, dribbling or leakage of urine, stress urinary incontinence (SUI), urge incontinence, urinary exertional incontinence, reflex incontinence, passive incontinence, and overflow incontinence; and sexual dysfunction, in men or women, including without limitation sexual dysfunction caused by psychological and/or physiological factors, erectile dysfunction, premature ejaculation, vaginal dryness, lack of sexual excitement, inability to obtain orgasm, and psycho-sexual dysfunction, including without limitation, inhibited sexual desire, inhibited sexual excitement, inhibited female orgasm, inhibited male orgasm, functional dyspareunia, functional vaginismus, and atypical psychosexual dysfunction.

In one embodiment, the neurological disorder is excessive daytime sleepiness. In another embodiment, the neurological disorder is cognitive impairment. In another embodiment, the neurological disorder is mood disorders. In another embodiment, the neurological disorder is movement disorders. In another embodiment, the neurological disorder is schizophrenia. In another embodiment, the neurological disorder is attention disorders. In another embodiment, the neurological disorder is anxiety disorder. In another embodiment, the neurological disorder is seizure. In another embodiment, the neurological disorder is epilepsy. In another embodiment, the neurological disorder is vertigo. In another embodiment, the neurological disorder is pain. In another embodiment, the neurological disorder is neuropathic pain. In another embodiment, the neuropathic pain is diabetic neuropathy. In another embodiment, the neurological disorder is sleeping disorder. In another embodiment, the neurological disorder is insomnia. In another embodiment, the neurological disorder is substance abuse.

In one embodiment, the neurological disorder is a neurodegenerative disease. In one embodiment, the neurodegenerative disease is Parkinson's disease. In another embodiment, the neurodegenerative disorder is Alzheimer's disease.

In one embodiment, the disorder is obesity, and the therapeutically effective amount of compound to supply to a patient is sufficient so that said patient feels satiated. In another embodiment, the disorder is diabetes. In another embodiment, the disorder is metabolic diseases. In another embodiment, the disorder is a disease effecting the enteric system.

In one embodiment, the compounds described herein treat, prevent, and/or manage a central nervous disorder, without causing addiction to said compounds.

Any suitable route of administration can be employed for providing the patient with a therapeutically or prophylactically effective dose of an active ingredient. For example, oral, mucosal (e.g., nasal, sublingual, buccal, rectal, vaginal), parenteral (e.g., intravenous, intramuscular), transdermal, and subcutaneous routes can be employed. Exemplary routes of administration include oral, transdermal, and mucosal. Suitable dosage forms for such routes include, but are not limited to, transdermal patches, ophthalmic solutions, sprays, and aerosols. Transdermal compositions can also take the form of creams, lotions, and/or emulsions, which can be included in an appropriate adhesive for application to the skin or can be included in a transdermal patch of the matrix or reservoir type as are conventional in the art for this purpose. An exemplary transdermal dosage form is a “reservoir type” or “matrix type” patch, which is applied to the skin and worn for a specific period of time to permit the penetration of a desired amount of active ingredient. The patch can be replaced with a fresh patch when necessary to provide constant administration of the active ingredient to the patient.

The amount to be administered to a patient to treat, prevent, and/or manage the disorders described herein will depend upon a variety of factors including the activity of the particular compound employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount required. For example, the physician or veterinarian could start doses of the compounds employed at levels lower than that required in order to achieve the desired therapeutic effect and gradually increase the dosage until the desired effect is achieved.

In general, a suitable daily dose of a compound provided herein will be that amount of the compound which is the lowest dose effective to produce a therapeutic or prophylactic effect. Such an effective dose will generally depend upon the factors described above. Generally, oral, intravenous, intracerebroventricular and subcutaneous doses of the compounds provided herein for a patient will range from about 0.005 mg per kilogram to about 5 mg per kilogram of body weight per day. In one embodiment, the oral dose of a compound provided herein will range from about 10 mg to about 300 mg per day. In another embodiment, the oral dose of a compound provided herein will range from about 20 mg to about 250 mg per day. In another embodiment, the oral dose of a compound provided herein will range from about 100 mg to about 300 mg per day. In another embodiment, the oral dose of a compound provided herein will range from about 10 mg to about 100 mg per day. In another embodiment, the oral dose of a compound provided herein will range from about 25 mg to about 50 mg per day. In another embodiment, the oral dose of a compound provided herein will range from about 50 mg to about 200 mg per day. Each of the above-recited dosage ranges may be formulated as a single or multiple unit dosage formulations.

In some embodiments, the compound disclosed herein may be used in combination with one or more second active agent(s) to treat, prevent, and/or manage a disorder described herein. In one embodiment, the second active agent is known in the art, such as, e.g., those described in http://www.fda.gov/; The Merck Manual, 18th ed. 2006; and PDR: Physician Desk Reference 2010, 64th ed. 2009; the contents of each of which are hereby incorporated by reference in their entireties. In one embodiment, the second active agent is lurasidone, olanzapine, risperidone, aripiprazole, donepezil, rivastigmine, memantine, amphetamine, methylphenidate, atomoxetine, modafinil, sertraline, fluoxetine, or L-DOPA. In one embodiment, the second active agent includes, but is not limited to, lurasidone, olanzapine, risperidone, aripiprazole, donepezil, rivastigmine, memantine, amphetamine, methylphenidate, atomoxetine, modafinil, sertraline, fluoxetine, or L-DOPA.

### 5. Pharmaceutical Compositions and Dosage Forms

Pharmaceutical compositions can be used in the preparation of individual, single unit dosage forms. Pharmaceutical compositions and dosage forms provided herein comprise a compound provided herein, or a pharmaceutically acceptable salt or stereoisomer thereof, or a clathrate or prodrug thereof. Pharmaceutical compositions and dosage forms can further comprise one or more excipients.

Pharmaceutical compositions and dosage forms provided herein can also comprise one or more additional active ingredients. Examples of optional second, or additional, active ingredients are also disclosed herein.

Single unit dosage forms provided herein are suitable for oral, mucosal (e.g., nasal, sublingual, vaginal, buccal, or rectal), parenteral (e.g., subcutaneous, intravenous, bolus injection, intramuscular, or intra-arterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms are used will vary from one another and will be readily apparent to those skilled in the art. See, e.g., Remington's Pharmaceutical Sciences, 18th ed., Mack Publishing, Easton Pa. (1990).

In one embodiment, pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided are pharmaceutical compositions and dosage forms that contain little, if any, lactose other mono- or disaccharides. As used herein, the term “lactose-free” means that the amount of lactose present, if any, is insufficient to substantially increase the degradation rate of an active ingredient.

Lactose-free compositions can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. In one embodiment, lactose-free dosage forms comprise active ingredients, microcrystalline cellulose, pre-gelatinized starch, and magnesium stearate.

Also provided are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are, in one embodiment, packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Also provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. In one embodiment, dosage forms comprise a compound provided herein in an amount of from about 0.10 to about 500 mg. In other embodiments, dosage forms comprise a compound provided herein in an amount of about 0.1, 1, 2, 5, 7.5, 10, 12.5, 15, 17.5, 20, 25, 50, 100, 150, 200, 250, 300, 350, 400, 450, or 500 mg.

In other embodiments, dosage forms comprise the second active ingredient in an amount of 1 to about 1000 mg, from about 5 to about 500 mg, from about 10 to about 350 mg, or from about 50 to about 200 mg. Of course, the specific amount of the second active agent will depend on the specific agent used, the diseases or disorders being treated or managed, and the amount(s) of a compound provided herein, and any optional additional active agents concurrently administered to the patient.

5.1 Oral Dosage Forms

Pharmaceutical compositions that are suitable for oral administration can be provided as discrete dosage forms, such as, but not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's The Science and Practice of Pharmacy, 21st ed., Lippincott Williams & Wilkins (2005).

Oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In one embodiment, oral dosage forms are tablets or capsules, in which case solid excipients are employed. In another embodiment, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is, in one embodiment, present in from about 50 to about 99 weight percent of the pharmaceutical composition or dosage form.

Disintegrants may be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients may be used to form solid oral dosage forms. The amount of disintegrant used varies based upon the type of formulation, and is readily discernible to those of ordinary skill in the art. In one embodiment, pharmaceutical compositions comprise from about 0.5 to about 15 weight percent of disintegrant, or from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants may be used in an amount of less than about 1 weight percent of the pharmaceutical compositions or dosage forms into which they are incorporated.

In one embodiment, a solid oral dosage form comprises a compound provided herein, and optional excipients, such as anhydrous lactose, microcrystalline cellulose, polyvinylpyrrolidone, stearic acid, colloidal anhydrous silica, and gelatin.

5.2 Controlled Release Dosage Forms

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active agents provided herein. In one embodiment, provided are single unit dosage forms suitable for oral administration such as, but not limited to, tablets, capsules, gelcaps, and caplets that are adapted for controlled-release.

In one embodiment, controlled-release pharmaceutical products improve drug therapy over that achieved by their non-controlled counterparts. In another embodiment, the use of a controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

In another embodiment, the controlled-release formulations are designed to initially release an amount of drug (active ingredient) that promptly produces the desired therapeutic or prophylactic effect, and gradually and continually release of other amounts of drug to maintain this level of therapeutic or prophylactic effect over an extended period of time. In one embodiment, in order to maintain a constant level of drug in the body, the drug can be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.3 Parenteral Dosage Forms

Parenteral dosage forms can be administered to patients by various routes including, but not limited to, subcutaneous, intravenous (including bolus injection), intramuscular, and intra-arterial. In some embodiments, administration of a parenteral dosage form bypasses patients' natural defenses against contaminants, and thus, in these embodiments, parenteral dosage forms are sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms. For example, cyclodextrin and its derivatives can be used to increase the solubility of a compound provided herein. See, e.g., U.S. Pat. No. 5,134,127, which is incorporated herein by reference.

5.4 Topical and Mucosal Dosage Forms

Topical and mucosal dosage forms provided herein include, but are not limited to, sprays, aerosols, solutions, emulsions, suspensions, eye drops or other ophthalmic preparations, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed herein are well known to those skilled in the pharmaceutical arts, and depend on the particular tissue to which a given pharmaceutical composition or dosage form will be applied. In one embodiment, excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms. Examples of additional ingredients are well known in the art. See, e.g., Remington's Pharmaceutical Sciences, 16th and 18th eds., Mack Publishing, Easton Pa. (1980 & 1990).

The pH of a pharmaceutical composition or dosage form may also be adjusted to improve delivery of one or more active ingredients. Also, the polarity of a solvent carrier, its ionic strength, or tonicity can be adjusted to improve delivery. Compounds such as stearates can also be added to pharmaceutical compositions or dosage forms to alter the hydrophilicity or lipophilicity of one or more active ingredients so as to improve delivery. In other embodiments, stearates can serve as a lipid vehicle for the formulation, as an emulsifying agent or surfactant, or as a delivery-enhancing or penetration-enhancing agent. In other embodiments, salts, stereoisomers, solvates, prodrugs, or clathrates of the active ingredients can be used to further adjust the properties of the resulting composition.

### 6. Kits

In one embodiment, active ingredients provided herein are not administered to a patient at the same time or by the same route of administration. In another embodiment, provided are kits which can simplify the administration of appropriate amounts of active ingredients.

In one embodiment, a kit comprises a dosage form of a compound provided herein. Kits can further comprise one or more second active ingredients as described herein, or a pharmacologically active mutant or derivative thereof, or a combination thereof.

In other embodiments, kits can further comprise devices that are used to administer the active ingredients. Examples of such devices include, but are not limited to, syringes, drip bags, patches, and inhalers.

Kits can further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### V. EXAMPLES

Certain embodiments are illustrated by the following non-limiting examples.

A. Synthesis of Compounds

In the examples below, unless otherwise indicated, all temperatures are set forth in degrees Celsius and all parts and percentages are by weight. Reagents may be purchased from commercial suppliers, such as Sigma-Aldrich Chemical Company, and may be used without further purification unless otherwise indicated. Reagents may also be prepared following standard literature procedures known to those skilled in the art. Solvents may be purchased from Aldrich in Sure-Seal bottles and used as received. All solvents may be purified using standard methods known to those skilled in the art, unless otherwise indicated.

The reactions set forth below were done generally at ambient temperature, unless otherwise indicated. The reaction flasks were fitted with rubber septa for introduction of substrates and reagents via syringe. Analytical thin layer chromatography (TLC) was performed using glass-backed silica gel pre-coated plates (Merck Art 5719) and eluted with appropriate solvent ratios (v/v). Reactions were assayed by TLC or LCMS, and terminated as judged by the consumption of starting material. Visualization of the TLC plates was done with UV light (254 wavelength) or with an appropriate TLC visualizing solvent, such as basic aqueous KMnO₄ solution activated with heat. Flash column chromatography (See, e.g., Still et al., J. Org. Chem., 43: 2923 (1978)) was performed using silica gel 60 (Merck Art 9385) or various MPLC systems.

The compound structures in the examples below were confirmed by one or more of the following methods: proton magnetic resonance spectroscopy, mass spectroscopy, elemental microanalysis, and melting point. Proton magnetic resonance (¹H NMR) spectra were determined using a NMR spectrometer operating at a certain field strength. Chemical shifts are reported in parts per million (ppm, δ) downfield from an internal standard, such as TMS. Alternatively, ¹H NMR spectra were referenced to signals from residual protons in deuterated solvents as follows: CDCl₃=7.25 ppm; DMSO-d₆=2.49 ppm; C₆D₆=7.16 ppm; CD₃OD=3.30 ppm. Peak multiplicities are designated as follows: s, singlet; d, doublet; dd, doublet of doublets; t, triplet; dt, doublet of triplets; q, quartet; br, broadened; and m, multiplet. Coupling constants are given in Hertz (Hz). Mass spectra (MS) data were obtained using a mass spectrometer with APCI or ESI ionization.

### 1. Compound 1 ((S)-10-bromo-2,3,5,6,13,13a-hexahydro-1H-benzo[4,5]imidazo[1,2-d]pyrrolo[2,1-g][1,4]diazepine)

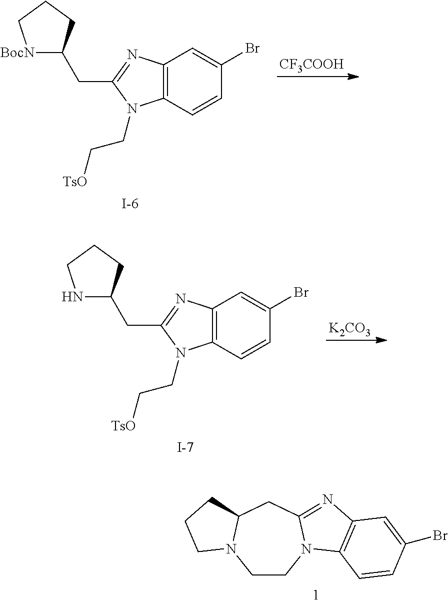

Intermediate I-6 (3.9 g, 6.7 mmol, 1.0 eq) was dissolved in dichloromethane, neat trifluoroacetic acid (3 mL) was added and the reaction mixture was stirred at room temperature overnight. The excess solvent and trifluoroacetic acid were removed by evaporation. The crude intermediate I-7 was dissolved in a mixture of isopropanol and water (1:4) and solid K₂CO₃ (1.9 g, 13 mmol, 4.0 eq) was added. The reaction mixture was refluxed for 6 hours, cooled to room temperature and extracted with ethyl acetate. The combined organic layers were dried over anhydrous Na₂SO₄ and the solids were removed by filtration. The filtrate was concentrated by evaporation to give compound 1 (1.0 g, 50%). ¹H-NMR (400 MHz, CD₃OD) δ: 7.96 (1H, s), 7.89 (1H, d), 7.72 (1H, d), 5.19 (1H, m), 4.10 (1H, m), 3.80 (4H, m), 3.56 (1H, m), 3.23 (1H, m), 2.50 (1H, m), 2.08 (3H, m). MS (ESI): m/z 306 (M+H⁺).

### 2. Compound 2 ((S)-4-(2,3,5,6,13,13a-hexahydro-1H-benzo[4,5]imidazo[1,2-d]pyrrolo[2,1-g][1,4]diazepin-10-yl)benzonitrile)

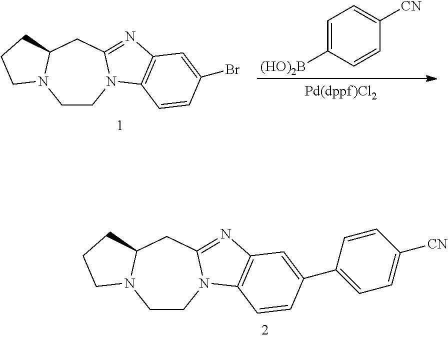

Compound 1 (100 mg, 0.30 mmol, 1.0 eq) and 4-cyanophenylboronic acid (66 mg, 0.45 mmol, 1.5 eq) were dissolved in DMF (2 mL) and solid K₂CO₃ (83 mg, 0.60 mmol, 2.0 eq) and Pd(dppf)Cl₂ (10 mg, 0.03 mmol, 0.1 eq) were added. The reaction mixture was heated under microwave irradiation at 120° C. for 60 minutes. The solids were removed by filtration; the filtrate was diluted with ethyl acetate and washed with aqueous HCl (1.0 M). The organic layer was discarded and the aqueous layer was basified with aqueous solution of NaHCO₃ to pH ˜8.0. The aqueous layer was extracted with ethyl acetate, the combined organic layers were dried over anhydrous Na₂SO₄, the solids were removed by filtration and the filtrate was concentrated by evaporation. The crude reaction product was purified with preparative thin-layer chromatography to give compound 2 (12 mg, 10%). ¹H-NMR (400 MHz, CDCl₃) δ: 7.91 (1H, s), 7.81 (4H, m), 7.55 (1H, d), 7.36 (1H, s), 4.48 (1H, m), 4.20 (1H, m), 3.42 (2H, m), 3.24 (1H, m), 2.97 (1H, m), 2.43 (2H, m), 2.36 (1H, m), 2.13 (1H, m), 2.01 (1H, m), 1.90 (2H, m). MS (ESI): m/z 329 (M+H⁺).

### 3. Compound 3 ((S)-N-(4-(2,3,5,6,13,13a-hexahydro-1H-benzo[4,5]imidazo[1,2-d]pyrrolo[2,1-g][1,4]diazepin-10-yl)phenyl)acetamide)

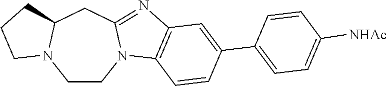

This compound was prepared in 5% yield (6 mg) as described for compound 2 but using 4-acetamidophenylboronic acid as the starting material. ¹H-NMR (400 MHz, CDCl₃) δ: 7.87 (1H, s), 7.66 (4H, m), 7.48 (1H, m), 7.38 (1H, m), 7.30 (1H, m), 4.46 (1H, m), 4.20 (1H, m), 3.42 (2H, m), 3.23 (1H, m), 2.98 (1H, m), 2.42 (2H, m), 2.36 (1H, m), 2.20 (3H, s), 2.13 (1H, m), 1.81 (1H, m), 1.75 (2H, m). MS (ESI): m/z 361 (M+H⁺).

### 4. Compound 4 ((5)-10-(furan-2-yl)-2,3,5,6,13,13a-hexahydro-1H-benzo[4,5]imidazo[1,2-d]pyrrolo[2,1-g][1,4]diazepine)

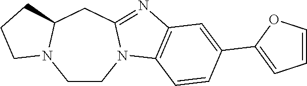

This compound was prepared in 40% yield (42 mg) as described for compound 2 but using furan-2-yl-boronic acid as the starting material. ¹H-NMR (400 MHz, CDCl₃) δ: 7.99 (1H, s), 7.75 (1H, d), 7.47 (1H, s), 7.26 (1H, d), 6.62 (1H, d), 6.47 (1H, d), 4.43 (1H, m), 4.17 (1H, m), 3.47 (2H, m), 3.26 (1H, m), 2.96 (1H, m), 2.42 (2H, m), 2.38 (1H, m), 2.26 (1H, m), 1.86 (1H, m), 1.65 (2H, m). MS (ESI): m/z 294 (M+H⁺).

### 5. Compound 5 ((S)-5-(2,3,5,6,13,13a-hexahydro-1H-benzo[4,5]imidazo[1,2-d]pyrrolo[2,1-g][1,4]diazepin-10-yl)picolinonitrile)

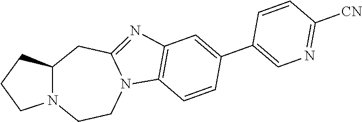

This compound was prepared in 11% yield (15 mg) as described for compound 2 but using intermediate I-8 and 5-bromopicolinonitrile as starting materials. ¹H-NMR (400 MHz, CDCl₃) δ: 9.00 (1H, d, 2.4 Hz), 8.05 (1H, dd, 2.4 Hz, 8.4 Hz), 7.92 (1H, s), 7.51 (1H, d, 2.0 Hz), 7.46 (1H, d, 2.4 Hz), 7.42 (1H, d, 2.4 Hz), 4.46 (1H, m), 4.22 (1H, m), 3.45 (2H, m), 3.24 (1H, m), 3.01 (1H, m), 2.48 (2H, m), 2.32 (1H, m), 2.16 (1H, m), 1.91 (1H, m), 1.76 (2H, m). MS (ESI): m/z 330 (M+H⁺).

### 6. Compound 6 ((S)-10-(pyrazin-2-yl)-2,3,5,6,13,13a-hexahydro-1H-benzo[4,5]imidazo[1,2-d]pyrrolo[2,1-g][1,4]diazepine)

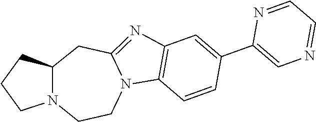

This compound was prepared in 4% yield (5 mg) as described for compound 2 but using intermediate I-8 and 2-bromopyrazine as starting materials. ¹H-NMR (400 MHz, CDCl₃) δ: 9.09 (1H, s), 8.63 (1H, d), 8.47 (1H, d), 8.31 (1H, s), 8.00 (1H, dd), 7.40 (1H, d), 4.49 (1H, m), 4.22 (1H, m), 3.46 (2H, m), 3.24 (1H, m), 2.98 (1H, m), 2.46 (2H, m), 2.36 (1H, m), 2.22 (1H, m), 1.86 (1H, m), 1.75 (1H, m). MS (ESI): m/z 306 (M+H⁺).

### 7. Compound 7 (11-bromo-1,2,3,4,6,7,14,14a-octahydrobenzo-[4,5]imidazo[1,2-d]pyrido[2,1-g][1,4]diazepine)

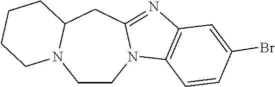

This compound was prepared via intermediate I-12 using the same reaction sequence that was described for the preparation of compound 1 which proceeded through intermediate I-5 and I-6. Intermediate I-12 (2.0 g, 4.4 mmol) as the starting material gave compound 7 (450 mg, 32% overall). ¹H-NMR (400 MHz, CDCl₃) δ: 7.81 (1H, d, J=2.0 Hz), 7.32 (1H, m), 7.1 (1H, d, J=8.4 Hz), 4.27 (2H, m), 3.17 (3H, m), 2.95 (1H, m), 2.48 (1H, m), 2.23 (2H, m), 1.83-1.25 (7H, m). MS (ESI): m/z 320.7 (M+H⁺).

### 8. Compound 8 (4-(1,2,3,4,6,7,14,14a-octahydrobenzo[4,5]imidazo-[1,2-d]pyrido[2,1-g][1,4]diazepin-11-yl)benzonitrile)

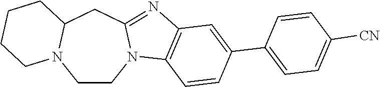

This compound was prepared in 26% yield (40 mg) as described for compound 2 but using compound 7 as the starting material. ¹H-NMR (400 MHz, CDCl₃) δ: 7.90 (1H, d, J=1.6 Hz), 7.71 (4H, m), 7.49 (1H, m), 7.35 (1H, m), 4.35-4.25 (2H, m), 3.19-3.14 (3H, m), 2.96 (1H, m), 2.53-2.46 (1H, m), 2.26-2.20 (2H, m), 1.83-1.29 (6H, m). MS (ESI): m/z 343.7 (M+H⁺).

### 9. Compound 9 (11-(pyrazin-2-yl)-1,2,3,4,6,7,14,14a-octahydrobenzo[4,5]imidazo[1,2-d]pyrido[2,1-g][1,4]diazepine)

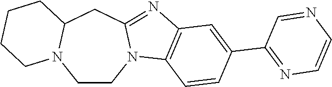

This compound was prepared in 90% yield (60 mg) as described for compound 6 but using intermediate I-13 as the starting material. ¹H-NMR (400 MHz, CDCl₃) δ: 9.14 (2H, d, J=1.6 Hz), 8.66 (1H, t), 8.49 (1H, d, J=2.4 Hz), 8.30 (1H, d, J=1.2 Hz), 8.03 (1H, m), 7.61 (1H, d, J=8.4 Hz), 4.61 (1H, m), 4.30 (1H, m), 3.26-3.17 (2H, m), 3.09-2.97 (2H, m), 2.53-2.47 (1H, m), 2.32-2.26 (2H, m), 1.87-1.38 (6H, m). MS (ESI): m/z 320.7 (M+H⁺).

### 10. Compound 10 (11-(1H-imidazol-1-yl)-1,2,3,4,6,7,14,14a-octahydrobenzo[4,5]imidazo[1,2-d]pyrido[2,1-g][1,4]diazepine)

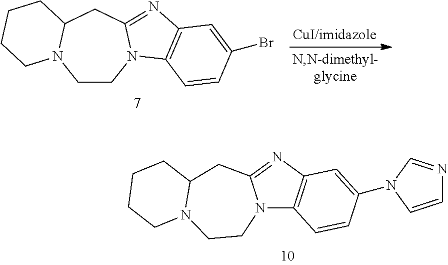

Compound 7 (50 mg, 0.16 mmol, 1.0 eq) and imidazole (16 mg, 0.24 mmol, 1.5 eq) were dissolved in DMSO (0.5 mL), and solid CuI (3 mg, 0.016 mmol, 0.1 eq), N,N-dimethyl glycine (3.2 mg, 0.031 mmol, 0.2 eq) and K₃PO₄.3H₂O (67 mg, 0.31 mmol, 2.0 eq) were added. The reaction mixture was heated at 110° C. overnight, the solids were removed by filtration and the filtrate was extracted and ethyl acetate. The organic layer was washed with water, the combiner organic layers were dried over anhydrous Na₂SO₄, the solids were removed by filtration and filtrate was concentrated. The crude reaction product was purified silicagel chromatography to give compound 10 (4.4 mg, 9%). ¹H-NMR (400 MHz, CDCl₃) δ: 8.11 (1H, s), 7.71 (1H, d, J=1.2 Hz), 7.62-7.57 (2H, m), 7.46 (1H, dd), 7.15 (1H, s), 4.62-4.57 (1H, m), 4.33-4.27 (1H, m), 3.29-3.17 (2H, m), 3.09-2.97 (2H, m), 2.53-2.47 (1H, m), 2.32-2.25 (2H, m), 1.86-1.40 (6H, m). MS (ESI): m/z 308.7 (M+H⁺).

### 11. Compound 11 (11-(4-methylpiperazin-1-yl)-1,2,3,4,6,7,14,14a-octahydrobenzo[4,5]imidazo[1,2-d]pyrido[2,1-g][1,4]diazepine)

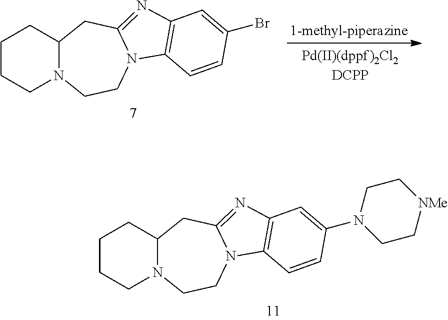

Compound 7 (60 mg, 0.19 mmol, 1.0 eq) and 1-methylpiperazine (38 mg, 0.38 mmol, 2.0 eq) were dissolved in toluene (0.5 mL) and solid Pd(dppf)₂Cl₂ (6 mg, 0.0037 mmol, 0.1 eq), DCCP (6 mg, 0.019 mmol, 0.1 eq) and t-BuONa (36 mg, 0.38 mmol, 2.0 eq) were charged. The reaction mixture was heated under microwave irradiation at 120° C. for 2 hours and the solids were removed by filtration. The filtrate was extracted with ethyl acetate and the organic layer was washed with water and brine. The combined organic layers were dried over anhydrous MgSO₄, the solids were removed by filtration and the filtrate was concentrated. The crude reaction product was purified by preparative TLC to give compound 11 (8 mg, 13%). ¹H-NMR (400 MHz, CDCl₃) δ: 7.36 (1H, d, J=8.8 Hz), 7.14 (1H, d, J=1.2 Hz), 7.07 (1H, dd), 4.49-4.44 (1H, m), 4.24-4.18 (1H, m), 3.21-3.09 (6H, m), 3.02-2.95 (2H, m), 2.71 (4H, m), 2.45 (4H, m), 2.30-2.21 (2H, m), 1.84-1.41 (6H, m). MS (ESI): m/z 340.7 (M+H⁺).

### 12. Compound 12 (9-bromo-2,3,5,6,13,13a-hexahydro-1H-benzo[4,5]imidazo[1,2-d]pyrrolo[1,2-a][1,4]diazepine)

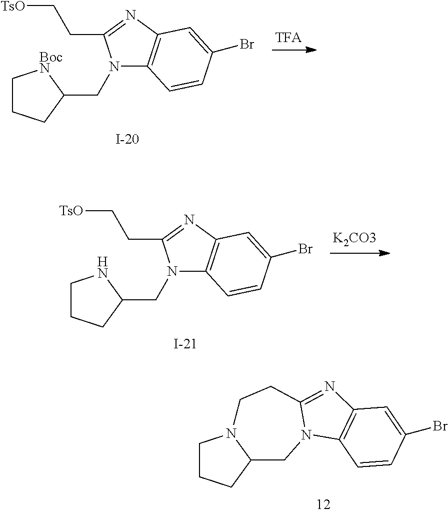

Intermediate I-20 (0.46 g 0.7 mmol, 1.0 eq) was dissolved in dichloromethane (2 mL), trifluoroacetic acid (1 mL) was added and the reaction mixture was stirred at room temperature overnight. Excess solvent and trifluoroacetic acid were removed by evaporation. The crude intermediate I-21 was dissolved in a 1:4 mixture of isopropanol and water (2 mL : 8 mL) and solid K₂CO₃ (0.42 g, 1.4 mmol, 2.0 eq) was added. The reaction mixture refluxed for 6 hours, cooled to room temperature, ethyl acetate was added and solvent phases were allowed to separate. The organic layer was dried over anhydrous Na₂SO₄, the solids were removed by filtration and the filtrate was concentrated by evaporation to give compound 12 (150 mg, 50%). ¹H-NMR (CD₃OD) δ: 7.82 (s, 1H), 7.34 (dd, 1H, J=8.4 Hz, 1.6 Hz), 7.13 (d, 1H, J=8.4 Hz), 4.35 (m, 1H), 3.88 (m, 1H), 3.45 (m, 2H), 3.33 (m, 2H) 2.40 (m, 3H), 2.20 (m, 1H), 2.08 (m, 3H). (CI): m/z 306 (M+H⁺).

### 13. Compound 13 (4-(2,3,5,6,13,13a-hexahydro-1H-benzo[4,5]imidazo[1,2-d]pyrrolo[1,2-a][1,4]diazepin-9-yl)benzonitrile)

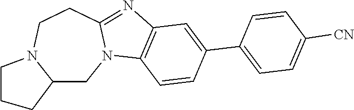

This compound was prepared in 38% yield (20 mg) as described for compound 2 but using compound 12 as the starting material. ¹H-NMR (400 MHz, CDCl₃) δ: 7.91 (s, 1H), 7.7 (m, 4H), 7.49 (dd, 1H, J=8.4, 1.6 Hz), 7.36 (d, 1H, J=8.4 Hz), 4.43 (m, 1H), 3.92 (m, 1H), 3.39 (m, 2H), 3.24 (m, 2H), 2.43 (m, 2H), 2.33 (m, 1H), 2.13 (m, 1H), 2.01 (m, 1H), 1.90 (m, 2H). MS (CI): m/z 329 (M+H⁺).

### 14. Compound 14 (9-(furan-2-yl)-2,3,5,6,13,13a-hexahydro-1H-benzo[4,5]imidazo[1,2-d]pyrrolo[1,2-a][1,4]diazepine)

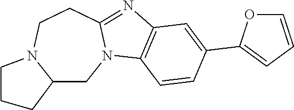

This compound was prepared in 42% yield (21 mg) as described for compound 4 but using compound 12 as the starting material. ¹H-NMR (400 MHz, CDCl₃) δ: 7.99 (s, 1H), 7.63 (dd, 1H, J=8.0 Hz, 1.2 Hz), 7.47 (s, 1H), 7.23 (s, 1H), 6.62 (d, 1H, J=2.4), 6.47 (dd, 1H, J=3.2, 1.6 Hz), 4.37 (m, 1H), 3.87 (m, 1H), 3.47 (m, 2H), 3.26 (m, 2H), 2.41 (m, 2H), 2.38 (m, 1H), 2.26 (m, 1H), 1.92 (m, 1H), 1.76 (m, 2H). (ESI): m/z 294 (M+H⁺).

### 15. Compound 15 (11-(imidazo[1,2-a]pyridin-6-yl)-1,2,3,4,6,7,14,14a-octahydrobenzo[4,5]imidazo[1,2-d]pyrido[2,1-g][1,4]diazepine)

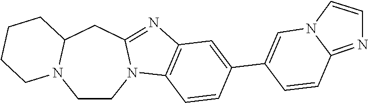

This compound was prepared in 81% yield (58 mg) as described for compound 2 but using intermediate I-13 and 6-bromoimidazo[1,2-a]pyridine as the starting materials. ¹H-NMR (400 MHz, CDCl₃) δ: 8.49 (1H, s), 7.73 (1H, s), 7.63 (1H, s), 7.44 (3H, s), 7.34 (2H, m), 4.37 (1H, m), 4.08 (1H, m), 2.90 (4H, m), 2.29 (1H, m), 2.08 (2H, m), 1.65-1.19 (6H, m). (ESI): m/z 358.7 (M+H)⁺.

### 16. Compound 16 (11-(1H-benzo[d]imidazol-1-yl)-1,2,3,4,6,7,14,14a-octahydrobenzo[4,5]imidazo[1,2-d]pyrido[2,1-g][1,4]diazepine)

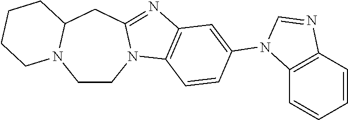

This compound was prepared in 90% yield (50 mg) as described for compound 10 but using 1H-benzimidazole as the starting material. ¹H-NMR (400 MHz, CDCl₃) δ: 8.13 (1H, s), 7.89 (1H, m), 7.81 (1H, m), 7.51 (1H, m), 7.41-7.28 (4H, m), 4.35 (2H, m), 3.22-3.16 (2H, m), 2.97 (1H, m), 2.53 (1H, m), 2.24 (2H, m), 1.85-1.31 (6H, m). (ESI): m/z 358.7 (M+H)⁺.

### 17. Compound 17 (4-((1,2,3,4,6,7,14,14a-octahydrobenzo[4,5]imidazo[1,2-d]pyrido[2,1-g][1,4]diazepin-11-yl)methyl)morpholine)

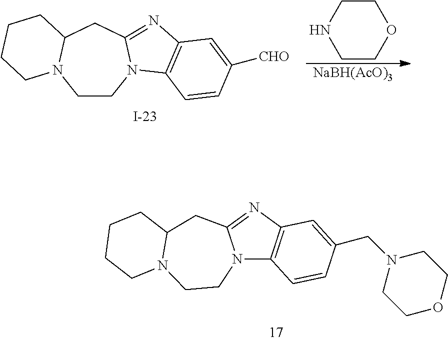

Intermediate I-23 (80 mg, 0.30 mmol, 1.0 eq) was dissolved in dichloromethane (2 mL) and morpholine (52 mg, 0.6 mmol, 2.0 eq) was added followed by glacial acetic acid (1 drop). The reaction mixture was stirred at room temperature for 30 minutes, and solid NaBH(OAc)₃ (254 mg, 1.2 mmol. 4.0 eq) was added portionwise. The reaction mixture was stirred at room temperature overnight, aqueous NaHCO₃ solution was added and the pH was adjusted to 8. The crude reaction mixture was extracted with dichloromethane, the combined organic layers were dried over anhydrous Na₂SO₄, the solids were removed by filtration and the filtrate was concentrated by evaporation. The crude reaction product was purified by preparative thin layer chromatography to give compound 17 (30 mg, 29%). ¹H-NMR (400 MHz, CDCl₃) δ: 7.53 (1H, m), 7.27 (1H, m), 4.51 (1H, m), 4.23 (1H, m), 3.66 (4H, m), 3.60 (2H, s), 3.15 (2H, m), 3.03-2.93 (2H, m), 2.44 (5H, m), 2.24 (2H, m), 1.83-1.27 (6H, m). (ESI): m/z 341.7 (M+H)⁺.

### 18. Compound 18 (3-chloro-13-cyclobutyl-6,7,8,9,10,11-hexahydro-7,10-epiminobenzo[4,5]imidazo[1,2-a]azocine)

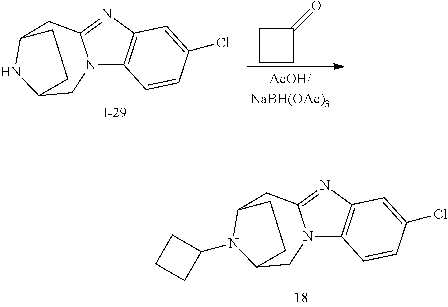

Acetic acid (0.9 g, 15 mmol, 1.5 eq) was added to a solution of intermediate I-29 (2.5 g, 10 mmol) in dichloromethane (40 mL) and the reaction mixture was stirred for 5 minutes at room temperature. Neat cyclobutanone (1.1 g, 15 mmol, 1.5 eq) was added and the reaction mixture was stirred for 20 minutes. Solid NaBH(OAc)₃ (3.2 g, 15 mmol, 1.5 eq) was added, and the reaction mixture was for 2 hours. The reaction mixture was quenched with aqueous solution of NaHCO₃, and extracted with dichloromethane. The combined organic layers were dried over anhydrous Na₂SO₄, the solids were removed by filtration and the filtrate was concentrated by evaporation. The crude product was purified by silica gel chromatography to give compound 18 (2.7 g, 89%) as a pale yellow solid. ¹H-NMR (CDCl₃) δ: 7.64 (s, J=1.6 Hz, 1H), 7.19 (dd, J=8.0 Hz, 1H), 7.11 (d, J=8.0 Hz, 1H), 4.17 (d, J=14.0 Hz, 1H), 4.08 (dd, J=14.0 Hz, 1H), 3.62 (s, 1H), 3.52 (s, 1H), 3.26-3.33 (m, 3H), 2.13-2.17 (m, 2H), 1.94-2.12 (m, 4H), 1.70-1.86 (m, 3H), 1.24 (m, 1H). MS (ESI): m/z 302.0 (M+H⁺).

### 19. Compound 19 (13-cyclobutyl-3-(furan-2-yl)-6,7,8,9,10,11-hexahydro-7,10-epiminobenzo[4,5]imidazo[1,2-a]azocine)

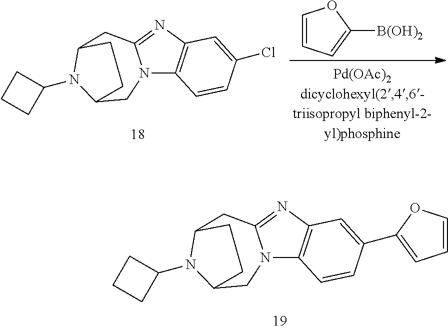

Compound 18 (150 mg, 0.5 mmol, 1.0 eq), Pd(OAc)₂ (10 mg, 0.05 mmol, 0.1 eq), dicyclohexyl(2′,4′,6′-triisopropylbiphenyl-2-yl)phosphine (30 mg, 0.1 mmol, 0.2 eq), KF (141 mg, 1.5 mmol, 3.0 eq), furan-2-ylboronic acid (112 mg, 1.0 mmol, 2.0 eq) and 1,4-dioxane (3 mL) were mixed under nitrogen atmosphere and heated under microwave irradiation at 110° C. for 1.5 hours. The solids were removed by filtration, the filtrate was concentrated by evaporation and the crude reaction product was purified by preparative RP-HPLC to give compound 19 (80 mg, 49%) as a white solid. ¹H-NMR (CDCl₃) δ: 7.96 (s, 1H), 7.58 (dd, J=8.0 Hz, 1H), 7.47 (s, 1H), 7.20 (d, J=8.0 Hz, 1H), 6.62 (m, 1H), 6.47 (m, 1H), 4.10-4.20 (m, 2H), 3.63 (s, 1H), 3.54 (s, 1H), 3.27-3.36 (m, 3H), 2.10-2.18 (m, 2H), 1.94-2.04 (m, 4H), 1.72-1.86 (m, 2H), 1.48 (t, J=9.2 Hz, 1H), 1.28 (t, J=9.2 Hz, 1H). MS (ESI): m/z 334.1 (M+H⁺).

### 20. Compound 20 (4-(13-cyclobutyl-6,7,8,9,10,11-hexahydro-7,10-epiminobenzo[4,5]imidazo[1,2-a]azocin-3-yl)benzonitrile)

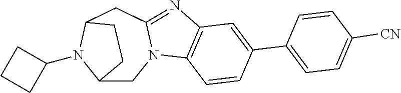

This compound was prepared in 50% yield (19 mg) as described for compound 19 but using 4-cyanophenylboronic acid as the starting material. ¹H-NMR (CDCl₃) δ: 7.88 (s, 1H), 7.72 (s, 4H), 7.47 (dd, J=8.0 Hz, 1H), 7.30 (d, J=8.0 Hz, 1H), 4.18-4.20 (m, 2H), 3.65 (s, 1H), 3.55 (s, 1H), 3.30-3.35 (m, 3H), 2.11-2.16 (m, 2H), 1.93-2.05 (m, 4H), 1.66-1.84 (m, 2H), 1.48 (m, 2H). MS (ESI): m/z 369.1 (M+H⁺).

### 21. Compound 21 (N-(4-(13-cyclobutyl-6,7,8,9,10,11-hexahydro-7,10-epiminobenzo[4,5]imidazo[1,2-a]azocin-3-yl)phenyl)acetamide)

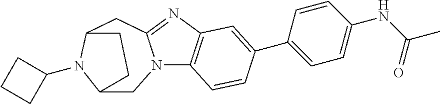

This compound was prepared in 45% yield (18 mg) as described for compound 19 but using 4-acetamidophenylboronic acid as the starting material. ¹H-NMR (CDCl₃) δ: 7.84 (s, 1H), 7.58 (m, 4H), 7.43-7.47 (m, 2H), 7.25 (s, 1H), 4.17-4.18 (m, 2H), 3.64 (s, 1H), 3.54 (s, 1H), 3.29-3.35 (m, 3H), 2.21 (s, 3H), 2.12-2.18 (m, 2H), 1.91-2.03 (m, 4H), 1.71-1.86 (m, 2H), 1.48 (t, J=10.4 Hz, 1H), 1.32 (t, J=10.4 Hz, 1H). MS (ESI): m/z 401.1 (M+H⁺).

### 22. Compound 22 (13-cyclobutyl-3-(1H-indol-5-yl)-6,7,8,9,10,11-hexahydro-7,10-epiminobenzo[4,5]imidazo[1,2-a]azocine)

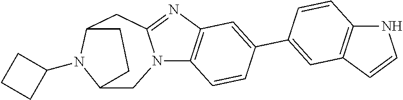

This compound was prepared in 55% yield (22 mg) as described for compound 19 but using 1H-indol-5-yl-boronic acid as the starting material. ¹H-NMR (CDCl₃) δ: 8.28 (s, 1H), 7.93 (s, 1H), 7.89 (s, 1H), 7.46-7.56 (m, 3H), 7.24-7.28 (m, 2H), 6.62 (s, 1H), 4.16-4.20 (m, 2H), 3.66 (s, 1H), 3.56 (s, 1H), 3.33-3.37 (m, 3H), 2.13-2.17 (m, 2H), 1.98-2.05 (m, 4H), 1.72-1.85 (m, 2H), 1.52 (t, J=8.8 Hz, 1H), 1.36 (t, J=8.8 Hz, 1H). MS (ESI): m/z 383.1 (M+H⁺).

### 23. Compound 23 ((4-(13-cyclobutyl-6,7,8,9,10,11-hexahydro-7,10-epiminobenzo[4,5]imidazo[1,2-a]azocin-3-yl)phenyl)-methanamine)

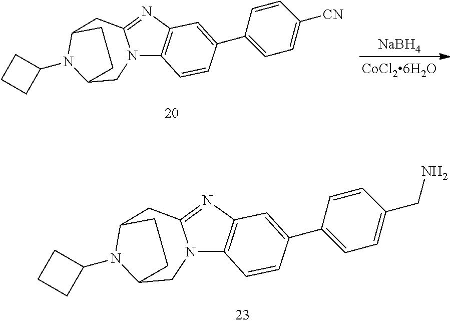

CoCl₂.6H₂O (48 mg, 0.2 mmol, 1.0 eq) and compound 20 (80 mg, 0.2 mmol, 1.0 eq) were dissolved a mixture of THF and water (10 mL:5 mL), and the reaction mixture was cooled to 0° C. on an ice bath. Two drops of glacial acetic acid were added, and the reaction mixture was stirred for additional 10 minutes. Solid NaBH₄ (24 mg, 0.6 mmol, 3.0 eq) was added, and the reaction mixture was stirred for additional 2 hours. Aqueous ammonia (2 mL) was added, solids were removed by filtration and the filtrate was extracted with dichloromethane. The combined organic layers were dried over anhydrous sodium sulfate, the solids were removed by filtration and the filtrate was concentrated by evaporation. The crude reaction product was purified by preparative RP-HPLC to give compound 23 (10 mg, 22%) as white solid. ¹H-NMR (CDCl₃) δ: 7.76 (d, J=1.6 Hz, 1H), 7.63 (s, 1H), 7.61 (s, 1H), 7.52-7.55 (dd, J=8.4 Hz, 1H), 7.41-7.47 (m, 3H), 4.18 (m 2H), 3.85 (s, 2H), 3.68 (s, 1H), 3.42 (s, 1H), 3.40 (m, 3H), 2.15-2.21 (m, 2H), 1.96-2.08 (m, 6H), 1.76-1.83 (m, 2H), 1.37 (t, J=8.8 Hz, 1H), 1.24 (t, J=8.8 Hz, 1H). MS (ESI): m/z 373.1 (M+H⁺).

### 24. Compound 24 (4-(13-cyclobutyl-6,7,8,9,10,11-hexahydro-7,10-epiminobenzo[4,5]imidazo[1,2-a]azocin-3-yl)-2-fluorobenzonitrile)

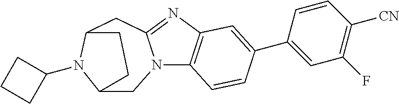

This compound was prepared in 49% yield (80 mg) as described for compound 2 but using intermediate I-30 and 4-bromo-2-fluorobenzonitrile as the starting materials. ¹H-NMR (CDCl₃) δ: 7.88 (s, 1H), 7.67 (t, J=8.0 Hz, 1H), 7.51-7.53 (dd, J=8.0 Hz, 1H), 7.47 (s 1H), 7.45 (s, 1H), 7.31 (d, J=8.4 Hz, 1H) 4.17-4.24 (m, 2H), 3.68 (s, 1H), 3.58 (s, 1H), 3.32-3.35 (m, 3H), 2.15-2.18 (m, 2H), 1.98-2.07 (m, 4H), 1.74-1.85 (m, 2H), 1.47 (t, J=8.0 Hz, 2H) 1.30 (t, J=8.0 Hz, 1H). MS (ESI): m/z 387.1 (M+H⁺).

### 25. Compound 25 (8-chloro-3-cyclobutyl-2,3,4,5-tetrahydro-1H-1,5-ethanobenzo[4,5]imidazo[1,2-d][1,4]diazepine)

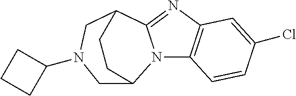

This compound was prepared in 57% yield (1.0 g) as described for compound 18 but using intermediate I-36 as the starting material. ¹H-NMR (400 MHz, CDCl₃) δ: 7.69 (s, 1H), 7.18 (s, 2H), 4.67 (t, J=5.2 Hz, 1H), 3.49 (t, J=4.8 Hz, 1H), 3.08 (m, 2H), 2.75 (m, 1H), 2.40 (m, 2H), 2.00 (m, 4H), 1.82 (m, 2H), 1.70 (m, 2H). MS (ESI): m/z 302.1 (M+H⁺).

### 26. Compound 26 (4-(3-cyclobutyl-2,3,4,5-tetrahydro-1H-1,5-ethanobenzo[4,5]imidazo[1,2-d][1,4]diazepin-8-yl)benzonitrile)

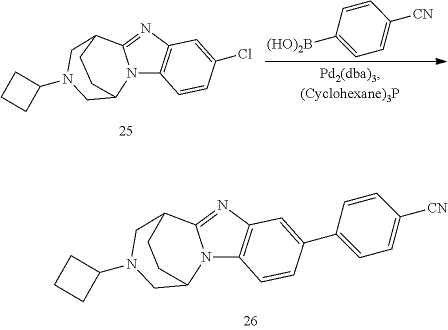

Compound 25 (37 mg, 0.12 mmol, 1.0 eq), 4-cyanophenylboronic acid (26 mg, 0.18 mmol, 1.5 eq), Pd₂(dba)₃ (11 mg, 0.012 mmol, 0.1 eq), (cyclohexane)₃P (10 mg, 0.036 mmol, 0.3 eq) and KF (24 mg, 0.42 mmol, 3.5 eq) were dissolved in 1,4-dioxane (2 mL), and the reaction mixture was heated under microwave irradiation under an argon atmosphere at 100° C. for 1 hour. The reaction mixture was cooled to room temperature, the solids were removed by filtration and the filtrate was extracted with dichloromethane. The combined organic layers were dried over anhydrous Na₂SO₄, the solids were removed by filtration and the filtrate was concentrated by evaporation. The crude reaction product was purified by silica gel chromatography to give compound 26 (22 mg, 49%) as a white solid. ¹H-NMR (400 MHz, CDCl₃) δ: 7.95 (d, J=1.2 Hz, 1H), 7.73 (s, 4H), 7.47 (dd, J=2 Hz, J=8 Hz, 1H), 7.37 (d, J=8.4 Hz, 1H), 4.75 (m, 1H), 3.55 (m, 1H), 3.13 (m, 2H), 2.77 (m, 1H), 2.43 (m, 2H), 2.05 (m, 4H), 1.88 (m, 4H), 1.66 (m, 2H). MS (ESI): m/z 369.0 (M+H⁺).

### 27. Compound 27 (N-(4-(3-cyclobutyl-2,3,4,5-tetrahydro-1H-1,5-ethanobenzo[4,5]imidazo[1,2-d][1,4]diazepin-8-yl)phenyl)acetamide)

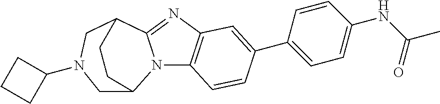

This compound was prepared in 52% yield (21 mg) as described for compound 26 but using 4-acetamidophenylboronic acid as the starting material. ¹H-NMR (400 MHz, CD₃OD) δ: 8.02 (s, 1H), 7.90 (m, 2H), 7.68 (m, 4H), 5.58 (m, 1H), 4.13 (m, 1H), 3.86 (m, 3H), 3.63 (m, 1H), 3.25 (m, 2H), 2.67 (m, 2H), 2.35 (m, 6H), 2.14 (s, 3H), 1.81 (m, 2H). MS (ESI): m/z 401.1 (M+H⁺).

### 28. Compound 28 (3-cyclobutyl-8-(1H-indol-5-yl)-2,3,4,5-tetrahydro-1H-1,5-ethanobenzo[4,5]imidazo[1,2-d][1,4]diazepine)

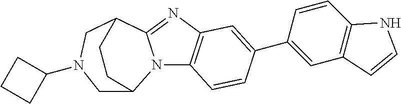

This compound was prepared in 11% yield (4 mg) as described for compound 26 but using 1H-indol-5-yl-boronic acid as the starting material. ¹H-NMR (400 MHz, CD₃OD) δ: 10.52 (s, 1H), 7.93 (s, 1H), 7.80 (m, 3H), 7.44 (d, J=8 Hz, 1H), 7.37 (dd, J=2 Hz, J=8 Hz, 1H), 7.24 (s, 1H), 6.46 (d, J=3.2 Hz, 1H), 5.41 (m, 1H), 3.91 (m, 1H), 3.56 (m, 2H), 3.37 (m, 1H), 2.92 (m, 2H), 2.51 (m, 2H), 2.15 (m, 6H), 1.78 (m, 2H). MS (ESI): m/z 383.1 (M+H⁺).

### 29. Compound 29 (3-cyclobutyl-8-(furan-2-yl)-2,3,4,5-tetrahydro-1H-1,5-ethanobenzo[4,5]imidazo[1,2-d][1,4]diazepine)

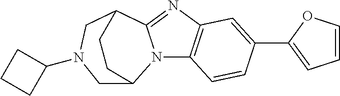

This compound was prepared in 19% yield (6 mg) as described for compound 26 but using furan-2-yl-boronic acid as the starting material. ¹H-NMR (400 MHz, CD₃OD) δ: 8.08 (s, 1H), 7.93 (d, J=8.8 Hz, 1H), 7.83 (d, J=8.8 Hz, 1H), 7.63 (d, J=1.6 Hz, 1H), 6.92 (d, J=2.8 Hz, 1H), 6.58 (m, 1H), 5.45 (m, 1H), 3.99 (m, 1H), 3.71 (m, 2H), 3.51 (m, 1H), 3.08 (m, 2H), 2.60 (m, 2H), 2.25 (m, 6H), 1.80 (m, 2H). MS (ESI): m/z 334.1 (M+H⁺).

### 30. Compound 30 (3-cyclobutyl-8-(pyrazin-2-yl)-2,3,4,5-tetrahydro-1H-1,5-ethanobenzo[4,5]imidazo[1,2-d][1,4]diazepine)

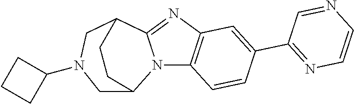

This compound was prepared in 25% yield (27 mg) as described for compound 2 but using intermediate I-37 and 2-bromopyrazine as starting materials. ¹H-NMR (400 MHz, CDCl₃) δ: 9.25 (d, J=0.8 Hz, 1H), 8.75 (m, 1H), 8.59 (d, J=2.8 Hz, 1H), 8.55 (d, J=0.5 Hz, 1H), 8.30 (dd, J=1.2 Hz, J=8.4 Hz, 1H), 7.94 (d, J=8.4 Hz, 1H), 5.51 (m, 1H), 4.01 (m, 1H), 3.79 (m, 2H), 3.60 (m, 1H), 3.19 (m, 2H), 2.61 (m, 2H), 2.30 (m, 6H), 1.82 (m, 2H). MS (ESI): m/z 346.1 (M+H⁺).

### 31. Compound 31 ((4-(3-cyclobutyl-2,3,4,5-tetrahydro-1H-1,5-ethanobenzo[4,5]imidazo[1,2-d][1,4]diazepin-8-yl)phenyl)methanamine)

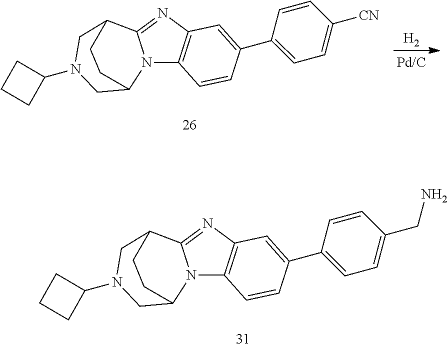

Compound 26 (15 mg, 0.04 mmol, 1.0 eq) was dissolved in methanol (2 mL) and three drops of concentrated aqueous HCl were added followed by palladium on carbon (10 mg, 5% Pd on carbon), and the reaction mixture was stirred under hydrogen atmosphere (1 atm) at room temperature for 2 hours. The solids were removed by filtration, and the filtrate was concentrated by evaporation. The crude reaction product was purified by preparative RP-HPLC to give compound 31 (5 mg, 31%) as a pale yellow solid. ¹H-NMR (400 MHz, CD₃OD) δ: 8.00 (s, 1H), 7.81 (m, 4H), 7.58 (d, J=8 Hz, 2H), 5.39 (m, 1H), 4.20 (s, 2H), 3.88 (t, J=4 Hz, 1H), 3.60 (m, 2H), 3.36 (m, 1H), 2.93 (m, 2H), 2.56 (m, 2H), 2.16 (m, 6H), 1.80 (m, 2H). MS (ESI): m/z 373.1 (M+H⁺).

### 32. Compound 32 (8-chloro-3-cyclobutyl-2,3,4,5-tetrahydro-1H-1,4-methanobenzo[4,5]imidazo[1,2-d][1,4]diazepine)

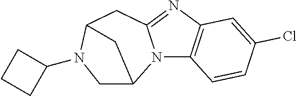

This compound was prepared in 80% yield (500 mg) as described for compound 18 but using intermediate I-48 as the starting material. ¹H-NMR (400 MHz, CD₃OD) δ: 7.67 (d, 2H, J=8.4 Hz), 7.39 (d, 1H, J=8.4 Hz), 5.53 (s, 1H), 4.50 (br, 1H), 3.95 (m, 2H), 3.65 (s, 2H), 3.52 (d, 1H, J=12.0 Hz), 2.76 (m, 1H), 2.56 (d, 1H, J=13.2 Hz), 2.32 (m, 3H), 2.18 (m, 1H), 1.85 (m, 2H). MS (ESI): m/z 288 (M+H⁺).

### 33. Compound 33 (4-(3-cyclobutyl-2,3,4,5-tetrahydro-1H-1,4-methanobenzo[4,5]imidazo[1,2-d][1,4]diazepin-8-yl)benzonitrile)

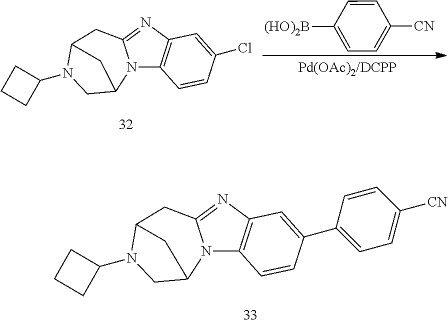

Compound 32 (65 mg, 0.23 mmol, 1.0 eq), 4-cyanophenylboronic acid (100 mg, 0.68 mmol, 3.0 eq), Pd(OAc)₂ (5 mg, 0.023 mmol, 0.1 eq), DCPP (32 mg, 0.068 mmol, 0.3 eq) and KF.H₂O (52 mg, 0.68 mmol, 3.0 eq) were dissolved in dioxane (2 mL) under nitrogen atmosphere, and the reaction mixture was heated under microwave irradiation at 120° C. for 3 hours. The crude reaction mixture was extracted with ethyl acetate, and the organic layer was washed with brine. The combined organic layers were dried over anhydrous Na₂SO₄, the solids were removed by filtration and the filtrate was concentrated by evaporation. The crude reaction product was purified by silica gel chromatography to give compound 33 (25 mg, 31%). ¹H-NMR (400 MHz, CDCl₃) δ: 8.02 (s, 1H), 7.77 (d, 2H, J=8.4 Hz), 7.73 (d, 2H, J=8.4 Hz), 7.66 (d, 1H, J=8.4 Hz), 7.54 (d, 1H, J=8.4 Hz), 5.21 (s, 1H), 4.63 (s, 1H), 4.06 (s, 1H), 3.78 (m, 1H), 3.52 (m, 2H), 3.21 (m, 1H), 2.92 (m, 1H), 2.34 (m, 2H), 2.17 (m, 2H), 2.00 (m, 1H), 1.85 (m, 1H), 1.78 (m, 1H). MS (ESI): m/z 355 (M+H⁺).

### 34. Compound 34 (3-cyclobutyl-8-(imidazo[1,2-a]pyridin-6-yl)-2,3,4,5-tetrahydro-1H-1,4-methanobenzo[4,5]imidazo[1,2-d][1,4]diazepine)

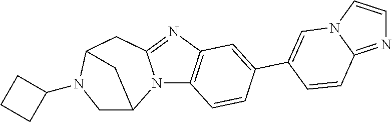

This compound was prepared in 29% yield (12 mg) as described for compound 2 but using intermediate I-49 and 6-bromoimidazo[1,2-c]pyridine as starting materials. ¹H-NMR (400 MHz, CDCl₃) δ: 8.32 (s, 1H), 7.85 (s, 1H), 7.70 (m, 3H), 7.50 (d, 1H, J=8.0 Hz), 7.40 (m, 2H), 4.90 (br, 1H), 3.85 (m, 1H), 3.35 (m, 1H), 3.25 (d, 1H, J=17.2 Hz), 3.10 (m, 2H), 2.95 (d, 1H, J=10.4 Hz), 2.33 (m, 1H), 2.14 (d, 1H, J=12.0 Hz), 2.05 (m, 1H), 2.00 (m, 1H), 1.75 (m, 4H). MS (ESI): m/z 370 (M+H⁺).

### 35. Compound 35 (8-chloro-3-cyclobutyl-2,3,4,5-tetrahydro-1H-2,5-methanobenzo[4,5]imidazo[1,2-d][1,4]diazepine)

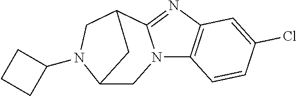

This compound was prepared in 60% yield (3.8 g) as described for intermediate I-28 but using intermediate I-56 as the starting material. ¹H-NMR (400 MHz, CD₃OD) δ: 7.57 (s, 1H), 7.40 (d, J=7.6 Hz, 1H), 7.24 (d, J=7.2 Hz, 1H), 4.08 (d, J=2 Hz, 2H), 3.84-3.86 (m, 1H), 3.63 (m, 1H), 3.39-3.47 (m, 1H), 3.12 (d, J=9.6 Hz, 1H), 2.99 (d, J=9.2 Hz, 1H), 2.24-2.29 (m, 1H), 2.05-2.12 (m, 2H), 1.87-2.08 (m, 3H), 1.65-1.80 (m, 2H). MS (CI): m/z 288 (M+H⁺).

### 36. Compound 36 (4-(3-cyclobutyl-2,3,4,5-tetrahydro-1H-2,5-methanobenzo[4,5]imidazo[1,2-d][1,4]diazepin-8-yl)benzonitrile)

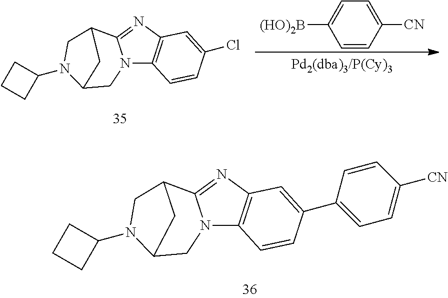

Compound 35 (58 mg, 0.2 mmol, 1.0 eq), 4-cyanobenzobronic acid (35 mg, 0.24 mmol, 1.2 eq), Pd₂(dba)₃ (18 mg, 0.1 eq), P(Cy)₃ (20 mg, 0.2 eq) and KF (32 mg, 0.56 mmol, 0.28 eq) were dissolved in a mixture of dioxane and water (1.6 mL:0.4 mL), and the reaction mixture was heated under microwave irradiation at 110° C. for 1 hour. The solids were removed by filtration, and the filtrate was concentrated by evaporation. The crude reaction product was purified by preparative TLC to give compound 36 (41 mg, 60%) as yellow oil. ¹H-NMR (400 MHz, CDCl₃) δ: 7.89 (s, 1H), 7.69-7.73 (m, 4H), 7.45 (d, J=8.4 Hz, 1H), 7.33 (d, J=8.4 Hz, 1H), 3.97-4.08 (m, 2H), 3.69-3.77 (m, 2H), 3.35-3.40 (m, 1H), 3.15 (d, J=9.6 Hz, 1H), 2.97-3.01 (m, 1H), 2.19-2.25 (m, 1H), 2.05-2.22 (m, 3H), 1.82-2.95 (m, 2H), 1.64-1.80 (m, 2H). MS (CI): m/z 355 (M+H⁺).

### 37. Compound 37 (3-cyclobutyl-8-(imidazo[1,2-a]pyridin-6-yl)-2,3,4,5-tetrahydro-1H-2,5-methanobenzo[4,5]imidazo[1,2-d][1,4]diazepine)

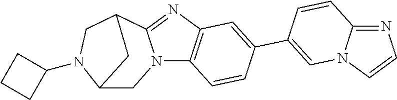

This compound was prepared in 50% yield (15 mg) as described for compound 2 but using intermediate I-57 and 6-bromoimidazo[1,2-a]pyridine as starting materials. ¹H-NMR (400 MHz, CD₃OD) δ: 8.74 (s, 1H), 7.86-7.91 (m, 2H), 7.52-7.69 (m, 5H), 4.12 (d, J=2 Hz, 2H), 3.84-3.86 (m, 1H), 3.65-3.68 (m, 1H), 3.39-3.47 (m, 1H), 3.12 (d, J=9.6 Hz, 1H), 3.00-3.04 (m, 1H), 2.26-2.29 (m, 1H), 1.91-2.17 (m, 5H), 1.65-1.80 (m, 2H). MS (CI): m/z 352 (M+H⁺).

### 38. Compound 38 (8-(1H-benzo[d]imidazol-1-yl)-3-cyclobutyl-2,3,4,5-tetrahydro-1H-2,5-methanobenzo[4,5]imidazo[1,2-d][1,4]diazepine)

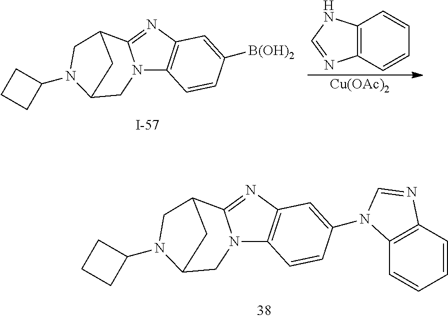

Intermediate I-57 (20 mg, 0.67 mmol, 1.0 eq), benzimidazole (23 mg, 0.2 mmol, 2.0 eq), Cu(OAc)₂ (4 mg, 0.02 mmol, 0.1 eq) were dissolved in EtOH (3 mL), and the reaction mixture was stirred at room temperature for 1 hour. The solids were removed by filtration, and the filtrate was extracted with ethyl acetate. The combined organic layers were dried over anhydrous Na₂SO₄, the solids were removed by filtration and the filtrated was concentrated by evaporation. The crude reaction product was purified by reverse phase silica gel chromatography to give compound 38 (6 mg, 23%) as yellow oil. ¹H-NMR (400 MHz, CDCl₃) δ: 8.13 (s, 1H), 7.88 (d, J=6 Hz, 1H), 7.81 (s, 1H), 7.49 (d, J=6.4 Hz, 1H), 7.40 (d, J=6.8 Hz, 1H), 7.31-7.36 (m, 3H), 4.05-4.09 (m, 2H), 3.71-3.80 (m, 2H), 3.37-3.40 (m, 1H), 3.18 (d, J=9.6 Hz, 1H), 2.98-3.02 (m, 1H), 2.00-2.11 (m, 4H), 1.71-1.94 (m, 4H). MS (CI): m/z 353 (M+H⁺).

### 39. Compound 39 (4-((3-cyclobutyl-2,3,4,5-tetrahydro-1H-2,5-methanobenzo[4,5]imidazo[1,2-d][1,7]diazepin-8-yl)methyl)morpholine)

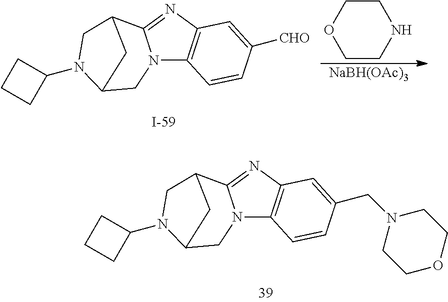

Intermediate I-59 (104 mg, 0.37 mmol, 1.0 eq), morpholine (64 mg, 0.74 mmol, 2.0 eq) and acetic acid (22 mg, 0.37 mmol, 1.0 eq) were dissolved in dry dichloromethane (10 mL), and the reaction mixture was stirred for 1 hour. 4 Å molecular sieves were added, and the reaction mixture was stirred for another 1 hour. Solid NaBH(OAc)₃ (117 mg, 0.56 mmol, 1.5 eq) was added, and the reaction mixture was stirred overnight. The solids were removed by filtration and the filtrate was concentrated by evaporation. The crude reaction product was purified by silica gel column chromatography to give compound 39 (38 mg, 30%) as yellow oil. ¹H-NMR (400 MHz, CD₃OD) δ: 7.58 (s, 1H), 7.38 (d, J=8 Hz, 1H), 7.28 (dd, J₁=8.4 Hz, J₂=1.6 Hz, 1H), 4.07 (d, J=2 Hz, 2H), 3.81-3.83 (m, 1H), 3.61-3.71 (m, 7H), 3.39-3.47 (m, 1H), 3.09 (d, J=9.6 Hz, 1H), 2.97-3.01 (m, 1H), 2.48-2.50 (m, 4H), 2.23-2.26 (m, 1H), 2.06-2.13 (m, 3H), 1.94-1.97 (m, 1H), 1.88-1.89 (m, 1H), 1.65-1.77 (m, 2H). MS (CI) m/z 343 (M+H⁺).

### 40. Compound 40 (8-bromo-3-cyclobutyl-4-methyl-2,3,4,5-tetrahydro-1H-benzo[4,5]imidazo[1,2-d][1,4]diazepine)

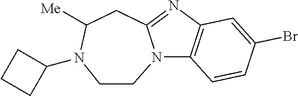

This compound was prepared in 73% yield (260 mg) as described for compound 18 but using intermediate I-64 as the starting material. ¹H-NMR (400 MHz, CDCl₃) δ: 7.84 (m, 1H), 7.32-7.35 (d, 1H, J=2 Hz), 7.09-7.11 (d, 1H, J=8.8 Hz), 4.13-4.17 (m, 2H), 3.34-3.38 (m, 2H), 3.20-3.29 (m, 2H), 2.87-2.92 (m, 1H), 2.68-2.72 (m, 1H), 2.06-2.11 (m, 2H), 1.85-1.94 (m, 2H), 1.64-1.94 (m, 2H), 0.78-0.80 (m, 3H). MS (ESI): m/z 333 (M+H⁺).

### 41. Compound 41 (4-(3-cyclobutyl-4-methyl-2,3,4,5-tetrahydro-1H-benzo[4,5]imidazo[1,2-d][1,4]diazepin-8-yl)benzonitrile)

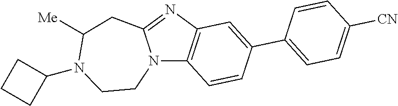

This compound was prepared in 30% yield (20 mg) as described for compound 2 but using compound 40 as the starting material. ¹H-NMR (400 MHz, CDCl₃) δ: 7.94 (s, 1H), 7.72 (m, 3H), 7.53 (m, 1H), 7.36-7.38 (d, 2H, J=8 Hz), 4.46-4.71 (m, 2H), 3.45-3.87 (m, 5H), 2.87-2.93 (m, 1H), 2.42-2.51 (m, 2H), 2.22-2.25 (m, 2H), 1.70-1.93 (m, 2H), 1.00-1.02 (m, 3H). MS (ESI): m/z 356 (M+H⁺).

### 42. Compound 42 (8-bromo-3-cyclobutyl-2-methyl-2,3,4,5-tetrahydro-1H-benzo[4,5]imidazo[1,2-d][1,4]diazepine)

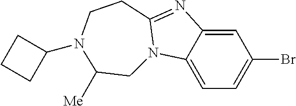

This compound was prepared in 58% yield (2.3 g) as described for compound 18 but using intermediate I-71 as the starting material. ¹H-NMR (400 MHz, CDCl₃) δ: 7.82 (d, 1H, J=1.6 Hz), 7.34 (dd, 1H, J₁=1.6 Hz, J₂=8.4 Hz), 7.09 (d, 1H, J=8.4 Hz), 4.33 (br, 1H), 4.16-4.11 (m, 1H), 3.22 (br, 1H), 3.20-3.16 (m, 3H), 2.87-2.84 (m, 1H), 2.60-2.53 (m, 1H), 2.11-2.07 (m, 2H), 1.87-1.85 (m, 2H), 1.72-1.62 (m, 2H), 0.65 (d, 3H, J=6.8 Hz). MS (ESI): m/z 334 (M+H⁺).

### 43. Compound 43 (4-(3-cyclobutyl-2-methyl-2,3,4,5-tetrahydro-1H-benzo[4,5]imidazo[1,2-d][1,4]diazepin-8-yl)benzonitrile)

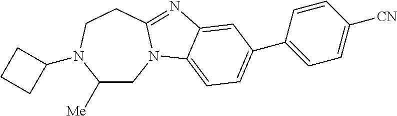

This compound was prepared in 38% yield (20 mg) as described for compound 2 but using compound 42 as the starting material. ¹H-NMR (400 MHz, CD₃OD) δ: 7.85 (m, 3H), 7.77 (d, 2H, J=8.4 Hz), 7.60 (d, 1H, J=8.4 Hz), 7.54 (d, 1H, J=8.4 Hz), 4.44-4.35 (m, 2H), 3.38-3.32 (m, 2H), 3.27-3.20 (m, 1H), 3.10-3.05 (m, 1H), 2.94-2.90 (m, 1H), 2.64 (t, 1H, J=12.8 Hz), 2.16-2.12 (m, 2H), 1.93-1.88 (m, 2H), 1.75-1.70 (m, 2H), 0.68 (d, 3H, J=6.8 Hz). MS (ESI): m/z 357 (M+H⁺).

### 44. Compound 44

### 45. Compound 45

### (4-((7S,10R)-13-cyclobutyl-6,7,8,9,10,11-hexahydro-7,10-epiminobenzo[4,5]imidazo[1,2-a]azocin-3-yl)benzonitrile and 4-((7R,10S)-13-cyclobutyl-6,7,8,9,10,11-hexahydro-7,10-epiminobenzo[4,5]imidazo[1,2-a]azocin-3-yl)benzonitrile)

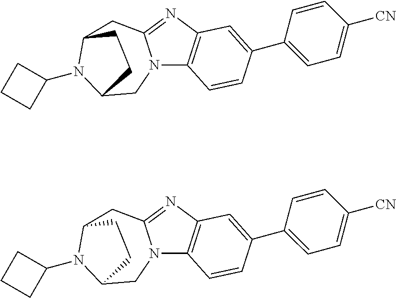

Enantiomers of compound 20 were separated by chiral chromatography. Compound 20 was dissolved in methanol, and the solution was filtered through a 0.5 μm filter cartridge. The isocratic normal-phase method used a mixture of 5% methanol, 5% ethanol and 0.1% diethylamine in hexanes. The column was a ChiralPak AS™ column (ChiralTech Technologies™) in a 1.0×25.0 cm format with a mobile phase flow of 100 mL/minute. The enantiomers of compound 20 were isolated as two separate peaks during chiral separation. The faster eluting peak was designated as compound 44. The more slowly eluting peak was designated as compound 45.

### 46. Compound 46

### 47. Compound 47

### ((S)-4-(1,2,3,4,6,7,14,14a-octahydrobenzo[4,5]imidazo[1,2-d]pyrido[2,1-g][1,4]diazepin-11-yl)benzonitrile and (R)-4-(1,2,3,4,6,7,14,14a-octahydrobenzo[4,5]imidazo[1,2-d]pyrido[2,1-g][1,4]diazepin-11-yl)benzonitrile)

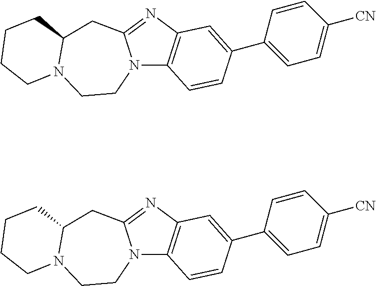

Enantiomers of compound 8 were separated by chiral chromatography. Compound 8 was dissolved in methanol, and the solution was filtered through a 0.5 μm filter cartridge. The isocratic SFC method used a mixture of 45% methanol in CO₂. The column was a Chiracel OD-H™ (Chiral Technologies™) in a 3.0×25.0 cm format with a mobile phase flow of 80 g/minute. The enantiomers of compound 8 were isolated as two separate peaks during chiral separation. The faster eluting peak was designated as compound 46. The more slowly eluting peak was designated as compound 47.

### 48. Compound 48 ((R)-11-(imidazo[1,2-a]pyridin-6-yl)-1,2,3,4,6,7,14,14a-octahydrobenzo[4,5]imidazo[1,2-d]pyrido[2,1-g][1,4]diazepine)

### 49. Compound 49 ((S)-11-(imidazo[1,2-a]pyridin-6-yl)-1,2,3,4,6,7,14,14a-octahydrobenzo[4,5]imidazo[1,2-d]pyrido[2,1-g][1,4]diazepine)

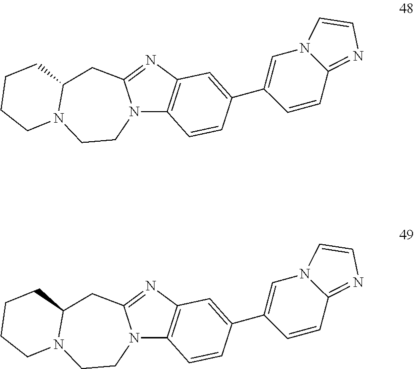

Enantiomers of compound 15 were separated by chiral chromatography. Compound 15 was dissolved in methanol, and the solution was filtered through a 0.5 μm filter cartridge. The isocratic SFC method used a mixture of 60% isopropanol with 0.5% isopropyl amine in CO₂. The column was a RegisPack™ (Regis Technologies™) in a 3.0×25.0 cm format with a mobile phase flow of 80 g/minute. The enantiomers of compound 15 were isolated as two separate peaks during chiral separation. The faster eluting peak was designated as compound 48. The more slowly eluting peak was designated as compound 49.

The absolute configuration of compound 48 was determined by vibrational circular dichroism. Compounds 48 and 49 were each dissolved in CDCl₃ (5 mg sample in 125 μL CDCl₃) and placed in a 100-μm pathlength cell with BaF₂ windows. IR and VCD spectra were recorded on a ChiralIR™ VCD spectrometer equipped with DualPEM accessory (BioTools, Inc., Jupiter, Fla.) at 4 cm⁻¹ resolution, with the instrument optimized at 1400 cm⁻¹ and 4-h collection for each sample and solvent. The (R)-configuration of compound 48 was built with Hyperchem (Hypercube, Inc., Gainesville, Fla.). A conformational search with Spartan 06 (Wavefunction, Inc., Irvine, Calif.) yielded 4 possible conformers. Geometry, frequency and IR and VCD intensity calculations were carried out at the DFT level (B3LYP functional/6-31G(d) basis set) with Gaussian 03 (Gaussian Inc., Wallingford, Conn.). Two low energy conformers were identified; the other two conformers with a different pucker of the 7-membered ring were over 2 kcal/mol higher in energy, presumably not significantly populated at room temperature. The calculated frequencies were scaled by 0.97, and the IR and VCD intensities were converted to Lorentzian bands with 6-cm⁻¹ half-width for comparison to experimental values. The calculated spectra were compared to solvent-subtracted IR and VCD spectra of both enantiomers. Observed VCD pattern for compound 48 agrees well with the calculation for (R)-configuration. This VCD investigation established assignment of the absolute configuration of compound 48 as (R).

### 50. Compound 50

### 51. Compound 51

### ((S)-4-((1,2,3,4,6,7,14,14a-octahydrobenzo[4,5]imidazo[1,2-d]pyrido[2,1-g][1,4]diazepin-11-yl)methyl)morpholine and (R)-4-((1,2,3,4,6,7,14,14a-octahydrobenzo[4,5]imidazo[1,2-d]pyrido[2,1-g][1,4]diazepin-11-yl)methyl)morpholine)

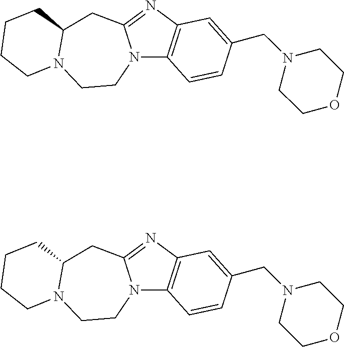

Enantiomers of compound 17 were separated by chiral chromatography. Compound 17 was dissolved in methanol, and the solution was filtered through a 0.5 μm filter cartridge. The isocratic SFC method used a mixture of 40% methanol with 2% isopropyl amine in CO₂. The column was a Chiracel AD-H™ (Chiral Technologies™) in a 3.0×25.0 cm format with a mobile phase flow of 80 g/minute. The enantiomers of compound 17 were isolated as two separate peaks during chiral separation. The faster eluting peak was designated as compound 50. The more slowly eluting peak was designated as compound 51.

### 52. Compound 52 (8-bromo-2-cyclobutyl-1,2,3,4-tetrahydrobenzo[4,5]imidazo[1,2-a]pyrazine)

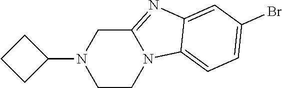

This compound was prepared in 58% yield (350 mg) as described for intermediate I-28 but using intermediate I-73 as the starting material. ¹H-NMR (400 MHz, CDCl₃) δ: 7.83 (d, 1H, J=2.0), 7.34 (m, 1H), 7.16 (d, 1H, J=8.4), 4.08 (t, 2H, J=5.6, 5.2), 3.79 (s, 2H), 3.03 (m, 1H), 2.89 (t, 2H, J=5.6, 5.6), 2.16 (m, 2H), 1.98 (m, 2H), 1.78 (m, 2H). MS (ESI): m/z 306 (M+H⁺).

### 53. Compound 53 (4-(2-cyclobutyl-1,2,3,4-tetrahydrobenzo-[4,5]imidazo[1,2-a]pyrazin-8-yl)benzonitrile)

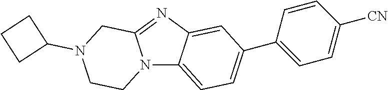

This compound was prepared in 47% yield (15 mg) as described for compound 33 but using compound 52 as the starting material. ¹H-NMR (400 MHz, CDCl₃) δ: 7.92 (s, 1H), 7.73 (s, 4H), 7.50 (m, 1H), 7.41 (d, 1H, J=8.4), 4.17 (t, 2H, J=5.6, 6.4), 3.85 (s, 2H), 3.07 (m, 1H), 2.94 (t, 2H, J=6.0, 5.6), 2.19 (m, 2H), 1.99 (m, 2H), 1.80 (m, 2H). MS (ESI): m/z 329 (M+H⁺).

### 54. Compound 54 (2-cyclobutyl-8-(imidazo[1,2-a]pyridin-6-yl)-1,2,3,4-tetrahydrobenzo[4,5]imidazo[1,2-a]pyrazine)

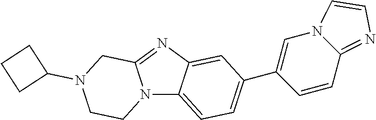

This compound was prepared in 54% yield (13 mg) as described for compound 2 but using intermediate I-74 and 6-bromoimidazo[1,2-a]pyridine as starting materials. ¹H-NMR (400 MHz, CDCl₃) δ: 8.33 (s, 1H), 7.86 (s, 1H), 7.66 (m, 3H), 7.43 (m, 3H), 4.17 (t, 2H, J=5.6, 5.6), 3.84 (s, 2H), 3.09 (m, 1H), 2.94 (t, 2H, J=5.2, 5.6), 2.20 (m, 2H), 2.01 (m, 2H), 1.79 (m, 2H). MS (ESI): m/z 344 (M+H⁺).

### 55. Compound 55 (8-(1H-benzo[d]imidazol-1-yl)-2-cyclobutyl-1,2,3,4-tetrahydrobenzo[4,5]imidazo[1,2-a]pyrazine)

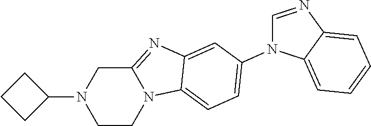

This compound was prepared in 30% yield (10 mg) as described for compound 10 but using compound 52 and 1H-benzimidazole as the starting material. ¹H-NMR (400 MHz, CDCl₃) δ: 8.15 (s, 1H), 7.90 (m, 1H), 7.82 (d, 1H, J=2.0), 7.49 (m, 2H), 7.38 (m, 3H), 4.21 (t, 2H, J=5.2, 5.6), 3.86 (s, 2H), 3.08 (m, 1H), 2.96 (t, 2H, J=6.0, 5.6), 2.20 (m, 2H), 2.00 (m, 2H), 1.82 (m, 2H). MS (ESI): m/z 344 (M+H⁺).

### 56. Compound 56 (4-((2-cyclobutyl-1,2,3,4-tetrahydrobenzo-[4,5]imidazo[1,2-a]pyrazin-8-yl)methyl)morpholine)

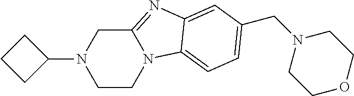

This compound was prepared in 19% yield (12 mg) as described for compound 17 but using intermediate I-76 as the starting material. ¹H-NMR (400 MHz, CDCl₃) δ: 7.62 (s, 1H), 7.26 (s, 2H), 4.10 (m, 2H), 3.79 (s, 2H), 3.70 (t, 4H, J=4.8, 4.4), 3.64 (s, 2H), 3.03 (m, 1H), 2.89 (t, 2H, J=5.6, 5.6), 2.48 (t, 4H, J=4.0, 4.4), 2.17 (m, 2H), 1.97 (m, 2H), 1.78 (m, 2H). MS (ESI): m/z 327 (M+H⁺).

### 57. Compound 57 (10-chloro-3-cyclobutyl-1,2,3,4,5,6-hexahydrobenzo[4,5]imidazo[1,2-a][1,5]diazocine)

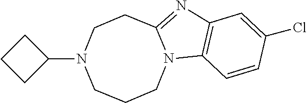

This compound was prepared in 70% yield (900 mg) as described for intermediate I-28 but using intermediate I-85 as the starting material. ¹H-NMR (400 MHz, CDCl₃) δ: 7.67 (s, 1H), 7.19 (m, 2H), 4.39 (t, 2H, J=5.6 Hz), 3.13˜3.09 (m, 3H), 2.78˜2.76 (m, 2H), 2.07˜2.00 (m, 4H), 1.77˜1.57 (m, 6H). MS (ESI): m/z 290 (M+H⁺).

### 58. Compound 58 (4-(3-cyclobutyl-1,2,3,4,5,6-hexahydrobenzo[4,5]imidazo[1,2-a][1,5]diazocin-10-yl)benzonitrile)

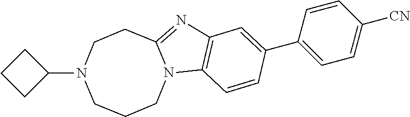

This compound was prepared in 20% yield (15 mg) as described for compound 33 but using compound 57 as the starting material. ¹H-NMR (400 MHz, CDCl₃): δ 8.05˜8.03 (m, 1H), 7.77˜7.71 (m, 4H), 7.61 (d, 1H, J=8.8 Hz), 7.55˜7.45 (m, 1H), 4.74 (s, 2H), 3.90˜3.00 (m, 7H), 2.55˜2.50 (m, 3H), 2.27˜2.23 (m, 3H), 1.96˜1.88 (m, 1H), 1.74˜1.67 (m, 1H). MS (ESI): m/z 357 (M+H⁺).

### 59. Compound 59 (3-cyclobutyl-10-(imidazo[1,2-a]pyridin-6-yl)-1,2,3,4,5,6-hexahydrobenzo[4,5]imidazo[1,2-a][1,5]diazocine)

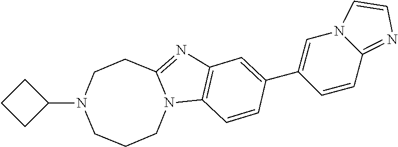

This compound was prepared in 61% yield (30 mg) as described for compound 2 but using intermediate I-86 and 6-bromoimidazo[1,2-a]pyridine as starting materials. ¹H-NMR (400 MHz, CDCl₃): δ 8.31 (s, 1H), 7.86 (d, 1H, J=0.8 Hz), 7.70˜7.64 (m, 3H), 7.49 (dd, 1H, J₁=1.6 Hz, J₂=8.8 Hz), 7.44˜7.37 (m, 2H), 4.45 (t, 2H, J=6.0 Hz), 3.16˜3.13 (m, 3H), 2.78 (t, 2H, J=5.6 Hz), 2.13˜2.10 (m, 2H), 2.07˜2.00 (m, 2H), 1.84˜1.79 (m, 4H), 1.69˜1.59 (m, 2H), MS (ESI): m/z 372 (M+H⁺).

### 60. Compound 60 (13-cyclobutyl-3-(imidazo[1,2-a]pyridin-6-yl)-6,7,8,9,10,11-hexahydro-7,10-epiminobenzo[4,5]imidazo[1,2-a]azocine)

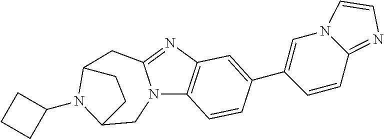

This compound was prepared in 49% yield (80 mg) as described for compound 2 but using intermediate I-30 and 6-bromoimidazo[1,2-a]pyridine as starting materials. ¹HNMR (400 MHz, CDCl₃): δ 8.31 (s, 1H), 7.84 (s, 1H), 7.65-7.70 (m, 3H), 7.42-7.50 (m, 2H), 7.30 (d, J=8.4 Hz, 1H), 4.21 (m, 2H), 3.75 (s, 1H) 3.66 (s, 1H), 3.30-3.35 (m, 3H), 2.12-2.18 (m, 2H), 1.94-2.07 (m, 4H), 1.74-1.85 (m, 2H), 1.47 (t, J=8.0 Hz, 2H) 1.30 (t, J=8.0 Hz, 1H). MS (ESI): m/z 384.1 (M+H⁺).

### 61. Compound 61 (3-(1H-benzo[d]imidazol-1-yl)-13-cyclobutyl-6,7,8,9,10,11-hexahydro-7,10-epiminobenzo[4,5]imidazo[1,2-a]azocine)

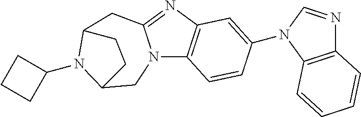

This compound was prepared in 45% yield (8 mg) as described for compound 38 but using intermediate I-88 as the starting material. ¹HNMR (400 MHz, CD₃OD): δ 8.45 (s, 1H), 7.79-7.82 (m, 2H), 7.73 (d, J=8.4 Hz, 1H), 7.54-7.59 (m, 2H), 7.38-7.41 (m, 2H), 4.63 (m, 1H), 4.43 (m, 1H), 3.94 (m, 1H), 3.81 (m, 1H), 3.34-3.51 (m, 3H), 2.28 (m, 2H), 2.11-2.23 (m, 4H), 1.80-1.95 (m, 2H), 1.31-1.48 (m, 2H). MS (ESI): m/z 384.1 (M+H⁺).

### 62. Compound 62 (4-((13-cyclobutyl-6,7,8,9,10,11-hexahydro-7,10-epiminobenzo[4,5]imidazo[1,2-a]azocin-3-yl)methyl)morpholine)

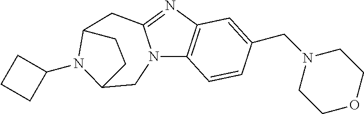

This compound was prepared in 50% yield (14 mg) as described for compound 39 but using intermediate I-90 as the starting material. ¹HNMR (400 MHz, CDCl₃): δ 7.60 (s, 1H), 7.25 (d, J=8.4 Hz, 1H), 7.16 (d, J=8.4 Hz, 1H), 4.14-4.18 (m, 2H), 3.72 (m, 4H), 3.64 (s, 3H), 3.55 (s, 1H) 3.28-3.34 (m, 3H), 2.50 (m, 4H), 2.10-2.17 (m, 2H), 1.97-2.04 (m, 4H), 1.72-1.97 (m, 2H), 1.47 (m, 1H) 1.28 (m, 1H). MS (ESI): m/z 367 (M+H⁺).

### 63. Compound 63 (11-((3,4-dihydroisoquinolin-2(1H)-yl)methyl)-1,2,3,4,6,7,14,14a-octahydrobenzo[4,5]imidazo[1,2-d]pyrido[2,1-g][1,4]diazepine)

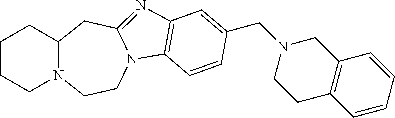

This compound was prepared in 60% yield (1.9 g) as described for compound 17 but using 1,2,3,4-tetrahydroisoquinoline as the starting material. ¹HNMR (400 MHz, CD₃OD): δ 7.62 (s, 1H), 7.43 (d, J=8.4 Hz, 1H), 7.34 (m, J=8.4 Hz, 1H), 7.05-7.09 (m, 3H), 6.94 (m, J=6.4 Hz, 1H), 4.49-4.58 (m, 1H), 4.21-4.29 (m, 1H), 3.81 (s, 2H), 3.63 (s, 2H), 3.12-3.18 (m, 2H), 2.77-3.05 (m, 6H), 2.40-2.50 (m, 1H), 2.20-2.30 (m, 2H), 1.75-1.86 (m, 2H), 1.47-1.70 (m, 3H), 1.38 (brs, 1H). MS (ESI): m/z 387.7 (M+H⁺).

### 64. Compound 64 ((R)-11-((3,4-dihydroisoquinolin-2(1H)-yl)methyl)-1,2,3,4,6,7,14,14a-octahydrobenzo[4,5]imidazo[1,2-d]pyrido[2,1-g][1,4]diazepine)

### 65. Compound 65 ((S)-11-((3,4-dihydroisoquinolin-2(1H)-yl)methyl)-1,2,3,4,6,7,14,14a-octahydrobenzo[4,5]imidazo[1,2-d]pyrido[2,1-g][1,4]diazepine

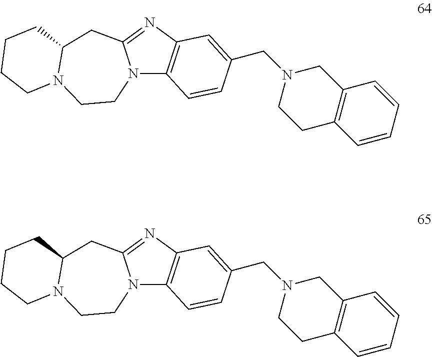

Enantiomers of compound 63 were separated by chiral chromatography. Compound 63 was dissolved in methanol, and the solution was filtered through a 0.5 μm filter cartridge. The isocratic SFC method used a mixture of 45% isopropanol with 0.5% isopropyl amine in CO₂ as the mobile phase. The column was a RegisPack™ column (Regis Technologies™) in a 3.0×25.0 cm format with a mobile phase flow of 80 mL/min. The enantiomers of compound 63 were isolated as two separate peaks during chiral separation. The faster eluting peak was designated as compound 64. The more slowly eluting peak was designated as compound 65. The absolute configuration of compound 64 was determined by vibrational circular dichroism as described for compound 48.

### 66. Compound 66 (4-(2,3,4,6,7,8,15,15a-octahydro-1H-benzo[4,5]imidazo[1,2-a]pyrido[2,1-d][1,5]diazocin-12-yl)benzonitrile)

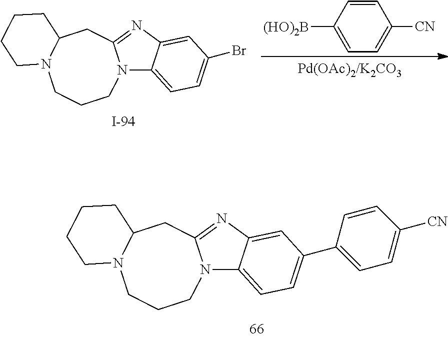

Intermediate I-94 (80 mg, 0.24 mmol, 1.0 eq), 4-cyanophenylboronic acid (53 mg, 0.36 mmol, 1.5 eq), Pd(OAc)₂ (8 mg, 10% w/w), dicyclohexyl(2′,4′,6′-triisopropylbiphenyl-2-yl)phosphine (8 mg, 10% w/w) and potassium carbonate (99 mg, 0.72 mmol, 2.0 eq) were dissolved in DMF (3 mL) in a microwave tube that was flushed with argon. The reaction mixture was heated to 100° C. under microwave irradiation for 90 minutes. Ethyl acetate was added and solids were removed by filtration through a short plug of silica gel. The filtrate was washed with water, the combined organic layers were dried over anhydrous Na₂SO₄, the solids were removed by filtration, and the filtrate was concentrated by evaporation. The crude reaction product was purified by preparative reverse-phase chromatography to give compound 66 (20 mg, 26%). ¹H-NMR (400 MHz, CDCl₃): δ 7.93 (d, 1H, J=1.6 Hz), 7.73 (m, 4H), 7.44 (dd, 1H, J=1.2 Hz), 7.37 (dd, 1H, J=1.2 Hz), 4.45 (m, 1H), 4.26 (m, 1H), 3.23 (dd, 1H, J=3.6 Hz), 2.92 (m, 1H), 2.89 (m, 1H), 2.67 (m, 1H), 2.49 (m, 1H), 2.35 (m, 1H), 1.93 (m, 2H), 1.72 (m, 4H), 1.38 (m, 3H). MS (ESI): m/z 372 (M+H⁺).

### 67. Compound 67 (12-(imidazo[1,2-a]pyridin-6-yl)-2,3,4,6,7,8,15,15a-octahydro-1H-benzo[4,5]imidazo[1,2-a]pyrido[2,1-d][1,5]diazocine)

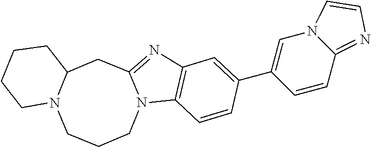

This compound was prepared in 26% yield (20 mg) as described for compound 2 but using intermediate I-95 and 6-bromoimidazo[1,2-a]pyridine as starting materials. ¹HNMR (400 MHz, CD₃OD): δ 8.93 (s, 1H), 8.09 (s, 1H), 7.96 (m, 2H), 7.81 (m, 2H), 7.74 (d, 1H, J=8.4 Hz), 7.68 (d, 1H, J=8.4 Hz), 4.64 (m, 2H), 3.69 (m, 2H), 3.60 (m, 2H), 3.31 (m, 2H), 2.41 (m, 2H), 2.17 (m, 1H), 1.72 (m, 6H).

### 68. Compound 68 (4-((2,3,4,6,7,8,15,15a-octahydro-1H-benzo[4,5]imidazo[1,2-a]pyrido[2,1-d][1,5]diazocin-12-yl)methyl)morpholine)

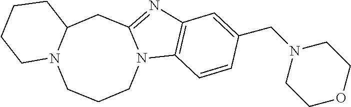

This compound was prepared in 49% yield (28 mg) as described for compound 39 but using intermediate I-97 as the starting material. ¹H-NMR (400 MHz, CD₃OD): δ 7.55 (s, 1H), 7.42 (d, 1H, J=8.0 Hz), 7.25 (d, 1H, J=0.8 Hz), 4.33 (m, 2H), 3.73 (s, 2H), 3.63 (m, 1H), 3.38 (m, 1H), 2.81 (m, 6H), 2.60 (t, 1H, J=4.8 Hz), 2.00 (m, 2H), 1.80 (m, 3H), 1.50 (m, 3H). MS (ESI): m/z 355 (M+H⁺).

### 69. Compound 69 (4-((2,3,4,6,7,8,15,15a-octahydro-1H-benzo[4,5]imidazo[1,2-a]pyrido[2,1-d][1,5]diazocin-12-yl)methyl)morpholine)

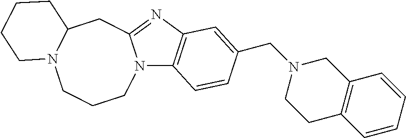

This compound was prepared 32% yield (25 mg) as described for compound 17 but using intermediate I-97 and 1,2,3,4-tetrahydroisoquinoline as starting materials. ¹H-NMR (400 MHz, CD₃OD): δ 7.56 (d, 1H, J=0.8 Hz), 7.44 (d, 1H, J=4.4 Hz), 7.27 (dd, 1H, J=1.6 Hz), 7.01 (m, 3H), 6.89 (m, 1H), 4.26 (m, 2H), 3.82 (s, 2H), 3.64 (m, 2H), 2.82 (m, 6H), 2.61 (m, 2H), 2.30 (m, 2H), 1.82 (m, 1H), 1.54 (m, 5H), 1.26 (m, 3H). MS (ESI): m/z 401 (M+H⁺).

### 70. Compound 70

### 71. Compound 71

### ((R)-12-(imidazo[1,2-a]pyridin-6-yl)-2,3,4,6,7,8,15,15a-octahydro-1H-benzo[4,5]imidazo[1,2-a]pyrido[2,1-d][1,5]diazocine and (5)-12-(imidazo[1,2-a]pyridin-6-yl)-2,3,4,6,7,8,15,15a-octahydro-1H-benzo[4,5]imidazo[1,2-a]pyrido[2,1-d][1,5]diazocine)

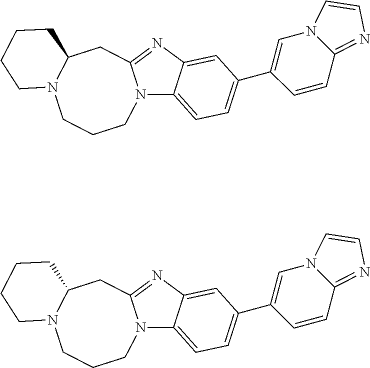

Enantiomers of compound 67 were separated by chiral chromatography. Compound 67 was dissolved in methanol, and the solution was filtered through a 0.5 μm filter cartridge. The isocratic SFC method used a mixture of 50% isopropanol with 1% isopropyl amine in CO₂ as the mobile phase. The column was a RegisPack™ column (Regis Technologies™) in a 3.0×25.0 cm format with a mobile phase flow of 80 mL/min. The enantiomers of compound 67 were isolated as two separate peaks during chiral separation. The faster eluting peak was designated as compound 70. The more slowly eluting peak was designated as compound 71.

### 72. Compound 72 (10-bromo-2,3,5,6,13,13a-hexahydro-1H-benzo[4,5]imidazo[1,2-d]pyrrolo[2,1-g][1,4]diazepine)

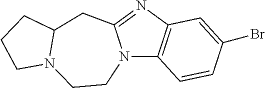

1.5 g of the title compound was prepared as described for compound 1 via intermediates I-4, I-5 and I-6 but using racemic 2-(1-(tert-butoxycarbonyl)pyrrolidin-2-yl)acetic acid as the starting material. ¹H-NMR (400 MHz, CD₃OD): δ 7.90 (d, J=1.6 Hz, 1H), 7.60-7.68 (m, 2H), 5.03-5.08 (dd, J₁=16.4 Hz, J₂=4.4 Hz, 1H), 4.54-4.61 (m, 1H), 4.10-4.16 (dd, J₁=12.8 Hz, J₂=4.4 Hz, 1H), 3.88-3.94 (m, 1H), 3.62-3.77 (m, 3H), 3.45-3.51 (m, J=12.8 Hz, 1H), 3.28-3.35 (m, 1H), 2.51-2.55 (m, 1H), 2.03-2.22 (m, 3H). MS (ESI): m/z 478 (M+H⁺).

### 73. Compound 73 (4-(2,3,5,6,13,13a-hexahydro-1H-benzo[4,5]imidazo[1,2-d]pyrrolo[2,1-g][1,4]diazepin-10-yl)benzonitrile)

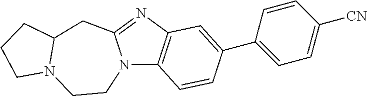

This compound was prepared in 50% yield (54 mg) as described for compound 2 but using compound 72 as the starting material. ¹H-NMR (400 MHz, CDCl₃): δ 7.91 (s, 1H), 7.72 (s, 1H), 7.48-7.51 (dd, J₁=8.0 Hz, J₂=4.0 Hz, 1H), 7.35-7.37 (d, J=8.4 Hz, 1H), 4.44-4.48 (dd, J₁=14.0 Hz, J₂=4.4 Hz, 1H), 4.17-4.24 (m, 1H), 3.38-3.48 (m, 2H), 3.22-3.26 (t, J=8.0 Hz, 1H), 2.96-3.03 (m, 1H), 2.97 (1H, m), 2.43 (2H, m), 2.36 (1H, m), 2.13 (1H, m), 2.01 (1H, m), 1.90 (2H, m), 2.27-2.45 (m, 3H), 2.10-2.17 (m, 1H), 1.89-1.94 (m, 1H), 1.65-1.80 (m, 2H). MS (ESI): m/z 329 (M+H⁺).

### 74. Compound 74 (10-(imidazo[1,2-a]pyridin-6-yl)-2,3,5,6,13,13a-hexahydro-1H-benzo[4,5]imidazo[1,2-d]pyrrolo[2,1-g][1,4]diazepine)

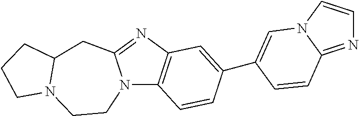

This compound was prepared in 30% yield (41 mg) as described for compound 2 but using intermediate I-98 and 6-bromoimidazo[1,2-a]pyridine as starting materials. ¹HNMR (400 MHz, CDCl₃): δ 8.31 (s, 1H), 7.86 (d, J=1.2 Hz, 1H), 7.64-7.70 (m, 3H), 7.43-7.49 (m, 2H), 7.34-7.36 (d, J=8.4 Hz, 1H), 4.43-4.48 (dd, J₁=14.4 Hz, J₂=4.0 Hz, 1H), 4.16˜4.23 (m, 1H), 3.37-3.49 (m, 2H), 3.21-3.25 (t, J=7.6 Hz, 1H), 2.95-3.02 (m, 1H), 2.27-2.45 (m, 3H), 2.10-2.17 (m, 1H), 1.89-1.94 (m, 1H), 1.65-1.80 (m, 2H). MS (ESI): m/z 344 (M+H⁺).

### 75. Compound 75 (4-((2,3,5,6,13,13a-hexahydro-1H-benzo[4,5]imidazo[,2-d]pyrrolo[2,1-g][1,4]diazepin-10-yl)methyl)morpholine)

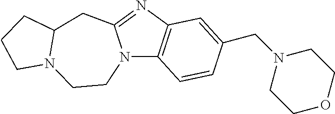

This compound was prepared in 50% yield (82 mg) as described for compound 39 but using intermediate I-100 as the starting material. ¹H-NMR (400 MHz, CDCl₃): δ 7.61-7.62 (d, J=0.4 Hz, 1H), 7.20-7.26 (m, 2H), 4.38-4.42 (dd, J₁=14.4 Hz, J₂=4.0 Hz, 1H), 4.09-4.16 (m, 1H), 3.67-3.70 (t, J=4.4 Hz, 2H), 3.60 (s, 2H), 3.32-3.43 (m, 2H), 3.18-3.23 (m, 1H), 2.90-2.97 (m, 1H), 2.34-2.46 (m, 6H), 2.23-2.30 (m, 1H), 2.06-2.13 (m, 1H), 1.84-1.91 (m, 1H), 1.62-1.77 (m, 2H). MS (ESI): m/z 327 (M+H⁺).

### 76. Compound 76 (10-((3,4-dihydroisoquinolin-2(1H)-yl)methyl)-2,3,5,6,13,13a-hexahydro-1H-benzo[4,5]imidazo[1,2-d]pyrrolo[2,1-g][1,4]diazepine)

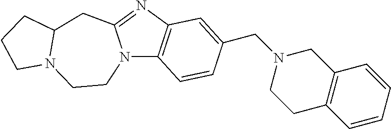

This compound was prepared in 50% yield (93 mg) as described for compound 17 but using intermediate I-100 and 1,2,3,4-tetrahydroisoquinoline as starting materials. ¹H-NMR (400 MHz, CDCl₃): δ 7.66-7.68 (d, J=0.8 Hz, 1H), 7.32-7.34 (dd, J₁=8.4 Hz, J₂=1.6 Hz, 1H), 7.19-7.20 (d, J=8.0 Hz, 1H), 7.02-7.10 (m, 3H), 6.91-6.93 (m, 1H), 4.34-4.39 (dd, J₁=10.4 Hz, J₂=4.0 Hz, 1H), 4.06-4.12 (m, 1H), 3.77 (s, 2H), 3.62 (s, 2H), 3.39-3.43 (d, J=15.6 Hz, 1H), 3.28-3.32 (dd, J₁=13.6 Hz, J₂=4.4 Hz, 1H), 3.16-3.20 (t, J=8.0 Hz, 1H), 2.84-2.95 (m, 3H), 2.72-2.95 (t, J=10.0 Hz, 2H), 2.21-2.38 (m, 3H), 2.04-2.11 (m, 1H), 1.82-1.88 (m, 1H), 1.62-1.72 (m, 2H). MS (ESI): m/z 373 (M+H⁺).

### 77. Compound 77

### 78. Compound 78

### ((R)-10-(imidazo[1,2-a]pyridin-6-yl)-2,3,5,6,13,13a-hexahydro-1H-benzo[4,5]imidazo[1,2-d]pyrrolo[2,1-g][1,4]diazepine compound and (S)-10-(imidazo[1,2-a]pyridin-6-yl)-2,3,5,6,13,13a-hexahydro-1H-benzo[4,5]imidazo[1,2-d]pyrrolo[2,1-g][1,4]diazepine)

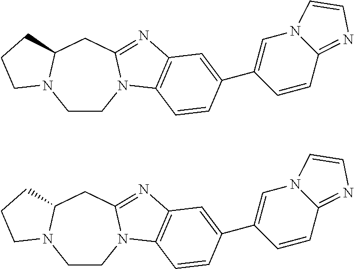

Enantiomers of compound 74 were separated by chiral chromatography. Compound 74 was dissolved in methanol, and the solution was filtered through a 0.5 μm filter cartridge. The isocratic SFC method used a mixture of 45% isopropanol with 1% isopropyl amine in CO₂ as the mobile phase. The column was a RegisPack™ column (Regis Technologies™) in a 3.0×25.0 cm format with a mobile phase flow of 80 mL/min. The enantiomers of compound 74 were isolated as two separate peaks during chiral separation. The faster eluting peak was designated as compound 77. The more slowly eluting peak was designated as compound 78.

### 79. Compound 79 (11-bromo-1,2,3,5,6,7,14,14a-octahydrobenzo[4,5]imidazo[1,2-a]pyrrolo[2,1-d][1,5]diazocine)

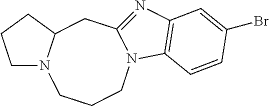

This compound was prepared in 45% yield (1.6 g) as described for compound 1 but using intermediate I-105 as the starting material. ¹H-NMR (400 MHz, CDCl₃): δ 7.83 (d, J=1.2 Hz, 1H), 7.34 (dd, J₁=2.0 Hz, J₂=1.6, 1H), 7.18 (d, J=4.0 Hz, 3H), 4.51-4.54 (m, 1H), 4.25˜4.26 (m, 1H), 3.30 (d, J=2.0 Hz, 1H), 2.80-2.86 (m, 1H), 2.70-2.74 (m, 2H), 2.51˜2.55 (m, 1H), 2.06˜2.14 (m, 1H), 1.70˜1.81 (m, 6H). MS (ESI): m/z 321 (M+H⁺).

### 80. Compound 80 (4-(1,2,3,5,6,7,14,14a-octahydrobenzo[4,5]imidazo[1,2-a]pyrrolo[2,1-d][1,5]diazocin-11-yl)benzonitrile)

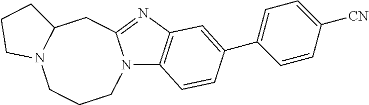

This compound was prepared in 50% yield (85 mg) as described for compound 2 but using compound 79 as the starting material. ¹H-NMR (400 MHz, CDCl₃): δ 7.92 (d, J=1.2 Hz, 1H), 7.72 (s, 1H), 7.49 (dd, J₁=2.0 Hz, J₂=1.6 Hz, 1H), 7.41 (d, J=8.4 Hz, 1H), 4.58˜4.60 (m, 1H), 4.30˜4.34 (m, 1H), 3.31˜3.34 (d, J=12 Hz, 1H), 3.20 (m, 1H), 2.74˜2.79 (m, 3H), 2.56˜2.58 (m, 1H), 2.17˜2.19 (m, 1H), 1.73˜1.88 (m, 6H). MS (ESI): m/z 343 (M+H⁺).

### 81. Compound 81 (11-(imidazo[1,2-a]pyridin-6-yl)-1,2,3,5,6,7,14,14a-octahydrobenzo[4,5]imidazo[1,2-a]pyrrolo[2,1-d][1,5]diazocine)

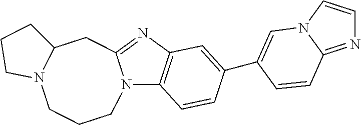

This compound was prepared in 40% yield (71 mg) as described for compound 2 but using intermediate I-106 and 6-bromoimidazo[1,2-a]pyridine as starting materials. ¹H-NMR (400 MHz, CDCl₃): δ 8.31 (s, 1H), 7.86 (d, J=1.2 Hz, 1H), 7.64˜7.69 (m, 3H), 7.37˜7.50 (m, 3H), 4.58˜4.65 (m, 1H), 4.30˜4.36 (m, 1H), 3.32˜3.36 (m, 1H), 3.21˜3.25 (t, J=7.6 Hz, 1H), 2.89˜2.95 (m, 1H), 2.75˜2.81 (m, 2H), 2.54˜2.61 (m, 1H), 2.15˜2.23 (m, 1H), 1.73˜1.98 (m, 6H). MS (ESI): m/z 358 (M+H⁺).

### 82. Compound 82

### 83. Compound 83

### ((R)-11-(imidazo[1,2-a]pyridin-6-yl)-1,2,3,5,6,7,14,14a-octahydrobenzo[4,5]imidazo[1,2-a]pyrrolo[2,1-d][1,5]diazocine compound and (S)-11-(imidazo[1,2-a]pyridin-6-yl)-1,2,3,5,6,7,14,14a-octahydrobenzo[4,5]imidazo[1,2-a]pyrrolo[2,1-d][1,5]diazocine)

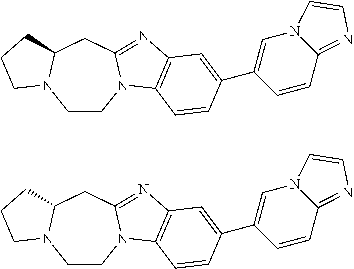

Enantiomers of compound 81 were separated by chiral chromatography. Compound 81 was dissolved in methanol, and the solution was filtered through a 0.5 μm filter cartridge. The isocratic SFC method used 43% of a 9:1 mixture of isopropanol: acetonitrile with 1% isopropyl amine in CO₂ as the mobile phase. The column was a RegisPack™ column (Regis Technologies™) in a 3.0×25.0 cm format with a mobile phase flow of 80 mL/min. The enantiomers of compound 81 were isolated as two separate peaks during chiral separation. The faster eluting peak was designated as compound 82. The more slowly eluting peak was designated as compound 83.

### 84. Compound 84 (4-((3-cyclobutyl-1,2,3,4,5,6-hexahydrobenzo[4,5]imidazo[1,2-a][1,5]diazocin-10-yl)methyl)morpholine)

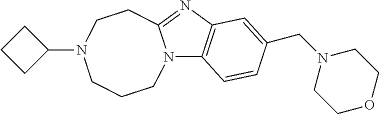

This compound was prepared in 63% yield (1.1 g) as described for compound 39 but using intermediate I-109 as the starting material. ¹H-NMR (400 MHz, CDCl₃): δ 7.62 (s, 1H), 7.21-7.26 (m, 2H), 4.39-4.42 (t, J=6.0 Hz), 3.69-3.72 (t, J=4.4 Hz, 4H), 3.62 (s, 2H), 3.09-3.12 (m, 3H), 2.75-2.78 (t, J=5.2 Hz, 2H), 2.47 (m, 4H), 1.98-2.08 (m, 4H), 1.58-1.83 (m, 6H). MS (ESI): m/z 355 (M+H⁺).

### 85. Compound 85 (3-cyclobutyl-10-((3,4-dihydroisoquinolin-2(1H)-yl)methyl)-1,2,3,4,5,6-hexahydrobenzo[4,5]imidazo[1,2-a][1,5]diazocine)

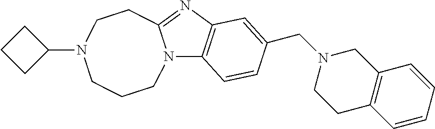

This compound was prepared in 56% yield (1.1 g) as described for compound 17 but using intermediate I-109 and 1,2,3,4-tetrahydroisoquinoline as the starting materials. ¹H-NMR (400 MHz, CDCl₃): δ 7.66 (s, 1H), 7.32-7.35 (m, 1H), 7.24-7.27 (m, 1H), 7.07-7.10 (m, 3H), 6.95-6.97 (m, 1H), 4.39-4.42 (t, J=6.0 Hz, 2H), 3.80 (s, 2H), 3.65 (s, 2H), 3.09-3.16 (m, 3H), 2.87-2.91 (t, J=6.0 Hz, 2H), 2.76-2.79 (t, J=6.0 Hz, 4H), 1.98-2.08 (m, 4H), 1.58-1.83 (m, 6H). MS (ESI): m/z 401 (M+H⁺).

### 86. Intermediates

### Intermediate I-2 (2-(4-bromo-2-nitrophenylamino)ethanol)

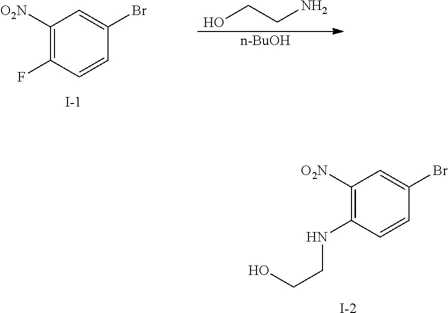

Intermediate I-1 (8.4 g, 40 mmol, 1.0 eq) was dissolved in n-BuOH (50 mL), and 2-aminoethanol (3.0 g, 50 mmol, 1.3 eq) was added. The reaction mixture was refluxed for 2 hours and the excess solvent was removed by evaporation. The crude intermediate I-2 (8.6 g, 95%) was used in the next step without further purification. MS (CI): m/z 261.0 (M+H⁺).

### Intermediate I-3 (2-(2-amino-4-bromophenylamino)ethanol)

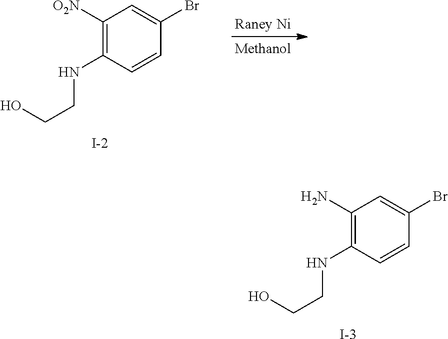

Intermediate I-2 (8.6 g, 33 mmol, 1.0 eq) was dissolved in methanol (5 mL), and Raney-Ni (0.5 g, 6% wt) was added in small portions until the starting material was consumed (after approximately 1 hour). The solids were removed by filtration, and the filtrate was concentrated by evaporation. The crude intermediate I-3 (7.6 g, 95%) was used in next step without further purification. MS (CI): m/z 231.0 (M+H⁺).

### Intermediate I-4 ((S)-tert-butyl 2-(2-(5-bromo-2-(2-hydroxyethylamino)phenylamino)-2-oxoethyl)pyrrolidine-1-carboxylate)

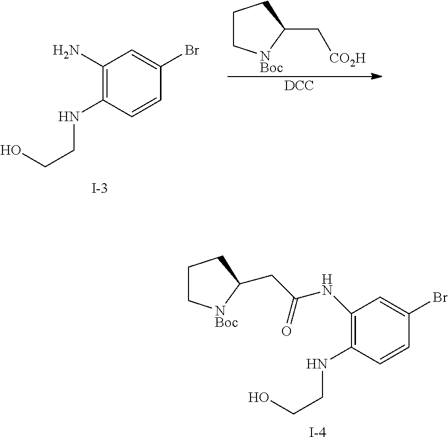

Intermediate I-3 (4.4 g, 19 mmol. 1.0 eq) and (S)-2-(1-(tert-butoxycarbonyl)pyrrolidin-2-yl)acetic acid (5.0 g, 20 mmol, 1.0 eq) were dissolved in dichloroethane (30 mL), and DCC (5.8 g, 28 mmol, 1.3 eq) was added. The reaction mixture was stirred for 16 hours at room temperature, the solid were removed by filtration and the filtrate was extracted with water and saturated NaCl solution. The combined organic layers were dried with anhydrous Na₂SO₄, and the filtrate was concentrated by evaporation. The crude intermediate I-4 (3.0 g, 60%) was used in the next step without further purification. MS (CI): m/z 444.0 (M+H⁺).

### Intermediate I-5 ((S)-tert-butyl 2-((5-bromo-1-(2-hydroxyethyl)-1H-benzo[d]imidazol-2-yl)methyl)pyrrolidine-1-carboxylate)

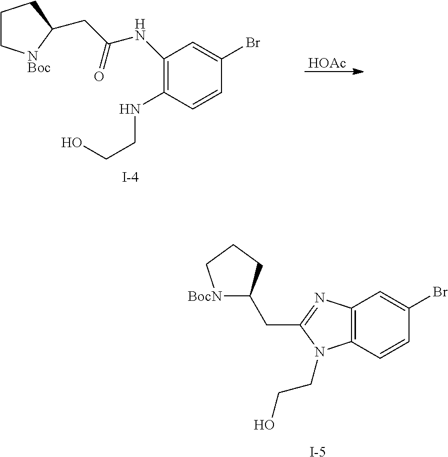

Intermediate I-4 (3.0 g, 7.0 mmol, 1.0 eq) was dissolved acetic acid (15 mL), and the reaction mixture was heated at 60° C. for 4 hours. The excess solvent was removed by evaporation, saturated aqueous NaHCO₃ was added carefully and pH was adjusted to ˜8.0. The aqueous solution was extracted with ethyl acetate and the organic layer was washed with water and an aqueous solution of NaCl. The combined organic layers were dried with anhydrous Na₂SO₄, the solids were removed by filtration and the filtrate was concentrated by evaporation. The crude intermediate I-5 (4.7 g, 100%) was used in the next step without further purification. MS (CI): m/z 426.0 (M+H⁺).

### Intermediate I-6 ((5)-tert-butyl 2-((5-bromo-1-(2-(tosyloxy)ethyl)-1H-benzo[d]imidazol-2-yl)methyl)pyrrolidine-1-carboxylate)

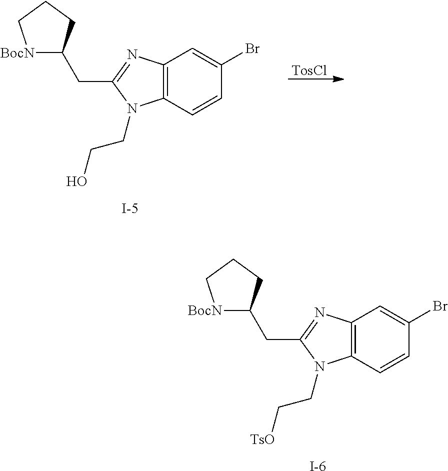

Intermediate I-5 (2.2 g, 5.0 mmol, 1.0 eq) was dissolved in dichloromethane (20 mL), and solid TosCl (1.5 g, 7.5 mmol, 1.5 eq) was added. Neat triethylamine (1.1 g, 10 mmol, 2.0 eq) was added dropwise over a period of 10 min, and the reaction mixture was stirred at room temperature overnight. The crude reaction mixture was washed with an aqueous NaCl solution, the organic layer was dried with anhydrous Na₂SO₄ and the solids were removed by filtration. The crude product was purified by silica gel chromatography to give intermediate I-6 (3.9 g, 60%). MS (CI): m/z 580.0 (M+H⁺).

### Intermediate I-8 ((S)-10-(4,4,5,5-tetramethyl-1,3,2-dioxaborolan-2-yl)-2,3,5,6,13,13a-hexahydro-1H-benzo[4,5]imidazo[1,2-d]pyrrolo[2,1-g][1,4]diazepine)

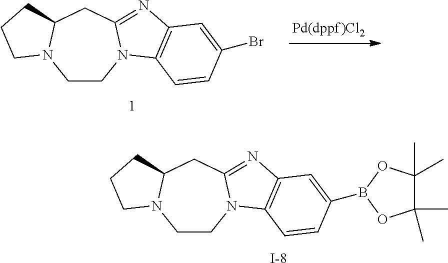

Compound 1 (0.80 g, 2.6 mmol, 1.0 eq) and 4,4,4′,4′,5,5,5′,5′-octamethyl-2,2′-bi(1,3,2-dioxaborolane) (0.99 g, 3.9 mmol, 1.5 eq) were dissolved in DMF (2 mL), and solid KOAc (0.52 g, 5.2 mmol, 2.0 eq) and Pd(dppf)Cl₂(80 mg, 0.26 mmol, 0.1 eq) were added. The reaction mixture was heated under microwave irradiation at 120° C. for 30 minutes. The solids were removed by filtration, and the filtrate was extracted with ethyl acetate. The organic layer was washed with water and an aqueous NaCl solution. The combined organic layers were dried over anhydrous Na₂SO₄, and the solids were removed by filtration. The filtrate was concentrated by evaporation, and the crude reaction product was purified by silicagel chromatography to give intermediate I-8 (600 mg, 64%). MS (CI): m/z 354.0 (M+H⁺).

### Intermediate I-11 (2-(1-(tert-butoxycarbonyl)piperidin-2-yl)acetic acid)

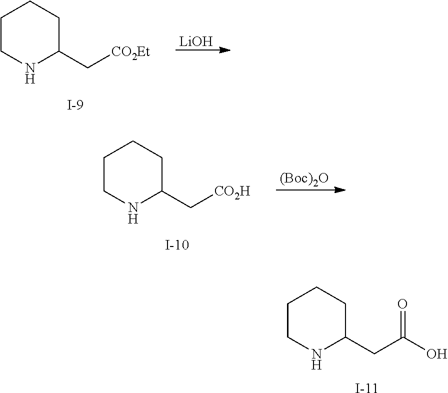

Intermediate I-9 (5.0 g, 29 mmol, 1.0 eq) was dissolved in a 2:1 mixture of THF and H₂O (10 mL:5 mL), and solid LiOH (4.9 g, 120 mmol, 4.0 eq) was added. The reaction mixture was heated under microwave irradiation at 90° C. for 1 hour. To this crude intermediate I-10 was added di-tert-butyl dicarbonate (13 g, 58 mmol, 2.0 eq), and the reaction mixture was stirred at room temperature overnight. The crude reaction mixture was acidified to pH ˜3 by adding aqueous solution of HCl (1.0 M) and extracted with ethyl acetate. The organic layer was washed with water and an aqueous NaCl solution, the combined organic layers were dried over anhydrous Na₂SO₄, the solids were removed by filtration and the filtrate was concentrated. The crude I-11 was obtained as a white solid (7.0 g, 93%). MS (ESI): m/z 144.7 (M-Boc+H⁺).

### Intermediate I-12 (2-(1-(tert-butoxycarbonyl)piperidin-2-yl)acetic acid)

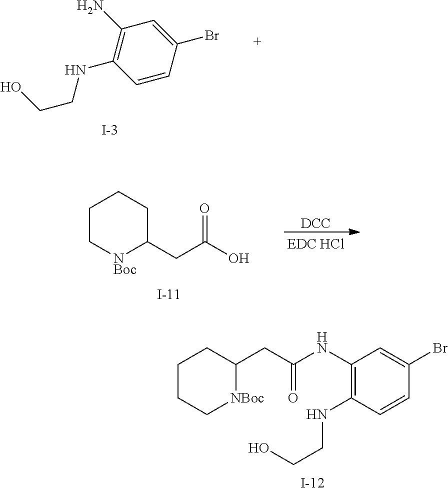

Intermediate I-3 (2.0 g, 8.7 mmol, 1.0 eq) and intermediate I-11 (2.5 g, 10 mmol, 1.2 eq) were dissolved in dichloromethane (30 mL), and solid DCC (1.7 g, 12 mmol, 1.5 eq) and EDC-HCl (2.4 g, 12 mmol, 1.5 eq) were added. The mixture was stirred at room temperature overnight, the solids were removed by filtration and the filtrate was washed with water and saturated aqueous NaCl solution. The combined organic layers were dried over anhydrous Na₂SO₄, the solids were removed by filtration and the filtrate was concentrated. The crude intermediate I-12 (2.0 g, 50%) was used in the following step without further purification. MS (ESI): m/z 456.7 (M+H⁺).

### Intermediate I-13 (11-(4,4,5,5-tetramethyl-1,3,2-dioxaborolan-2-yl)-1,2,3,4,6,7,14,14a-octahydrobenzo[4,5]imidazo[1,2-d]pyrido[2,1-g][1,4]diazepine)

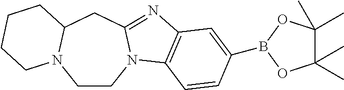

This intermediate was prepared in 33% yield (77 mg) as described for intermediate I-8 but using compound 7 as the starting material.

### Intermediate I-15 (tert-butyl 2-((4-bromo-2-nitrophenylamino)methyl)pyrrolidine-1-carboxylate)

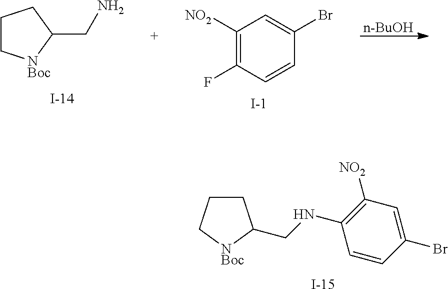

Intermediate I-14 (3.7 g, 17 mmol, 1.0 eq) was dissolved in n-BuOH (50 mL), and intermediate I-1 (4.0 g, 20 mmol, 1.2 eq) was added. The reaction mixture was refluxed for 1 hour, the excess solvent was removed by evaporation and hexane was added to the crude residue. The solid was collected by filtration and recrystallized to give intermediate I-15 (5.0 g, 65%) as an orange solid. MS (CI): m/z 400.0 (M+H)⁺.

### Intermediate I-16 (tert-butyl 2-((2-amino-4-bromophenylamino)methyl)pyrrolidine-1-carboxylate)

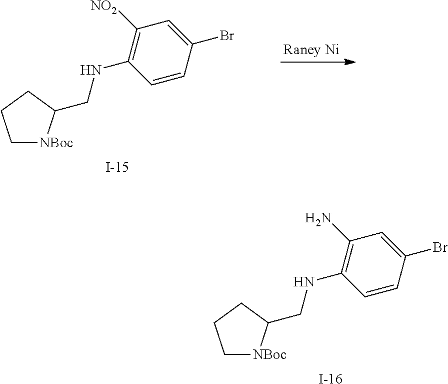

Intermediate I-15 (3.0 g, 7.5 mmol, 1.0 eq) was dissolved in MeOH (15 mL), and hydrazine hydrate (85% in water, 0.66 g, 17 mmol, 2.5 eq) was added. A suspension of Raney Nickel (0.3 g, 10%) was added dropwise until all I-15 was consumed. The solids were removed by filtration, and the filtrate was concentrated by evaporation to give I-16 (2.7 g, 100%) as a gray solid. MS (CI): m/z 370.0 (M+H)⁺.

### Intermediate I-17 (tert-butyl 2-((4-bromo-2-(3-methoxy-3-oxopropanamido) phenylamino)methyl)pyrrolidine-1-carboxylate

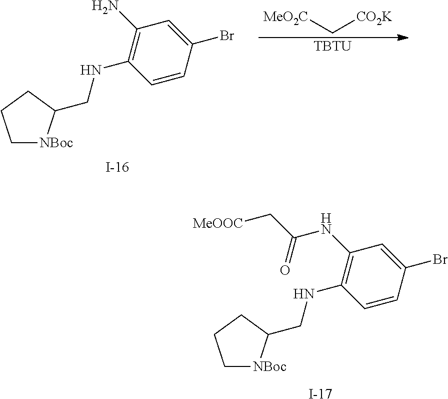

Intermediate I-16 (3.1 g, 8.5 mmol, 1.0 eq) was dissolved in dichloromethane (20 mL), potassium 3-methoxy-3-oxopropanoate (1.3 g, 8.5 mmol, 1.0 eq) was added followed by TBTU (4.1 g, 13 mmol, 1.5 eq), and the reaction mixture was stirred under a nitrogen atmosphere at room temperature overnight. The crude reaction mixture was diluted with dichloromethane, the solids were removed by filtration and the filtrate was washed with water. The combined organic layers were dried over anhydrous Na₂SO₄, the solids were removed by filtration and the filtrate was concentrated by evaporation to give I-17 (2.0 g, 50%) as a white solid. MS (ESI): m/z 472.0 (M+H)⁺.

### Intermediate I-18 (tert-butyl 2-((5-bromo-2-(2-methoxy-2-oxoethyl)-1H-benzo[d]imidazol-1-yl)methyl)pyrrolidine-1-carboxylate)

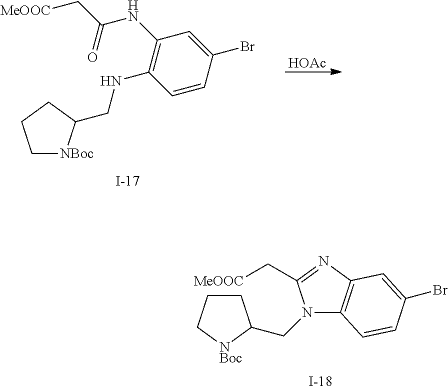

Intermediate I-17 (2.0 g, 4.2 mmol, 1.0 eq) was dissolved acetic acid (5 mL), and the reaction mixture was heated at 60° C. overnight. The excess solvent was removed by evaporation to give I-18 (1.1 g, 60%) as a gray solid. MS (ESI): m/z 454 (M+H)⁺.

### Intermediate I-19 (tert-butyl 2-((5-bromo-2-(2-hydroxyethyl)-1H-benzo[d]imidazol-1-yl)methyl)pyrrolidine-1-carboxylate)

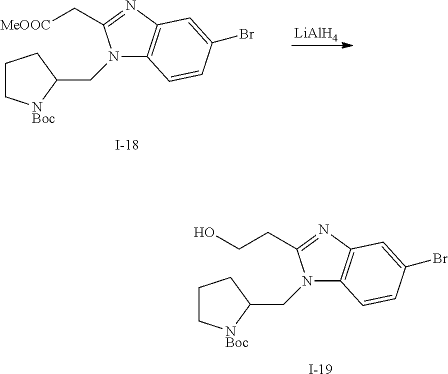

To a suspension of LiAlH₄ (0.43 g, 11 mmol, 2.0 eq) in THF (10 mL) at −78° C. was added drop wise a solution of intermediate I-18 (2.0 g, 5.7 mmol, 1.0 eq) in THF (5 mL). The reaction mixture was warmed to room temperature and stirred overnight. The reaction was quenched by addition of water, and the crude reaction mixture was extracted with ethyl acetate. The combined organic layers were dried over anhydrous Na₂SO₄, the solids were removed by filtration and the filtrate was concentrated by evaporation to give intermediate I-19 (0.48 g, 20%) as a gray solid. MS (ESI): m/z 424.0 (M+H)⁺.

### Intermediate I-20 (tert-butyl 2-((5-bromo-2-(2-(tosyloxy)ethyl)-1H-benzo[d]imidazol-1-yl)methyl)pyrrolidine-1-carboxylate)

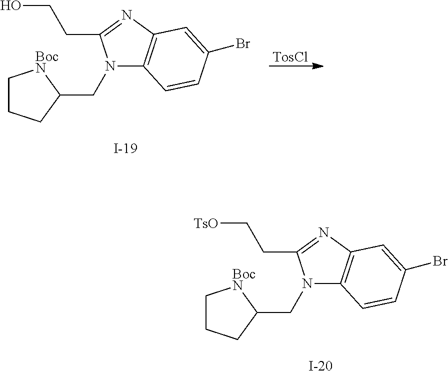

Intermediate I-19 (0.48 g, 1.1 mmol, 1.0 eq) was dissolved in dichloromethane (10 mL), and solid TsCl (0.31 g, 1.6 mmol, 1.5 eq) was added. Neat triethylamine (0.2 g, 2.0 mmol, 2.0 eq) was added dropwise over the course of 10 min, and the reaction mixture was stirred at room temperature overnight. The reaction mixture was washed with an aqueous NaCl solution, and the combined organic layers were dried over anhydrous Na₂SO₄. The solids were removed by filtration, the filtrate was concentrated by evaporation and the crude reaction product was purified by silica gel chromatography to give I-20 (0.46 g, 71%). MS (ESI): m/z 580 (M+H)⁺.

### Intermediate I-22 (1,2,3,4,6,7,14,14a-octahydrobenzo[4,5]imidazo[1,2-d]pyrido[2,1-g][1,4]diazepine-11-carbonitrile)

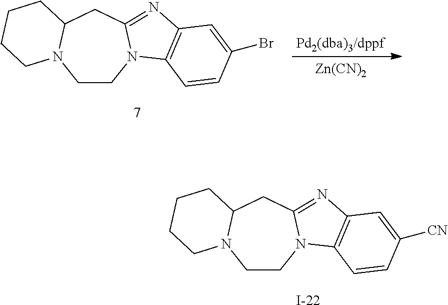

Compound 7 (200 mg, 0.63 mmol, 1.0 eq) was dissolved in DMF (0.5 mL), and Zn(CN)₂ (150 mg, 1.3 mmol, 2.0 eq) was added followed by Pd₂(dba)₃ (20 mg, 0.063 mmol, 0.1 eq) and dppf (20 mg, 0.06 mmol, 0.1 eq). The reaction mixture was heated under microwave irradiation at 130° C. for 1 hour. The solids were removed by filtration, the filtrate was extracted with ethyl acetate and the organic layer was washed with water. The combined organic layers were dried over anhydrous Na₂SO₄, the solids were removed by filtration and the filtrate was concentrated by evaporation. The crude intermediate I-22 (160 mg, 96%) was used in the next step without further purification. (ESI): m/z 267.7 (M+H)⁺.

### Intermediate I-23 (1,2,3,4,6,7,14,14a-octahydrobenzo[4,5]imidazo[1,2-d]pyrido[2,1-g][1,4]diazepine-11-carbaldehyde)

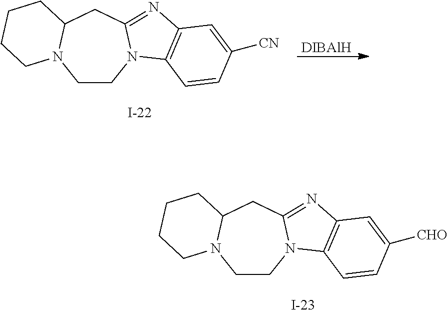

Intermediate I-22 (160 mg, 0.6 mmol, 1.0 eq) was dissolved in THF (10 mL) at −78° C., and a solution of DIBAL-H (1.0 M in THF, 2.4 mL, 2.4 mmol, 4.0 eq) was added dropwise. The reaction mixture was stirred at −78° C. for 1 hour, and aqueous NH₄Cl solution was added carefully. The crude reaction mixture was extracted with dichloromethane, and the organic layer was washed with water. The combined organic layers were dried over anhydrous Na₂SO₄, the solids were removed by filtration and the filtrate was concentrated by evaporation. The crude reaction product was purified by preparative thin layer chromatography to give intermediate I-23 (80 mg, 50%). (ESI): m/z 270.7 (M+H)⁺.

### Intermediate I-25 (8-benzyl-8-azabicyclo[3.2.1]octan-3-one)

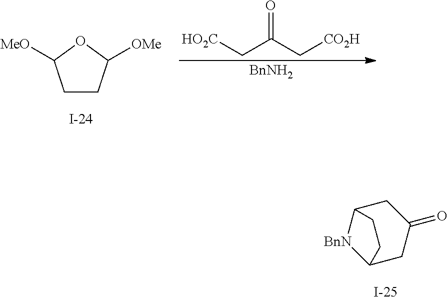

Concentrated hydrochloric acid (3 mL) was added to a stirred suspension of intermediate I-24 (8.2 g, 62 mmol, 1 eq) in water (17 mL), the reaction mixture was stirred for additional 20 minutes and diluted with water (25 mL). In a separate flask cooled to 0° C. on an ice bath, concentrated hydrochloric acid (9 mL) was added slowly to a solution of benzyl amine (10 g, 93 mmol, 1.5 eq) in water (35 mL), and this solution was added to the above solution of I-24. A solution of 1,3-acetonedicarboxylic acid (10 g, 68 mmol, 1.1 eq) in water (40 mL) was added followed by a solution of sodium hydrogen phosphate (4.4 g, 31 mmol, 5.0 eq) in water (20 mL). The acidity was adjusted from pH ˜1 to pH ˜4.5 using a solution of NaOH (40% in water). The resulting cloudy and pale-yellow solution was stirred overnight at room temperature. The reaction mixture was acidified to pH ˜3 from pH ˜7.5 using aqueous HCl solution (50% in water) and stirred at 85° C. for 2 hours. The crude reaction mixture was cooled to room temperature, basified to pH ˜12 using a solution of NaOH (40% in water) and extracted with dichloromethane. The combined organic layers were washed with brine, dried over anhydrous MgSO₄, the solids were removed by filtration and the filtrate was concentrated. The crude reaction product was purified by silica gel chromatography to give I-25 as a yellow oil (8.0 g, 60%). ¹H-NMR (CDCl₃) δ: 7.59 (d, J=7.2 Hz, 2H), 7.31-7.40 (m, 3H), 4.04 (s, 2H), 3.75 (s, 2H), 3.13 (d, J=12.0 Hz, 2H), 2.22-2.30 (m, 4H), 1.75-1.80 (m, 2H). MS (ESI): ink 216 (M+H⁺).

### Intermediate I-26 (9-benzyl-3,9-diazabicyclo[4.2.1]nonan-4-one)

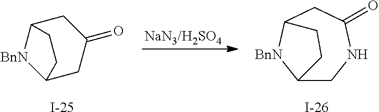

Intermediate I-25 (1.5 g, 7.0 mmol, 1.0 eq) was dissolved in chloroform (15 mL) at −5° C., and concentrated H₂SO₄ (3.5 mL) was added drop wise to maintain the reaction temperature below 5° C. Solid NaN₃ (0.91 g, 13.9 mmol, 2.0 eq) was added carefully, and the mixture was stirred at 20° C. overnight and at 50° C. for an additional 2 hours. The reaction mixture was cooled to room temperature, and slurry of ice in water (12 mL) was slowly added. The reaction mixture was neutralized with solid NaOH to pH ˜7 and stirred overnight at 25° C. A solution of NaOH (4 mL, 4 M in water) was added, and the reaction mixture was extracted with dichloromethane. The combined organic layers were dried over anhydrous MgSO4, the solids were removed by filtration and the filtrate was concentrated by evaporation to give I-26 as a brown solid (1.3 g, 81%). ¹H-NMR (DMSO-d₆) δ: 7.24-7.39 (m, 5H), 3.58-3.46 (m, 3H), 3.31 (t, J=5.6 Hz, 1H), 3.24 (t, J=5.6 Hz, 1H), 2.82-2.92 (m, 2H), 2.46-2.52 (m, 2H), 2.03-2.17 (m, 2H), 1.83-1.87 (m, /H), 1.71-1.82 (m, /H). MS (ESI): m/z 231.0 (M+H⁺).

### Intermediate I-27 (9-benzyl-3-(4-chloro-2-nitrophenyl)-3,9-diazabicyclo[4.2.1]nonan-4-one)

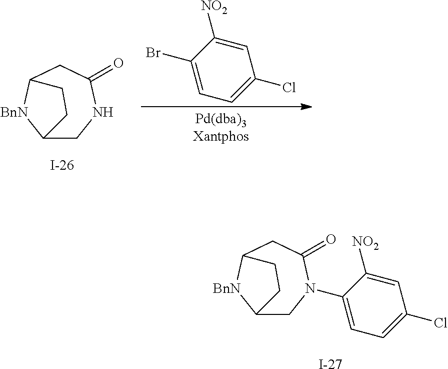

A flame-dried flask was charged with Pd₂(dba)₃ (0.78 g, 0.85 mmol, 3 mol % of Pd), Xantphos (1.4 g, 2.5 mmol, 9 mol %), intermediate I-26 (7.7 g, 34 mmol, 1.0 eq) and Cs₂CO₃ (13.6 g, 42 mmol, 1.3 eq). 1-bromo-4-chloro-2-nitrobenzene (7.9 g, 34 mmol, 1.0 eq) and 1,4-dioxane (30 mL) were added under nitrogen atmosphere, and the reaction mixture was stirred at 100° C. for 35 hours. The reaction mixture was cooled to room temperature, diluted with dichloromethane (50 mL), the solids were removed by filtration and the filtrate was concentrated by evaporation. The crude reaction product was purified by silica gel chromatography to give intermediate I-27 as pale-yellow oil (8.7 g, 67%). ¹H-NMR (CDCl₃) δ: 7.97 (s, 1H), 7.60 (d, J=7.6 Hz, 1H), 7.40 (d, J=7.2 Hz, 2H), 7.35 (t, J=7.2 Hz, 2H), 7.23-7.29 (m, 2H), 4.28 (d, J=14.8 Hz, 1H), 3.75 (q, J=13.2 Hz, 2H), 3.44 (s, 1H), 3.34 (s, 1H), 3.18 (m, 1H), 3.02 (d, J=12.0 Hz, 1H), 2.71 (dd, J=5.6 Hz, 1H), 2.19 (s, 2H), 1.98 (d, J=11.2 Hz, 1H), 1.81 (d, J=11.2 Hz, 1H). MS (ESI): m/z 386.0 (M+H⁺).

### Intermediate I-28 (13-benzyl-3-chloro-6,7,8,9,10,11-hexahydro-7,10-epiminobenzo[4,5]imidazo[1,2-a]azocine)

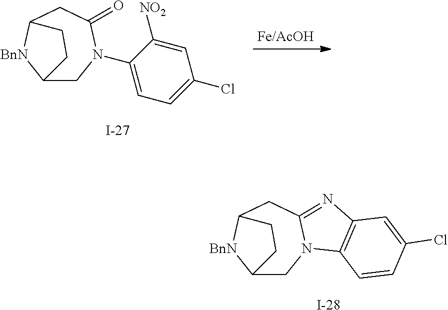

Elemental iron (4.0 g, 71 mmol, 3.0 eq) was added to a solution of intermediate I-27 (8.7 g, 23 mmol, 1.0 eq) in acetic acid (50 mL), and the reaction mixture was refluxed for 30 minutes. Excess acetic acid was removed by evaporation, and residue was diluted with ethyl acetate and washed with aqueous solution of NaHCO₃. The combined organic layers were washed with saturated aqueous solution of NaHCO₃ and aqueous saturated solution of NaCl, the combined organic layers were dried over anhydrous Na₂SO₄, the solids were removed by filtration and the filtrate was concentrated by evaporation to give intermediate I-28 (6.6 g, 86%) which was used in the following step without further purification. MS (ESI): m/z 338.0 (M+H⁺).

### Intermediate I-29 (3-chloro-6,7,8,9,10,11-hexahydro-7,10-epiminobenzo[4,5]imidazo[1,2-a]azocine)

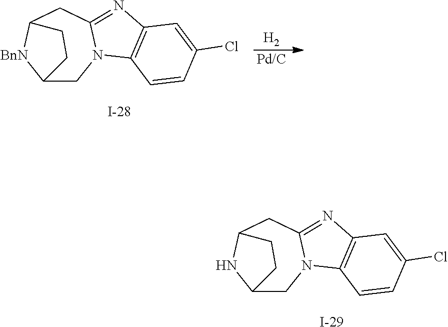

Intermediate I-28 (4.4 g, 13 mmol, 1.0 eq) was dissolved with acetic acid, palladium on carbon (1.0 g, 0.1 eq of Pd) was added and the reaction mixture was stirred under hydrogen atmosphere (1 atm) at room temperature for 4 hours. Palladium on carbon was removed by filtration through Kieselguhr™, and the filtrate was concentrated by evaporation to give intermediate I-29 (3.3 g, 88% purity as determined by LC-MS) as yellow oil which was used in next step without further purification. MS (ESI): m/z 248 (M+H⁺).

### Intermediate I-30 (13-cyclobutyl-3-(4,4,5,5-tetramethyl-1,3,2-dioxaborolan-2-yl)-6,7,8,9,10,11-hexahydro-7,10-epiminobenzo[4,5]imidazo[1,2-a]azocine)

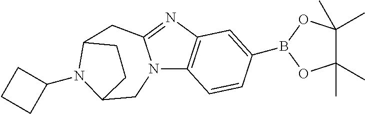

This intermediate was prepared in 45% yield (80 mg) as described for compound 19 but using 4,4,4′,4′,5,5,5′,5′-octamethyl-2,2′-bi(1,3,2-dioxaborolane) as the starting material. MS (ESI): m/z 394.1 (M+H⁺).

### Intermediate I-32 (3-benzyl-3-azabicyclo[3.2.1]octan-8-one)

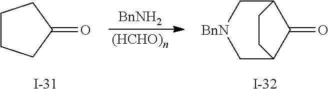

A 2-L round-bottom flask equipped with condenser and nitrogen inlet was charged with benzyl amine (127 g, 1.2 mol, 1.0 eq) and methanol (800 mL), and glacial acetic acid (127 g, 1.2 mol, 1.0 eq) was added drop wise. Intermediate I-31 (100 g, 1.2 mol, 1.0 eq) was added followed by paraformaldehyde (107 g, 3.6 mol, 3.0 eq) and the reaction mixture was stirred at 80° C. for 3 hours and then at room temperature for additional 18 hours. Excess solvent was removed by evaporation, the residue was diluted with ethyl acetate and solid sodium metabisulphite (104 g) was added. The crude reaction mixture was stirred for additional 1.5 hours and extracted with ethyl acetate. The combined organic layers were dried over anhydrous Na₂SO₄, the solids were removed by filtration and the filtrate was concentrated by evaporation. The crude reaction product was purified by silica gel chromatography to give intermediate I-32 (47.2 g, 18%) as yellow oil. ¹H-NMR (400 MHz, CDCl₃) δ: 7.35 (m, 4H), 7.26 (m, 1H), 3.59 (s, 2H), 2.93 (m, 2H), 2.39 (d, J=10.8 Hz, 2H), 2.50 (m, 2H), 1.93 (m, 2H), 1.81 (m, 2H). MS (ESI): m/z 216.1 (M+H⁺).

### Intermediate I-33 (3-benzyl-3,6-diazabicyclo[3.2.2]nonan-7-one)

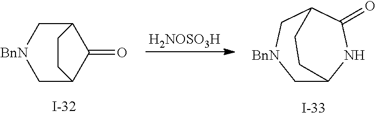

Intermediate I-32 (10 g, 4.7 mmol, 1.0 eq) and hydroxylamine O-sulfonic acid (18.1 g, 18.6 mmol, 4.0 eq) were dissolved in formic acid (30 mL), and the reaction mixture was refluxed for 24 hour. Formic acid was removed by evaporation, and water was added to the residue. The resulting suspension was extracted with dichloromethane, and the combined organic layers were washed sequentially with 10% aqueous NaHCO₃, water and brine. The combined organic layers were dried over anhydrous Na₂SO₄, the solids were removed by filtration and the filtrate was concentrated by evaporation. The crude reaction product was purified by silica gel chromatography to give intermediate I-33 (1.6 g, 15%). ¹H-NMR (400 MHz, CDCl₃) δ: 7.31 (m, 4H), 7.24 (m, 1H), 3.55 (m, 3H), 2.89 (q, J=6.4 Hz, 2H), 2.49 (m, 1H), 2.31 (d, J=8.4 Hz, 1H), 2.22 (m, 2H), 2.08 (m, 1H), 1.80 (m, 2H). MS (ESI): m/z 231.1 (M+H⁺).

### Intermediate I-34 (3-benzyl-6-(4-chloro-2-nitrophenyl)-3,6-diazabicyclo[3.2.2]nonan-7-one)

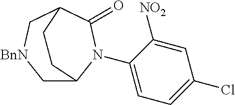

This intermediate was prepared in 62% yield (120 mg) as described for intermediate I-27 but using intermediate I-33 as the starting material. ¹H-NMR (400 MHz, DMSO-d₆) δ: 8.03 (d, J=2 Hz, 1H), 7.82 (dd, J=2.4 Hz, J=8.8 Hz, 1H), 7.52 (m, 1H), 7.35 (m, 4H), 7.26 (m, 1H), 4.22 (m, 1H), 3.60 (m, 2H), 2.87 (m, 2H), 2.68 (m, 2H), 2.42 (d, J=8 Hz, 1H), 2.21 (m, 3H), 1.80 (m, 1H). MS (ESI): m/z 386.1 (M+H⁺).

### Intermediate I-35 (3-benzyl-8-chloro-2,3,4,5-tetrahydro-1H-1,5-ethanobenzo[4,5]imidazo[1,2-d][1,4]diazepine)

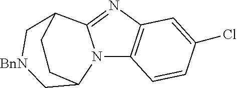

This intermediate was prepared in 58% yield (350 mg) as described for intermediate I-28 but using intermediate I-34 as the starting material. ¹H-NMR (400 MHz, CDCl₃) δ: 7.70 (d, J=2 Hz, 1H), 7.32 (m, 5H), 7.18 (m, 2H), 4.64 (m, J=5.2 Hz, 1H), 3.52 (m, 3H), 3.13 (m, 2H), 2.38 (m, 4H), 1.90 (m, 2H). MS (ESI): m/z 338.2 (M+H⁺).

### Intermediate I-36 (8-chloro-2,3,4,5-tetrahydro-1H-1,5-ethanobenzo[4,5]imidazo[1,2-d][1,4]diazepine)

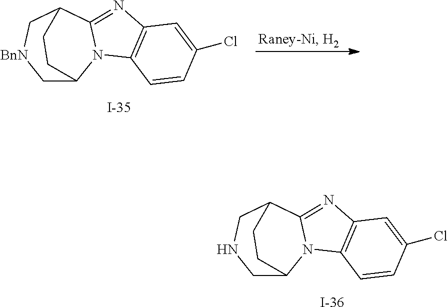

Intermediate I-35 (3.5 g, 10.4 mmol, 1.0 eq) was dissolved in acetic acid (20 mL), and a slurry of Raney-Ni (500 mg) was added. The reaction mixture was stirred under a hydrogen atmosphere (1 atm) at room temperature for 48 hours. The solids were removed by filtration, the filtrate was diluted with water, the pH was adjusted to ˜8 with a solution of NaOH (1 M in water), and the crude reaction mixture was extracted with dichloromethane and washed with brine. The combined organic layers were dried over anhydrous Na₂SO₄, the solids were removed by filtration and the filtrate was concentrated by evaporation. The crude reaction product was purified by silica gel chromatography to give intermediate I-36 (1.5 g, 79%) as a pale yellow solid. ¹H-NMR (400 MHz, CDCl₃) δ: 7.72 (m, 1H), 7.24 (m, 2H), 4.63 (d, J=5.2 Hz, 1H), 3.47 (s, 1H), 3.12 (m, 3H), 3.02 (m, 1H), 2.33 (m, 2H), 2.09 (m, 2H). MS (ESI): m/z 247.9 (M+H⁺).

### Intermediate I-37 (3-cyclobutyl-8-(4,4,5,5-tetramethyl-1,3,2-dioxaborolan-2-yl)-2,3,4,5-tetrahydro-1H-1,5-ethanobenzo[4,5]imidazo[1,2-d][1,4]diazepine)

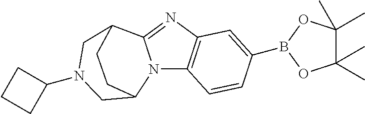

This intermediate was prepared in 45% yield (280 mg) as described for compound 19 but using compound 25 and 4,4,4′,4′,5,5,5′,5′-octamethyl-2,2′-bi(1,3,2-dioxaborolane) as the starting material. ¹H-NMR (400 MHz, CDCl₃) δ: 8.21 (s, 1H), 7.70 (d, J=8 Hz, 1H), 7.28 (d, J=8 Hz, 1H), 4.70 (m, 1H), 3.53 (m, 1H), 3.08 (m, 2H), 2.74 (m, 1H), 2.39 (m, 2H), 2.00 (m, 6H), 1.85 (m, 2H), 1.68 (m, 2H), 1.38 (s, 12H). MS (ESI): m/z 394.2 (M+H⁺).

### Intermediate I-39 (cyclopenta-1,3-diene)

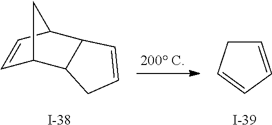

Intermediate I-38 (140 g, 1.1 mol) was placed in a flask fitted with a 30 cm Vigreux™ column, a condenser and a receiving flask cooled to −78° C. The reaction mixture was heated to 200° C., and intermediate I-39 (48g, 69%) was collected in the receiving flask as colorless oil.

### Intermediate I-40 (Benzyl 2-azabicyclo[2.2.1]hept-5-ene-2-carboxylate)

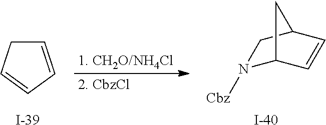

Intermediate I-39 (48 g, 0.73 mol, 1.0 eq) and ammonium chloride (116 g, 2.2 mol, 3.0 eq) were mixed in water (400 mL), and a formaldehyde solution (37% in water, 88 mL, 1.1 mol, 1.5 eq) was added. The reaction mixture was stirred at room temperature for 36 hours, neutralized with solid Na₂CO₃ and cooled to 0° C. To this mixture were added simultaneously benzyl chloroformate (124 g, 0.73 mol, 1.0 eq) and a solution of Na₂CO₃ (38.6 g, 0.364 mol, 0.5 eq) in water (400 mL) at such a rate that the addition of Na₂CO₃ was completed immediately after the addition of benzyl chloroformate. The reaction mixture was stirred for 2 hours at 0° C. The reaction mixture was diluted with H₂O (1 L) and extracted with dichloromethane (4×1 L). The combined organic layers were dried over anhydrous Na₂SO₄, the solids were removed by filtration and the filtrate was concentrated. The crude reaction product was purified by silica gel chromatography to give intermediate I-40 (35g, 21%). MS (ESI): m/z 203 (M+H⁺).

### Intermediate I-41 (Benzyl 5-hydroxy-2-azabicyclo[2.2.1]heptane-2-carboxylate)

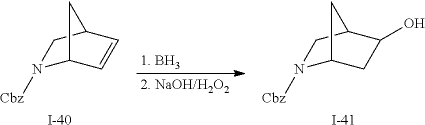

To a solution of intermediate I-40 (30 g, 131 mmol, 1.0 eq) in THF (500 mL) at −78° C. was added drop wise a solution of BH₃ (1.0 M in THF, 183 mL, 183 mmol, 1.4 eq), and the reaction mixture was stirred for additional 10 minutes. The reaction mixture was allowed to warm to room temperature and was stirred for additional 2.5 hours. The reaction mixture was quenched by sequential addition of water (80 mL), sodium hydroxide solution (3.0 M in water, 66 mL, 198 mmol, 1.5 eq), and hydrogen peroxide (30% solution in water, 30 mL, 262 mmol) and was stirred for additional 30 minutes. The solvent was removed by evaporation, the crude reaction product was dissolved in water and extracted with diethyl ether and the organic layer was washed with brine. The combined organic layers were dried over anhydrous MgSO₄, the solids were removed by filtration and the filtrate was concentrated by evaporation. The crude reaction product was purified by silica gel chromatography to give intermediate I-41 (9.7 g, 30%). MS (ESI): m/z 248 (M+H⁺).

### Intermediate I-42 (Benzyl 5-hydroxy-2-azabicyclo[2.2.1]heptane-2-carboxylate)

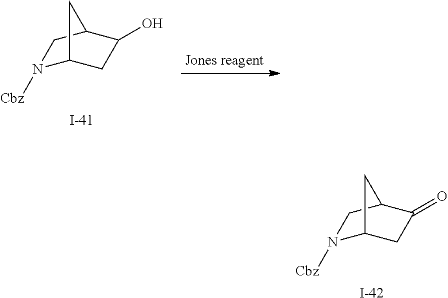

Intermediate I-41 (9.7 g, 39 mmol, 1.0 eq) was dissolved in acetone (400 mL) and cooled to 0° C. Jones reagent (18 mL, 47 mmol, 1.2 eq) [prepared from chromium trioxide (26.7 g), concentrated sulfuric acid (27.3 mL) and water (80 mL)] was added drop wise, and the reaction mixture was stirred for 3 hours. Excess chromic acid was removed by drop wise addition of propan-2-ol; the solution was basified with sodium hydroxide (3.0 M in water), and the solvent was removed by evaporation. The residue was dissolved in water, and the crude reaction product was extracted with dichloromethane. The combined organic layers were dried over anhydrous MgSO₄, the solids were removed by filtration and the filtrate was concentrated by evaporation. The crude reaction product was purified by silica gel chromatography to give intermediate I-42 (7.6 g, 80%). MS (ESI): m/z 246 (M+H⁺).

### Intermediate I-43 (Benzyl 5-(hydroxyimino)-2-azabicyclo[2.2.1]heptane-2-carboxylate)

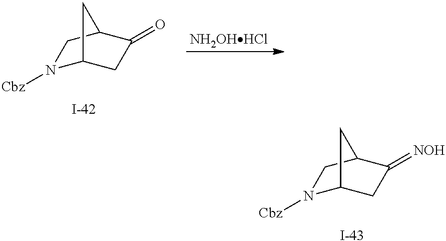

To a solution of intermediate I-42 (7.6 g, 31 mmol, 1.0 eq) in 95% aqueous ethanol (100 mL) was added solid sodium acetate (7.6 g, 93 mmol, 3.0 eq) and hydroxylamine hydrochloride (6.5 g, 93 mmol, 3.0 eq), and the reaction mixture was stirred at room temperature for 2 hours. Excess solvent was removed by evaporation, the residue was dissolved in saturated aqueous NaHCO₃ and the crude reaction product was extracted with ethyl acetate. The combined organic layers were dried over anhydrous MgSO₄, the solids were removed by filtration and the filtrate was concentrated. The crude intermediate I-43 (8.1 g, 96%) was used in the following step without further purification. MS (ESI): m/z 261 (M+H⁺).

### Intermediate I-44 (Benzyl 5-(tosyloxyimino)-2-azabicyclo[2.2.1]heptane-2-carboxylate)

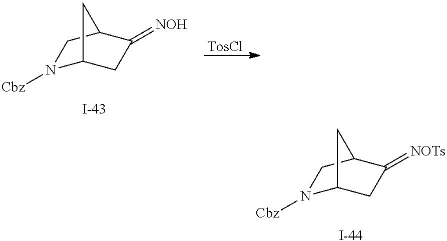

To a solution of I-43 (1.4 g, 5.2 mmol, 1.0 eq) in acetone (22 mL) was added drop wise a solution of Na₂CO₃ (1.7 g, 16 mmol, 3.0 eq) in H₂O (22 mL). The reaction mixture was stirred for 10 minutes, and a solution of TosCl (1.5 g, 7.8 mmol, 1.5 eq) in acetone (7 mL) was added drop wise. The reaction mixture was stirred for 16 hours, quenched by the addition of a saturated aqueous solution of NaHCO₃ and the crude reaction product was extracted with ethyl acetate. The combined organic layers were dried over anhydrous MgSO₄, the solids were removed by filtration and the filtrate was concentrated by evaporation. The crude intermediate I-44 (2.1 g, 100%) was used in the following step without further purification. MS (ESI): m/z 415 (M+H⁺).

### Intermediate I-45 (Benzyl 3-oxo-2,6-diazabicyclo[3.2.1]octane-6-carboxylate)

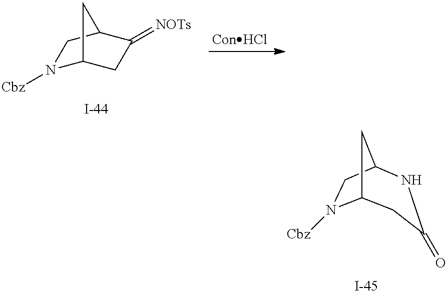

To a solution of I-44 (2.1 g, 5.2 mmol, 1.0 eq) in acetone (10 mL) was added concentrated aqueous HCl (3 mL), and the reaction was stirred at room temperature for 16 hours. Excess solvent was removed by evaporation, the residue was basified with saturated aqueous solution of NaHCO₃ and the crude reaction product was extracted with ethyl acetate. The combined organic layers were dried over anhydrous MgSO₄, the solids were removed by filtration and the filtrate was concentrated by evaporation. The crude reaction product was purified by silica gel chromatography to give intermediate I-45 (540 mg, 40%). ¹H-NMR (400 MHz, CDCl₃) δ: 7.58-7.69 (br, 1H), 7.27-7.36 (m, 5H), 5.06-518 (m, 2H), 4.23-4.35 (m, 1H), 3.92 (s, 1H), 3.60-3.71 (m, 1H), 3.41-3.46 (m, 1H), 2.67-2.90 (m, 1H), 2.44-2.49 (m, 1H), 2.00-2.07 (m, 1H), 1.93-1.96 (m, 1H). MS (ESI): m/z 261 (M+H⁺).

### Intermediate I-46 (Benzyl 2-(4-chloro-2-nitrophenyl)-3-oxo-2,6-diazabicyclo[3.2.1]octane-6-carboxylate)

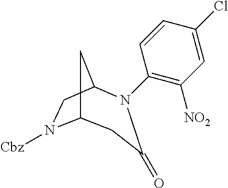

This intermediate was prepared in 69% yield (440 mg) as described for intermediate I-27 but using intermediate I-45 as the starting material. MS (ESI): m/z 416 (M+H⁺).

### Intermediate I-47 (benzyl 8-chloro-4,5-dihydro-1H-1,4-methanobenzo[4,5]imidazo[1,2-d][1,4]diazepine-3(2H)-carboxylate)

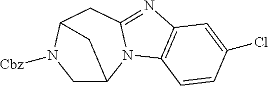

This intermediate was prepared in 70% yield (900 mg) as described for intermediate I-28 but using intermediate I-46 as the starting material. MS (ESI): m/z 342 (M+H⁺).

### Intermediate I-48 (8-chloro-2,3,4,5-tetrahydro-1H-1,4-methanobenzo[4,5]imidazo[1,2-d][1,4]diazepine)

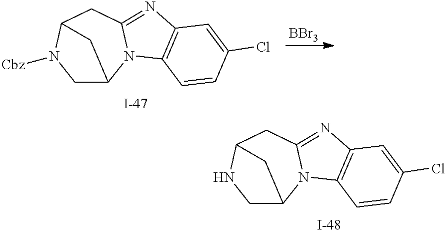

To a solution of intermediate I-47 (0.20 g, 0.58 mmol, 1.0 eq) in dichloromethane (3 mL) at −78° C. was added neat BBr₃ (0.26 mL, 2.9 mmol, 5.0 eq), and the reaction mixture was allowed to warm to room temperature. After TLC analysis indicated a complete consumption of the starting material, the reaction was quenched by adding methanol. Excess solvent was removed by evaporation, the residue was washed with ether and the crude intermediate I-48 (70 mg, 51%) was used in the following step without further purification. MS (ESI): m/z 234 (M+H⁺).

### Intermediate I-49 (3-cyclobutyl-8-(4,4,5,5-tetramethyl-1,3,2-dioxaborolan-2-yl)-2,3,4,5-tetrahydro-1H-1,4-methanobenzo[4,5]imidazo[1,2-d][1,4]diazepine)

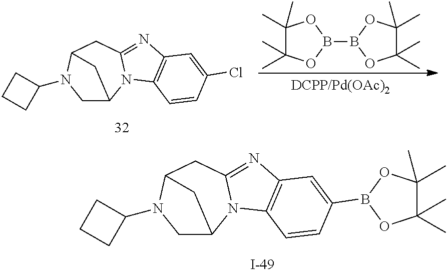

Compound 32 (0.12 g, 0.42 mmol, 1.0 eq), 4,4,4′,4′,5,5,5′,5′-octamethyl-2,2′-bi(1,3,2-dioxaborolane) (0.32 g, 1.3 mmol, 3.0 eq), Pd(OAc)₂ (15 mg, 0.063 mmol, 0.15 eq), DCPP (60 mg, 0.13 mmol, 0.3 eq) and KOAc (0.12 g, 1.26 mmol, 3.0 eq) were dissolved in dioxane (2 mL) under nitrogen atmosphere, and the reaction mixture was heated under microwave irradiation at 120° C. for 3 hours. The crude reaction mixture was extracted with ethyl acetate, and the organic layer was washed with brine. The combined organic layers were dried over anhydrous Na₂SO₄, the solids were removed by filtration and the filtrate was concentrated by evaporation. The crude reaction product was purified by silica gel chromatography to give intermediate I-49 (40 mg, 25%). MS (ESI): m/z 380 (M+H⁺).

### Intermediate I-51 (methyl 1-cyclobutyl-5-oxopyrrolidine-3-carboxylate)

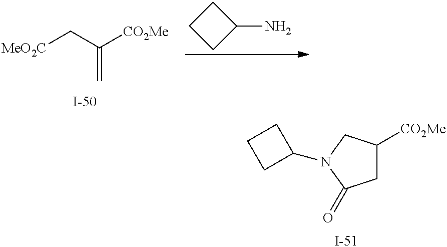

To a solution of intermediate I-50 (15.8 g, 0.1 mol, 1.0 eq) in methanol (50 mL) was added neat cyclobutanamine (7.1 g, 0.1 mol, 1.0 eq), and the reaction mixture was stirred for 16 hours. Excess solvent was removed by evaporation and the crude reaction product was purified by silica gel chromatography to give intermediate I-51 (17.7 g, 90%) as yellow oil. MS (CI): m/z 198 (M+H⁺).

### Intermediate I-52 (methyl 1-cyclobutyl-5-thioxopyrrolidine-3-carboxylate)

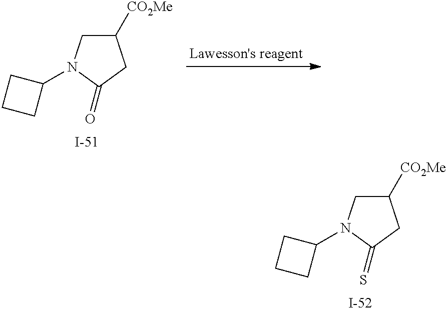

To a solution of I-51 (18 g, 90 mmol, 1.0 eq) in 100 mL THF was added Lawesson's reagent (18 g, 45 mmol, 0.5 eq) portionwise, and the reaction mixture was stirred for 3 hours. Excess solvent was removed by evaporation, and the residue was dissolved in ethyl acetate. The solution was washed with aqueous NaHCO₃ (10% in water) and brine, the combined organic layers were dried over anhydrous Na₂SO₄, the solids were removed by filtration and the filtrate was concentrated. The crude reaction product was purified by silica gel chromatography to give intermediate I-52 (18g, 95%). MS (CI): m/z 214 (M+H⁺).

### Intermediate I-53 (methyl 1-cyclobutyl-3-(methoxycarbonyl)-5-(methylthio)-3,4-dihydro-2H-pyrrolium iodide)

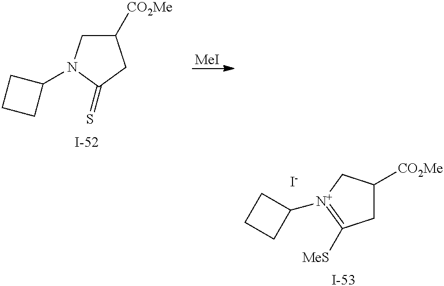

To intermediate I-52 (17.3 g, 85 mmol) was added methyl iodide (40 mL), and the reaction was stirred at 30° C. for 2 hours. Excess methyl iodide was removed by evaporation, the crude reaction product was washed with dry THF and dried under vacuum to give intermediate I-53 (30.2 g, 100%) as yellow oil that was used in the following step without further purification. MS (CI): m/z 356 (M+H⁺).

### Intermediate I-54 (methyl 1-cyclobutyl-5-(nitromethylene)pyrrolidine-3-carboxylate)

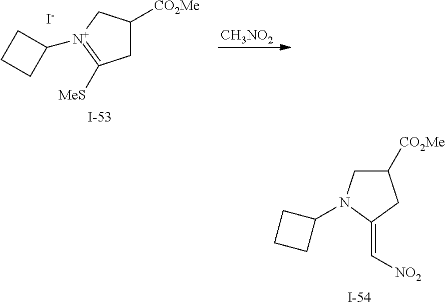

To a stirred solution of intermediate I-53 (30 g, 85 mmol, 1.0 eq) in dry DMF (100 mL) under nitrogen atmosphere was added dry Et₃N (9.5 g, 94 mmol, 1.1 eq) followed by freshly distilled nitromethane (26 g, 425 mmol, 5.0 eq), and the reaction mixture was stirred at room temperature for 12 hours. Excess solvent and nitromethane were removed by evaporation. The crude reaction product was purified by silica gel column chromatography to give intermediate I-54 (9.2 g, 45%) as yellow oil. MS (CI): m/z 241 (M+H⁺).

### Intermediate I-55 (6-cyclobutyl-3,6-diazabicyclo[3.2.1]octan-2-one)

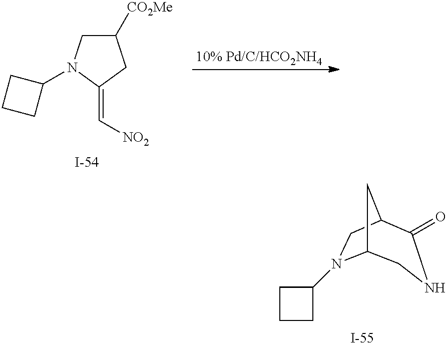

To a solution of intermediate I-54 (9.2 g, 38 mmol, 1.0 eq) and ammonium formate (49 g, 760 mmol, 20 eq) in methanol (150 mL) was added palladium on carbon (10% wt/wt, 7.0 g), and the reaction mixture was refluxed for 10 hours. The crude reaction mixture was filtered through a Celite bed, Celite was washed with methanol and the combined filtrate was concentrated by evaporation. The residue was dissolved in dichloromethane, and the solution was stirred for 30 minutes. The solids were removed by filtration, and the filtrate was concentrated by evaporation. The crude reaction product was purified by silica gel column chromatography to give intermediate I-55 (4.8 g, 70%) as a white solid. MS (CI): m/z 181 (M+H⁺).

### Intermediate I-56 (3-(4-chloro-2-nitrophenyl)-6-cyclobutyl-3,6-diazabicyclo[3.2.1]octan-2-one)

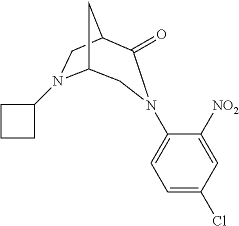

This intermediate was prepared in 70% yield (7.2 g) as described for intermediate I-27 but using intermediate I-55 as the starting material. MS (CI): m/z 336 (M+H⁺).

### Intermediate I-57 (3-cyclobutyl-8-(4,4,5,5-tetramethyl-1,3,2-dioxaborolan-2-yl)-2,3,4,5-tetrahydro-1H-2,5-methanobenzo[4,5]imidazo[1,2-d][1,4]diazepine)

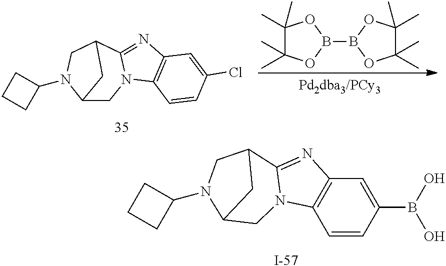

Compound 35 (373 mg, 1.3 mmol, 1.0 eq), bis(pinacolato)diboron (424 mg, 1.7 mmol, 1.3 eq), Pd₂dba₃ (120 mg, 0.13 mmol, 0.1 eq), PCy₃ (36 mg, 0.13 mmol, 0.1 eq) and KOAc (382 mg, 3.9 mmol, 3.0 eq) were dissolved in dioxane (10 mL) under nitrogen atmosphere, and the reaction mixture was heated under microwave irradiation at 130° C. under microwave for 2 hours. The solids were removed by filtration, the filtrate was concentrated by evaporation and the crude reaction product was purified by reverse phase silica gel chromatography to give intermediate I-57 (154 mg, 40%) as a white solid. MS (CI): m/z 298 (M+H⁺).

### Intermediate I-58 (3-cyclobutyl-2,3,4,5-tetrahydro-1H-2,5-methanobenzo[4,5]imidazo[1,2-d][1,4]diazepine-8-carbonitrile)

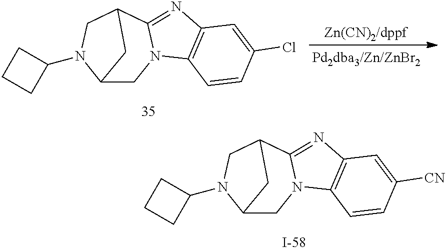

Compound 35 (500 mg, 1.8 mmol, 1.0 eq), Zn(CN)₂ (205 mg, 1.8 mmol, 1.0 eq), Zn(Br)₂ (395 mg, 1.8 mmol, 1.0 eq), Pd₂(dba)₃ (80 mg, 0.09 mmol, 0.05 eq), dppf (95 mg, 0.18 mmol, 0.1 eq) and Zn powder (30 mg, 0.45 mmol, 0.25 eq) were mixed in DMA (10 mL) under nitrogen atmosphere, and the reaction mixture was heated under microwave irradiation at 160° C. for 4 hours. The solids were removed by filtration, water was added to the filtrate and the crude reaction product was extracted with ethyl acetate. The combined organic layers were washed with water and brine, dried over anhydrous Na₂SO₄, the solids were removed by filtration and the filtrate was concentrated by evaporation. The crude reaction product was purified by silica gel column chromatography to give I-58 (300 mg, 62%) as a white solid. MS (CI): m/z 279 (M+H⁺).

### Intermediate I-59 (3-cyclobutyl-2,3,4,5-tetrahydro-1H-2,5-methanobenzo[4,5]imidazo[1,2-d][1,4]diazepine-8-carbaldehyde)

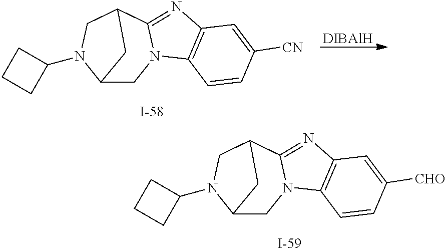

To a solution of intermediate I-58 (200 mg, 0.72 mmol, 1.0 eq) in dichloromethane (10 mL) at −78° C. was added drop wise a solution of DIBAL-H (1.0 M in dichloromethane, 2.1 mL, 2.1 mmol, 3.0 eq), and the mixture was stirred for additional 60 minutes. Saturated aqueous solution of NH₄Cl (1 mL) was added in one portion, and the reaction mixture was allowed to warm to room temperature. The crude reaction product was extracted with dichloromethane, the combined organic layers were washed with water and brine, and dried over anhydrous Na₂SO₄. The solids were removed by filtration and the filtrate was concentrated by evaporation. The crude reaction product was purified by silica gel column chromatography to give intermediate I-59 (160 mg, 80%) as a white solid. MS (CI): m/z 282 (M+H⁺).

### Intermediate I-60 (tert-butyl 4-(5-bromo-2-(2-hydroxyethylamino)phenylamino)-4-oxobutan-2-ylcarbamate)

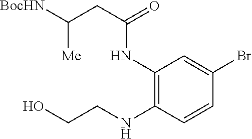

This intermediate was prepared in 18% yield (1.4 g) as described for intermediate I-4 but using 3-(tert-butoxycarbonylamino)butanoic acid as the starting material. MS (ESI): m/z 415 (M+H⁺).

### Intermediate I-61 (tert-butyl 1-(5-bromo-1-(2-hydroxyethyl)-1H-benzo[d]imidazol-2-yl)propan-2-ylcarbamate)

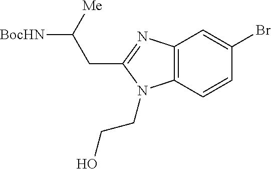

This intermediate was prepared in 47% yield (650 mg) as described for intermediate I-5 but using intermediate I-60 as the starting material. MS (ESI): m/z 397 (M+H⁺).

### Intermediate I-62 (2-(5-bromo-2-(2-(tert-butoxycarbonylamino)propyl)-1H-benzo[d]imidazol-1-yl)ethyl 4-methylbenzenesulfonate)

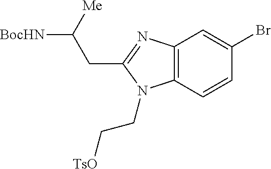

This intermediate was prepared in 90% yield (790 mg) as described for intermediate I-6 but using intermediate I-61 as the starting material. MS (ESI): m/z 551 (M+H⁺).

### Intermediate I-63 (2-(2-(2-aminopropyl)-5-bromo-1H-benzo[d]imidazol-1-yl)ethyl 4-methylbenzenesulfonate)

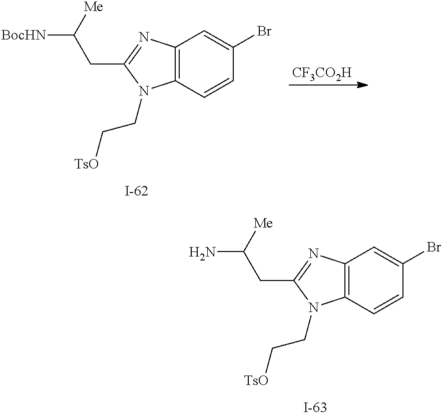

Intermediate I-62 (0.79 g, 1.4 mmol, 1.0 eq) was dissolved in dichloromethane (5 mL) and neat 2,2,2-trifluoroacetic acid (1.5 g, 5.0 eq) was added drop wise, and the reaction mixture was stirred at room temperature for 60 minutes. Excess 2,2,2-trifluoroacetic acid and solvent were removed by evaporation, and the residue was washed with ethyl ether to give the crude intermediate I-63 (0.64 g, 98%) that was used in the following step with further purification. MS (ESI): m/z 451(M+H⁺).

### Intermediate I-64 (8-bromo-4-methyl-2,3,4,5-tetrahydro-1H-benzo[4,5]imidazo[1,2-d][1,4]diazepine)

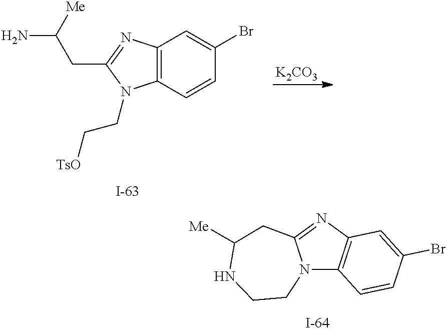

Intermediate I-63 (0.64 g, 1.4 mmol) was dissolved in a mixture of isopropanol and water (1:4 v/v, 12 mL), solid K₂CO₃ (1.5 g) was added, and the reaction mixture was refluxed for 2 hours. Excess solvent was removed by evaporation, and the residue was extracted with dichloromethane. The combined organic layers were dried over anhydrous Na₂SO₄, the solids were removed by filtration and the filtrate was concentrated. The crude reaction product was purified by silica gel column chromatography to give intermediate I-64 (0.3 g, 75%). MS (ESI): m/z 279 (M+H⁺).

### Intermediate I-65 (1-(4-bromo-2-nitrophenylamino)propan-2-ol)

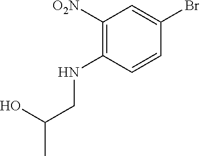

This intermediate was prepared in 100% yield (16.9 g) as described for intermediate I-2 but using 2-aminopropan-1-ol as the starting material. MS (ESI): m/z 275 (M+H⁺).

### Intermediate I-66 (1-(2-amino-4-bromophenylamino)propan-2-ol)

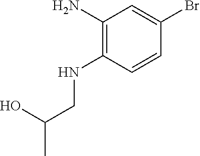

This intermediate was prepared in 75% yield (11.3 g) as described for intermediate I-16 but using intermediate I-65 as the starting material. MS (ESI): m/z 245 (M+H⁺).

### Intermediate I-67 (tert-butyl 3-(5-bromo-2-(2-hydroxypropylamino)phenylamino)-3-oxopropylcarbamate)

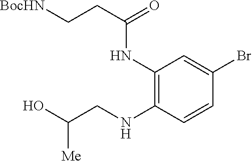

This intermediate was prepared in 34% yield (6.6 g) as described for intermediate I-4 but using intermediate I-66 and 3-(tert-butoxycarbonylamino)-propanoic acid as the starting materials. MS (ESI): m/z 416 (M+H⁺).

### Intermediate I-68 (tert-butyl 2-(5-bromo-1-(2-hydroxypropyl)-1H-benzo[d]imidazol-2-yl)ethylcarbamate)

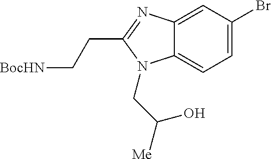

This intermediate was prepared in 100% yield (1.4 g) as described for intermediate I-5 but using intermediate I-67 as the starting material. MS (ESI): m/z 398 (M+H⁺).

### Intermediate I-69 (1-(5-bromo-2-(2-(tert-butoxycarbonylamino)ethyl)-1H-benzo[d]imidazol-1-yl)propan-2-yl methanesulfonate)

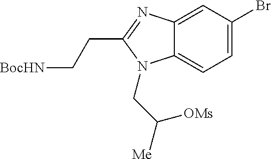

This intermediate was prepared in 79% yield (6.3 g) as described for intermediate I-6 but using intermediate I-68 and MsCl as the starting materials. MS (ESI): m/z 476 (M+H⁺).

### Intermediate I-70 (1-(2-(2-aminoethyl)-5-bromo-1H-benzo[d]imidazol-1-yl)propan-2-yl methanesulfonate)

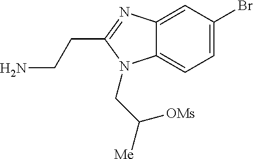

This intermediate was prepared in 100% yield (5.0 g) as described for intermediate I-63 but using intermediate I-69 as the starting material. MS (ESI): m/z 376 (M+H⁺).

### Intermediate I-71 (8-bromo-2-methyl-2,3,4,5-tetrahydro-1H-benzo[4,5]imidazo[1,2-d][1,4]diazepine)

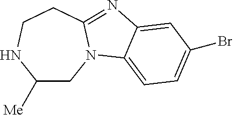

This intermediate was prepared in 90% yield (3.4 g) as described for intermediate I-64 but using intermediate I-70 as the starting material. MS (ESI): m/z 280 (M+H⁺).

### Intermediate I-72 (4-cyclobutylpiperazin-2-one)

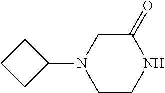

This intermediate was prepared in 52% yield (1.2 g) as described for compound 18 but using piperazin-2-one as the starting material. MS (ESI): m/z 155 (M+H⁺).

### Intermediate I-73 (1-(4-bromo-2-nitrophenyl)-4-cyclobutylpiperazin-2-one)

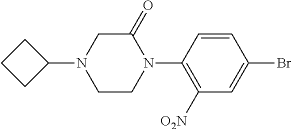

This intermediate was prepared in 26% yield (700 mg) as described for intermediate I-27 but using intermediate I-72 and 1,4-dibromo-2-nitrobenzene as the starting materials. MS (ESI): m/z 354 (M+H⁺).

### Intermediate I-74 (2-cyclobutyl-8-(4,4,5,5-tetramethyl-1,3,2-dioxaborolan-2-yl)-1,2,3,4-tetrahydrobenzo[4,5]imidazo[1,2-a]pyrazine)

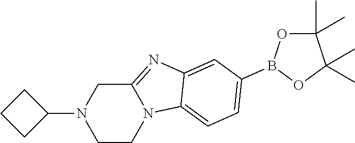

This intermediate was prepared in 43% yield (25 mg) as described for intermediate I-8 but using compound 52 as the starting material. MS (ESI): m/z 354 (M+H⁺).

### Intermediate I-75 (2-cyclobutyl-1,2,3,4-tetrahydrobenzo[4,5]imidazo[1,2-a]pyrazine-8-carbonitrile)

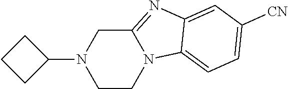

This compound was prepared in 56% yield (60 mg) as described for I-58 but using compound 52 as the starting material. MS (ESI): m/z 253 (M+H⁺).

### Intermediate I-76 (2-cyclobutyl-1,2,3,4-tetrahydrobenzo[4,5]imidazo[1,2-a]pyrazine-8-carbaldehyde)

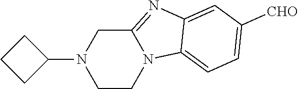

This intermediate was prepared in 83% yield (50 mg) as described for intermediate I-59 but using intermediate I-75 as the starting material. MS (ESI): m/z 256 (M+H⁺).

### Intermediate I-78 (1-benzyl 2-tert-butyl hydrazine-1,2-dicarboxylate)

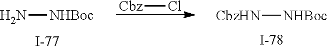

### To a solution of intermediate I-77 (20g, 150 mmol, 1 eq) in dichloromethane (400 mL) was added drop wise neat CbzCl (28 g, 166 mmol, 1.1 eq) over the period of 20 minutes, and the reaction mixture was stirred at room temperature overnight. Excess solvent was removed by evaporation, and the residue was diluted with water and extracted with ether. pH of the aqueous layer was adjusted to ˜8, and the aqueous layer was extracted with dichloromethane. The combined organic layers were dried over anhydrous Na₂SO₄, and the solids were removed by filtration. The filtrate was concentrated to give intermediate I-78 (38 g, 94%) that was used in the following step without further purification. MS (ESI): m/z 167 (M+H⁺).

### Intermediate I-79 (1-benzyl 2-tert-butyl pyrazolidine-1,2-dicarboxylate)

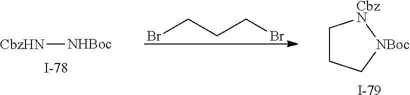

A suspension of sodium hydride (60% dispersion in mineral oil, 3.0 g, 75 mmol, 2.0 eq) in anhydrous DMF (120 mL) was cooled under nitrogen atmosphere to 0° C. on an ice/water bath. Intermediate I-78 (10 g, 38 mmol, 1.0 eq) was added portion wise, and the reaction mixture was stirred for 20 minutes. 1,3-Dibromopropane (7.5 g, 38 mmol, 1.0 eq) was added drop wise, and the reaction mixture was allowed to stir at room temperature overnight. Glacial acetic acid (0.5 mL) was added, and excess solvent was removed by evaporation. The residue was diluted with 50% saturated aqueous brine and extracted with diethyl ether. The combined organic layers were washed with brine, and dried over anhydrous MgSO₄; the solids were removed by filtration. The filtrate was concentrated by evaporation to give the crude intermediate I-79 (11 g, 95%) which was used in the following step without further purification. MS (ESI): m/z 307 (M+H⁺).

### Intermediate I-80 (benzyl pyrazolidine-1-carboxylate)

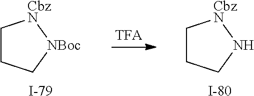

Intermediate I-79 (11 g, 35 mmol, 1.0 eq) was dissolved in neat trifluoroacetic acid (10 mL) under nitrogen atmosphere at room temperature, and the reaction mixture was stirred vigorously for 10 minutes. Excess solvent was removed by evaporation, and the residue was dissolved in water and extracted with a 1:1 mixture of ethyl acetate and hexane. The organic phase was back-extracted with aqueous hydrochloric acid (1.0 M), and the combined aqueous phases were basified with aqueous NaOH (50%). The basified aqueous layer was extracted with dichloromethane, the combined organic layers were dried over anhydrous Na₂SO₄ and the solids were removed by filtration. The filtrate was concentrated by evaporation to give intermediate I-80 (5.5 g, 74%) that was used in the following step without further purification. MS (ESI): m/z 207 (M+H⁺).

### Intermediate I-81 (benzyl 2-(3-chloropropanoyl)pyrazolidine-1-carboxylate)

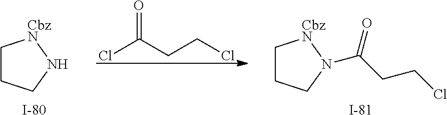

A solution of intermediate I-80 (8.6 g, 41 mmol, 1.0 eq) and diisoproylethylamine (5.3 g, 41 mmol, 1.0 eq) in dichloromethane (100 mL) was cooled under nitrogen atmosphere to 0° C. on an ice/water bath. A solution of 3-chloropropionyl chloride (5.2 g, 41 mmol, 1.0 eq) in dichloromethane (30 mL) was added drop wise over 45 minutes, and the reaction mixture was stirred for additional 60 minutes. The reaction was quenched by addition of aqueous hydrochloric acid (1.0 M), and the reaction mixture was extracted with dichloromethane. The combined organic layers were washed with aqueous HCl (1.0M), dried over anhydrous MgSO₄ and the solids were removed by filtration. The filtrate was concentrated by evaporation, and the crude reaction product was purified by silica gel column chromatography to give intermediate I-81 (10 g, 81%). MS (ESI): m/z 297 (M+H⁺).

### Intermediate I-82 (tetrahydropyrazolo[1,2-a]pyrazol-1(5H)-one)

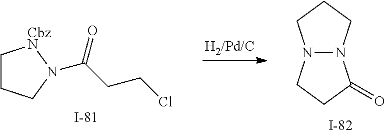

To a solution of intermediate I-81 (10 g, 34 mmol, 1.0 eq) in absolute ethanol (200 mL) was added palladium on carbon (10 wt %, 1.0 g), and the reaction mixture was stirred under atmosphere of hydrogen (1 atm) overnight. The solids were removed by filtration, and the filtrate was concentrated by evaporation to give intermediate I-82 as the HCl salt (5.3 g, 97%). MS (ESI): m/z 127 (M+H⁺).

### Intermediate I-83 (1,5-diazocan-2-one)

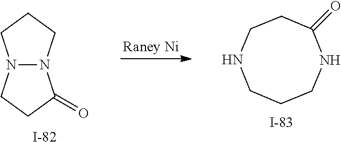

To a solution of intermediate I-82 (5.0 g, 31.6 mmol, 1.0 eq) in absolute ethanol (25 mL) was added a slurry of Raney nickel (4 g, wet weight), and the reaction mixture was stirred under atmosphere of hydrogen (1 atm) for 4 days. The solids were removed by filtration, and the filtrate was concentrated by evaporation to give intermediate I-83 as the HCl salt (5.1 g, 99%). MS (ESI): m/z 129 (M+H⁺).

### Intermediate I-84 (5-cyclobutyl-1,5-diazocan-2-one)

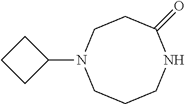

This intermediate was prepared in 60% yield (4.4 g) as described for compound 18 but using intermediate I-83 as the starting material. MS (ESI): m/z 183 (M+H⁺).

### Intermediate I-85 (1-(4-chloro-2-nitrophenyl)-5-cyclobutyl-1,5-diazocan-2-one)

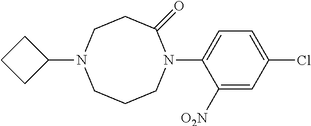

This intermediate was prepared in 65% yield (1.2 g) as described for intermediate I-27 but using intermediate I-84 as the starting material. MS (ESI): m/z 338 (M+H⁺).

### Intermediate I-86 (3-cyclobutyl-10-(4,4,5,5-tetramethyl-1,3,2-dioxaborolan-2-yl)-1,2,3,4,5,6-hexahydrobenzo[4,5]imidazo[1,2-a][1,5]diazocine)

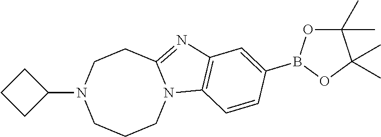

This intermediate was prepared in 37% yield (50 mg) as described for intermediate I-8 but using compound 57 as the starting material. MS (ESI): m/z 381 (M+H⁺).

### Intermediate I-87 (13-cyclobutyl-3-(difluoroboryl)-6,7,8,9,10,11-hexahydro-7,10-epiminobenzo[4,5]imidazo[1,2-a]azocine, fluoride salt)

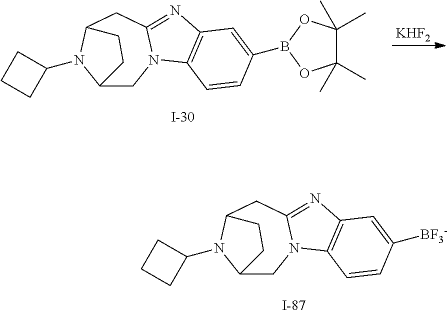

Potassium hydrogen fluoride (70 mg, 0.9 mmol, 6.0 eq) in water (2 mL) was added to a solution of intermediate I-30 (60 mg, 0.15 mmol, 1.0 eq) in methanol (3 mL), and the resulting white slurry was stirred at room temperature for 2 hours. The crude reaction mixture was concentrated by evaporation to give intermediate I-87 that was used in the following step without further purification. MS (ESI): m/z 374 (M+H⁺).

### Intermediate I-88 (13-cyclobutyl-6,7,8,9,10,11-hexahydro-7,10-epiminobenzo[4,5]imidazo[1,2-a]azocin-3-yl)boronic acid)

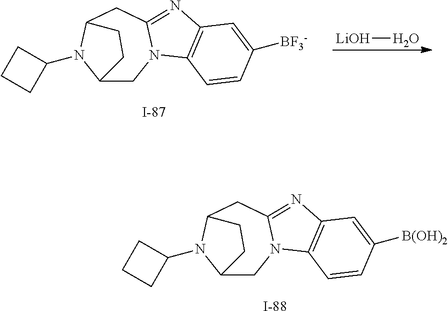

To a solution of intermediate I-87 (56 mg, 0.15 mmol, 1.0 eq) in water (1 mL) and acetonitrile (2 mL) was added LiOH (38 mg, 0.90 mmol, 6.0 eq), and the resulting solution was stirred at room temperature for 16 hours. Saturated aqueous solution of ammonium chloride (4 mL) and hydrochloric acid (1.0 M in water, 1 mL) were added, and the crude reaction mixture was concentrated by evaporation. The crude reaction product was purified by reverse phase column chromatography to give intermediate I-88 (40 mg, 99% yield calculated from intermediate I-30). MS (ESI) m/z: 312.1 (M+H⁺).

### Intermediate I-89 (13-cyclobutyl-6,7,8,9,10,11-hexahydro-7,10-epiminobenzo[4,5]imidazo[1,2-a]azocine-3-carbonitrile)

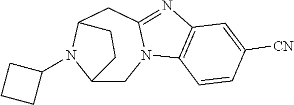

This intermediate was prepared in 45% yield (150 mg) as described for intermediate I-58 but using compound 18 as the starting material. MS (ESI): m/z 293.1 (M+H⁺).

### Intermediate I-90 (13-cyclobutyl-6,7,8,9,10,11-hexahydro-7,10-epiminobenzo[4,5]imidazo[1,2-a]azocine-3-carbaldehyde)

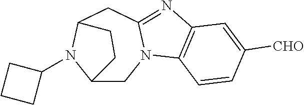

This intermediate was prepared in 17% yield (25 mg) as described for intermediate I-59 but using intermediate I-89 as the starting material. MS (ESI): m/z 296.1 (M+H⁺).

### Intermediate I-91 (tert-butyl 2-(2-((5-bromo-2-((3-hydroxypropyl)amino)phenyl)amino)-2-oxoethyl)piperidine-1-carboxylate)

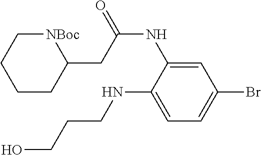

This intermediate was prepared in 67% yield (128 mg) as described for intermediate I-12 but using intermediate I-102 as the starting material. ¹HNMR (400 MHz, CD₃OD): δ 7.72 (s, 1H), 7.49 (d, 1H, J=8.8 Hz), 7.39 (d, 1H, J=8.8 Hz), 4.69 (m, 1H), 4.19 (s, 2H), 4.13 (m, 2H), 2.58 (m, 2H), 3.15 (m, 2H), 1.77 (m, 5H), 1.27 (m, 12H). MS (ESI): m/z 470, 472 (M+H⁺).

### Intermediate I-92 (tert-butyl 2-((5-bromo-1-(3-hydroxypropyl)-1H-benzo[d]imidazol-2-yl)methyl)piperidine-1-carboxylate)

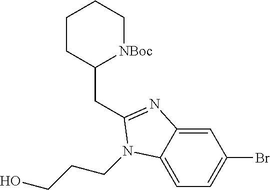

This intermediate was prepared as described for intermediate I-5 but using intermediate I-91 as the starting material. MS (ESI): m/z 452, 454 (M+H⁺).

### Intermediate I-93 (tert-butyl 2-((5-bromo-1-(3-(tosyloxy)propyl)-1H-benzo[d]imidazol-2-yl)methyl)piperidine-1-carboxylate)

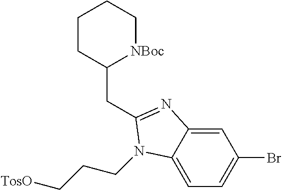

This intermediate was prepared in 60% yield (87 mg, over two steps) as described for intermediate I-6 but using intermediate I-92 as the starting material. MS (ESI): m/z 606, 608 (M+H⁺).

### Intermediate I-94 (12-bromo-2,3,4,6,7,8,15,15a-octahydro-1H-benzo[4,5]imidazo[1,2-a]pyrido[2,1-d][1,5]diazocine)

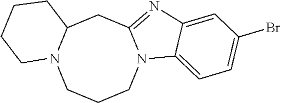

This intermediate was prepared in 79% yield (265 mg, over two steps) as described for compound 1 but using intermediate I-93 as the starting material. MS (ESI): m/z 334, 336 (M+H⁺).

### Intermediate I-95 (12-(4,4,5,5-tetramethyl-1,3,2-dioxaborolan-2-yl)-2,3,4,6,7,8,15,15a-octahydro-1H-benzo[4,5]imidazo[1,2-a]pyrido[2,1-d][1,5]diazocine)

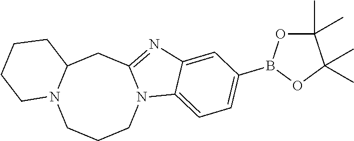

This intermediate was prepared (80 mg) as described for intermediate I-8 but using intermediate I-94 as the starting material. MS (ESI): m/z 382(M+H⁺).

### Intermediate I-96 (2,3,4,6,7,8,15,15a-octahydro-1H-benzo[4,5]imidazo[1,2-a]pyrido[2,1-d][1,5]diazocine-12-carbonitrile)

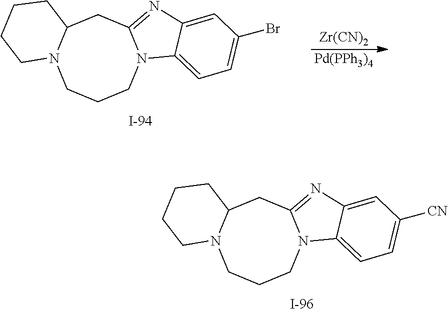

Intermediate I-94 (200 mg, 0.60 mmol, 1.0 eq), zinc cyanide (110 mg, 1.2 mmol, 2.0 eq) and Pd(PPh₃)₄ (20 mg, 10% w/w) were dissolved in dry DMF (3 mL) in a microwave tube that was flushed with argon. The reaction mixture was heated at 130° C. under microwave irradiation for 8 hours. Ethyl acetate was then added, and the solids were removed by filtration through a short plug of silica gel. The filtrate was washed with water, the combined organic layers were dried over anhydrous Na₂SO₄, the solids were removed by filtration, and the filtrate was concentrated by evaporation. The crude reaction product was purified by preparative reverse-phase HPLC to give intermediate I-96 (45 mg, 65%). MS (ESI): m/z 281 (M+H⁺).

### Intermediate I-97 (2,3,4,6,7,8,15,15a-octahydro-1H-benzo[4,5]imidazo[1,2-a]pyrido[2,1-d][1,5]diazocine-12-carbaldehyde)

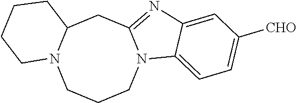

This intermediate was prepared (45 mg) as described for intermediate I-59 but using intermediate I-96 as the starting material. MS (ESI): m/z 284 (M+H⁺).

### Intermediate I-98 (10-(4,4,5,5-tetramethyl-1,3,2-dioxaborolan-2-yl)-2,3,5,6,13,13a-hexahydro-1H-benzo[4,5]imidazo[1,2-d]pyrrolo[2,1-g][1,4]diazepine)

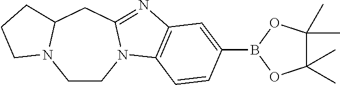

This intermediate was prepared in 40% yield (140 mg) as described for intermediate I-8 but using compound 72 as the starting material. MS (ESI): m/z 354 (M+H⁺).

### Intermediate I-99 (2,3,5,6,13,13a-hexahydro-1H-benzo[4,5]imidazo[1,2-d]pyrrolo[2,1-g][1,4]diazepine-10-carbonitrile)

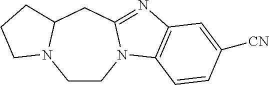

This intermediate was prepared in 60% yield (453 mg) as described for intermediate I-96 but using compound 72 as the starting material. MS (ESI): m/z 253 (M+H⁺).

### Intermediate I-100 (2,3,5,6,13,13a-hexahydro-1H-benzo[4,5]imidazo[1,2-d]pyrrolo[2,1-g][1,4]diazepine-10-carbaldehyde)

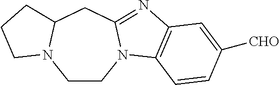

This intermediate was prepared in 70% yield (321 mg) as described for intermediate I-59 but using intermediate I-99 as the starting material. MS (ESI): m/z 256(M+H⁺).

### Intermediate I-101 (3-((4-bromo-2-nitrophenyl)amino)propan-1-ol)

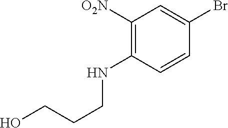

This intermediate was prepared in 93% yield (25.5 g) as described for intermediate I-2 but using 3-aminopropan-1-ol as the starting material. MS (ESI): m/z 274 (M+H⁺).

### Intermediate I-102 (3-((2-amino-4-bromophenyl)amino)propan-1-ol)

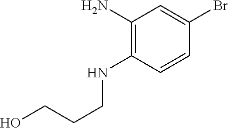

This intermediate was prepared in 96% yield (14.1 g) as described for intermediate I-3 but using intermediate I-101 as the starting material. MS (ESI): m/z 245 (M+H⁺).

### Intermediate I-103 (tert-butyl 2-(2-((5-bromo-2-((3-hydroxypropyl)amino)phenyl)amino)-2-oxoethyl)pyrrolidine-1-carboxylate)

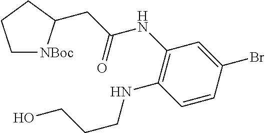

This intermediate was prepared in 60% yield (13.7 g) as described for intermediate I-4 but using racemic 2-(1-(tert-butoxycarbonyl)pyrrolidin-2-yl)acetic acid and intermediate I-102 as starting materials. MS (ESI): m/z 456 (M+H⁺).

### Intermediate I-104 (tert-butyl 2-((5-bromo-1-(3-hydroxypropyl)-1H-benzo[d]imidazol-2-yl)methyl)pyrrolidine-1-carboxylate)

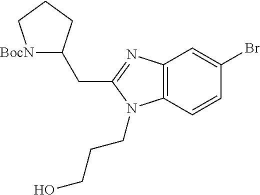

This intermediate was prepared in 75% yield (6.6 g) as described for intermediate I-5 but using intermediate I-103 as the starting material. MS (ESI): m/z 439 (M+H⁺).

### Intermediate I-105 (tert-butyl 2-((5-bromo-1-(3-(tosyloxy)propyl)-1H-benzo[d]imidazol-2-yl)methyl)pyrrolidine-1-carboxylate)

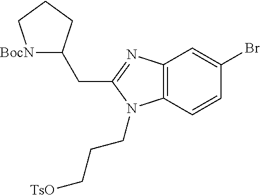

This intermediate was prepared in 84% yield (7.4 g) as described for intermediate I-6 but using intermediate I-104 as the starting material. MS (ESI): m/z 592 (M+H⁺).

### Intermediate I-106 (11-(4,4,5,5-tetramethyl-1,3,2-dioxaborolan-2-yl)-1,2,3,5,6,7,14,14a-octahydrobenzo[4,5]imidazo[1,2-a]pyrrolo[2,1-d][1,5]diazocine)

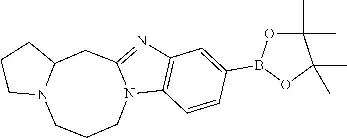

This intermediate was prepared in 50% yield (184 mg) as described for intermediate I-8 but using compound 79 as the starting material. MS (ESI): m/z 368 (M+H⁺).

### Intermediate I-107 (4-(5-cyclobutyl-2-oxo-1,5-diazocan-1-yl)-3-nitrobenzonitrile)

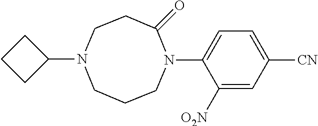

This intermediate was prepared in 36% yield (6.8 g) as described for intermediate I-27 but using intermediate I-84 and 4-bromo-3-nitrobenzonitrile as the starting materials. MS (ESI): m/z 329 (M+H⁺).

### Intermediate I-108 (3-cyclobutyl-1,2,3,4,5,6-hexahydrobenzo[4,5]imidazo[1,2-a][1,5]diazocine-10-carbonitrile)

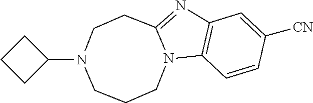

This intermediate was prepared in 75% yield (6.8 g) as described for intermediate I-28 but using intermediate I-107 as the starting material. MS (ESI): m/z 281 (M+H⁺).

### Intermediate I-109 (3-cyclobutyl-1,2,3,4,5,6-hexahydrobenzo[4,5]imidazo[1,2-a][1,5]diazocine-10-carbaldehyde)

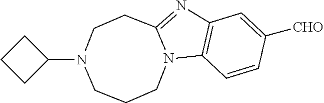

This intermediate was prepared in 65% yield (2.9 g) as described for intermediate I-59 but using intermediate I-108 as the starting material. MS (ESI): m/z 284(M+H⁺).

B. Histamine H3 In Vitro Assay

H3 GTPγS assay (SPA method) was performed at EuroScreen (Belgium, ES-392-C) using conventional methods. Briefly, cells expressing the human histamine H3 receptor were homogenized in 15 mM Tris-HCl pH 7.5, 2 mM MgCl₂, 0.3 mM EDTA, and 1 mM EGTA. Membranes were washed twice in the above tris buffer, collected by centrifugation (40,000×g, 25 mM), and re-suspended in 75 mM Tris-HCl pH 7.5, 12.5 mM MgCl₂, 0.3 mM EDTA, 1 mM EGTA, and 250 mM sucrose. Membranes were frozen in liquid nitrogen until use. On the day of the assay, membranes were thawed and diluted in assay buffer (20 mM HEPES pH 7.4, 100 mM NaCl, 10 μg/ml saponin, 1 mM MgCl₂) to give 500 μg/ml and mixed (v/v) with GDP in assay buffer for a final GDP concentration of 10 μM and incubated on ice for at least 15 mM. PVT-WGA beads (Amersham, RPNQ001) were diluted in assay buffer at 50 mg/mL and mixed with GTPγ[³⁵S] (Amersham, SJ1308) diluted in assay buffer to give ˜25,000 dpm/10 μL and mixed vol/vol just before the start of the reaction. The reaction was started by adding 50 μL of test compound, 20 μL of the membranes:GDP mix, 10 μL of buffer, and 20 μL of the GTPγ[³⁵S]:beads mix in a 96 well plate Optiplate™ (PerkinElmer, 6005299) covered with topseal (TopCount™, PerkinElmer), mixed with an orbital shaker for 2 min, incubated for 1 hour at room temperature, centrifuged for 10 min at 2000 rpm, incubated for 1 h at room temperature, and counted for 1 min in a TopCount™ reader (PerkinElmer). Dose response curves and IC₅₀ values (concentration to inhibit the reaction by 50%) were calculated by nonlinear regression using XLfit software (IDBS).

For antagonists testing, 10 μL of a reference agonist (R-γ-Me-Histamine) instead of 10 μL buffer was added at a concentration corresponding to the EC₈₀ (30 nM). Control ligands were R-γ-Me-Histamine (Tocris, 0569), Imetit (Sigma, I-135), Thioperamide (Tocris, 0644), and Clobenpropit (Tocris, 0754) diluted in assay buffer.

The compounds provided herein were tested in the histamine H3 in vitro assay. In one embodiment, the respective HCl salts of the compounds provided herein were prepared using standard chemical procedures and tested in the histamine H3 in vitro assay. The functional potency of the compounds (as indicated by their IC₅₀s) are shown in Table 1.

TABLE 1

| Compound | Potency |
|---|---|
| 1 | (++) |
| 2 | (+++) |
| 3 | (+++) |
| 4 | (++) |
| 5 | (+++) |
| 6 | (+) |
| 7 | (+++) |
| 8 | (++++) |
| 9 | (++++) |
| 10 | (++++) |
| 11 | (++++) |
| 12 | (+) |
| 13 | (++) |
| 14 | (+) |
| 15 | (++++) |
| 16 | (++++) |
| 17 | (++++) |
| 18 | (++) |
| 19 | (+++) |
| 20 | (++++) |
| 21 | (++++) |
| 22 | (+++) |
| 23 | (++++) |
| 24 | (++++) |
| 25 | (++) |
| 26 | (+++) |
| 27 | (+++) |
| 28 | (+) |
| 29 | (++) |
| 30 | (+++) |
| 31 | (+++) |
| 32 | (+) |
| 33 | (++) |
| 34 | (++) |
| 35 | (++) |
| 36 | (++) |
| 37 | (+) |
| 38 | (++) |
| 39 | (+) |
| 40 | (+++) |
| 41 | (++++) |
| 42 | (++) |
| 43 | (+++) |
| 44 | (++++) |
| 45 | (++) |
| 46 | (+++) |
| 47 | (++++) |
| 48 | (++++) |
| 49 | (+++) |
| 50 | (++++) |
| 51 | (+++) |
| 52 | (+) |
| 53 | (++) |
| 54 | (++) |
| 55 | (++) |
| 56 | (++++) |
| 57 | (+++) |
| 58 | (++++) |
| 59 | (++++) |
| 60 | (++++) |
| 61 | (+++) |
| 62 | (+++) |
| 64 | (++++) |
| 65 | (+++) |
| 66 | (++++) |
| 67 | (++++) |
| 68 | (+++) |
| 69 | (+++) |
| 70 | (++++) |
| 71 | (+++) |
| 72 | (+++) |
| 73 | (++++) |
| 74 | (++++) |
| 75 | (++++) |
| 76 | (++++) |
| 77 | (++++) |
| 78 | (+++) |
| 79 | (+++) |
| 80 | (++++) |
| 81 | (++++) |
| 82 | (++++) |
| 83 | (++++) |
| 84 | (++++) |
| 85 | (++++) |

Legend for Table 1
(++++) represents an IC₅₀ of ≦10 nM;
(+++) represents an IC₅₀ of ≦100 nM;
(++) represents an IC₅₀ of ≦1 μM; and
(+) represents an IC₅₀ of ≧1 μM.

C. Rat H3 Ex Vivo Receptor Occupancy Assay

This assay was designed to measure occupancy of H3 receptors by compounds provided herein by measuring the competition of binding to the H3 receptor with a radiolabeled ligand known to bind to the H3 receptor. More specifically, receptor occupancy was determined by ex vivo binding studies in rat cortical membranes.

Adult male Sprague-Dawley rats were treated with vehicle or compound at four different dose levels. After an appropriate pre-treatment time (typically 30 min to 1 hr), membranes to be tested were prepared from brain frontal cortex membranes by homogenization in ice-cold assay buffer. Frontal cortical membranes (400 μL containing an equivalent of 5 mg wet weight of tissue/tube) were incubated with 50 82 L of [³H] R-α-methylhistamine (final concentration 2 nM) and either 50 μL of buffer (total binding) or 50 μL of imetit (10 μM; non-specific binding) for 30 min at 25° C. in triplicates. Membrane bound radioactivity was recovered by filtration under vacuum through Skatron 11731 filters, pre-soaked in 0.5% PEI. Filters were rapidly washed with ice-cold 50 mM Tris buffer (wash setting 9,9,0) and radioactivity was determined by liquid scintillation counting (1 mL Packard MV Gold Scintillator). The amount of radioactivity in the membranes at the different doses was used generate a dose response curve and to calculate the ED₅₀ (the dose that results in 50% receptor occupancy).

The ex vivo potency of exemplary compounds (as indicated by their ED₅₀s) are summarized in Table 2. ED₅₀ is the amount of drug at which 50% of the H3 receptors are occupied by the drug as measured by the amount of radioactivity measured at that dose relative to that measured when no drug (or vehicle) was administered.

TABLE 2

| Compound | ED₅₀ (mg/kg) |
|---|---|
| 48 | (++) |
| 59 | (++) |
| 64 | (+) |
| 84 | (+) |
| 85 | (++) |

Legend for Table 2:
(++) represents an ED₅₀ of ≦1.0 mg/kg; and
(+) represents an ED₅₀ of ≧1.0 mg/kg.

D. Rat EEG Assay

The rat EEG assay was designed to measure electrical signals from the brain as a proxy for in vivo biological activity at the H3 receptor. For example, without being limited to a particular theory, antagonism at the H3 receptor has been shown to be associated with wake promotion and increase in the high frequencies of the EEG signals (See, e.g., Parmentier et al., Biochemical Pharmacology 73 (2007):1157-1171; Le et al., Journal of Pharmacology and Experimental Therapeutics 325 (2008):902-909).

Specifically, animals were housed in a temperature-controlled recording room under a 12 hour/12 hour light/dark cycle, with food and water available ad libitum. Eight male Sprague-Dawley rats were implanted with chronic recording devices for continuous recordings of electroencephalograph (EEG) via telemetry (Data Sciences Inc). A repeated measures design was employed in which each rat received eight separate dosings of the compound being tested with a minimum of 3 days between doses. Dosing occurred during the middle of the rats' normal inactive period, and the EEG data collected during the first 6 hours post-dosing were scored and analyzed. EEG data were scored visually in 10 second epochs for wake, REM, and non-REM states. Scored data were analyzed and expressed as time spent in each state per hour. Cumulative time spent in wake, non-REM, and REM states were calculated for the 6 hour recording period. To determine whether any of the pharmacological treatments affected the consolidation of behavioral states, the duration and number of bouts for each state were calculated in hourly bins. A “bout” consisted of a minimum of two consecutive 10-second epochs of a given state and ended with any single state change epoch. The EEG spectra during wake and non-REM sleep were analyzed offline with a fast Fourier transform algorithm (NeuroScore software, Data Sciences Inc) on all epochs without a visually detectable artifact. Wake EEG spectra were analyzed in 1 Hz bins. Data were analyzed using two-way repeated-measures ANOVA.

Compounds 48 and 59 were tested and were shown to be wake promoting in rats. For both compounds, there was an increase in the time spent in wake, a decrease in the time spent in non-REM sleep, and no change in the time spent in REM sleep relative to vehicle treated animals. The increase in time spent in wake was achieved in a consolidated manner with an increase in the wake bout duration and a decrease in the number of wake and non-REM sleep bouts. The spectral analysis showed a shift to the higher frequencies with an increase in the normalized power in the 40-60 Hz range. Accordingly, compounds 48 and 59 displayed in vivo biological activity that is consistent with activity at the H3 receptor.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the disclosure and are encompassed by the appended claims.

All of the patents, patent applications and publications referred to herein are incorporated by reference herein in their entireties. Citation or identification of any reference in this application is not an admission that such reference is available as prior art to this application. The full scope of the disclosure is better understood with reference to the appended claims.

What is claimed:

1. A compound of formula (I): 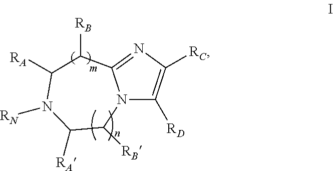
or a pharmaceutically acceptable salt or stereoisomer thereof, wherein
R_{N}, R_{A}, R_{A}′, R_{B}, and R_{B}′ are each independently a bond, hydrogen, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, or (5 to 10 membered)-heteroaryl, each of which may be optionally substituted with one or more R′; and (i) one pair of R_{N} and R_{A}, or R_{N} and R_{A}′, or R_{A} and R_{B}, or R_{A}′ and R_{B}′ together with the atoms to which they are attached form an optionally substituted 3-, 4-, 5-, 6-, or 7-membered non-aromatic ring; or (ii) one pair of R_{N} and R_{B}, or R_{N} and R_{B}′, or R_{A} and R_{A}′, or R_{A} and R_{B}′, or R_{B} and R_{A}′, or R_{B} and R_{B}′ are taken together to form a 1-, 2-, or 3-atom bridge;
R_{C} and R_{D} are each independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)-heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R″; or R_{C} and R_{D} together may form a ring;
each occurrence of R′ is independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₂; or two R′ substituents together may form a 3 to 10 membered ring;
each occurrence of R″ is independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₁; or two R″ substituents together may form a 3 to 10 membered ring;
each occurrence of R₁ is independently hydrogen, halogen, cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, —S(O)₂NR₃R₄, (C₁-C₁₀)alkyl optionally substituted with one or more R₂, (C₃-C₁₀)cycloalkyl optionally substituted with one or more R₂, (C₆-C₁₂)aralkyl optionally substituted with one or more R₂, (6 to 10 membered)aryl optionally substituted with one or more R₂, (C₁-C₁₀)heteroalkyl optionally substituted with one or more R₂, (C₃-C₁₀)heterocycloalkyl optionally substituted with one or more R₂, or (5 to 10 membered)heteroaryl optionally substituted with one or more R₂;
each occurrence of R₂ is independently hydrogen, (C₁-C₆)alkyl optionally substituted with one or more R₃, (C₃-C₆)cycloalkyl optionally substituted with one or more R₃, halogen, cyano, ═O, —OR₃, —NR₃R₄, —N(R₃)C(O)R₄, —C(O)NR₃R₄, —C(O)R₃, —C(O)OR₃, —OC(O)R₃, —S(O)_{q}R₃, or —S(O)₂NR₃R₄;
R₃ and R₄ are each independently hydrogen, (C₁-C₆)alkyl, (C₃-C₆)cycloalkyl, (C₇-C₁₀)aralkyl; (C₁-C₆)heteroalkyl, (C₃-C₆)heterocycloalkyl, (6 to 10 membered)aryl, or (5 to 10 membered)heteroaryl; or R₃ and R₄ together may form a 3 to 10 membered ring;
q is 0, 1, or 2;
m is 1 or 2; and
n is 1, 2, or 3.

2. The compound of claim 1, wherein R_{C} and R_{D} together form a phenyl ring optionally substituted with one or more R″.

3. The compound of claim 2, wherein m is 1, n is 1, and R_{A} and R_{A}′ together form a 1-, 2-, or 3-atom bridge, which is optionally substituted with one or more R′.

4. The compound of claim 3, wherein the compound is:

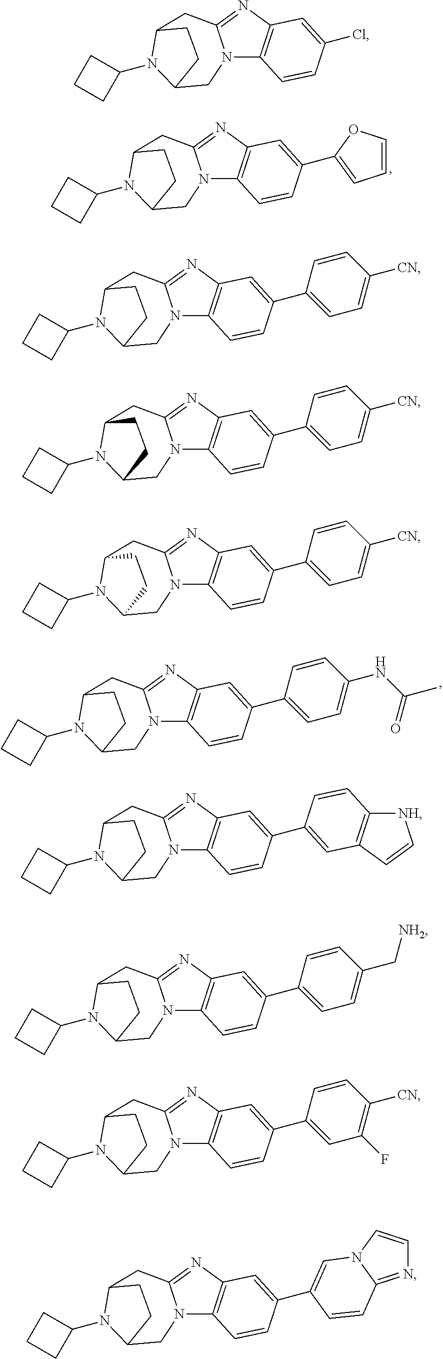

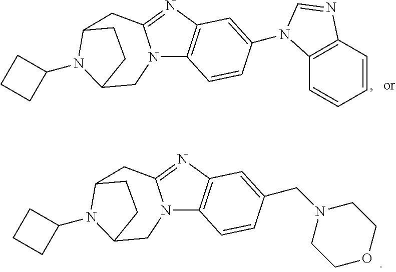

5. The compound of claim 2, wherein m is 1, n is 1, and R_{A} and R_{B}′ together form a 1-, 2-, or 3-atom bridge, which is optionally substituted with one or more R′.

6. The compound of claim 5, wherein the compound is:

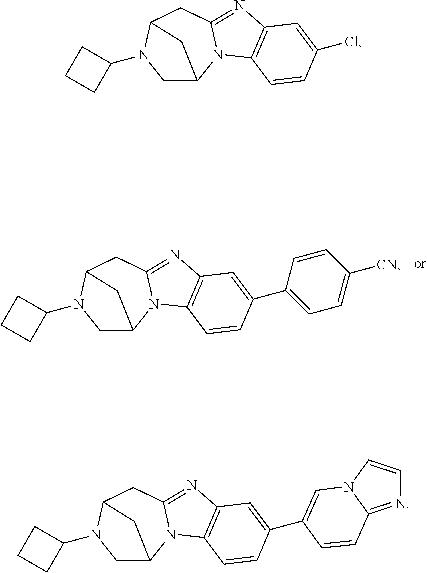

7. The compound of claim 2, wherein m is 1, n is 1, and R_{B} and R_{A}′ together form a 1-, 2-, or 3-atom bridge, which is optionally substituted with one or more R′.

8. The compound of claim 7, wherein the compound is:

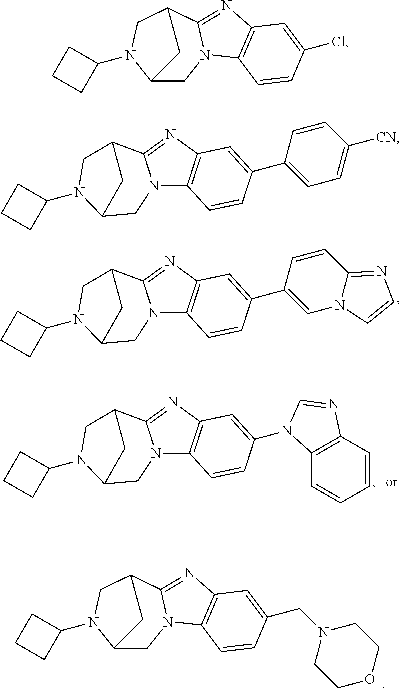

9. The compound of claim 2, wherein m is 1, n is 1, and R_{B} and R_{B}′ together form a 1-, 2-, or 3-atom bridge, which is optionally substituted with one or more R′.

10. The compound of claim 9, wherein the compound is:

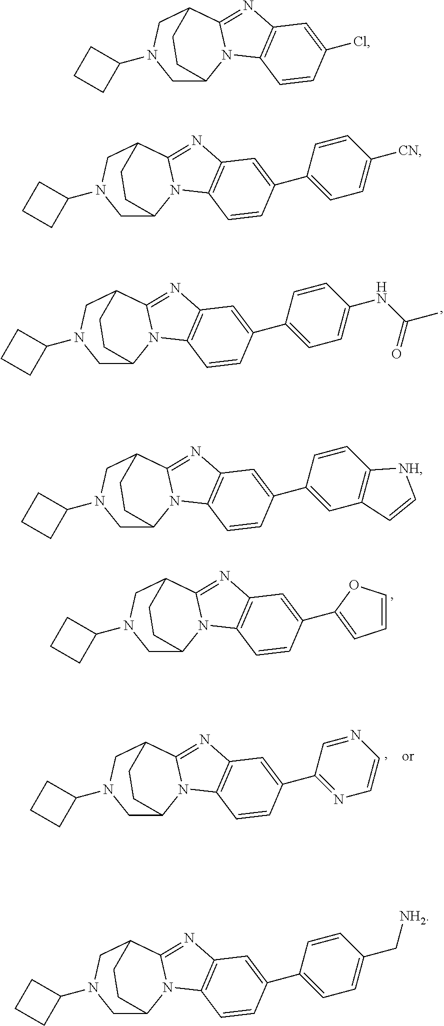

11. The compound of claim 2, having formula (Ia): 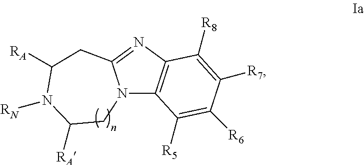
or a pharmaceutically acceptable salt or stereoisomer thereof, wherein
R₅, R₆, R₇ and R₈ are each independently hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)-heterocycloalkyl, (5 to 10 membered)heteroaryl, hydroxyl, alkoxyl, aminoalkyl, amino, imino, amido, carbonyl, thiol, sulfinyl, or sulfonyl, each of which may be optionally substituted with one or more R₁; or two adjacent R₅, R₆, R₇, and R₈ may together form a 3 to 10 membered ring.

12. The compound of claim 11, wherein R_{N} and R_{A}′ together with the atoms to which they are attached form a 3-, 4-, 5-, 6-, or 7-membered non-aromatic ring, which is optionally substituted with one or more R′.

13. The compound of claim 12, wherein the compound is:

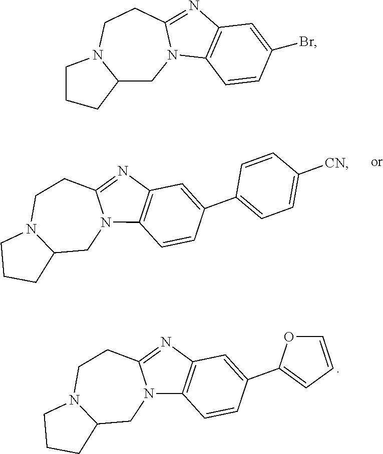

14. The compound of claim 11, wherein R_{N} and R_{A} together with the atoms to which they are attached form a 3-, 4-, 5-, 6-, or 7-membered non-aromatic ring, which is optionally substituted with one or more R′.

15. The compound of claim 11, having formula (II): 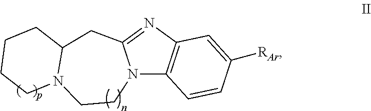
or a pharmaceutically acceptable salt or stereoisomer thereof, wherein
R_{Ar} is hydrogen, halogen, cyano, (C₁-C₁₀)alkyl, (C₁-C₁₀)alkenyl, (C₃-C₁₀)cycloalkyl, (6 to 10 membered)aryl, (C₁-C₁₀)heteroalkyl, (C₃-C₁₀)heterocycloalkyl, (5 to 10 membered)-heteroaryl, hydroxyl, alkoxyl, aminoalkyl, amino, imino, amido, carbonyl, thiol, sulfinyl, or sulfonyl, each of which may be optionally substituted with one or more R₁; n is 1 or 2; and p is 0, 1, or 2.

16. The compound of claim 15, wherein p is 0 and n is 1.

17. The compound of claim 16, wherein the compound is:

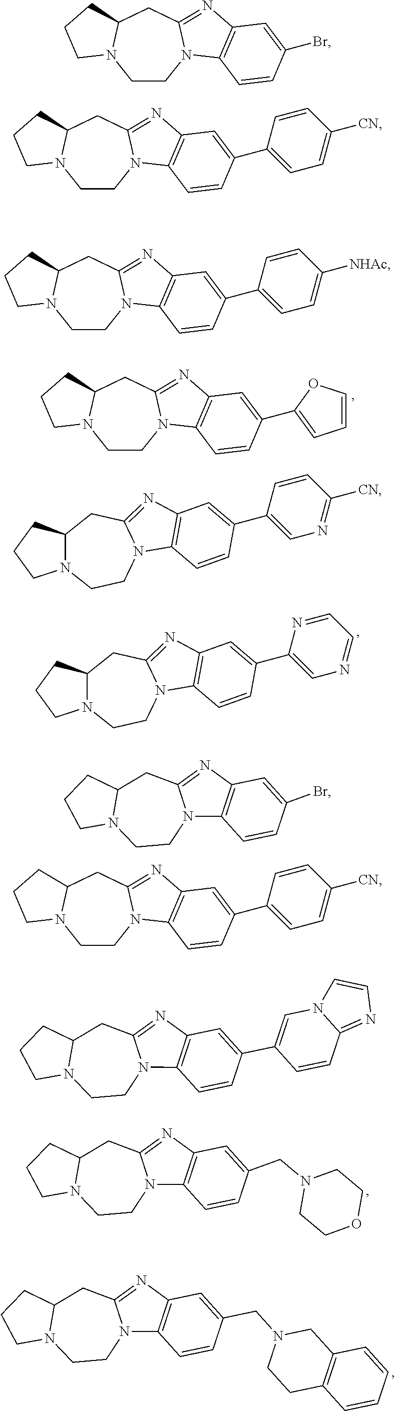

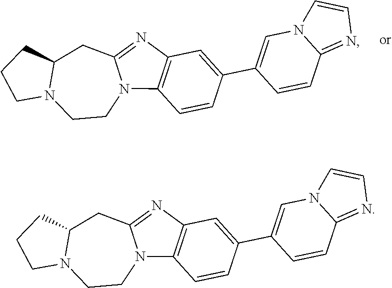

18. The compound of claim 15, wherein p is 1 and n is 1.

19. The compound of claim 18, wherein the compound is:

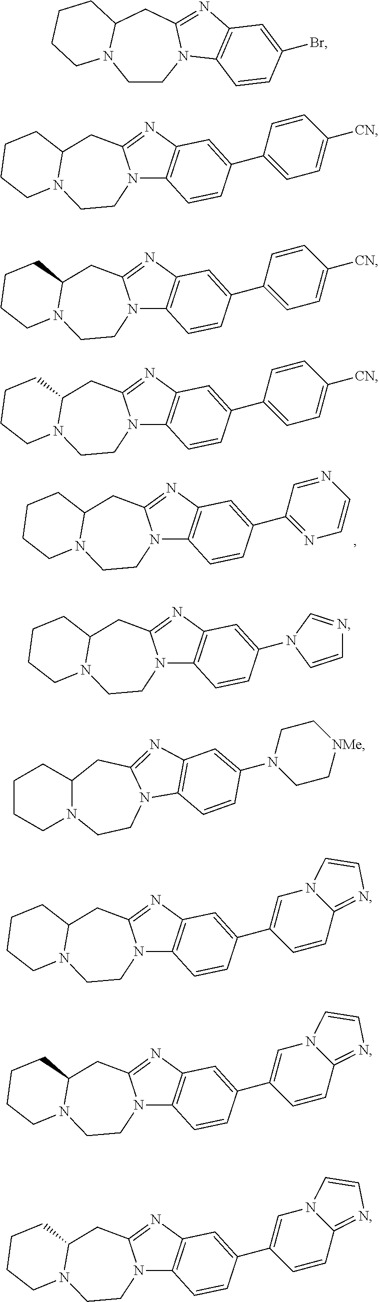

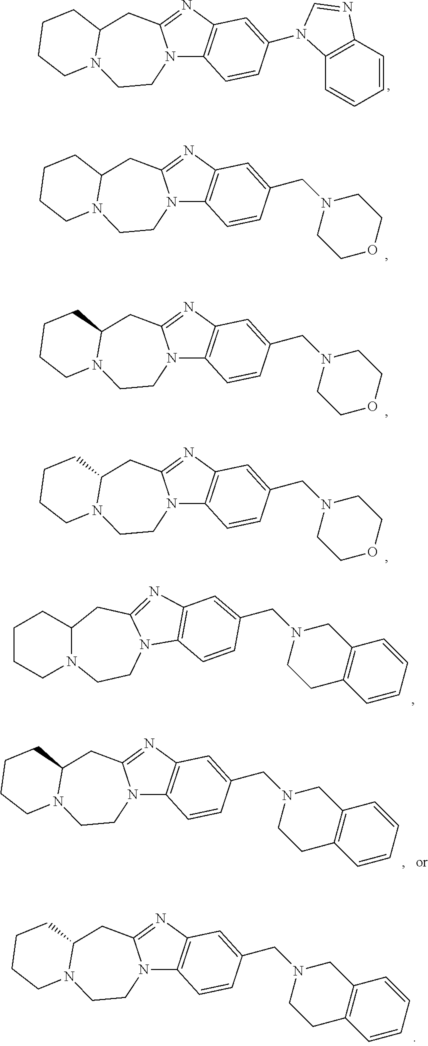

20. The compound of claim 15, wherein p is 1 and n is 2.

21. The compound of claim 20, wherein the compound is:

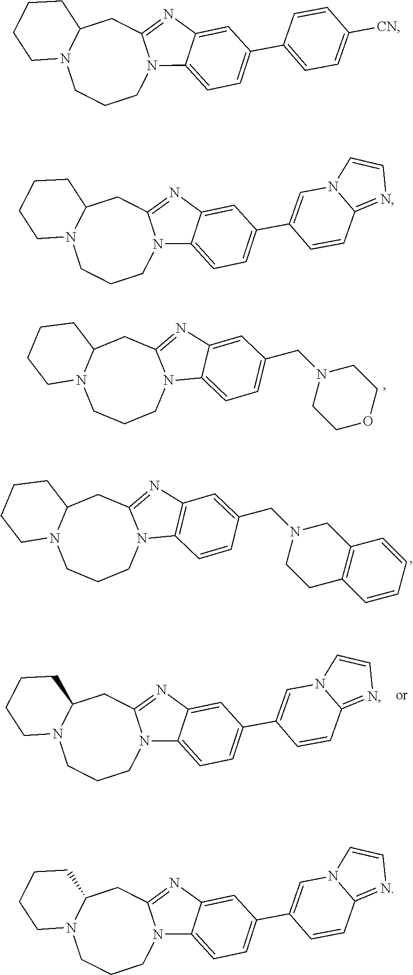

22. The compound of claim 15, wherein p is 0 and n is 2.

23. The compound of claim 22, wherein the compound is:

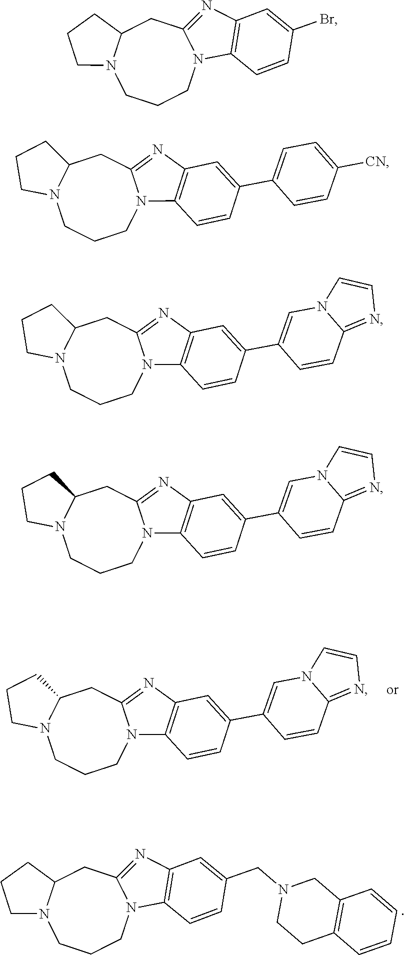

24. A pharmaceutical composition comprising a compound of claim 1, or a pharmaceutically acceptable salt or stereoisomer thereof.

25. The pharmaceutical composition of claim 24, which comprises a pharmaceutically acceptable excipient or carrier.

26. The pharmaceutical composition of claim 24, which further comprises one or more additional active agents.

27. A method of reducing the activity of a histamine receptor, said method comprising contacting said histamine receptor and a compound of claim 1, or a pharmaceutically acceptable salt or stereoisomer thereof.

28. The method of claim 27, wherein said histamine receptor is a H3 receptor.

29. A method of treating, preventing, or managing a disorder related to histamine H3 receptor comprising administering to a subject a therapeutically or prophylactically effective amount of a compound of claim 1, or a pharmaceutically acceptable salt or stereoisomer thereof.

30. The method of claim 29, wherein said subject is a human.

31. The method of claim 29, wherein said disorder is neurological disorder, neurodegenerative disease, schizophrenia, Alzheimer's disease, Parkinson's disease, affective disorder, attention deficit hyperactivity disorder (ADHD), psychosis, convulsion, seizure, vertigo, epilepsy, narcolepsy, pain, neuropathic pain, sensitization accompanying neuropathic pain, psychosis, mood disorder, depression, anxiety, excessive daytime sleepiness, narcolepsy, multiple sclerosis, jet lag, drowsy side effect of medications, insomnia, substance abuse, cognitive impairment, impairment of learning, impairment of memory, impairment of attention, vigilance or speed of response, metabolic disorder, diabetes, obesity, disorder related to satiety, disorder of gastric activity, disorder of enteric system, disorder of exocrine pancreatic system, acid secretion, digestive disorder, disorder of gut motility; movement disorder, restless leg syndrome (RLS), or Huntington's disease.
